(19) United States
(12) Patent Application Publication
(10) Pub. No.: US 2024/0294498 A1
(43) Pub. Date: Sep. 5, 2024

(54) FACTOR XIIA INHIBITORS

(71) Applicant: Lunac Therapeutics Ltd, Leeds (GB)

(72) Inventors: Richard James Foster, Leeds (GB); Sonia Abas Prades, Leeds (GB); James Nigel Ayres, Leeds (GB); Helen Philippou, Leeds (GB); Trevor Perrior, Leeds (GB); Alan Naylor, Leeds (GB); Philip Spencer Fallon, Saffron Walden (GB); Anna Hopkins, Saffron Walden (GB); Alicia Galvan Alvarez, Saffron Walden (GB); Gabriel Negoita-Giras, Saffron Walden (GB); John Joseph May, Saffron Walden (GB); Daniel John Brough, Saffron Walden (GB); Paolo Ricci, Saffron Walden (GB)

(21) Appl. No.: 18/564,763

(22) PCT Filed: May 27, 2022
(86) PCT No.: PCT/GB2022/051363

§ 371 Date: Nov. 28, 2023

(30) Foreign Application Priority Data
May 28, 2021 (GB) 2107722.7

(51) Int. Cl. C07D 401/14; A61K 31/454; A61K 31/4545; A61K 31/496; A61K 31/501; A61K 31/5386; A61K 31/551; C07D 409/14; C07D 413/14; C07D 471/04; C07D 487/08; C07D 498/08

(52) U.S. Cl. CPC C07D 401/14; A61K 31/454; A61K 31/4545; A61K 31/496; A61K 31/501; A61K 31/5386; A61K 31/551; C07D 409/14; C07D 413/14; C07D 471/04; C07D 487/08; C07D 498/08

(57) ABSTRACT

The compounds of the invention are modulators of the Factor XII (e.g. Factor XIIa). In particular, the compounds are inhibitors of Factor XIIa and may be useful as anticoagulants.

This invention relates to compounds and methods of treatment (or prevention) using the compounds. The invention also relates to processes and methods for producing the compounds of the invention. The compounds of the invention are modulators of the Factor XII (e.g. Factor XIIa). In particular, the compounds are inhibitors of Factor XIIa and may be useful as anticoagulants.

### BACKGROUND

Cardiovascular disease is the leading cause of death in the developed world, affecting millions of people worldwide every year. The disease is generally caused by atherosclerosis of the arterial wall, which develops over many years and is characterised by inflammation of the endothelium, sub-endothelial lipid deposition, macrophage infiltration and plaque development. In the acute phase of the disease, the atherosclerotic plaque becomes unstable and ruptures, triggering thrombosis. The development of a thrombus (blood clot) that occludes the blood vessel and consequently deprives the tissue of oxygen constitutes the main precipitating event leading to morbidity and mortality. Blood clot formation is initiated by activation and aggregation of platelets. The platelet plug is consolidated by the activation of coagulation and formation of a fibrin network. Arterial occlusion by the thrombus leads to tissue death downstream, and, depending upon where this occurs, is associated with the development of myocardial infarction, stroke or claudication.

Thrombosis in the venous circulation has a different aetiology as it does not depend on atherosclerosis, but is triggered by circulatory stasis due to immobilisation and is often associated with naturally occurring deficiencies of coagulation inhibitors (e.g. antithrombin, protein C and S) and with surgical procedures. Venous thrombosis usually occurs in the leg or arm (deep vein thrombosis, DVT) and can lead to emboli (thrombus fragments) blocking downstream smaller vessels particularly in the lung (pulmonary embolism, PE). Other triggers of DVT include cancer, nephrotic syndrome, antiphospholipid syndrome and heart failure.

Thrombosis is a very serious condition and is associated with up to 25,000 and 200,000 fatalities in the UK alone on an annual basis for venous and arterial thrombosis respectively. In January 2010, the UK National Institute for Health and Clinical Excellence (NICE) published new guidelines to increase screening for early signs of thrombosis in patients admitted to hospital.

Current medications to treat or prevent thrombosis target either platelets or coagulation. Generally, antiplatelet drugs are used in the prevention of arterial disease, whereas anticoagulants are used in the prevention of stroke in patients with atrial fibrillation, deep venous thrombosis (DVT) and pulmonary embolism (PE). The largest clinical problem associated with current anticoagulant use is the risk of bleeding. As many as 1 to 3% of patients experience major bleeding or 15-18% patients experience minor bleeding whilst on anticoagulation therapy, dependent upon patient group and choice of anticoagulation.

Warfarin and heparin (encompassing all of its derivatives) are the most commonly used anticoagulant drugs. Warfarin, the oldest approved long-term oral anticoagulant, requires regular monitoring via prothrombin time (PT) clotting assays to determine optimal dosage, which places a major burden on the healthcare system and patient quality of life. Warfarin is non-specific and targets several coagulation enzymes, whereas heparin, which is administered subcutaneously or intravenously, targets activated factor X (FXa) and/or thrombin depending on its molecular weight. Furthermore, the non-vitamin K oral anticoagulants (NOACs) on the market or in development that target thrombin or FXa, also carry a significant risk of bleeding which is comparable to that of heparin and warfarin with the exception of intracranial haemorrhage where NOACs have better outcome than warfarin. However, gastrointestinal bleeding is increased with NOACs compared with low-molecular-weight heparin and vitamin K antagonist that encompasses warfarin [New Oral Anticoagulants Increase Risk for Gastrointestinal Bleeding: A Systematic Review and Meta-analysis Holster I L, Valkhoff V E, Kuipers E J, Tjwa E T Gastroenterology. 2013 July; 145(1):105-112].

Therefore, there is a large unmet clinical need for a novel anticoagulant that is not associated with bleeding. This goal has been an aspiration for the field for more than 6 decades. However, it was always assumed that anticoagulation leads to an unavoidable risk of bleeding because the mechanisms involved in thrombosis were considered the same as those involved in haemostasis.

Factor XII (FXII) was identified 50 years ago as a coagulation protein in the intrinsic pathway of blood coagulation as FXII deficient patients had marked prolongation of the in vitro surface-activated coagulation time. However, series of investigations have convincingly shown that FXII has no role in normal haemostasis. Evidence within the last decade has identified FXII as essential for thrombus formation in vivo (Renne T, Pozgajova M, Gruner S, Schuh K, Pauer H U, Burfeind P, Gailani D, Nieswandt B. Defective thrombus formation in mice lacking coagulation factor XII. J Exp Med 2005; 202:271-281; Kleinschnitz C, Stoll G, Bendszus M, Schuh K, Pauer H U, Burfeind P, Renne C, Gailani D, Nieswandt B, Renne T. Targeting coagulation factor XII provides protection from pathological thrombosis in cerebral ischemia without interfering with hemostasis. J Exp Med 2006; 203:513-518; Renne T, Nieswandt B, Gailani D. The intrinsic pathway of coagulation is essential for thrombus stability in mice. Blood Cells Mol Dis 2006; 36:148-151; Hagedorn I, Schmidbauer S, Pleines I, Kleinschnitz C, Kronthaler U, Stoll G, Dickneite G, Nieswandt B. Factor XIIa inhibitor recombinant human albumin Infestin-4 abolishes occlusive arterial thrombus formation without affecting bleeding. Circulation 2010; 121:1510-1517 and Matafonov A, Leung P Y, Gailani A E, Grach S L, Puy C, Cheng Q, Sun M F, McCarty O J, Tucker E I, Kataoka H, Renne T, Morrissey J H, Gruber A, Gailani D. Factor XII inhibition reduces thrombus formation in a primate thrombosis model. Blood. 2014; 13; 123(11):1739-46). A unique characteristic of FXII is that its deficiency does not incur bleeding, unlike deficiencies in all other coagulation factors. Therefore, FXIIa is a highly attractive target for the discovery of an anticoagulant with the potential for a greatly improved safety profile.

Studies have challenged dogma in the haemostasis and thrombosis field by demonstrating novel mechanisms in thrombosis involving FXII. These studies provide clear evidence that FXII is necessary for thrombus development whilst not essential for haemostasis. FXII deficient mice were remarkably protected against thrombosis when challenged with collagen and epinephrine infusion, whilst showing no prolongation of bleeding time during surgery or tail-clipping. Similar protection against thrombosis was observed in mesenteric arterioles exposed to FeCl₃ and in the aorta after mechanical injury. Infusion of human FXII in these models restored the development of thrombi. The ground breaking nature of these findings is illustrated by the debate on FXII function and the role of the contact coagulation pathway activated by FXIIa that, for several years dominated the field. This debate was fuelled by the fact that FXII deficiency does not lead to bleeding whereas deficiency in every other coagulation protease does, which led to the belief that FXII was not required for physiological coagulation and that FXII activation was an in vitro phenomenon.

However, studies have shown that FXII is activated by negatively charged surfaces and the surface of activated platelets (Zakharova et al, PLoS One. 2015 Feb. 17; 10(2):e0116665). These in vivo and in vitro studies demonstrate that FXII plays a hitherto unrecognised role in thrombosis. The generation of FXIIa stabilises the thrombus through enhanced thrombin generation, fibrin deposition and direct prothrombotic effects on fibrin structure. This mechanism does not appear to play a role in normal haemostasis, since FXII deficiency is phenotypically silent in humans as well as mice, making FXII an ideal target for the development of a new anticoagulant to treat thrombosis.

The effectiveness of FXII deficiency in reducing thrombosis has been shown in several different in vivo thrombosis models. In addition to the models mentioned above, the role of FXII in thrombosis has been demonstrated in a murine model of thrombosis induced by ligation of the carotid artery and a murine model of cerebral microvascular thrombosis secondary to transient occlusion of the middle cerebral artery. Brain infarct sizes were significantly reduced in FXII deficient mice and restored to large infarcts by the infusion of human FXII. Inhibition of FXII has also been shown to reduce risk of venous thrombosis. One study has demonstrated that a Kunitz-type inhibitor of contact activation isolated from the tick salivary glands (Ir-CPI) effectively reduces thrombosis in mouse and rat models of venous thrombosis induced by vessel ligation. This inhibitory protein was also effective in reducing PE in a murine model induced by infusion with collagen and epinephrine, and in a murine model of dorsal skin arteriole thrombosis. Again, there was no effect on bleeding time in the animals treated with Ir-CPI. Inhibition of FXIIa with H-D-Pro-Phe-Arg-chloromethylketone (PCK) has also been shown to protect against thrombosis. These studies provide preclinical proof of concept that inhibition of FXIIa is efficacious in the treatment of thrombosis.

Magnus Larsson et al., “A Factor XIIa Inhibitory Antibody Provides Thromboprotection in Extracorporeal Circulation Without Increasing Bleeding Risk” Sci Transl Med 6, 222ra17 (2014); demonstrated that recombinant fully human antibody 3F7 binds into the FXIIa enzymatic pocket. 3F7 interfered with FXIIa-mediated coagulation, abolished thrombus formation under flow, and blocked experimental thrombosis in mice and rabbits. In rabbits 3F7 provided thromboprotection as efficiently as heparin, but unlike heparin, 3F7 treatment did not impair the haemostatic capacity and did not increase bleeding from wounds. Larsson et al conclude that targeting of FXIIa is a safe mode of thromboprotection in bypass systems, and provides a clinically relevant anticoagulation strategy that is not complicated by excess bleeding.

Dabigatran, apixaban, rivaroxaban, edoxaban and betrixaban are approved for short-term use as oral FXa/thrombin inhibitors, respectively. Dabigatran is 3-({2-[(4-carbamimidoyl-phenylamino)-methyl]-1-methyl-1H-benzoimidazole-5-carbonyl}-pyridin-2-yl-amino)-propionic acid;

Dabigatran is also approved for long term prevention of stroke in patients with atrial

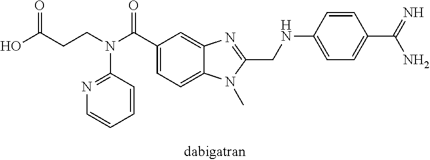 [SMILES: Cn1c(CNc2ccc(C(=N)N)cc2)nc2cc(C(=O)N(CCC(=O)O)c3ccccn3)ccc21]

fibrillation (AF) and is described in U.S. Pat. No. 6,087,380.

Rivaroxaban is (S)-5-chloro-N-{[2-oxo-3-[4-(3-oxomorpholin-4-yl)phenyl]oxazolidine-5-yl]methyl} thiophene-2-carboxamide;

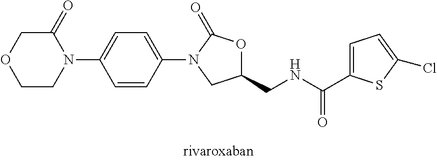 [SMILES: O=C(NC[C@H]1CN(c2ccc(N3CCOCC3=O)cc2)C(=O)O1)c1ccc(Cl)s1]

Rivaroxaban is also approved for reducing stroke risk in patients with nonvalvular AF. Rivaroxaban has shown superiority of once-daily rivaroxaban over warfarin in protecting AF patients from stroke and non-CNS systemic embolism. Rivaroxaban also demonstrates comparable major and non-major clinically relevant bleeding, as well as significantly lower rates of intracranial haemorrhage vs. warfarin. Rivaroxaban, is described in U.S. Pat. No. 7,157,456.

Apixaban is also factor Xa inhibitor approved for use in preventing stroke and systemic embolism in patients with nonvalvular atrial fibrillation.

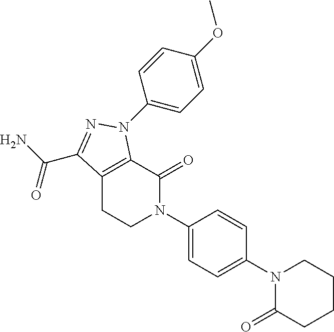 [SMILES: COc1ccc(-n2nc(C(N)=O)c3c2C(=O)N(c2ccc(N4CCCCC4=O)cc2)CC3)cc1]

Apixaban is 1-(4-methoxyphenyl)-7-oxo-6-[4-(2-oxopiperidin-1-yl)phenyl]-4,5-dihydropyrazolo[5,4-c]pyridine-3-carboxamide:

Apixaban is described in U.S. Pat. No. 6,413,980.

Edoxaban is N′-(5-chloropyridin-2-yl)-N²-((1S,2R,4S)-4-[(dimethylamino)carbonyl]-2-{[(5-methyl-4,5,6,7-tetrahydrothiazolo[5,4-c]pyridin-2-yl)carbonyl]amino}cyclohexyl)ethanediamide;

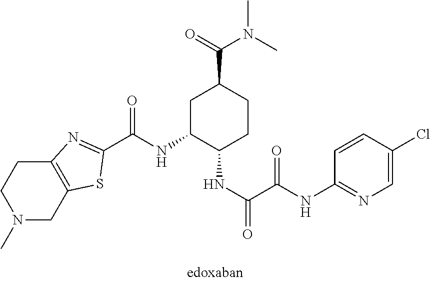 [SMILES: CN1CCc2nc(C(=O)N[C@@H]3C[C@@H](C(=O)N(C)C)CC[C@@H]3NC(=O)C(=O)Nc3ccc(Cl)cn3)sc2C1]

Edoxaban is another factor Xa inhibitor approved for use in preventing stroke and systemic embolism in patients with nonvalvular atrial fibrillation and for the treatment of deep vein thrombosis. Edoxaban is described in U.S. Pat. No. 7,365,205.

Betrixaban is N-(5-chloropyridin-2-yl)-2-[4-(N,N-dimethylcarbamimidoyl)benzamido]-5-methoxybenzamide:

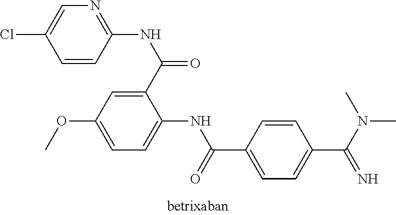 [SMILES: COc1ccc(NC(=O)c2ccc(C(=N)N(C)C)cc2)c(C(=O)Nc2ccc(Cl)cn2)c1]

Betrixaban is a factor Xa inhibitor approved for use in preventing venous thromboembolism in patients with moderate to severe restricted mobility. Betrixaban is described in U.S. Pat. No. 6,376,515.

Recent surveys of the cardiovascular pipelines of major pharmaceutical companies have not revealed any oral inhibitors of FXIIa. Infestin-4 is a biological agent produced by CSL Behring that targets FXIIa, and shows efficacy in a FeCl₃-induced model of thrombosis in mice and rabbits. Other antibody approaches targeting FXII(a) have also shown in vivo efficacy. However, if infestin-4 or the antibody approaches were successful, they would require intravenous administration, which makes them less suitable for long-term anticoagulation.

As FXII deficiency in humans is asymptomatic, unlike other coagulation factor deficiencies that cause bleeding and that deficiency or inhibition of the activity of FXII show an anticoagulant effect; a selective FXIIa inhibitor, has the potential to reduce bleeding risk associated with currently available anticoagulant therapies.

European Patent application No. EP0672658 (Eli Lilly) describes phenylalanine proline derivatives that are useful as thrombin inhibitors.

International Patent application No. WO 2002/064559 (Merck) also describes phenylalanine proline derivatives that are useful as thrombin inhibitors. The compounds are selective inhibitors of cyclooxygenase-2 inhibition over cyclooxygenase-1.

International Patent application No. WO 02/50056 (Merck) describes benzylamine and cyclohexylamine derivatives that are useful as thrombin inhibitors.

International Patent application No. WO 2019/186164 (University of Leeds) describes compounds that are modulators of Factor XII.

It is an aim of aspects of the present invention to at least partially mitigate the problems associated with the prior art.

It is an aim of certain embodiments of the present invention to provide compounds that inhibit FXII activity, in particular FXIIa activity, for example the serine protease activity of FXIIa.

It is an aim of certain embodiments of the present invention to provide compounds that possess physicochemical and pharmacokinetic properties consistent with the potential for oral bioavailability

It is an aim of certain embodiments of this invention to provide compounds which exhibit reduced cytotoxicity or increased solubility relative to prior art compounds and existing therapies.

Another aim of certain embodiments of this invention is to provide compounds having a convenient pharmacokinetic profile and a suitable duration of action following dosing. A further aim of certain embodiments of this invention is to provide compounds in which the metabolised fragment or fragments of the drug after absorption are GRAS (Generally Regarded As Safe).

It is an aim of certain embodiments of this invention to provide a modulator of a target, where the embodiment selectively modulates the target over other targets. It is an aim of certain embodiments of the present invention to provide compounds that are selective FXIIa inhibitors. In particular, an aim of certain embodiments of the invention is to provide compounds that selectively inhibit FXIIa over thrombin and FXa.

Certain embodiments of the present invention satisfy some or all of the above aims.

### BRIEF SUMMARY OF THE DISCLOSURE

In accordance with the present invention there is provided a compound according to formula (I) and pharmaceutically acceptable salts thereof:

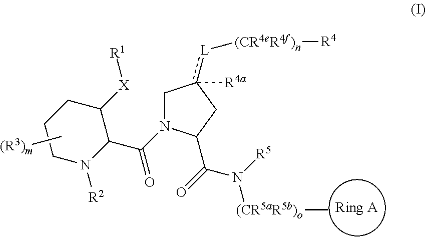

wherein
—X— is selected from: a bond, —C(O)—, C₁₋₃ alkylenyl and C₂ alkenylenyl;
R¹ is selected from: —NR^{1a}R^{1b}, a substituted or unsubstituted 5 to 10 membered monocyclic or bicyclic heteroaryl group having 1, 2 or 3 heteroatoms selected from O, N or S, a substituted or unsubstituted 6 to 10 membered monocyclic or bicyclic aryl group, a substituted or unsubstituted 3 to 10 membered monocyclic or bicyclic (fused, bridged or spiro) cycloalkyl group and a substituted or unsubstituted 3 to 10 membered monocyclic or bicyclic (fused, bridged or spiro) heterocyclic group having 1, 2 or 3 heteroatoms selected from O, N or S; wherein R^{1a} and R^{1b} are each independently selected from: substituted or unsubstituted C₁₋₆ alkyl, substituted or unsubstituted C₃₋₇ cycloalkyl, a substituted or unsubstituted 3 to 7 membered heterocyclic group having 1, 2 or 3 heteroatoms selected from O, N or S, substituted or unsubstituted —C₁₋₆ alkyl-C₃₋₇ cycloalkyl, a substituted or unsubstituted —C₁₋₆ alkyl-3 to 7 membered heterocyclic group having 1, 2 or 3 heteroatoms selected from O, N or S, and a substituted or unsubstituted —C₁₋₆ alkyl-5-6 membered heteroaromatic group having 1, 2 or 3 heteroatoms selected from O, N or S;
   wherein, when substituted, the substituent of R¹ is selected from: halo, ═O, —CN, —OH, C₁₋₆ alkyl, C₃₋₆ cycloalkyl, C₁₋₆ haloalkyl, —O—C₁₋₆ alkyl, —O—C₃₋₆ cycloalkyl, —O—C₁₋₆ haloalkyl, —C₁₋₆ alkyl-O—C₁₋₆ alkyl, —C₁₋₆ alkyl-O—C₃₋₆ cycloalkyl, —C₁₋₆ alkyl-O—C₁₋₆ haloalkyl, —NR^{1c}R^{1d}, —NR^{1c}(SO₂)R^{1d}, —NR^{1c}(C(O))R^{1d}, —C(O)NR^{1c}R^{1d}, —SO₂NR^{1c}R^{1d}, a 5 to 10 membered heteroaryl group having 1, 2 or 3 heteroatoms selected from O, N or S, and a 6 to 10 membered aryl group; wherein R^{1c} and R^{1d} are independently at each occurrence selected from: H, C₁₋₆ alkyl, C₁₋₆ haloalkyl, C₃₋₆ cycloalkyl, C₃₋₆ cyclohaloalkyl, 5 to 10 membered heteroaryl group having 1, 2 or 3 heteroatoms selected from O, N or S, and a 6 to 10 membered aryl group;
   wherein, when substituted, the substituent of R^{1a} and R^{1b} is selected from: halo, —CN, —OH, C₁₋₆ alkyl, C₃₋₇ cycloalkyl, C₁₋₆ haloalkyl, C₃₋₇ cyclohaloalkyl, —O—C₁₋₆ alkyl, —O—C₃₋₇ cycloalkyl, —O—C₁₋₆ haloalkyl, —O—C₃₋₇ cyclohaloalkyl, —C₁₋₆ alkyl-O—C₁₋₆ alkyl, —C₁₋₆ alkyl-O—C₃₋₇ cycloalkyl, —C₁₋₆ alkyl-O—C₁₋₆ haloalkyl, —C₁₋₆ alkyl-O—C₃₋₇ cyclohaloalkyl, —C₁₋₆ haloalkyl-O—C₁₋₆ alkyl, —C₁₋₆ haloalkyl-O—C₃₋₇ cycloalkyl, —C₁₋₆ haloalkyl-O—C₁₋₆ haloalkyl, —C₁₋₆ haloalkyl-O—C₃₋₇ cyclohaloalkyl, —NR^{1e}R^{1f}, —NH(═NH)NR^{1e}R^{1f}, —NR^{1e}(SO₂)R^{1f}, —NR^{1e}(C(O))R^{1f}, —C(O)NR^{1e}R^{1f}, and —SO₂NR^{1e}R^{1f}; R^{1e} and R^{1f} are independently at each occurrence selected from: H, C₁₋₆ alkyl, C₁₋₆ haloalkyl, C₃₋₆ cycloalkyl, C₃₋₆ cyclohaloalkyl, 5 to 10 membered heteroaryl group having 1, 2 or 3 heteroatoms selected from O, N or S, and a 6 to 10 membered aryl group; R² is selected from: H, C₁₋₆ alkyl, C₁₋₆ haloalkyl, C₃₋₆ cycloalkyl, and 3 to 6 membered heterocyloalkyl; R³ is selected from: halo, —CN, —OH, C₁₋₆ alkyl, C₁₋₆ haloalkyl, —O—C₁₋₆ alkyl, —O—C₁₋₆ haloalkyl, —NR^{3a}R^{3b}, —NR^{3a}(C(O))R^{3b}, and —C(O)NR^{3a}R^{3b}; wherein R^{3a} and R^{3b} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
m is selected from 0, 1, 2, or 3;
wherein the residue

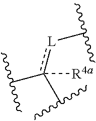

is selected from:

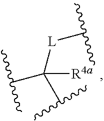

wherein L is selected from: bond, —O—, —NR^{4b}—, and —NR^{4c}C(O)—; and

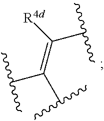

R^{4a} is selected from: H, —OH, halo, C₁₋₄ alkyl or C₁₋₄ haloalkyl;
R^{4b} is H, C₁₋₆ alkyl or —C(O)C₁₋₆ alkyl;
R^{4c} is H or C₁₋₆ alkyl;
R^{4d} is H or C₁₋₆ alkyl;
R^{4e} and R^{4V} are independently at each occurrence selected from: H, —CN, halo, C₁₋₄ alkyl, C₁₋₄ haloalkyl, —OR^{4g}, —NR^{4g}R^{4h}, C₃₋₈ cycloalkyl, 3 to 6 membered heterocyclic ring, 6 to 10 membered aryl, 5 to 10 membered heteroaryl, wherein the C₃₋₈ cycloalkyl, 3 to 6 membered heterocyclic ring, 6 to 10 membered aryl or 5 to 10 membered heteroaryl group is unsubstituted or substituted with 1, 2 or 3 R⁴ⁱ groups; wherein R^{4g} and R^{4h} are independently at each occurrence selected from: H and C₁₋₄ alkyl; and wherein R⁴ⁱ is independently at each occurrence selected from: halo, C₁₋₄ alkyl, C₁₋₄ haloalkyl, C₃₋₆ cycloalkyl, C₃₋₆ cyclohaloalkyl, —OR^{4j}, —NR^{4k}R^{4l}, —NR^{4k}(C(O))R^{4l}, —C(O)NR^{4k}R^{4l}, —CN, —C(O)R^{4g}, ═O, —SO₂R^{4g}, benzyl, phenyl, unsubstituted 5 or 6 membered heteroaryl, or methyl substituted 5 or 6 membered heteroaryl; R⁴ⁱ is selected from: H, C₁₋₄ alkyl, C₁₋₄ haloalkyl, phenyl or benzyl; R^{4k} and R^{4l} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
n is selected from 0, 1, 2, 3, or 4;
R⁴ is selected from: H, halo, —CN, C₁₋₄ alkyl, C₁₋₄ haloalkyl, —OR^{4g}, —NR^{4g}R^{4h}, a monocyclic or bicyclic 6 to 10 membered aryl, C₃₋₈ cycloalkyl, C₄₋₈ cycloalkenyl, 3 to 6 membered heterocyclic ring comprising 1, 2, or 3 heteroatoms selected from O, N or S, a monocyclic or bicyclic 5 to 10 membered heteroaryl comprising 1, 2, or 3 heteroatoms selected from O, N or S, a bicyclic (fused, bridged, or spiro) 6 to 10 membered cycloalkyl ring system, a bicyclic (fused, bridged, or spiro) 6 to 10 membered cycloalkenyl ring system and a bicyclic (fused, bridged, or spiro) 6 to 10 membered heterocyclic ring system comprising 1, 2, or 3 heteroatoms selected from O, N or S, wherein the C₃₋₈ cycloalkyl, C₄₋₈ cycloalkenyl, 3 to 6 membered heterocyclic ring, 6 to 10 membered aryl, 5 to 10 membered heteroaryl group, bicyclic (fused, bridged, or spiro) 6 to 10 membered cycloalkyl ring system, bicyclic (fused, bridged, or spiro) 6 to 10 membered cycloalkenyl ring system, or bicyclic (fused, bridged, or spiro) 6 to 10 membered heterocyclic ring system is unsubstituted or substituted with 1, 2 or 3 R⁴ⁱ;
R⁵ is H or C₁₋₆ alkyl;
o is selected from 1, 2 or 3;
R^{5a} and R^{5b} are independently at each occurrence selected from: H, substituted or unsubstituted C₁₋₆ alkyl, substituted or unsubstituted C₃₋₆ cycloalkyl, and substituted or unsubstituted C₁₋₆ haloalkyl, wherein each substituent is independently selected from halo, —OH, and —CN; Ring A is selected from a substituted or unsubstituted 5 to 10 membered monocyclic or bicyclic heteroaryl group having 1, 2 or 3 heteroatoms selected from O, N or S, a substituted or unsubstituted 6 to 10 membered monocyclic or bicyclic aryl group, and a substituted or unsubstituted monocyclic or bicyclic (fused, bridged, or spiro) 6 to 10 membered heterocyclic ring system comprising 1, 2, or 3 heteroatoms selected from O, N or S, wherein, when substituted, the heteroaryl group, aryl group, or heterocyclic ring system are substituted with 1, 2, or 3 substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d};
R^{5c} and R^{5d} are independently at each occurrence selected from: H, substituted or unsubstituted C₁₋₆ alkyl, substituted or unsubstituted C₃₋₆ cycloalkyl, and substituted or unsubstituted C₁₋₆ haloalkyl, wherein each substituent is independently selected from halo, —OH, and —CN.

In embodiments, —X— is selected from a bond, and —C(O)—.

In embodiments, —X— is —C(O)—.

In embodiments, R¹ is selected from —NR^{1a}R^{1b}, a substituted or unsubstituted 5 or 6 membered monocyclic heteroaryl group having 1, 2 or 3 heteroatoms selected from O, N or S, a substituted or unsubstituted 6 membered monocyclic aryl group, a substituted or unsubstituted 3 to 8 membered monocyclic or bicyclic (fused, bridged or spiro) cycloalkyl group, and a substituted or unsubstituted 3 to 8 membered monocyclic or bicyclic (fused, bridged or spiro) heterocyclic group having 1, 2 or 3 heteroatoms selected from O, N or S.

In embodiments, R¹ is selected from —NR^{1a}R^{1b}, a substituted or unsubstituted 5 or 6 membered monocyclic heteroaryl group having 1, 2 or 3 heteroatoms selected from O, N or S, and a substituted or unsubstituted 3 to 8 membered monocyclic or bicyclic (fused, bridged or spiro) heterocyclic group having 1, 2 or 3 heteroatoms selected from O, N or S.

In embodiments, R¹ is selected from —NR^{1a}R^{1b}, and a substituted or unsubstituted 3 to 8 membered monocyclic or bicyclic (fused, bridged or spiro) heterocyclic group having 1, 2 or 3 heteroatoms selected from O, N or S.

In embodiments, R¹ is a substituted or unsubstituted 3 to 8 membered monocyclic or bicyclic (fused, bridged or spiro) heterocyclic group having 1, 2 or 3 heteroatoms selected from O, N or S.

Optionally, R¹ is a substituted or unsubstituted 3 to 8 membered monocyclic or bicyclic (fused, bridged, or spiro) heterocyclic ring system comprising a nitrogen atom and 0, 1, or 2 additional heteroatoms selected from O, N or S. Further optionally, R¹ is a substituted or unsubstituted 3 to 8 membered monocyclic or bicyclic (fused, bridged, or spiro) heterocyclic ring system comprising a nitrogen atom and 0, 1, or 2 additional heteroatoms selected from O, N or S, wherein the heterocyclic ring system is connected to —X— via a nitrogen atom. Optionally, the additional heteroatoms are selected from O and N.

In embodiments, —X—R¹ has the structure:

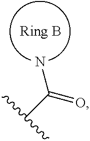

wherein Ring B is a substituted or unsubstituted monocyclic or bicyclic (fused, bridged, or spiro) 3 to 8 membered heterocyclic ring system comprising 0, 1, or 2 additional heteroatoms selected from O, N or S. Optionally, the additional heteroatoms are selected from O and N.

In embodiments, R¹ is a substituted or unsubstituted 3 to 8 membered monocyclic or bicyclic (fused, bridged, or spiro) heterocyclic ring system comprising a nitrogen atom and 0 or 1 additional heteroatoms selected from O, N or S, wherein the heterocyclic ring system is connected to —X— via a nitrogen atom. Optionally, the additional heteroatoms are selected from O and N.

In embodiments, R¹ is a substituted or unsubstituted 4 to 7 membered monocyclic heterocyclic group having 1, 2 or 3 heteroatoms selected from O, N or S. Optionally, R¹ is a substituted or unsubstituted 4 to 7 membered monocyclic heterocyclic ring system comprising a nitrogen atom and 0, 1, or 2 additional heteroatoms selected from O, N or S. Further optionally, R¹ is a substituted or unsubstituted 4 to 7 membered monocyclic heterocyclic ring system comprising a nitrogen atom and 0, 1, or 2 additional heteroatoms selected from O, N or S, wherein the heterocyclic ring system is connected to —X— via a nitrogen atom. Optionally, the additional heteroatoms are selected from O and N.

In embodiments, —X—R¹ has the structure:

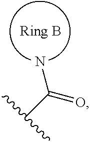

wherein Ring B is a substituted or unsubstituted monocyclic or bicyclic (fused, bridged, or spiro) 4 to 7 membered heterocyclic ring system comprising 0, 1, or 2 additional heteroatoms selected from O, N or S. Optionally, the additional heteroatoms are selected from O and N.

In embodiments, R¹ is a substituted or unsubstituted 4 to 7 membered monocyclic or bicyclic (fused, bridged, or spiro) heterocyclic ring system comprising a nitrogen atom and 0 or 1 additional heteroatoms selected from O, N or S, wherein the heterocyclic ring system is connected to —X— via a nitrogen atom. Optionally, the additional heteroatoms are selected from O and N.

In embodiments, R¹ is a substituted or unsubstituted 5 or 6 membered monocyclic heterocyclic group having 1, 2 or 3 heteroatoms selected from O, N or S. Optionally, R¹ is a substituted or unsubstituted 5 or 6 membered monocyclic heterocyclic ring system comprising a nitrogen atom and 0, 1, or 2 additional heteroatoms selected from O, N or S. Further optionally, R¹ is a substituted or unsubstituted 5 or 6 membered monocyclic heterocyclic ring system comprising a nitrogen atom and 0, 1, or 2 additional heteroatoms selected from O, N or S, wherein the heterocyclic ring system is connected to —X— via a nitrogen atom. Optionally, the additional heteroatoms are selected from O and N.

In embodiments, —X—R¹ has the structure:

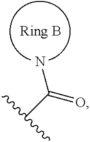

wherein Ring B is a substituted or unsubstituted monocyclic 5 or 6 membered heterocyclic ring system comprising 0, 1, or 2 additional heteroatoms selected from O, N or S. Optionally, the additional heteroatoms are selected from O and N.

In embodiments, R¹ is a substituted or unsubstituted 5 or 6 membered monocyclic heterocyclic ring system comprising a nitrogen atom and 0 or 1 additional heteroatoms selected from O, N or S, wherein the heterocyclic ring system is connected to —X— via a nitrogen atom. Optionally, the additional heteroatoms are selected from O and N.

In embodiments, R¹ is selected from a substituted or unsubstituted:

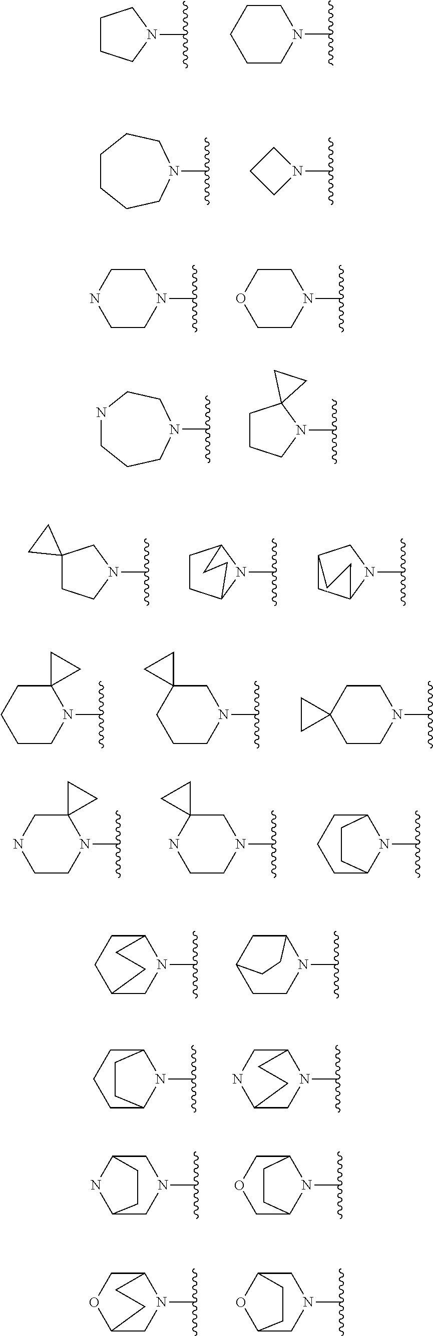

In embodiments, when substituted, the substituent of R¹ is selected from halo, C₁₋₆ alkyl, C₁₋₆ haloalkyl, —O—C₁₋₆ alkyl, —O—C₁₋₆ haloalkyl, —C₁₋₆ alkyl-O—C₁₋₆ alkyl, —C₁₋₆ alkyl-O—C₁₋₆ haloalkyl, and —NR^{1c}R^{1d}.

In embodiments, when substituted, the substituent of R¹ is selected from halo, C₁₋₃ alkyl, C₁₋₃ haloalkyl, —O—C₁₋₃ alkyl, —O—C₁₋₃ haloalkyl, —C₁₋₃ alkyl-O—C₁₋₃ alkyl, —C₁₋₃ alkyl-O—C₁₋₃ haloalkyl, and —NR^{1c}R^{1d}.

In embodiments, when substituted, the substituent of R¹ is selected from halo, C₁₋₆ alkyl, C₁₋₆ haloalkyl, —O—C₁₋₆ alkyl, and —C₁₋₆ alkyl-O—C₁₋₆ alkyl.

In embodiments, when substituted, the substituent of R¹ is selected from halo, C₁₋₃ alkyl, C₁₋₃ haloalkyl, —O—C₁₋₃ alkyl and —C₁₋₃ alkyl-O—C₁₋₃ alkyl.

In embodiments, when substituted, the substituent of R¹ is selected from halo, C₁₋₆ alkyl, C₁₋₆ haloalkyl, —O—C₁₋₆ alkyl, —O—C₁₋₆ haloalkyl, and —NR^{1c}R^{1d}.

In embodiments, when substituted, the substituent of R¹ is selected from halo, C₁₋₃ alkyl, C₁₋₃ haloalkyl, —O—C₁₋₃ alkyl, —O—C₁₋₃ haloalkyl, and —NR^{1c}R^{1d}.

In embodiments, when substituted, the substituent of R¹ is selected from halo, C₁₋₆ alkyl, C₁₋₆ haloalkyl, and —O—C₁₋₆ alkyl.

In embodiments, when substituted, the substituent of R¹ is selected from halo, C₁₋₃ alkyl, C₁₋₃ haloalkyl, and —O—C₁₋₃ alkyl.

In embodiments, when substituted, the substituent of R¹ is selected from F, Me, Et, CF₃, —OMe, —OEt and —NMe₂.

In embodiments, when substituted, the substituent of R¹ is selected from F, Me, Et, CF₃, —OMe and —OEt.

In embodiments, R¹ might have one or two substituents each independently as defined above.

In embodiments, R^{1c} and R^{1d} are independently at each occurrence selected from: H, C₁₋₆ alkyl, C₁₋₆ haloalkyl, C₃₋₆ cycloalkyl and C₃₋₆ cyclohaloalkyl.

In embodiments, R^{1c} and R^{1d} are independently at each occurrence selected from: H, C₁₋₃ alkyl, C₁₋₃ haloalkyl, C₃ cycloalkyl and C₃ cyclohaloalkyl.

In embodiments, R^{1c} and R^{1d} are independently at each occurrence selected from: H, C₁₋₆ alkyl, and C₃₋₆ cycloalkyl.

In embodiments, R^{1c} and R^{1d} are independently at each occurrence selected from: H, C₁₋₃ alkyl, and C₃ cycloalkyl.

In embodiments, R^{1c} and R^{1d} are independently at each occurrence selected from: H, Me and C₃ cycloalkyl.

In embodiments, R¹ is —NR^{1a}R^{1b}.

In embodiments, —X—R¹ has the structure:

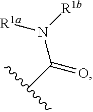

In embodiments, R^{1a} and R^{1b} are independently at each occurrence selected from: substituted or unsubstituted C₁₋₆ alkyl, substituted or unsubstituted C₃₋₇ cycloalkyl, a substituted or unsubstituted 3 to 7 membered heterocyclic group having 1, 2 or 3 heteroatoms selected from O, N or S, substituted or unsubstituted —C₁₋₆ alkyl-C₃₋₇ cycloalkyl, a substituted or unsubstituted —C₁₋₆ alkyl-3 to 7 membered heterocyclic group having 1, 2 or 3 heteroatoms selected from O, N or S.

In embodiments, R^{1a} and R^{1b} are independently at each occurrence selected from: substituted or unsubstituted C₁₋₃ alkyl, substituted or unsubstituted C₃₋₇ cycloalkyl, a substituted or unsubstituted 3 to 7 membered heterocyclic group having 1, 2 or 3 heteroatoms selected from O, N or S, substituted or unsubstituted —C₁₋₃ alkyl-C₃₋₇ cycloalkyl, a substituted or unsubstituted —C₁₋₃ alkyl-3 to 7 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl, substituted or unsubstituted C₃₋₆ cycloalkyl, a substituted or unsubstituted 3 to 6 membered heterocyclic group having 1, 2 or 3 heteroatoms selected from O, N or S, substituted or unsubstituted —C₁₋₃ alkyl-C₃₋₆ cycloalkyl, a substituted or unsubstituted —C₁₋₃ alkyl-3 to 6 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl, substituted or unsubstituted C₃ cycloalkyl, a substituted or unsubstituted 3 to 6 membered heterocyclic group having 1, 2 or 3 heteroatoms selected from O, N or S, a substituted or unsubstituted —C₁₋₃ alkyl-3 to 6 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl, substituted or unsubstituted C₃ cycloalkyl, a substituted or unsubstituted 4, 5 or 6 membered heterocyclic group having 1, 2 or 3 heteroatoms selected from O, N or S, and a substituted or unsubstituted —C₁₋₃ alkyl-4, 5, or 6 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl and substituted or unsubstituted C₃₋₆ cycloalkyl.

In embodiments R^{1a} and R^{1b} are independently at each occurrence selected from: substituted or unsubstituted C₁₋₃ alkyl and substituted or unsubstituted C₃ cycloalkyl.

In embodiments R^{1a} and R^{1b} are independently at each occurrence selected from: substituted or unsubstituted C₁₋₃ alkyl.

In embodiments R^{1a} and R^{1b} are independently at each occurrence selected from: substituted or unsubstituted Me and substituted or unsubstituted C₃ cycloalkyl.

In embodiments R^{1a} and R^{1b} are each Me. In embodiments R^{1a} is Me and R^{1b} is C₃ cycloalkyl.

In embodiments R^{1a} and R^{1b} are independently at each occurrence selected from: C₁₋₃ alkyl optionally substituted with —NR^{1e}R^{1f} and substituted or unsubstituted C₃ cycloalkyl.

In embodiments R^{1a} and R^{1b} are independently at each occurrence selected from: substituted or unsubstituted C₁₋₃ alkyl and a substituted or unsubstituted —C₁₋₃ alkyl-3 to 6 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl and a substituted or unsubstituted —C₁₋₃ alkyl-4, 5, or 6 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl and a substituted or unsubstituted —C₂ alkyl-4, 5, or 6 membered heterocyclic group having 1, 2 or 3 heteroatoms selected from O, N or S.

In embodiments R^{1a} and R^{1b} are independently at each occurrence selected from: substituted or unsubstituted Me and a substituted or unsubstituted —C₁₋₃ alkyl-4, 5, or 6 membered heterocyclic group having 1, 2 or 3 heteroatoms selected from O, N or S.

In embodiments R^{1a} and R^{1b} are independently at each occurrence selected from: substituted or unsubstituted Me and a substituted or unsubstituted —C₂ alkyl-4, 5, or 6 membered heterocyclic group having 1, 2 or 3 heteroatoms selected from O, N or S.

In embodiments R^{1a} and R^{1b} are independently at each occurrence selected from: substituted or unsubstituted C₁₋₃ alkyl and

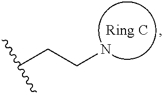 [SMILES: *CCN]

wherein Ring C is a substituted or unsubstituted monocyclic 3 to 6 membered heterocyclic ring system comprising 0, 1, or 2 additional heteroatoms selected from O, N or S.

In embodiments R^{1a} and R^{1b} are independently at each occurrence selected from: substituted or unsubstituted C₁₋₃ alkyl and

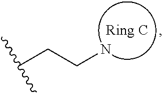 [SMILES: *CCN]

wherein Ring C is a substituted or unsubstituted monocyclic 4, 5 or 6 membered heterocyclic ring system comprising 0, 1, or 2 additional heteroatoms selected from O, N or S.

In embodiments R^{1a} and R^{1b} are independently at each occurrence selected from: substituted or unsubstituted Me and

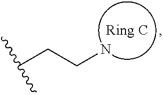 [SMILES: *CCN]

wherein Ring C is a substituted or unsubstituted monocyclic 3 to 6 membered heterocyclic ring system comprising 0, 1, or 2 additional heteroatoms selected from O, N or S.

In embodiments R^{1a} and R^{1b} are independently at each occurrence selected from: substituted or unsubstituted Me and

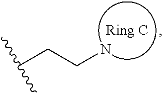 [SMILES: *CCN]

wherein Ring C is a substituted or unsubstituted monocyclic 4, 5 or 6 membered heterocyclic ring system comprising 0, 1, or 2 additional heteroatoms selected from O, N or S.

In embodiments R^{1a} and R^{1b} are independently at each occurrence selected from: substituted or unsubstituted C₁₋₃ alkyl and

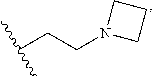 [SMILES: *CCN1CCC1 ; ","]

wherein the ring is substituted or unsubstituted.

In embodiments R^{1a} and R^{1b} are independently at each occurrence selected from: substituted or unsubstituted Me and

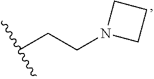 [SMILES: *CCN1CCC1 ; ","]

wherein the ring is substituted or unsubstituted.

In embodiments R^{1a} and R^{1b} are independently at each occurrence selected from: substituted or unsubstituted C₁₋₃ alkyl and

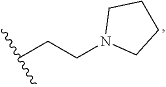 [SMILES: *CCN1CCCC1 ; ","]

wherein the ring is substituted or unsubstituted.

In embodiments R^{1a} and R^{1b} are independently at each occurrence selected from: substituted or unsubstituted Me and

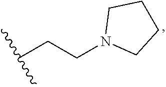 [SMILES: *CCN1CCCC1 ; ","]

wherein the ring is substituted or unsubstituted.

In embodiments one of R^{1a} and R^{1b} is selected from the group consisting of:

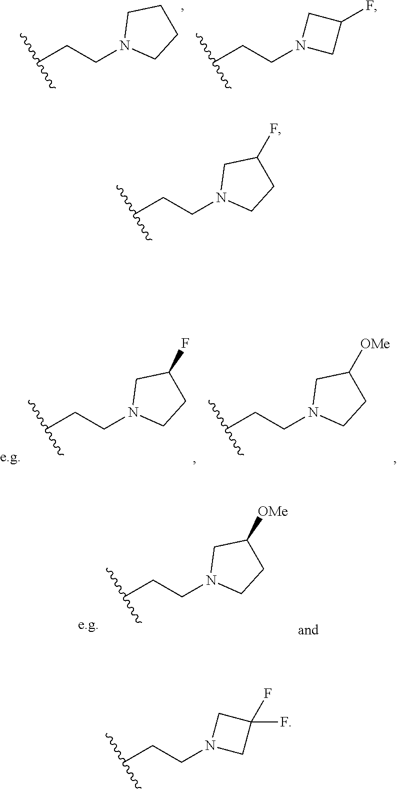

In embodiments R^{1a} and R^{1b} are independently at each occurrence selected from: substituted or unsubstituted C₁₋₃ alkyl and a substituted or unsubstituted 3 to 6 membered heterocyclic group having 1, 2 or 3 heteroatoms selected from O, N or S,

In embodiments R^{1a} and R^{1b} are independently at each occurrence selected from: substituted or unsubstituted C₁₋₃ alkyl and a substituted or unsubstituted 4, 5 or 6 membered heterocyclic group having 1, 2 or 3 heteroatoms selected from O, N or S,

In embodiments R^{1a} and R^{1b} are independently at each occurrence selected from: substituted or unsubstituted C₁₋₃ alkyl and a substituted or unsubstituted 5 or 6 membered heterocyclic group having 1, 2 or 3 heteroatoms selected from O, N or S,

In embodiments R^{1a} and R^{1b} are independently at each occurrence selected from: substituted or unsubstituted Me and a substituted or unsubstituted 3 to 6 membered heterocyclic group having 1, 2 or 3 heteroatoms selected from O, N or S,

In embodiments R^{1a} and R^{1b} are independently at each occurrence selected from: substituted or unsubstituted Me and a substituted or unsubstituted 4, 5 or 6 membered heterocyclic group having 1, 2 or 3 heteroatoms selected from O, N or S,

In embodiments R^{1a} and R^{1b} are independently at each occurrence selected from: substituted or unsubstituted Me and a substituted or unsubstituted 5 or 6 membered heterocyclic group having 1, 2 or 3 heteroatoms selected from O, N or S,

In embodiments R^{1a} and R^{1b} are independently at each occurrence selected from: substituted or unsubstituted C₁₋₃ alkyl and

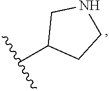 [SMILES: *C1CCNC1 ; ","]

wherein the ring is unsubstituted or is substituted at any position on the ring e.g.

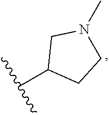 [SMILES: *C1CCN(C)C1 ; ","]

In embodiments R^{1a} and R^{1b} are independently at each occurrence selected from: substituted or unsubstituted Me and

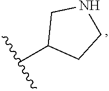 [SMILES: *C1CCNC1 ; ","]

wherein the ring is unsubstituted or is substituted at any position on the ring e.g.

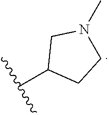 [SMILES: *C1CCN(C)C1 ; "."]

In embodiments R^{1a} and R^{1b} are independently at each occurrence selected from: substituted or unsubstituted C₁₋₃ alkyl and

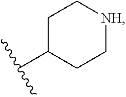 [SMILES: *C1CCNCC1]

wherein the ring is unsubstituted or is substituted at any position on the ring e.g.

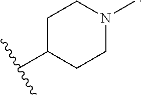 [SMILES: *C1CCN(C)CC1 ; "."]

In embodiments R^{1a} and R^{1b} are independently at each occurrence selected from: substituted or unsubstituted Me and

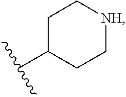 [SMILES: *C1CCNCC1]

wherein the ring is unsubstituted or is substituted at any position on the ring e.g.

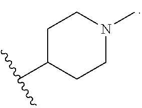 [SMILES: *C1CCN(C)CC1]

In embodiments one of R^{1a} and R^{1b} is selected from the group consisting of:

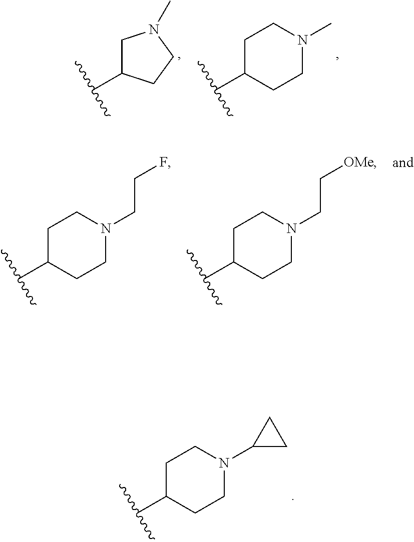

In embodiments, when substituted, the substituent of R^{1a} and R^{1b} is selected from: halo, —CN, —OH, C₁₋₆ alkyl, C₃₋₇ cycloalkyl, C₁₋₆ haloalkyl, —O—C₁₋₆ alkyl, —O—C₃₋₇ cycloalkyl, —O—C₁₋₆ haloalkyl, —C₁₋₆ alkyl-O—C₁₋₆ alkyl, —C₁₋₆ alkyl-O—C₃₋₇ cycloalkyl, —C₁₋₆ alkyl-O—C₁₋₆ haloalkyl, —NR^{1e}R^{1f}, —NR^{1e}(SO₂)R^{1f}, —NR^{1e}(C(O))R^{1f}, —C(O)NR^{1e}R^{1f}, and —SO₂NR^{1e}R^{1f}.

In embodiments, when substituted, the substituent of R^{1a} and R^{1b} is selected from: halo, —CN, —OH, C₁₋₃ alkyl, C₃₋₇ cycloalkyl, C₁₋₃ haloalkyl, —O—C₁₋₃ alkyl, —O—C₃₋₇ cycloalkyl, —O—C₁₋₃ haloalkyl, —C₁₋₃ alkyl-O—C₁₋₃ alkyl, —C₁₋₃ alkyl-O—C₃₋₇ cycloalkyl, —C₁₋₃ alkyl-O—C₁₋₃ haloalkyl, —NR^{1e}R^{1f}, —NR^{1e}(SO₂)R^{1f}, —NR^{1e}(C(O))R^{1f}, —C(O)NR^{1e}R^{1f}, and —SO₂NR^{1e}R^{1f}.

In embodiments, when R^{1a} and R^{1b} are substituted, each substituent is independently selected from: halo, C₁₋₃ alkyl, C₁₋₃ haloalkyl, —O—C₁₋₃ alkyl, —O—C₁₋₃ haloalkyl, —C₁₋₃ alkyl-O—C₁₋₃ alkyl, —C₁₋₃ alkyl-O—C₁₋₃ haloalkyl, C₃₋₆ cycloalkyl, and —NR^{1e}R^{1f}.

In embodiments, when R^{1a} and R^{1b} are substituted, each substituent is independently selected from: halo, C₁₋₃ alkyl, C₁₋₃ haloalkyl, —O—C₁₋₃ alkyl, —C₁₋₃ alkyl-O—C₁₋₃ alkyl, C₃ cycloalkyl and —NR^{1e}R^{1f}.

In embodiments, when R^{1a} and R^{1b} are substituted, each substituent is independently selected from: halo, C₁₋₃ alkyl, C₁₋₃ haloalkyl, —O—C₁₋₃ alkyl, —O—C₁₋₃ haloalkyl, C₃₋₆ cycloalkyl, and —NR^{1e}R^{1f}.

In embodiments, when R^{1a} and R^{1b} are substituted, each substituent is independently selected from: halo, C₁₋₃ alkyl, C₁₋₃ haloalkyl, —O—C₁₋₃ alkyl and C₃ cycloalkyl.

In embodiments, when R^{1a} and R^{1b} are substituted, each substituent is independently selected from F, Me, Et, CH₂F, CHF₂, CF₃, CH₂CH₂F, CH₂CHF₂, CH₂CF₃, —OMe, —OEt, CH₂OMe, CH₂CH₂OMe and cyclopropyl.

In embodiments, when R^{1a} and R^{1b} are substituted, each substituent is independently selected from F, Me, Et, CF₃, —OMe and —OEt.

In embodiments, when R^{1a} and R^{1b} are substituted, each substituent might be —NR^{1e}R^{1f}.

In embodiments, R^{1e} and R^{1f} are independently at each occurrence selected from: H, C₁₋₆ alkyl, and C₃₋₆ cycloalkyl.

In embodiments, R^{1e} and R^{1f} are independently at each occurrence selected from: H, C₁₋₃ alkyl, and C₃ cycloalkyl.

In embodiments, R^{1e} and R^{1f} are independently at each occurrence selected from: H, Me and C₃ cycloalkyl.

In embodiments, R^{1e} and R^{1f} are independently at each occurrence selected from: Me, Et, CH₂F, CHF₂, CF₃, CH₂CH₂F, CH₂CHF₂ and CH₂CF₃.

In embodiments, R¹ is a substituted or unsubstituted 5 or 6 membered monocyclic heteroaryl group having 1, 2 or 3 heteroatoms selected from O, N or S.

In embodiments, R¹ is a substituted or unsubstituted 5 or 6 membered monocyclic heteroaryl group having 1, 2 or 3 heteroatoms selected from O, N or S and X is a bond.

In embodiments, R¹ is selected from a substituted or unsubstituted:

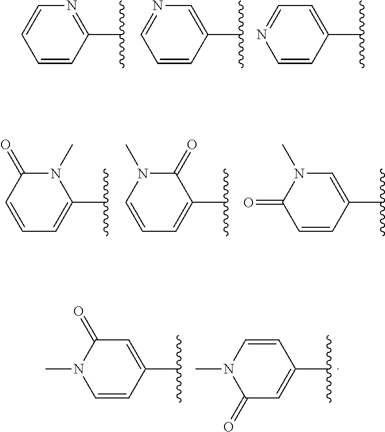

In embodiments, R² is selected from H, C₁₋₆ alkyl, and C₁₋₆ haloalkyl.

In embodiments, R² is selected from H, C₁₋₃ alkyl, and C₁₋₃ haloalkyl.

In embodiments, R² is selected from H, and C₁₋₆ alkyl.

In embodiments, R² is selected from H, and C₁₋₃ alkyl.

In embodiments, R² is selected from H, Me and Et.

In embodiments, R² is H.

In embodiments, R³ is selected from halo, —CN, —OH, C₁₋₃ alkyl, C₁₋₃ haloalkyl, —O—C₁₋₃ alkyl, —O—C₁₋₃ haloalkyl, —NR^{3a}R^{3b}, —NR^{3a}(C(O))R^{3b}, and —C(O)NR^{3a}R^{3b}.

In embodiments, R³ is selected from halo, —CN, —OH, C₁₋₆ alkyl, C₁₋₆ haloalkyl, —O—C₁₋₆ alkyl, —O—C₁₋₆ haloalkyl, and —NR^{3a}R^{3b}.

In embodiments, R³ is selected from halo, —CN, —OH, C₁₋₃ alkyl, C₁₋₃ haloalkyl, —O—C₁₋₃ alkyl, —O—C₁₋₃ haloalkyl, and —NR^{3a}R^{3b}.

In embodiments, R³ is selected from halo, C₁₋₆ alkyl, C₁₋₆ haloalkyl, —O—C₁₋₆ alkyl, and —O—C₁₋₆ haloalkyl.

In embodiments, R³ is selected from halo, C₁₋₃ alkyl, C₁₋₃ haloalkyl, —O—C₁₋₃ alkyl, and —O—C₁₋₃ haloalkyl.

In embodiments, R³ is selected from halo, Me, CF₃, —O-Me, and —O—CF₃.

In embodiments, R^{3a} and R^{3b} are independently at each occurrence selected from: H, C₁₋₆ alkyl, and C₃₋₆ cycloalkyl.

In embodiments, R^{3a} and R^{3b} are independently at each occurrence selected from: H, C₁₋₃ alkyl, and C₃ cycloalkyl.

In embodiments, R^{3a} and R^{3b} are independently at each occurrence selected from: H, Me and C₃ cycloalkyl.

In embodiments, m is selected from 0, or 1.

In embodiments, m is 0.

In embodiments, —X—R¹ has the structure:

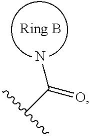

wherein Ring B is a substituted or unsubstituted monocyclic or bicyclic (fused, bridged, or spiro) 4 to 7 membered heterocyclic ring system comprising 0, 1, or 2 additional heteroatoms selected from O, N or S; wherein, when substituted, the substituent of R¹ is selected from F, Me, Et, CF₃, —OMe, —OEt and —NMe₂; R² is selected from H, Me and Et; and m is 0.

In embodiments, —X—R¹ has the structure:

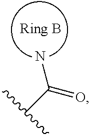

wherein Ring B is a substituted or unsubstituted monocyclic or bicyclic (fused, bridged, or spiro) 4 to 7 membered heterocyclic ring system comprising 0, 1, or 2 additional heteroatoms selected from 0, N or S; wherein, when substituted, the substituent of R¹ is selected from F, Me, Et, CF₃, —OMe and —OEt; R² is selected from H, Me and Et; and m is 0.

In embodiments, —X—R¹ has the structure:

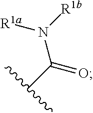

R^{1a} and R^{1b} are independently at each occurrence selected from: substituted or unsubstituted C₁₋₃ alkyl, substituted or unsubstituted C₃ cycloalkyl, a substituted or unsubstituted 4, 5 or 6 membered heterocyclic group having 1, 2 or 3 heteroatoms selected from O, N or S, a substituted or unsubstituted —C₁₋₃ alkyl-4, 5, or 6 membered heterocyclic group having 1, 2 or 3 heteroatoms 10 selected from O, N or S; R² is selected from H, Me and Et; and m is 0.

In embodiments, —X—R¹ has the structure:

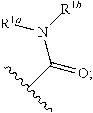

R^{1a} and R^{1b} are independently at each occurrence selected from: substituted or unsubstituted C₁₋₃ alkyl and substituted or unsubstituted C₃ cycloalkyl; R² is selected from H, Me and Et; and m is 0.

In embodiments, the residue is

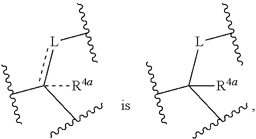

wherein L is selected from: bond, —NR^{4b}—, and —NR^{4c}C(O)—.

In embodiments, the residue

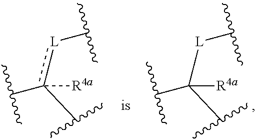

wherein L is selected from: bond, and —O—.

In embodiments, the residue

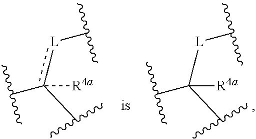

wherein L is a bond.

In embodiments, the residue

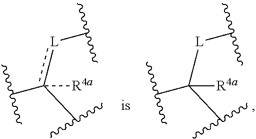

wherein L is a bond and R^{4a} is H.

In embodiments, the residue

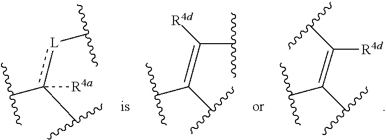

In embodiments, the residue is

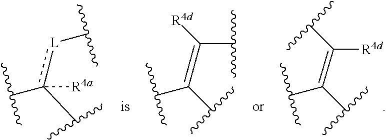

wherein R^{4d} is H.

In embodiments, R^{4a} is selected from H, OH or F.

In embodiments, R^{4a} is selected from: H, C₁₋₄ alkyl, or C₁₋₄ haloalkyl.

In embodiments, R^{4a} is selected from: H, C₁₋₃ alkyl, or C₁₋₃ haloalkyl.

In embodiments, R^{4a} is selected from: H, C₁₋₂ alkyl, or C₁₋₂ haloalkyl.

In embodiments, R^{4a} is selected from: H, C₁ alkyl, or C₁ haloalkyl (e.g. CF₃).

In embodiments, R^{4a} is CF₃.

In embodiments, R^{4a} is H.

In embodiments, R^{4b} is H, C₁₋₃ alkyl or —C(O)C₁₋₃ alkyl.

In embodiments, R^{4b} is H, Me or —C(O)Me.

In embodiments, R^{4b} is H.

In embodiments, R^{4c} is H or C₁₋₃ alkyl.

In embodiments, R^{4c} is H.

In embodiments, R^{4d} is H or C₁₋₃ alkyl.

In embodiments, R^{4d} is H.

In embodiments, R^{4e} and R^{4f} are independently selected from H and C₁₋₄ alkyl.

In embodiments, R^{4e} and R^{4f} are H.

In embodiments, R^{4g} and R^{4h} are independently at each occurrence selected from: H and C₁₋₃ alkyl.

In embodiments, R^{4g} and R^{4h} are independently at each occurrence selected from: H and C₁ alkyl.

In embodiments, R^{4g} and R^{4h} are H.

In embodiments, R⁴ⁱ is independently at each occurrence selected from: halo, C₁₋₄ alkyl, C₁₋₄ haloalkyl, C₃₋₆ cycloalkyl, C₃₋₆ cyclohaloalkyl, —OR^{4j}, —NR^{4k}(C(O))R⁴¹, —CN, —C(O)R^{4g}, ═O, unsubstituted 5 or 6 membered heteroaryl, or methyl substituted 5 or 6 membered heteroaryl.

In embodiments, R⁴ⁱ is independently at each occurrence selected from: halo, C₁₋₃ alkyl, C₁₋₃ haloalkyl, C₃₋₆ cycloalkyl, C₃₋₆ cyclohaloalkyl, —OR^{4j}, —C(O)R^{4g}, and ═O.

In embodiments, R⁴ⁱ is independently at each occurrence selected from: halo, C₁ alkyl, C₁ haloalkyl, C₃ cycloalkyl, C₃ cyclohaloalkyl, —OR^{4j}, —C(O)R^{4g}, and ═O.

In embodiments, R⁴ⁱ is independently at each occurrence selected from: halo, C₁ alkyl, C₁ haloalkyl, C₃ cycloalkyl, C₃ cyclohaloalkyl, and —OR^{4j}.

In embodiments, R⁴ⁱ is independently at each occurrence selected from: halo, C₁₋₄ alkyl, C₁₋₄ haloalkyl, —OR^{4j}, —NR^{4k}(C(O))R^{4l}, —CN, —C(O)R^{4g}, ═O, unsubstituted 5 or 6 membered heteroaryl, or methyl substituted 5 or 6 membered heteroaryl.

In embodiments, R⁴ⁱ is independently at each occurrence selected from: halo, C₁₋₃ alkyl, C₁₋₃ haloalkyl, —OR^{4j}, —C(O)R^{4g}, and ═O.

In embodiments, R⁴ⁱ is independently at each occurrence selected from: halo, Ci alkyl, Ci haloalkyl, —OR^{4j}, —C(O)R^{4g}, and ═O.

In embodiments, R⁴ⁱ is independently at each occurrence selected from: halo, Ci alkyl, Ci haloalkyl, and —OR^{4j}.

In embodiments, R^{4j} is selected from: H, C₁₋₄ alkyl, and C₁₋₄ haloalkyl.

In embodiments, R^{4j} is selected from: H, Ci alkyl, and Ci haloalkyl.

In embodiments, n is selected from 0, 1, or 2.

In embodiments, n is 1. In embodiments, n is 0.

In embodiments, R⁴ is selected from: a monocyclic or bicyclic 6 to 10 membered aryl, a monocyclic or bicyclic 5 to 10 membered heteroaryl, a bicyclic (fused, bridged, or spiro) 6 to 10 membered cycloalkyl ring system, a bicyclic (fused, bridged, or spiro) 6 to 10 membered cycloalkenyl ring system, and a monocyclic or bicyclic (fused, bridged, or spiro) 6 to 10 membered heterocyclic ring system comprising 1, 2, or 3 heteroatoms selected from O, N or S, wherein the monocyclic or bicyclic 6 to 10 membered aryl, the monocyclic or bicyclic 5 to 10 membered heteroaryl, the bicyclic (fused, bridged, or spiro) 6 to 10 membered cycloalkyl ring system, the bicyclic (fused, bridged, or spiro) 6 to 10 membered cycloalkenyl ring system or the monocyclic or bicyclic (fused, bridged, or spiro) 6 to 10 membered heterocyclic ring system is unsubstituted or substituted with 1, 2 or 3 R⁴ⁱ.

In embodiments, R⁴ is selected from: a monocyclic 6 membered aryl, a monocyclic 5 or 6 membered heteroaryl, a bicyclic (fused, bridged, or spiro) 8 to 10 membered cycloalkyl ring system, a bicyclic (fused, bridged, or spiro) 8 to 10 membered cycloalkenyl ring system, and a monocyclic or bicyclic (fused, bridged, or spiro) 8 to 10 membered heterocyclic ring system comprising 1, 2, or 3 heteroatoms selected from O, N or S, wherein the monocyclic 6 membered aryl, the monocyclic 5 or 6 membered heteroaryl, the bicyclic (fused, bridged, or spiro) 8 to 10 membered cycloalkyl ring system, the bicyclic (fused, bridged, or spiro) 8 to 10 membered cycloalkenyl ring system or the monocyclic or bicyclic (fused, bridged, or spiro) 8 to 10 membered heterocyclic ring system is unsubstituted or substituted with 1, 2 or 3 R⁴ⁱ.

In embodiments, R⁴ is selected from: a monocyclic 6 membered aryl, a monocyclic 5 or 6 membered heteroaryl, a bicyclic (fused, bridged, or spiro) 8 to 10 membered cycloalkyl ring system, a bicyclic (fused, bridged, or spiro) 8 to 10 membered cycloalkenyl ring system, and a monocyclic or bicyclic (fused, bridged, or spiro) 9 to 10 membered heterocyclic ring system comprising 1, 2, or 3 heteroatoms selected from O, N or S, wherein the monocyclic 6 membered aryl, the monocyclic 5 or 6 membered heteroaryl, the bicyclic (fused, bridged, or spiro) 8 to 10 membered cycloalkyl ring system, the bicyclic (fused, bridged, or spiro) 8 to 10 membered cycloalkenyl ring system or the monocyclic or bicyclic (fused, bridged, or spiro) 9 to 10 membered heterocyclic ring system is unsubstituted or substituted with 1, 2 or 3 R⁴ⁱ.

In embodiments, R⁴ is selected from: a monocyclic or bicyclic 6 to 10 membered aryl, a monocyclic or bicyclic 5 to 10 membered heteroaryl, and a monocyclic or bicyclic (fused, bridged, or spiro) 6 to 10 membered heterocyclic ring system comprising 1, 2, or 3 heteroatoms selected from O, N or S, wherein the monocyclic or bicyclic 6 to 10 membered aryl, the monocyclic or bicyclic 5 to 10 membered heteroaryl, or the monocyclic or bicyclic (fused, bridged, or spiro) 6 to 10 membered heterocyclic ring system is unsubstituted or substituted with 1, 2 or 3 R⁴ⁱ.

In embodiments, R⁴ is selected from: a monocyclic 6 membered aryl, a monocyclic 5 or 6 membered heteroaryl, and a monocyclic or bicyclic (fused, bridged, or spiro) 8 to 10 membered heterocyclic ring system comprising 1, 2, or 3 heteroatoms selected from O, N or S, wherein the monocyclic 6 membered aryl, the monocyclic 5 or 6 membered heteroaryl, or the monocyclic or bicyclic (fused, bridged, or spiro) 6 to 10 membered heterocyclic ring system is unsubstituted or substituted with 1, 2 or 3 R⁴ⁱ.

In embodiments, R⁴ is selected from: a monocyclic 6 membered aryl, a monocyclic 5 or 6 membered heteroaryl, and a bicyclic (fused, bridged, or spiro) 9 or 10 membered heterocyclic ring system comprising 1, 2, or 3 heteroatoms selected from O, N or S, wherein the monocyclic 6 membered aryl, the monocyclic 5 or 6 membered heteroaryl, or the bicyclic 9 or 10 membered heterocyclic ring system is unsubstituted or substituted with 1, 2 or 3 R⁴ⁱ.

In embodiments, R⁴ is C₁₋₄ haloalkyl.

In embodiments, R⁴ is H.

In embodiments,

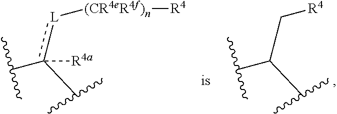

In embodiments,

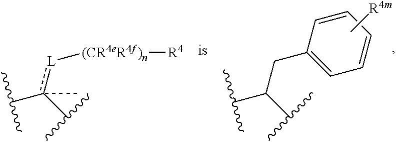

wherein R^{4m} is selected from Me, Et, Pr, F, Cl, CF₃, OMe, OCH₂F, OCHF₂, OCF₃,

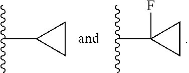

In embodiments

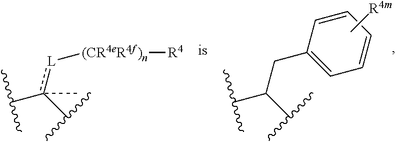

wherein R^{4m} is selected from Me, F, Cl, CF₃ and OMe.

In embodiments,

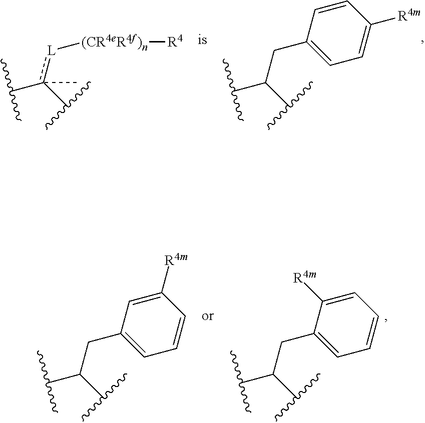

wherein R^{4m} is selected from Me, Et, Pr, F, Cl, CF₃, OMe, OCH₂F, OCHF₂, OCF₃,

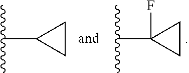

In embodiments,

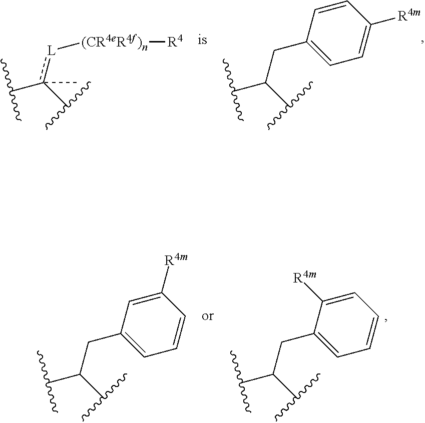

wherein R^{4m} is selected from Me, F, Cl, CF₃ and OMe.

In embodiments,

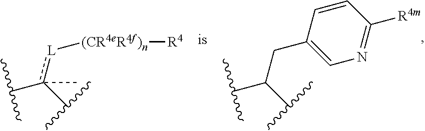

wherein R^{4m} is selected from Me, Et, Pr, F, Cl, CF₃, OMe, OCH₂F, OCHF₂, OCF₃,

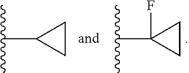

In embodiments,

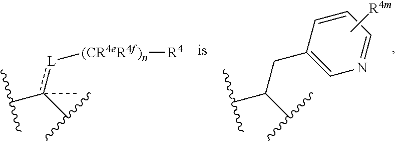

wherein R^{4m} is selected from Me, F, Cl, CF₃ and OMe.

In embodiments,

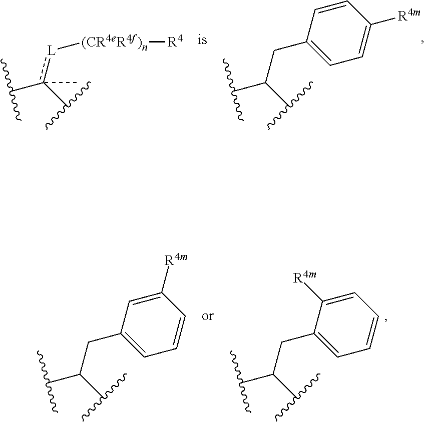

wherein R^{4m} is selected from Me, Et, Pr, F, Cl, CF₃, OMe, OCH₂F, OCHF₂, OCF₃,

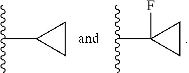

In embodiments,

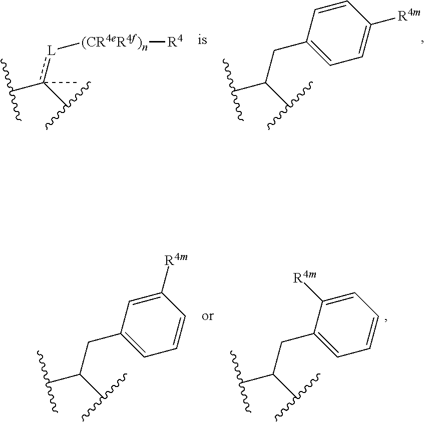

wherein R^{4m} is selected from Me, F, Cl, CF₃ and OMe.

In embodiments,

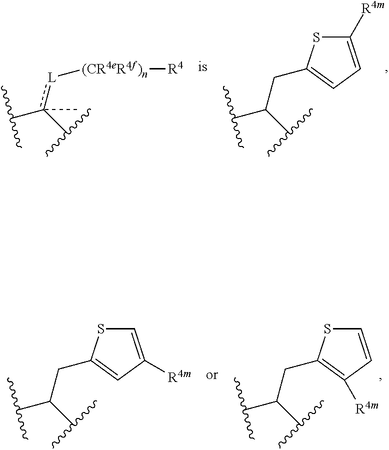

wherein R^{4m} is selected from Me, Et, Pr, F, Cl, CF₃, OMe, OCH₂F, OCHF₂, OCF₃,

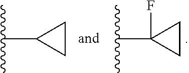

In embodiments,

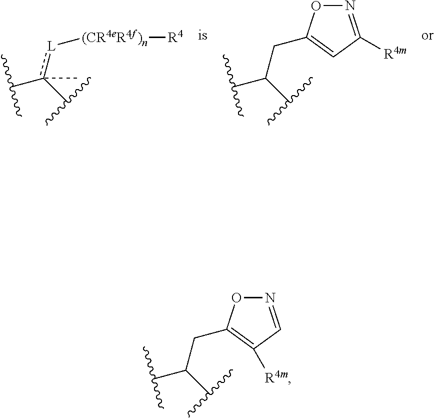

wherein R^{4m} is selected from Me, Et, Pr, F, Cl, CF₃, OMe, OCH₂F, OCHF₂, OCF₃,

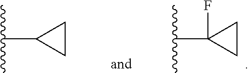

In embodiments,

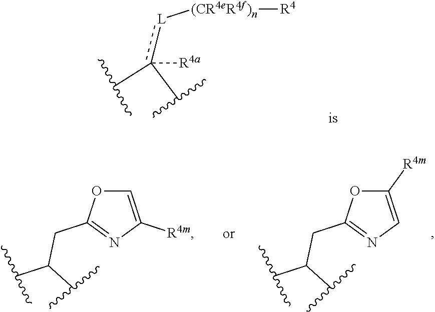

wherein R^{4m} is selected from Me, Et, Pr, F, Cl, CF₃, OMe, OCH₂F, OCHF₂, OCF₃,

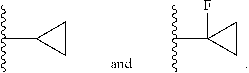

In embodiments,

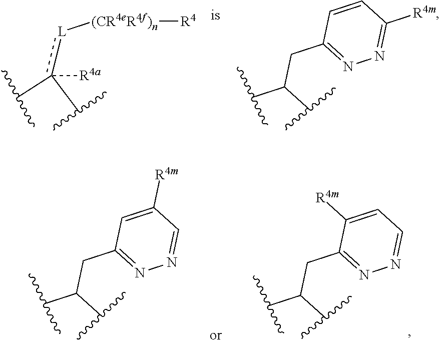

wherein R^{4m} is selected from Me, Et, Pr, F, Cl, CF₃, OMe, OCH₂F, OCHF₂, OCF₃,

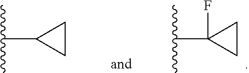

In embodiments

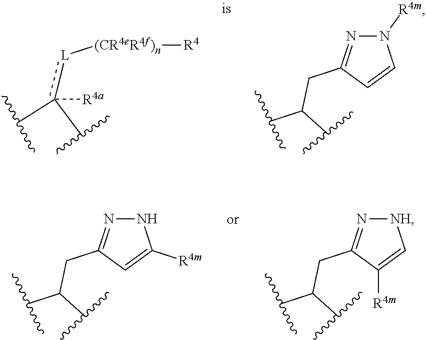

wherein R^{4m} is selected from Me, Et, Pr, F, Cl, CF₃, OMe, OCH₂F, OCHF₂, OCF₃,

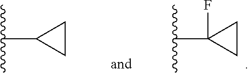

In embodiments

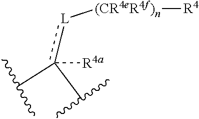

is selected from:

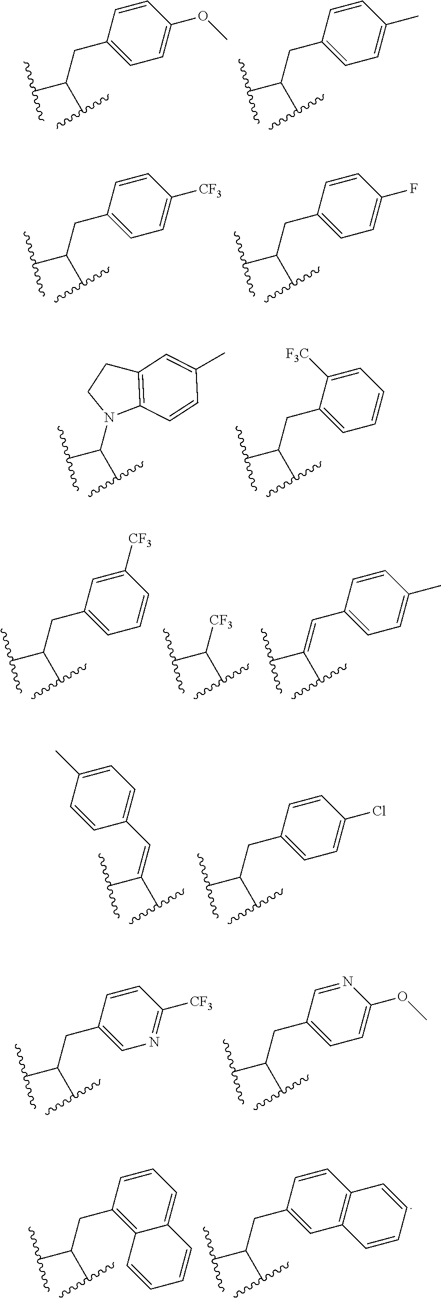

In embodiments

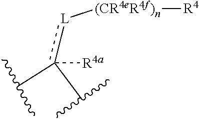

is selected from:

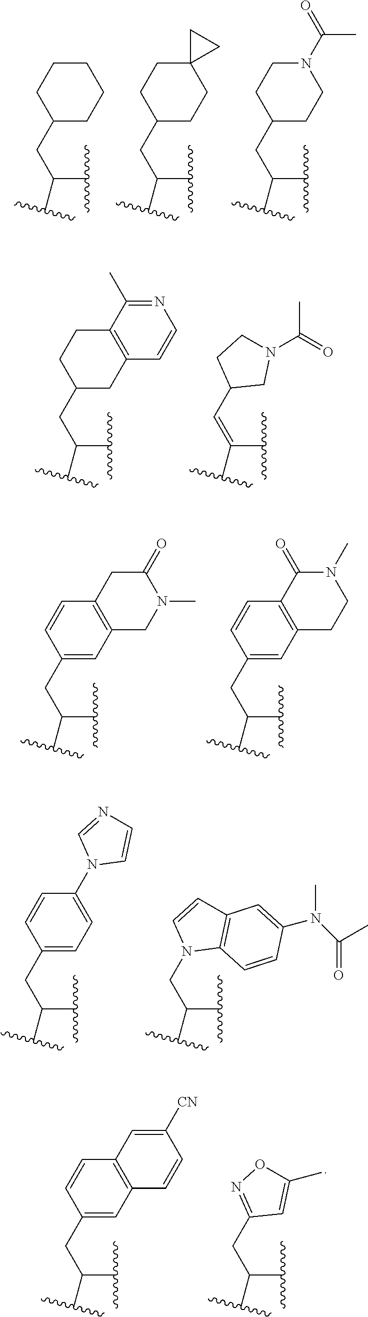

In embodiments

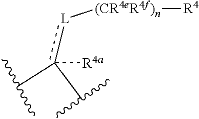

is selected from:

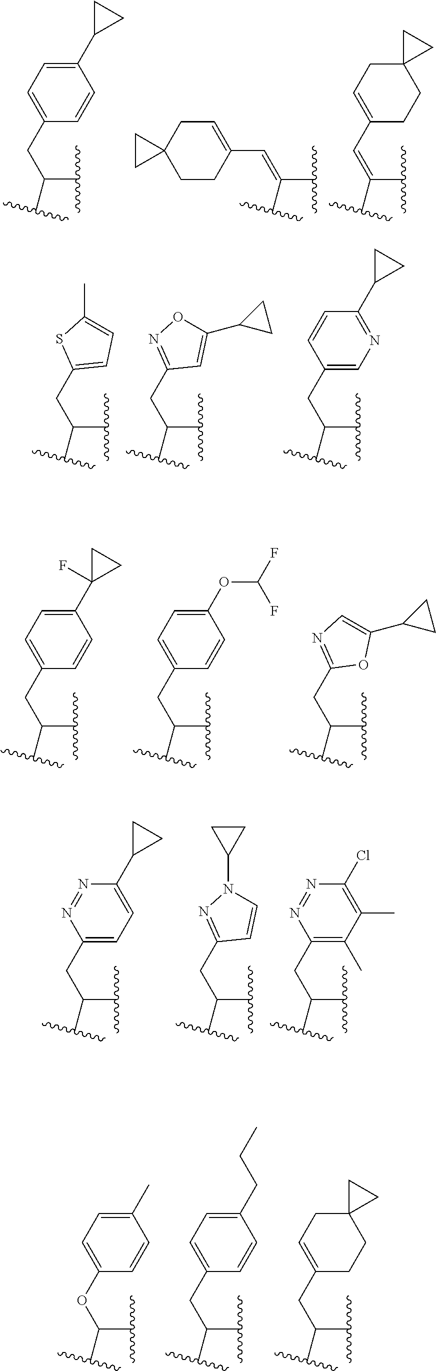

In embodiments, R⁵ is H or C₁₋₃ alkyl.

In embodiments, R⁵ is H.

In embodiments o is 1.

In embodiments o is 2.

In embodiments o is 3.

In embodiments, R^{5a} and R^{5b} are independently selected from H and C₁₋₄ alkyl.

In embodiments, R^{5a} and R^{5b} are H.

In embodiments, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Optionally, the heteroatoms are selected from O or N.

In embodiments, Ring A is selected from a substituted or unsubstituted 9 membered bicyclic heteroaryl group having 1, 2 or 3 heteroatoms selected from O, N or S, and a substituted or unsubstituted bicyclic (fused, bridged, or spiro) 9 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 substituents independently selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Optionally, the heteroatoms are selected from O or N.

In embodiments, Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Optionally, the heteroatoms are selected from O or N.

In embodiments,

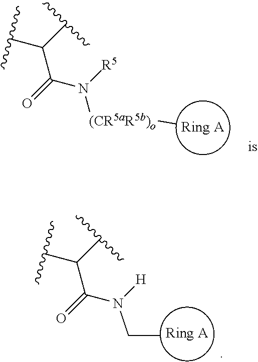

In embodiments,

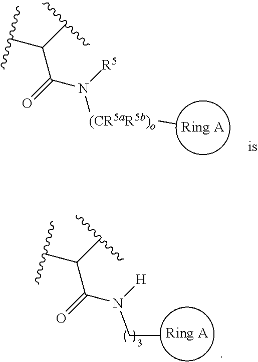

When Ring A is substituted, the bicyclic heteroaryl group or the bicyclic heterocyclic ring system are optionally substituted with 1, 2, or 3 substituents independently selected from: halo, C₁₋₆ alkyl, C₁₋₆ haloalkyl, deuterated C₁₋₆ alkyl, or —OR^{5c}.

When Ring A is substituted, the bicyclic heteroaryl group or the bicyclic heterocyclic ring system are optionally substituted with 1, 2, or 3 substituents independently selected from: halo, C₁₋₃ alkyl, C₁₋₃ haloalkyl, deuterated C₁₋₃ alkyl, or —OR^{5c}.

When Ring A is substituted, the bicyclic heteroaryl group or the bicyclic heterocyclic ring system are optionally substituted with 1, 2, or 3 substituents independently selected from: halo (e.g. F), C₁ alkyl, C₁ haloalkyl (e.g. CFH₂, CF₂H or CF₃), deuterated C₁ alkyl (e.g. —CD3), or —OR^{5c}.

When Ring A is substituted, the bicyclic heteroaryl group or the bicyclic heterocyclic ring system are optionally substituted with 1 or 2 substituents independently selected from: halo, C₁₋₆ alkyl, C₁₋₆ haloalkyl, deuterated C₁₋₆ alkyl, or —OR^{5c}.

When Ring A is substituted, the bicyclic heteroaryl group or the bicyclic heterocyclic ring system are optionally substituted with 1 or 2 substituents independently selected from: halo, C₁₋₃ alkyl, C₁₋₃ haloalkyl, deuterated C₁₋₃ alkyl, or —OR^{5c}.

When Ring A is substituted, the bicyclic heteroaryl group or the bicyclic heterocyclic ring system are optionally substituted with 1 or 2 substituents independently selected from: halo (e.g. F), C₁ alkyl, C₁ haloalkyl (e.g. CFH₂, CF₂H or CF₃), deuterated C₁ alkyl (e.g. —CD3), or —OR^{5c}.

When Ring A is substituted, the 5 to 6 membered monocyclic heteroaryl group having 1, 2 or 3 heteroatoms selected from O, N or S or the 6 membered monocyclic aryl group are optionally substituted with 1, 2, or 3 substituents independently selected from: halo, C₁₋₆ alkyl, C₁₋₆ haloalkyl, deuterated C₁₋₆ alkyl, or —OR^{5c}.

When Ring A is substituted, the 5 to 6 membered monocyclic heteroaryl group having 1, 2 or 3 heteroatoms selected from O, N or S or the 6 membered monocyclic aryl group are optionally substituted with 1, 2, or 3 substituents independently selected from: halo, C₁₋₃ alkyl, C₁₋₃ haloalkyl, deuterated C₁₋₃ alkyl, or —OR^{5c}.

When Ring A is substituted, the 5 to 6 membered monocyclic heteroaryl group having 1, 2 or 3 heteroatoms selected from O, N or S or the 6 membered monocyclic aryl group are optionally substituted with 1, 2, or 3 substituents independently selected from: halo (e.g. F), C₁ alkyl, C₁ haloalkyl (e.g. CFH₂, CF₂H or CF₃), deuterated C₁ alkyl (e.g. —CD₃), or —OR^{5c}.

When Ring A is substituted, the 5 to 6 membered monocyclic heteroaryl group having 1, 2 or 3 heteroatoms selected from O, N or S or the 6 membered monocyclic aryl group are optionally substituted with 1 or 2 substituents independently selected from: halo, C₁₋₆ alkyl, C₁₋₆ haloalkyl, deuterated C₁₋₆ alkyl, or —OR^{5c}.

When Ring A is substituted, the 5 to 6 membered monocyclic heteroaryl group having 1, 2 or 3 heteroatoms selected from O, N or S or the 6 membered monocyclic aryl group are optionally substituted with 1 or 2 substituents independently selected from: halo, C₁₋₃ alkyl, C₁₋₃ haloalkyl, deuterated C₁₋₃ alkyl, or —OR^{5c}.

When Ring A is substituted, the 5 to 6 membered monocyclic heteroaryl group having 1, 2 or 3 heteroatoms selected from O, N or S or the 6 membered monocyclic aryl group are optionally substituted with 1 or 2 substituents independently selected from: halo (e.g. F), C₁ alkyl, C₁ haloalkyl (e.g. CFH₂, CF₂H or CF₃), deuterated C₁ alkyl (e.g. —CD₃), or —OR^{5c}.

In embodiments, R^{5c} and R^{5d} are independently at each occurrence selected from: H, C₁₋₃ alkyl, C₃₋₆ cycloalkyl, and C₁₋₃ haloalkyl.

In embodiments, R^{5c} and R^{5d} are independently at each occurrence selected from: H, Me, cyclopropyl and CF₃.

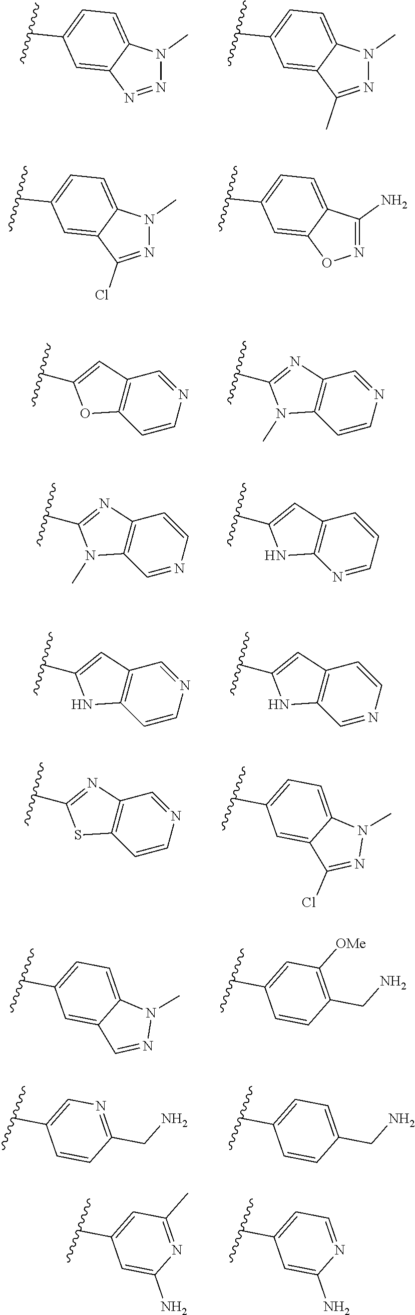

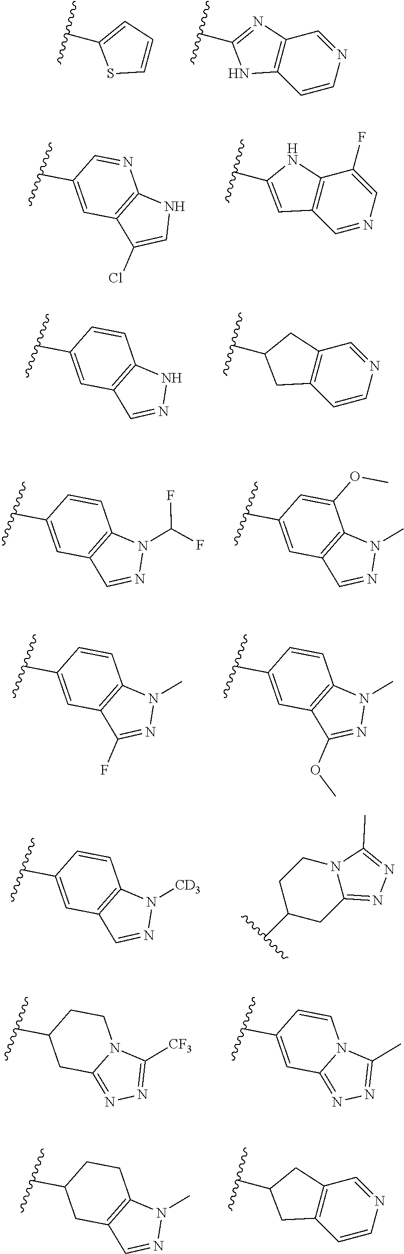

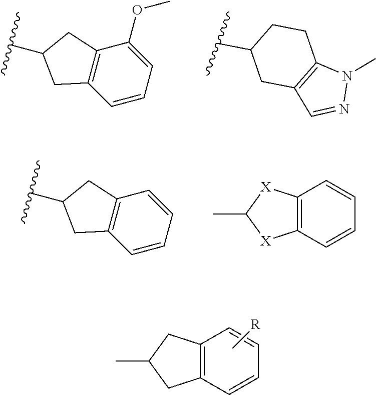

In embodiments, Ring A is selected from a substituted or unsubstituted:
each X is
independently O, N, or S R=alkoxy, Cl

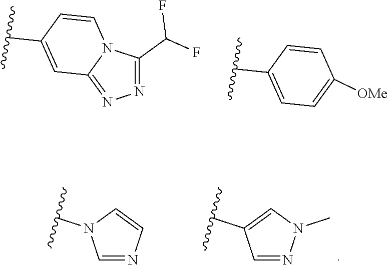

In embodiments Ring A is selected from a substituted or unsubstituted:

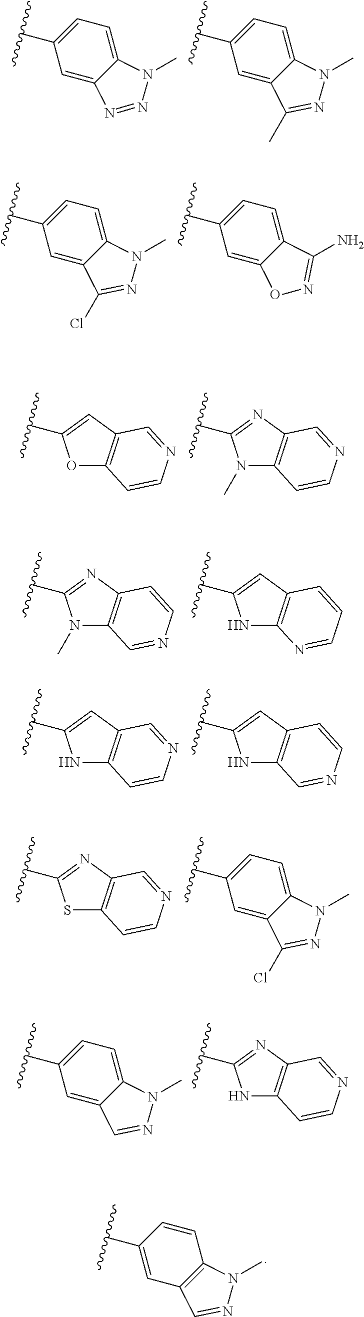

In embodiments Ring A is selected from a substituted or unsubstituted:

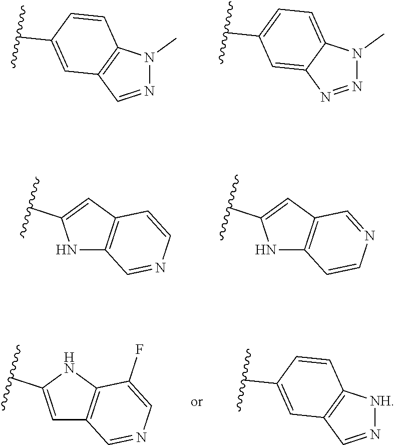 [SMILES: *c1ccc2c(cnn2C)c1 ; *c1ccc2c(c1)nnn2C ; *c1cc2ccncc2[nH]1 ; *c1cc2cnccc2[nH]1 ; *c1cc2cncc(F)c2[nH]1 ; *c1ccc2[nH]ncc2c1]

In embodiments Ring A is selected from a substituted or unsubstituted:

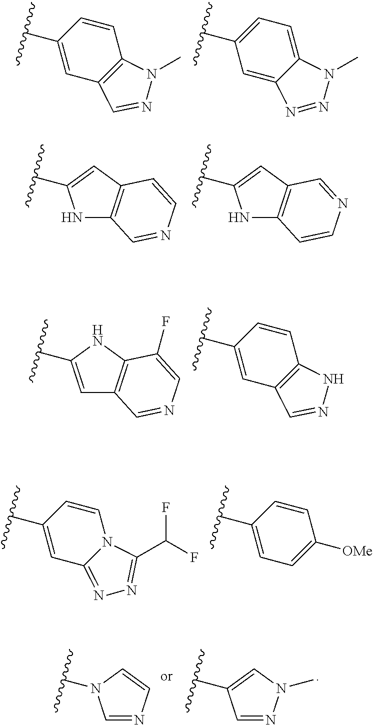

In embodiments Ring A is selected from a substituted or unsubstituted:

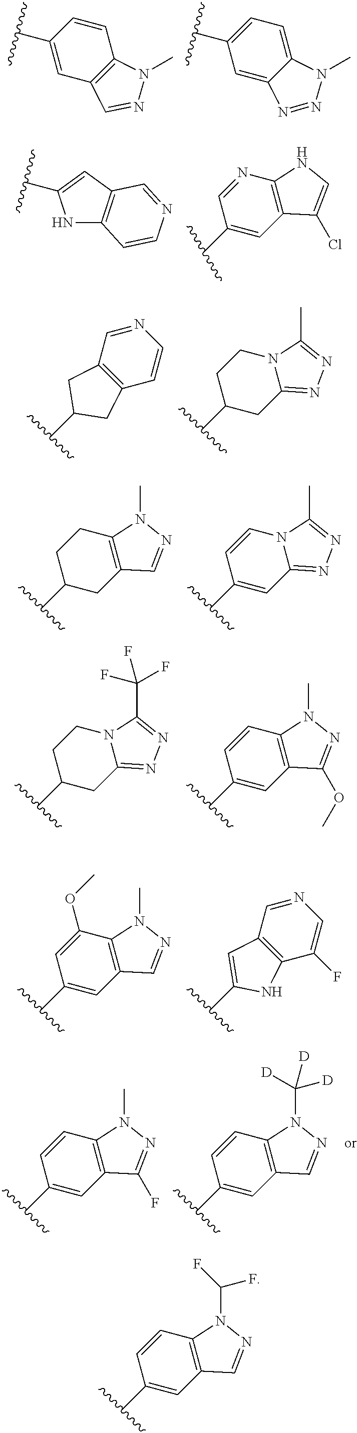

In embodiments Ring A is selected from a substituted or unsubstituted:

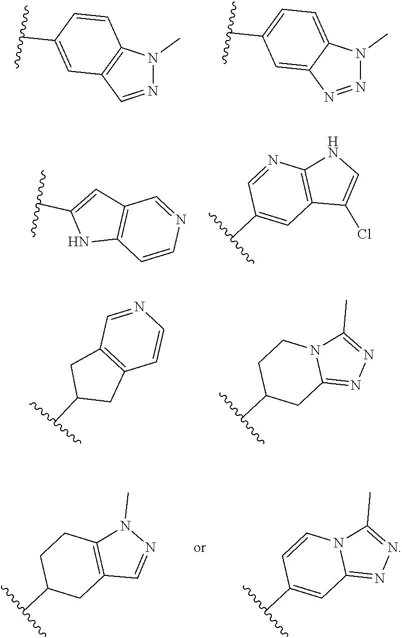

As will be evident to the skilled person, the compounds of the present invention contain a number of stereocenters. The present invention encompasses all possible stereoisomers of the present invention whether in a single stereoisomeric form or a mixture thereof.

The preferred stereochemistry of the piperidine group is as follows:

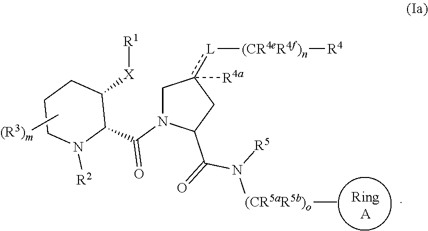

The preferred stereochemistry of the -L-(CR^{4e}R^{4f})ₙ—R⁴ group on the pyrrolidine is:

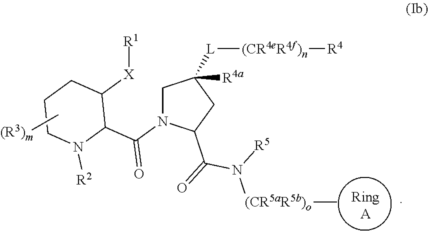

The preferred stereochemistry of the 2 position of the pyrrolidine ring is:

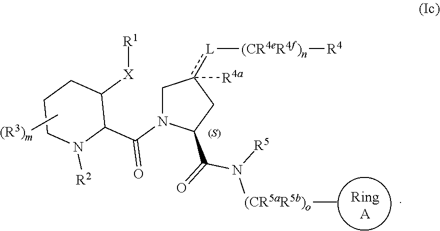

The preferred stereochemistry of the -L-(CR^{4e}R^{4f})ₙ—R⁴ group on the pyrrolidine has a trans relationship with the group at the 2 position, as depicted as follows:

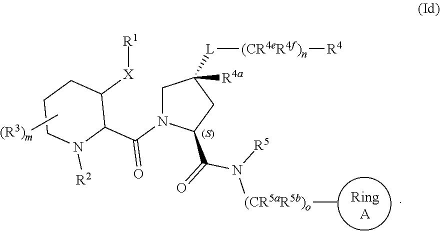

The preferred stereochemistry of the structure of formula (I) is as follows:

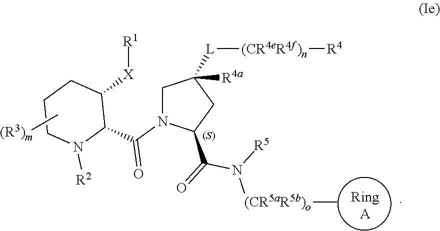

In embodiments, —X—R¹ has the structure:

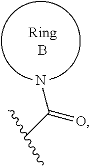 [SMILES: *C(N)=O]

wherein Ring B is a substituted or unsubstituted monocyclic or bicyclic (fused, bridged, or 5 spiro) 4 to 7 membered heterocyclic ring system comprising 0 or 1 additional heteroatoms selected from O, N or S; wherein, when substituted, the substituent of R¹ is selected from F, Me, Et, CF₃, —OMe, —OEt and —NMe₂; R² is selected from H, Me and Et; and m is 0; and

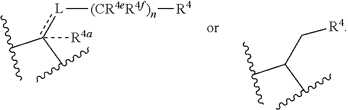

Optionally, wherein

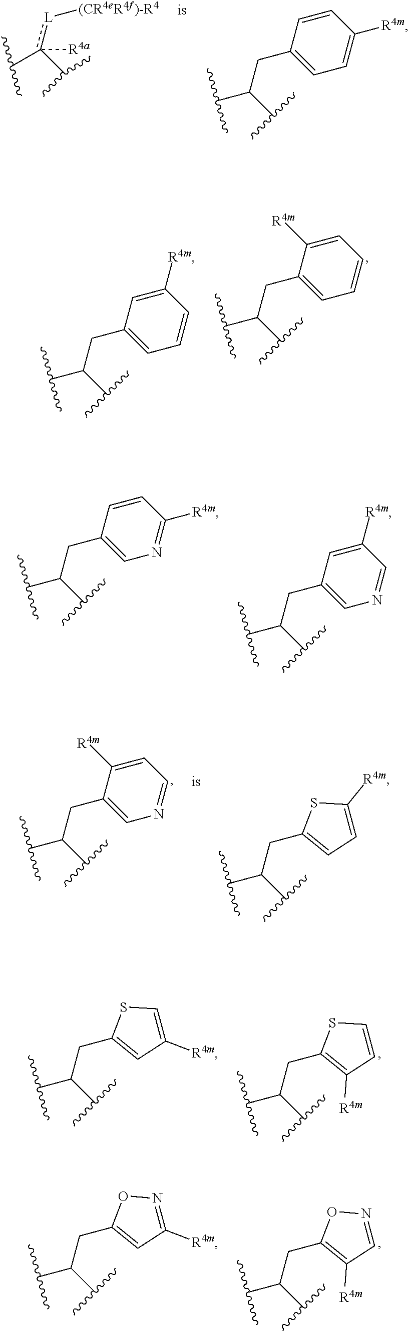

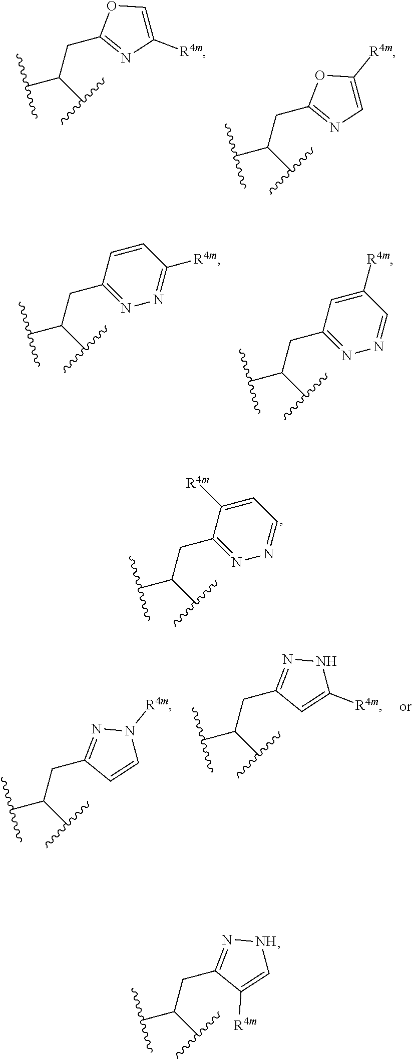

wherein R^{4m} is selected from Me, Et, Pr, F, Cl, CF₃, OMe, OCH₂F, OCHF₂, OCF₃,

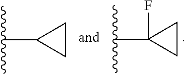

In embodiments, —X—R¹ has the structure:

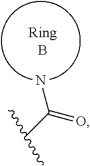 [SMILES: *C(N)=O]

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe, —OEt and —NMe₂; R² is selected from H, Me and Et; and m is 0; and

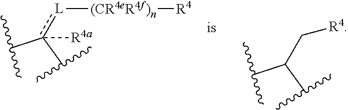

Optionally, wherein

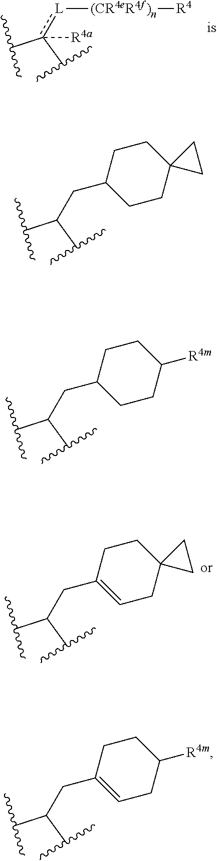

wherein R^{4m} is selected from Me, Et, Pr, F, Cl, CF₃, OMe, OCH₂F, OCHF₂, OCF₃,

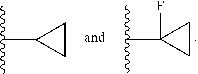

In embodiments, —X—R¹ has the structure:

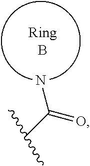 [SMILES: *C(N)=O]

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe, —OEt and —NMe₂; R² is selected from H, Me and Et; and m is 0; and

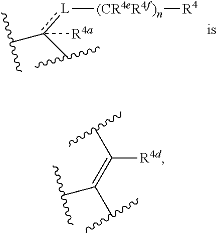

wherein R^{4d} is H. Optionally, wherein

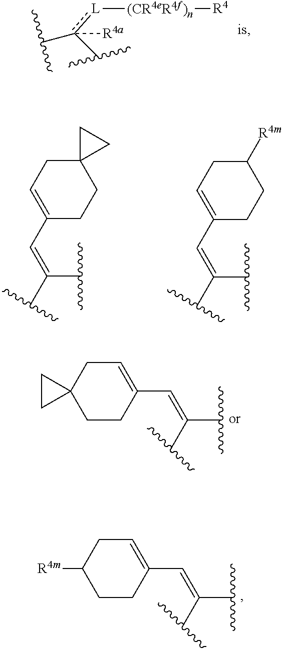

wherein R^{4m} is selected from Me, Et, Pr, F, Cl, CF₃, OMe, OCH₂F, OCHF₂, OCF₃,

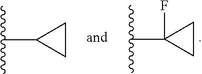

In embodiments, —X—R¹ has the structure:

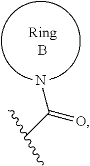 [SMILES: *C(N)=O]

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe and —OEt; R² is selected from H, Me and Et; and m is 0; and

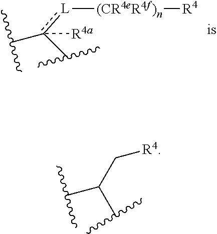

Optionally, wherein

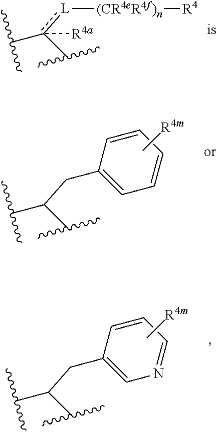

wherein R^{4m} is selected from Me, F, Cl, CF₃ and OMe. Further optionally, wherein

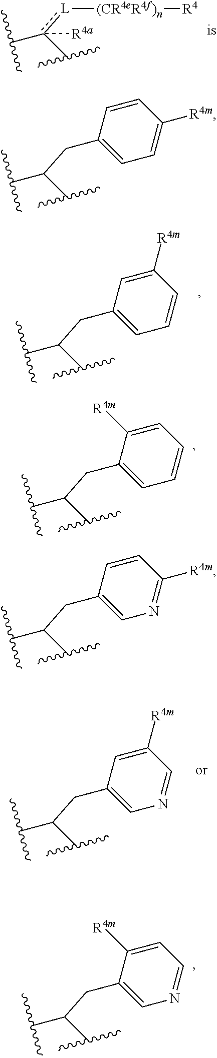

wherein R^{4m} is selected from Me, F, Cl, CF₃ and OMe.

In embodiments, —X—R¹ has the structure:

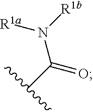

R^{1a} and R^{1b} are independently at each occurrence selected from: substituted or unsubstituted C₁₋₃ alkyl, optionally substituted with —NR^{1e}R^{1f}, and substituted or unsubstituted C₃ cycloalkyl; R² is selected from H, Me and Et; and m is 0; and

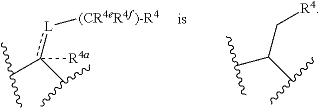

Optionally, wherein

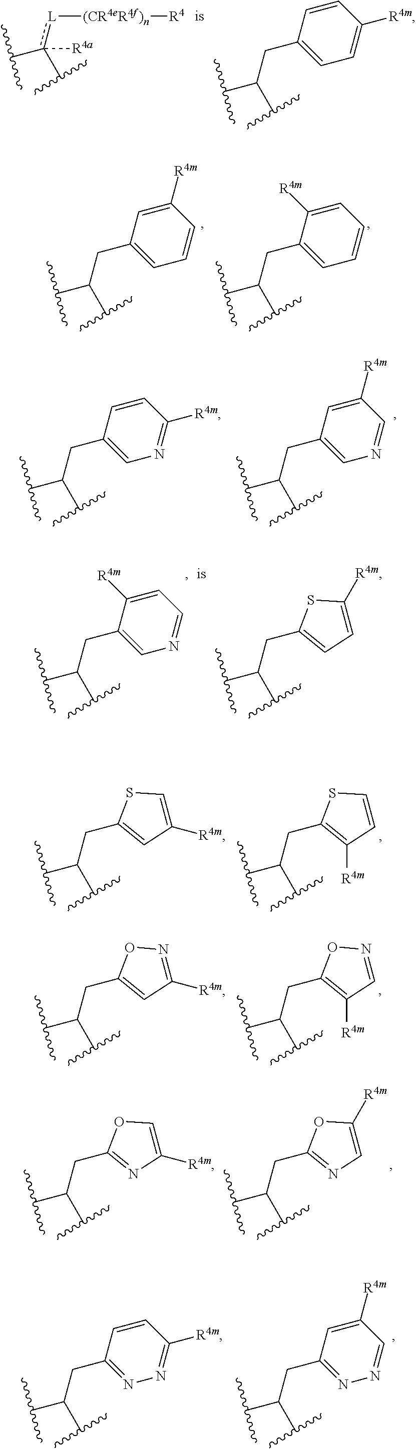

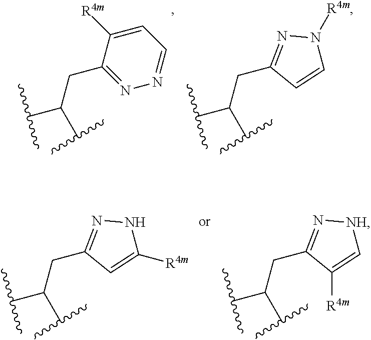

wherein R^{4m} is selected from Me, Et, Pr, F, Cl, CF₃, OMe, OCH₂F, OCHF₂, OCF₃,

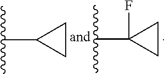

In embodiments, —X—R¹ has the structure:

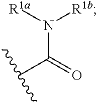

R^{1a} and R^{1b} are independently at each occurrence selected from: substituted or unsubstituted C₁₋₃ alkyl, optionally substituted with —NR^{1e}R^{1f}, and substituted or unsubstituted C₃ cycloalkyl; R² is selected from H, Me and Et; and m is 0; and

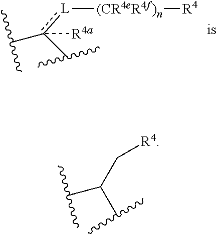

Optionally, wherein

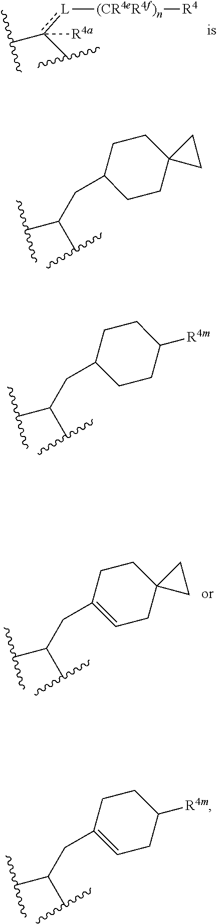

wherein R^{4m} is selected from Me, Et, Pr, F, Cl, CF₃, OMe, OCH₂F, OCHF₂, OCF₃,

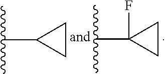

In embodiments, —X—R¹ has the structure:

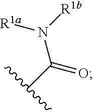

R^{1a} and R^{1b} are independently at each occurrence selected from: substituted or unsubstituted C₁₋₃ alkyl, optionally substituted with —NR^{1e}R^{1f}, and substituted or unsubstituted C₃ cycloalkyl; R² is selected from H, Me and Et; and m is 0; and

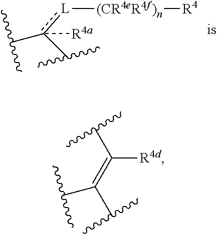

wherein R^{4d} is H. Optionally, wherein

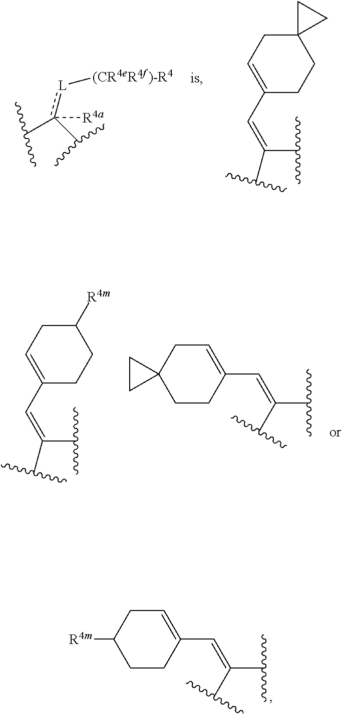

or wherein R^{4m} is selected from Me, Et, Pr, F, Cl, CF₃, OMe, OCH₂F, OCHF₂, OCF₃,

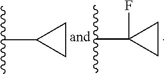

In embodiments, —X—R¹ has the structure:

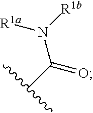

R^{1a} and R^{1b} are independently at each occurrence selected from: substituted or unsubstituted C₁₋₃ alkyl and substituted or unsubstituted C₃ cycloalkyl; R² is selected from H, Me and Et; and m is 0; and

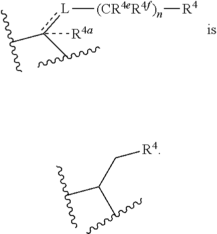

Optionally, wherein

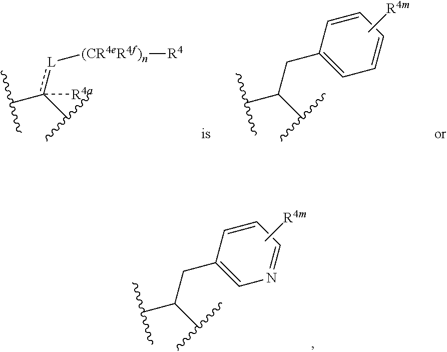

wherein R^{4m} is selected from Me, F, Cl, CF₃ and OMe. Further optionally, wherein

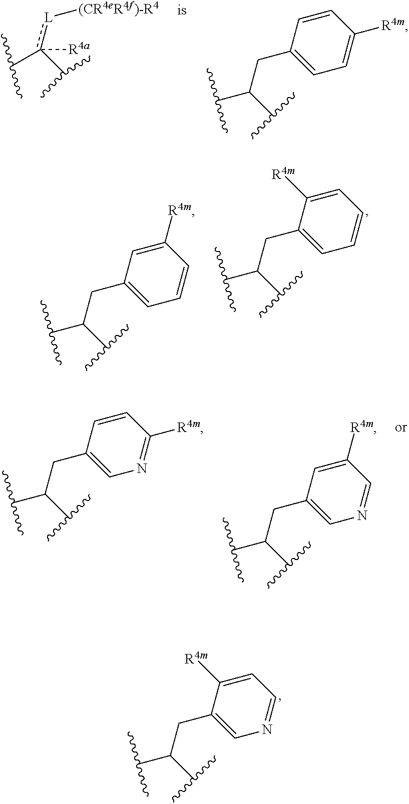

or wherein R^{4m} is selected from Me, F, Cl, CF₃ and OMe.

In embodiments, —X—R¹ has the structure:

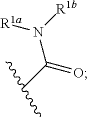

R^{1a} and R^{1b} are independently at each occurrence selected from: substituted or unsubstituted C₁₋₃ alkyl and

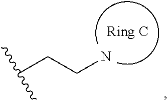 [SMILES: *CCN]

wherein Ring C is a substituted or unsubstituted monocyclic 4, 5 or 6 membered heterocyclic ring system comprising 0, 1, or 2 additional heteroatoms selected from O, N or S; R² is selected from H, Me and Et; and m is 0; and

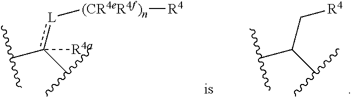

Optionally, wherein

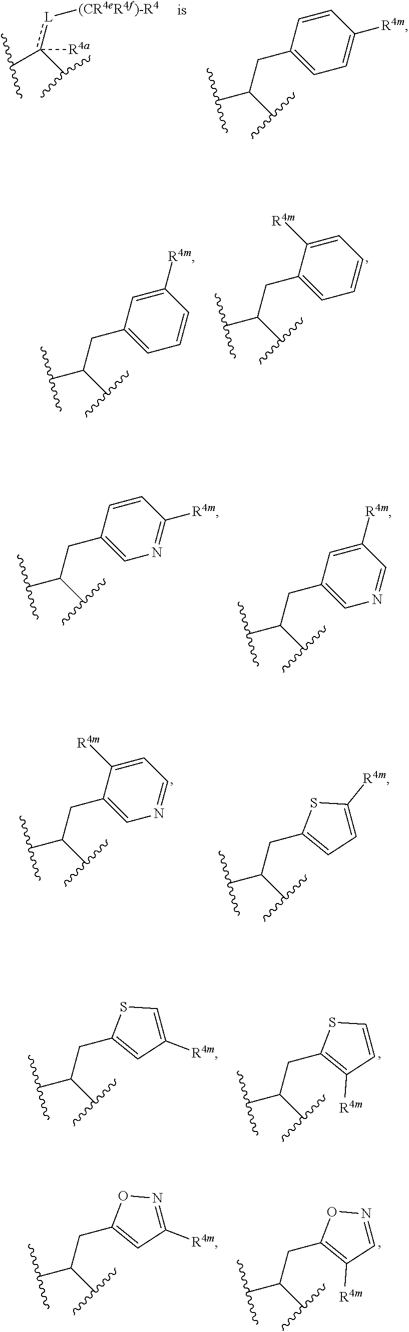

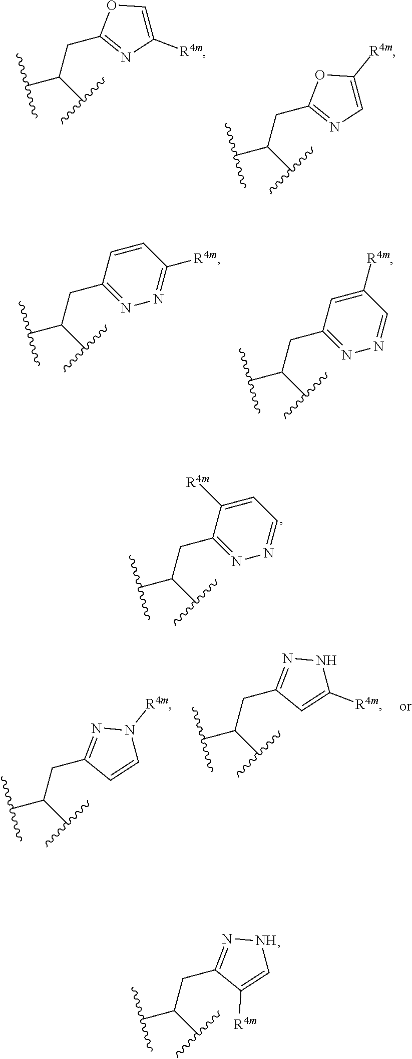

wherein R^{4m} is selected from Me, Et, Pr, F, Cl, CF₃, OMe, OCH₂F, OCHF₂, OCF₃,

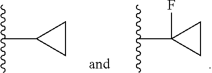

In embodiments, —X—R¹ has the structure:

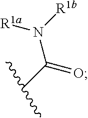

R^{1a} and R^{1b} are independently at each occurrence selected from: substituted or unsubstituted C₁₋₃ alkyl and

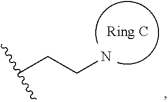 [SMILES: *CCN]

wherein Ring C is a substituted or unsubstituted monocyclic 4, 5 or 6 membered heterocyclic ring system comprising 0, 1, or 2 additional heteroatoms selected from O, N or S; R² is selected from H, Me and Et; and m is 0; and

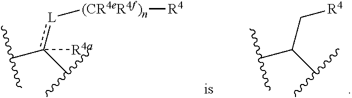

Optionally, wherein

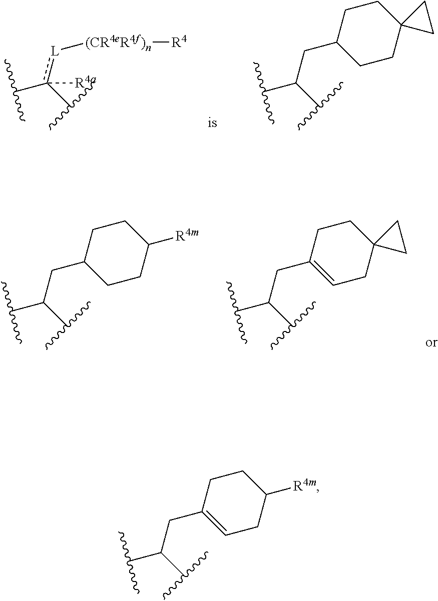

wherein R^{4m} is selected from Me, Et, Pr, F, Cl, CF₃, OMe, OCH₂F, OCHF₂, OCF₃,

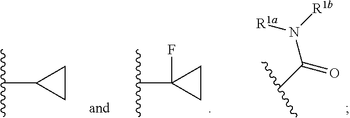

In embodiments, —X—R¹ has the structure:

R^{1a} and R^{1b} are independently at each occurrence selected from: substituted or unsubstituted C₁₋₃ alkyl and

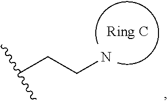 [SMILES: *CCN]

wherein Ring C is a substituted or unsubstituted monocyclic 4, 5 or 6 membered heterocyclic ring system comprising 0, 1, or 2 additional heteroatoms selected from O, N or S; R² is selected from H, Me and Et; and m is 0; and

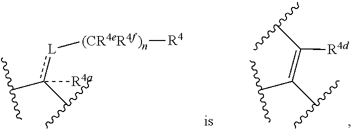

wherein R^{4d} is H. Optionally, wherein

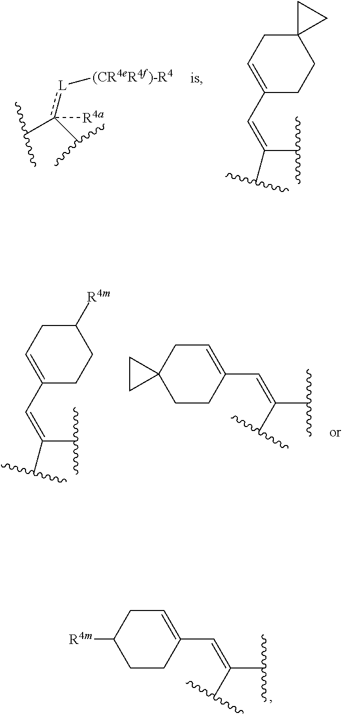

wherein R^{4m} is selected from Me, Et, Pr, F, Cl, CF₃, OMe, OCH₂F, OCHF₂, OCF₃,

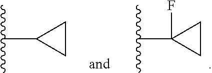

In embodiments, —X—R¹ has the structure:

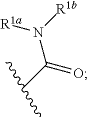

R^{1a} and R^{1b} are independently at each occurrence selected from: substituted or unsubstituted C₁₋₃ alkyl and a substituted or unsubstituted 4, 5 or 6 membered heterocyclic group having 1, 2 or 3 heteroatoms selected from O, N or S; R² is selected from H, Me and Et; and m is 0; and

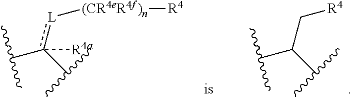

Optionally, wherein

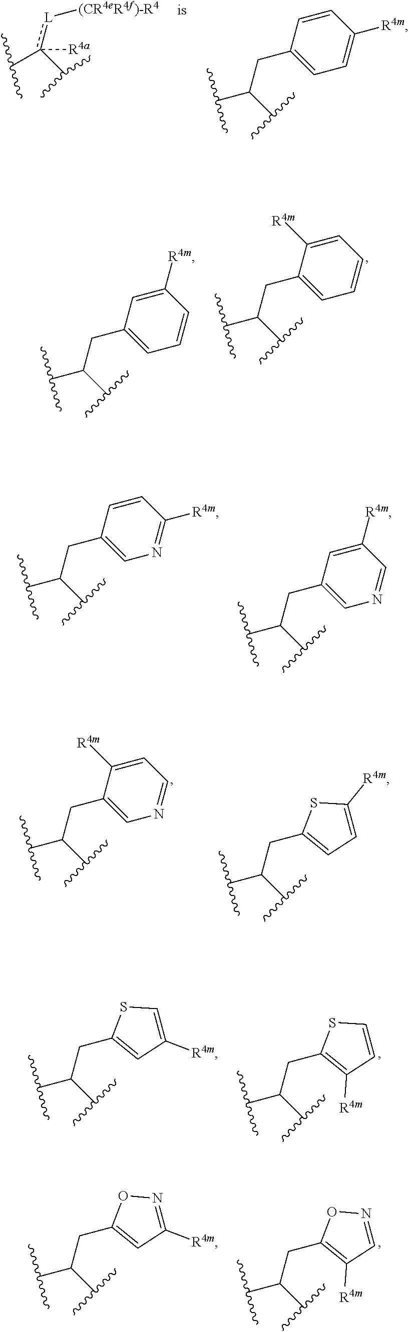

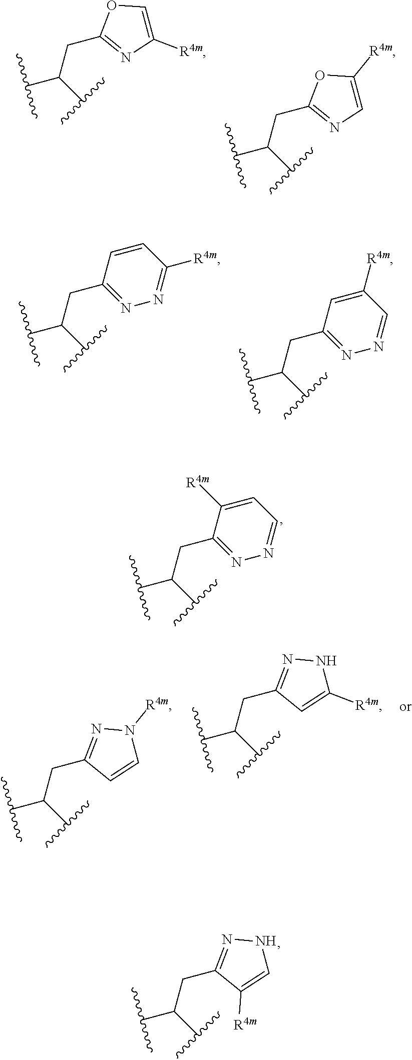

wherein R^{4m} is selected from Me, Et, Pr, F, Cl, CF₃, OMe, OCH₂F, OCHF₂, OCF₃,

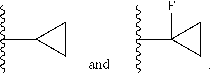

In embodiments, —X—R¹ has the structure:

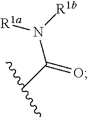

R^{1a} and R^{1b} are independently at each occurrence selected from: substituted or unsubstituted C₁₋₃ alkyl and a substituted or unsubstituted 4, 5 or 6 membered heterocyclic group having 1, 2 or 3 heteroatoms selected from O, N or S; R² is selected from H, Me and Et; and m is 0; and

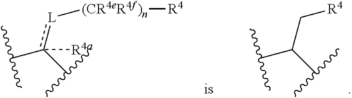

Optionally, wherein

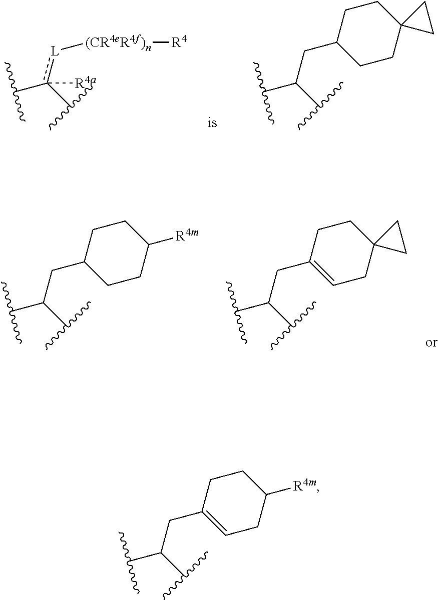

wherein R^{4m} is selected from Me, Et, Pr, F, Cl, CF₃, OMe, OCH₂F, OCHF₂, OCF₃, and

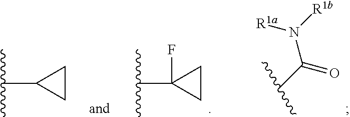

In embodiments, —X—R¹ has the structure:

R^{1a} and R^{1b} are independently at each occurrence selected from: substituted or unsubstituted C₁₋₃ alkyl and a substituted or unsubstituted 4, 5 or 6 membered heterocyclic group having 1, 2 or 3 heteroatoms selected from O, N or S; R² is selected from H, Me and Et; and m is 0; and

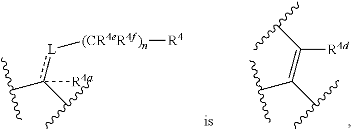

wherein R^{4d} is H. Optionally, wherein

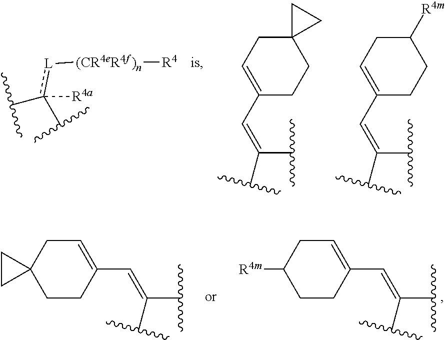

wherein R^{4m} is selected from Me, Et, Pr, F, Cl, CF₃, OMe, OCH₂F, OCHF₂, OCF₃,

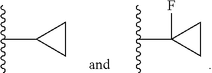

In embodiments, —X—R¹ has the structure:

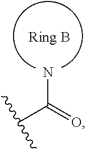

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe, —OEt and —NMe₂; R² is selected from H, Me and Et; and m is 0; and

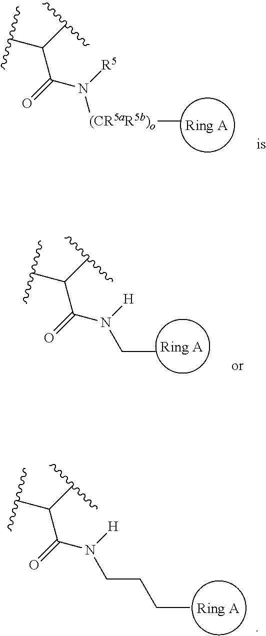

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Or optionally Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.

In embodiments, —X—R¹ has the structure:

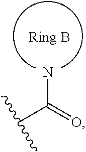

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe and —OEt; R² is selected from H, Me and Et; and m is 0; and

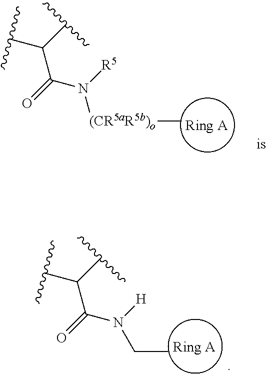

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.

In embodiments, —X—R¹ has the structure:

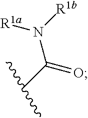

R^{1a} and R^{1b} are independently at each occurrence selected from: substituted or unsubstituted C₁₋₃ alkyl, optionally substituted with —NR^{1e}R^{1f}, and substituted or unsubstituted C₃ cycloalkyl; R² is selected from H, Me and Et; and m is 0; and

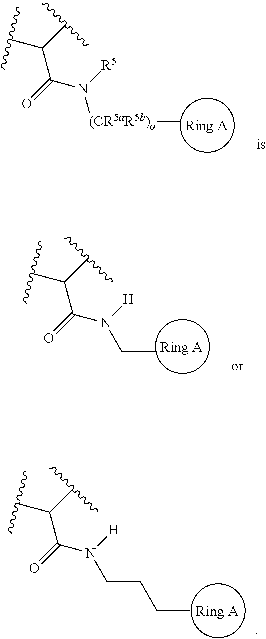

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Or optionally Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: 15 halo, —CN, C₁₋₆ alkyl, C₁₋₆ haloalkyl, C₃₋₆ cycloalkyl, deuterated C₁₋₆ alkyl, —OR⁵⁰, —NR^{5c}R^{5d} or C₁₋₄ alkyl substituted by —NR^{5c}R^{5d}.ln embodiments, —X—R¹ has the structure:

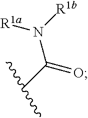

R^{1a} and R^{1b} are independently at each occurrence selected from: substituted or unsubstituted C₁₋₃ alkyl and substituted or unsubstituted C₃ cycloalkyl; R² is selected from H, Me and Et; and m is 0; and

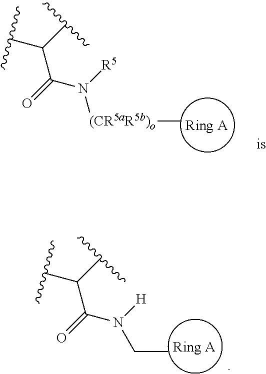

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR⁵⁰, —NR^{5c}R^{5d} or C₁₋₄ alkyl substituted by —NR^{5c}R^{5d}.

In embodiments, —X—R¹ has the structure:

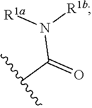

R^{1a} and R^{1b} are independently at each occurrence selected from: substituted or unsubstituted C₁₋₃ alkyl and a substituted or unsubstituted 4, 5 or 6 membered heterocyclic group having 1, 2 or 3 heteroatoms selected from O, N or S; R² is selected from H, Me and Et; and m is 0;

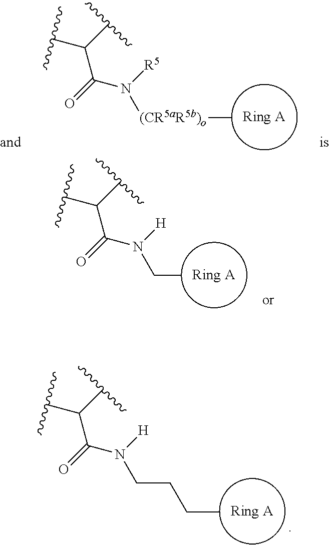

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Or optionally Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.

In embodiments,

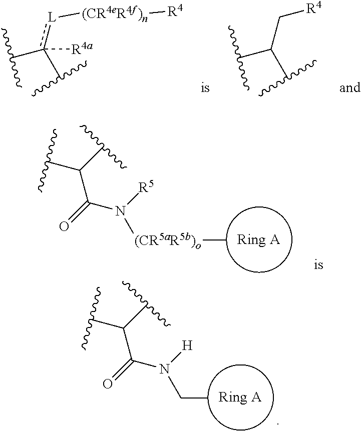

Optionally, wherein

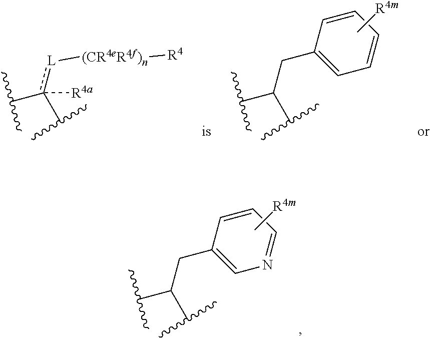

wherein R^{4m} is selected from Me, F, Cl, CF₃ and OMe. Further optionally, wherein

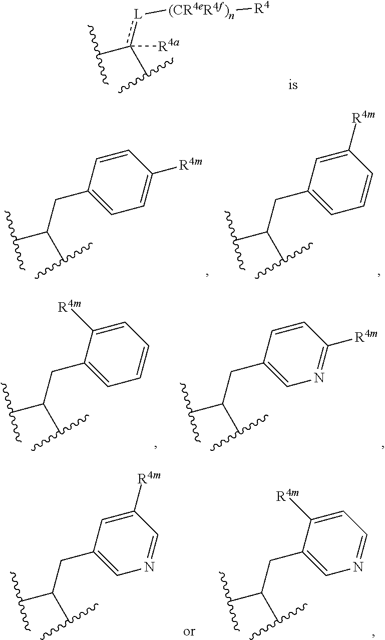

wherein R^{4m} is selected from Me, F, Cl, CF₃ and OMe. Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5C}, —NR^{5c}R^{5d} or C₁₋₄ alkyl substituted by —NR^{5c}R^{5d}.

In embodiments,

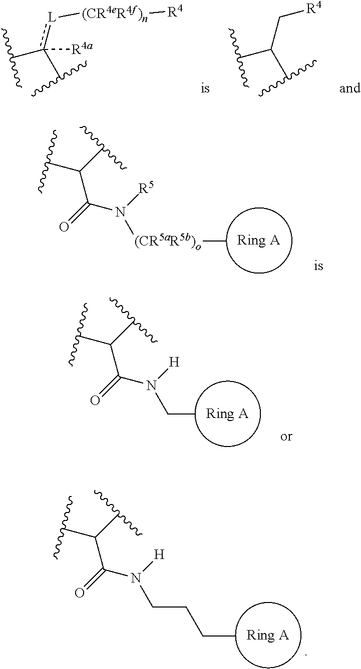

Optionally, wherein is

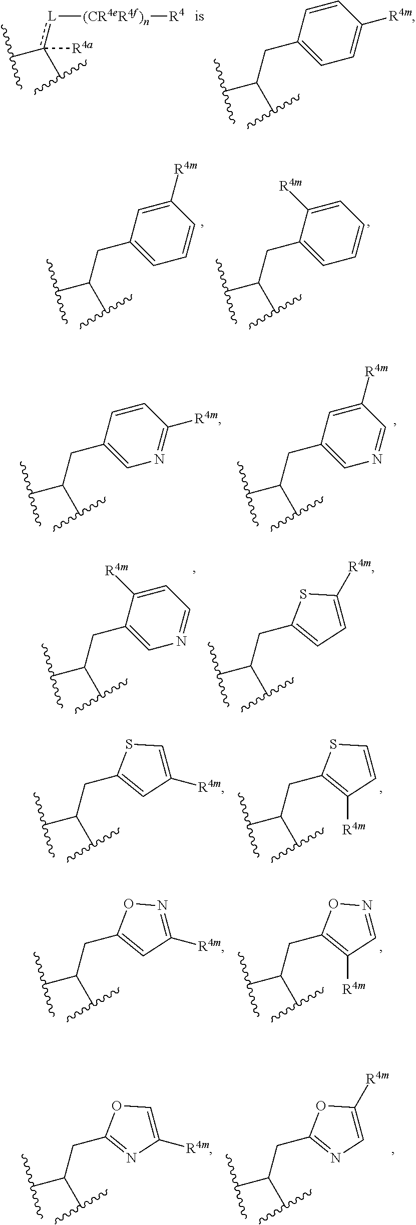

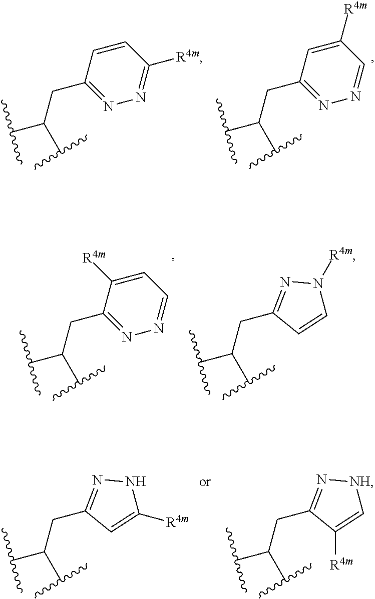

wherein R^{4m} is selected from Me, Et, Pr, F, Cl, CF₃, OMe, OCH₂F, OCHF₂, OCF₃, and

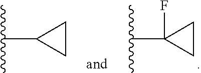

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR⁵⁰, —NR^{5c}R^{5d} or C₁₋₄ alkyl substituted by —NR^{5c}R^{5d}. Or optionally Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.

In embodiments,

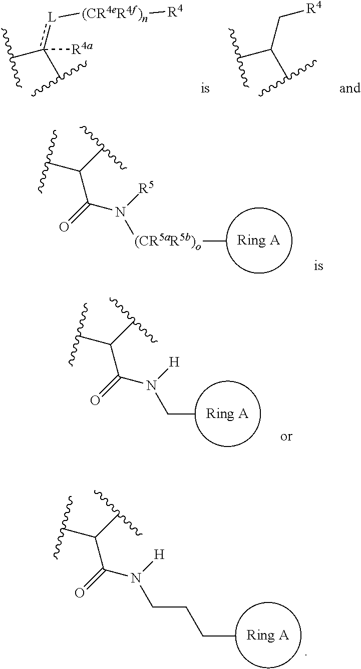

Optionally, wherein

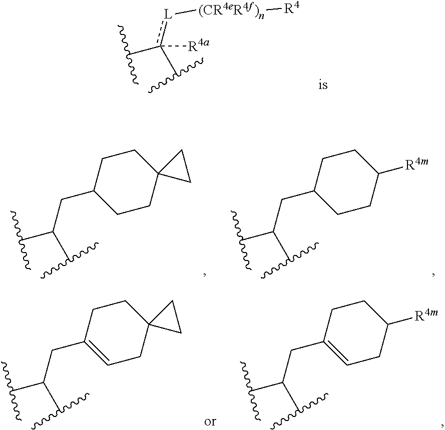

wherein R^{4m} is selected from Me, Et, Pr, F, Cl, CF₃, OMe, OCH₂F, OCHF₂, OCF₃,

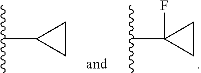

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Or optionally Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.

In embodiments,

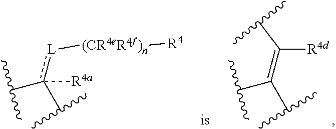

wherein R^{4d} is H; and

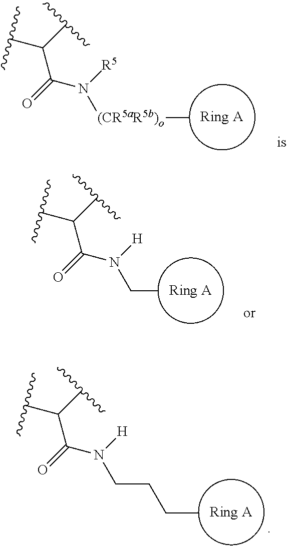

Optionally, wherein

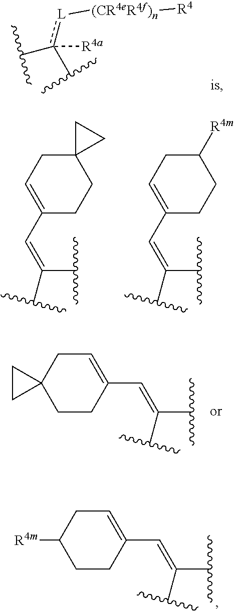

wherein R^{4m} is selected from Me, Et, Pr, F, Cl, CF₃, OMe, OCH₂F, OCHF₂, OCF₃,

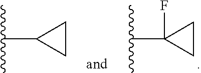

Optionally, Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}. Or optionally Ring A is selected from a substituted or unsubstituted 5 to 6 membered monocyclic heteroaryl group having 1, 2 or 3 heteroatoms selected from O, N or S, a substituted or unsubstituted 6 membered monocyclic aryl group, wherein, when substituted, the monocyclic heteroaryl group or the monocyclic aryl group are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.

In embodiments, —X—R¹ has the structure:

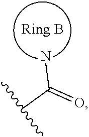 [SMILES: *C(N)=O]

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe and —OEt; R² is selected from H, Me and Et; and m is 0;

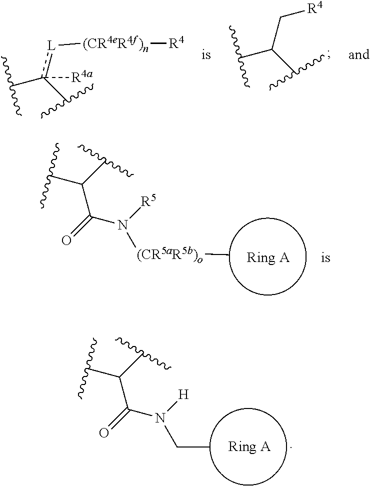

In embodiments, —X—R¹ has the structure:

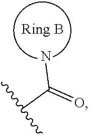 [SMILES: *C(N)=O]

wherein Ring B is a substituted or unsubstituted monocyclic or bicyclic (fused, bridged, or spiro) 4 to 7 membered heterocyclic ring system comprising 0 or 1 additional heteroatoms selected from O, N or S; wherein, when substituted, the substituent of R¹ is selected from F, Me, Et, CF₃, —OMe, —OEt and —NMe₂; R² is selected from H, Me and Et; and m is 0;

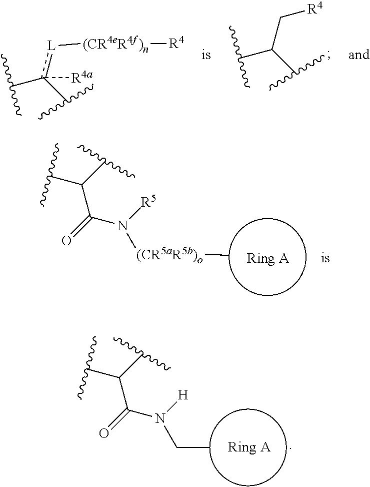

In embodiments, —X—R¹ has the structure:

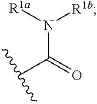

R^{1a} and R^{1b} are independently at each occurrence selected from: substituted or unsubstituted C₁₋₃ alkyl and substituted or unsubstituted C₃ cycloalkyl; R² is selected from H, Me and Et; and m is 0;

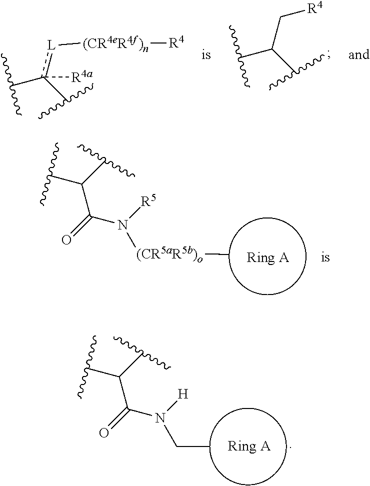

In embodiments, —X—R¹ has the structure:

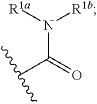

R^{1a} and R^{1b} are independently at each occurrence selected from: substituted or unsubstituted C₁₋₃ alkyl, optionally substituted with —NR^{1e}R^{1f}, and substituted or unsubstituted C₃ cycloalkyl; R² is selected from H, Me and Et; and m is 0;

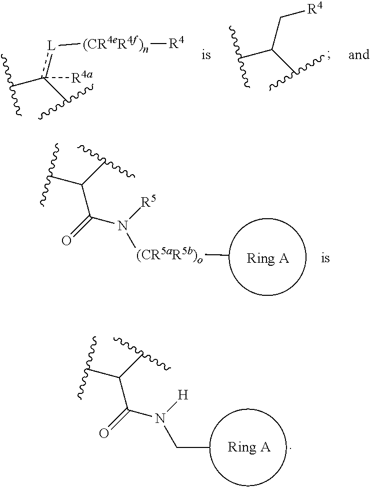

In embodiments, —X—R¹ has the structure:

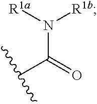

R^{1a} and R^{1b} are independently at each occurrence selected from: substituted or unsubstituted C₁₋₃ alkyl and a substituted or unsubstituted 4, 5 or 6 membered heterocyclic group having 1, 2 or 3 heteroatoms selected from O, N or S; R² is selected from H, Me and Et; and m is 0;

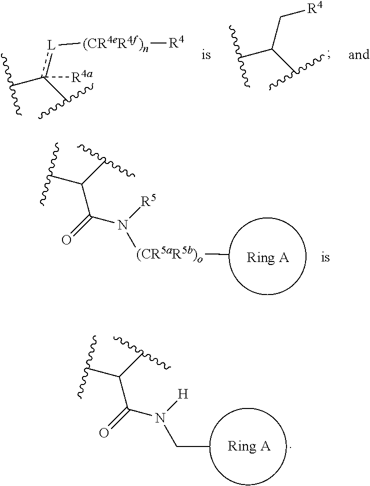

In embodiments, —X—R¹ has the structure:

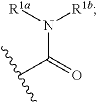

R^{1a} and R^{1b} are independently at each occurrence selected from: substituted or unsubstituted C₁₋₃ alkyl and

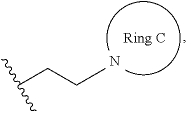

wherein Ring C is a substituted or unsubstituted monocyclic 4, 5 or 6 membered heterocyclic ring system comprising 0, 1, or 2 additional heteroatoms selected from O, N or S; R² is selected from H, Me and Et; and m is 0;

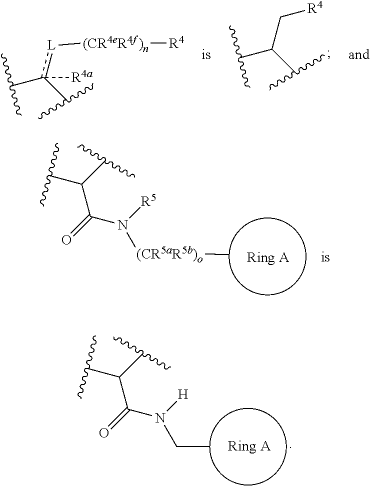

In embodiments the compounds of formula (I) are selected from:

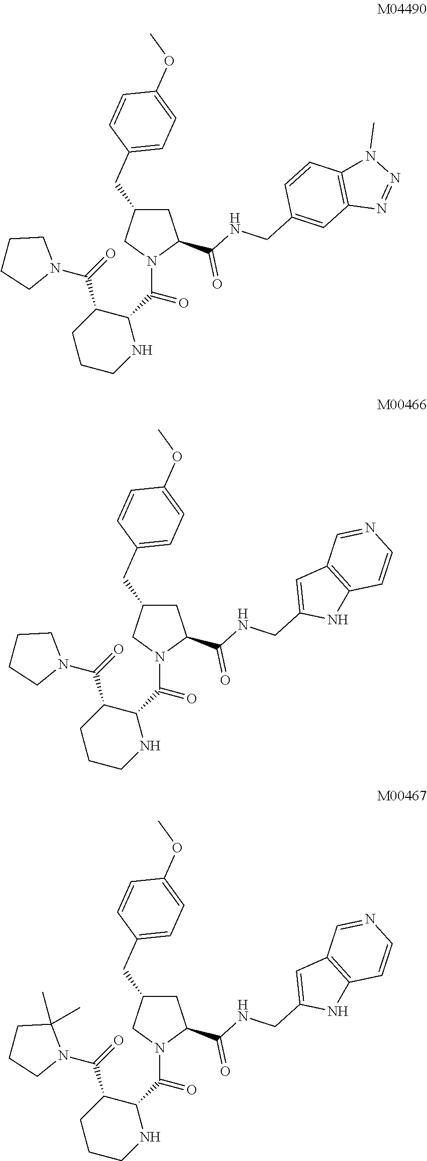 [SMILES: COc1ccc(C[C@@H]2C[C@@H](C(=O)NCc3ccc4c(c3)nnn4C)N(C(=O)[C@@H]3NCCC[C@@H]3C(=O)N3CCCC3)C2)cc1 ; COc1ccc(C[C@@H]2C[C@@H](C(=O)NCc3cc4cnccc4[nH]3)N(C(=O)[C@@H]3NCCC[C@@H]3C(=O)N3CCCC3)C2)cc1 ; COc1ccc(C[C@@H]2C[C@@H](C(=O)NCc3cc4cnccc4[nH]3)N(C(=O)[C@@H]3NCCC[C@@H]3C(=O)N3CCCC3(C)C)C2)cc1]

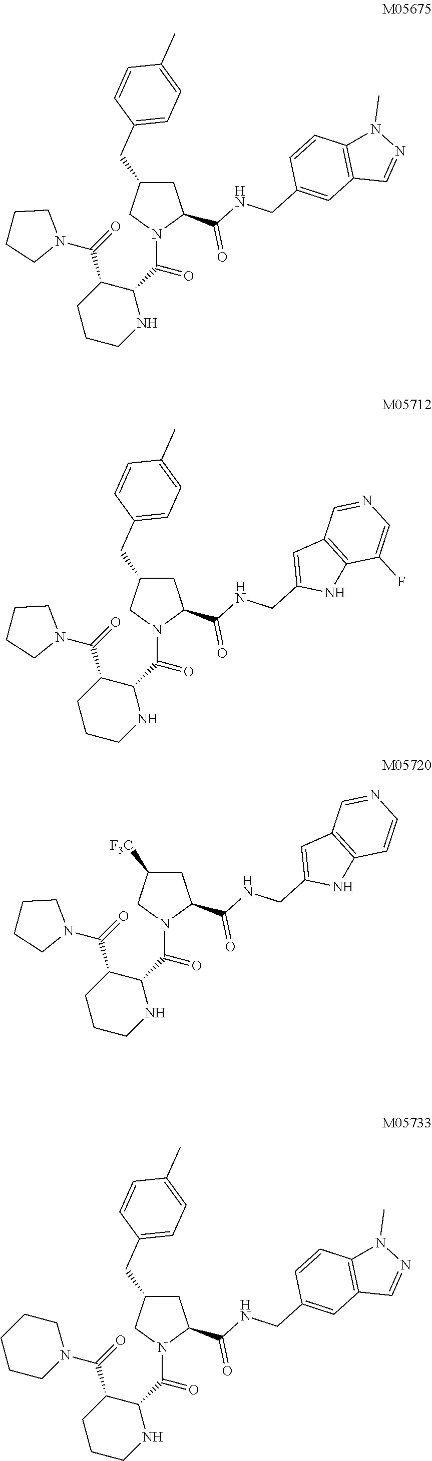 [SMILES: Cc1ccc(C[C@@H]2C[C@@H](C(=O)NCc3ccc4c(cnn4C)c3)N(C(=O)[C@@H]3NCCC[C@@H]3C(=O)N3CCCC3)C2)cc1 ; Cc1ccc(C[C@@H]2C[C@@H](C(=O)NCc3cc4cncc(F)c4[nH]3)N(C(=O)[C@@H]3NCCC[C@@H]3C(=O)N3CCCC3)C2)cc1 ; O=C(NCc1cc2cnccc2[nH]1)[C@@H]1C[C@H](C(F)(F)F)CN1C(=O)[C@@H]1NCCC[C@@H]1C(=O)N1CCCC1 ; Cc1ccc(C[C@@H]2C[C@@H](C(=O)NCc3ccc4c(cnn4C)c3)N(C(=O)[C@@H]3NCCC[C@@H]3C(=O)N3CCCCC3)C2)cc1]

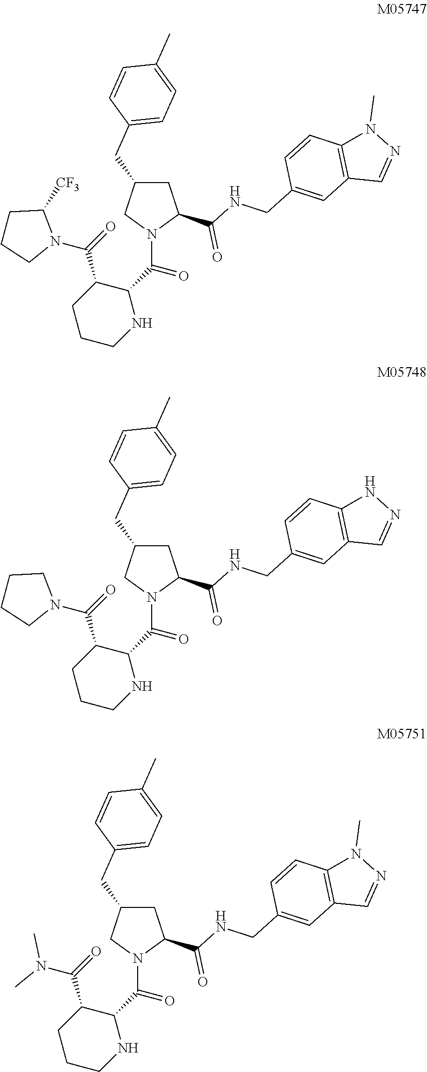 [SMILES: Cc1ccc(C[C@@H]2C[C@@H](C(=O)NCc3ccc4c(cnn4C)c3)N(C(=O)[C@@H]3NCCC[C@@H]3C(=O)N3CCC[C@@H]3C(F)(F)F)C2)cc1 ; Cc1ccc(C[C@@H]2C[C@@H](C(=O)NCc3ccc4[nH]ncc4c3)N(C(=O)[C@@H]3NCCC[C@@H]3C(=O)N3CCCC3)C2)cc1 ; Cc1ccc(C[C@@H]2C[C@@H](C(=O)NCc3ccc4c(cnn4C)c3)N(C(=O)[C@@H]3NCCC[C@@H]3C(=O)N(C)C)C2)cc1]

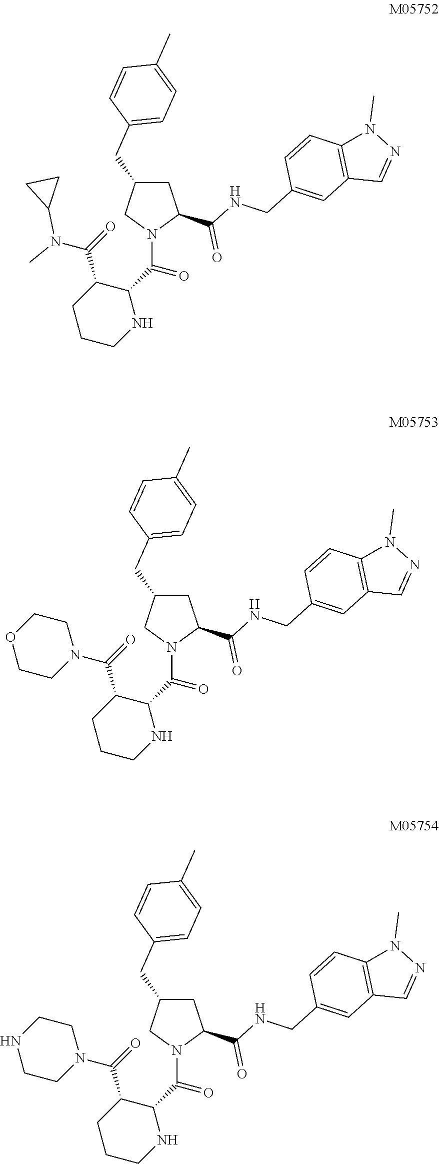 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CC3)C2)cc1 ; Cc1ccc(C[C@@H]2C[C@@H](C(=O)NCc3ccc4c(cnn4C)c3)N(C(=O)[C@@H]3NCCC[C@@H]3C(=O)N3CCOCC3)C2)cc1 ; Cc1ccc(C[C@@H]2C[C@@H](C(=O)NCc3ccc4c(cnn4C)c3)N(C(=O)[C@@H]3NCCC[C@@H]3C(=O)N3CCNCC3)C2)cc1]

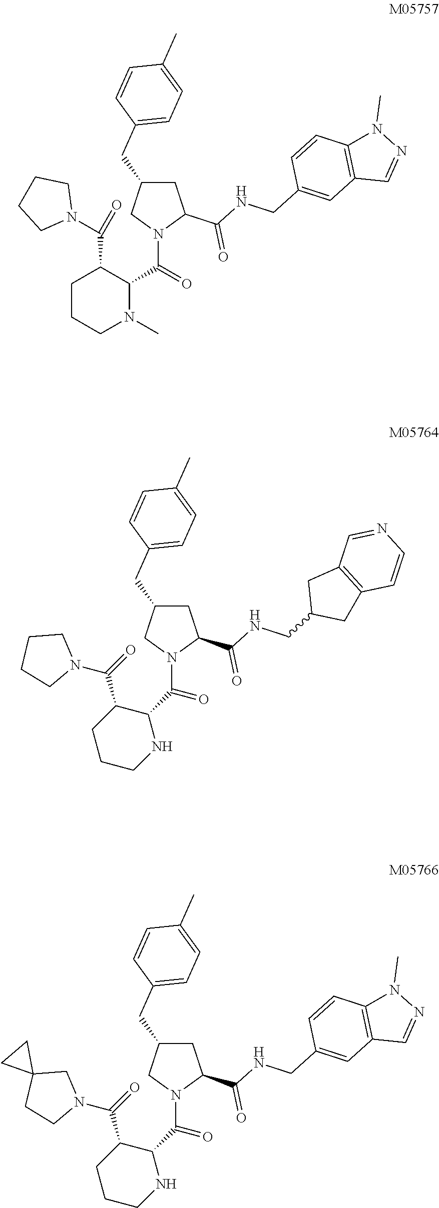 [SMILES: Cc1ccc(C[C@@H]2CC(C(=O)NCc3ccc4c(cnn4C)c3)N(C(=O)[C@H]3[C@@H](C(=O)N4CCCC4)CCCN3C)C2)cc1 ; Cc1ccc(C[C@@H]2C[C@@H](C(=O)NCC3Cc4ccncc4C3)N(C(=O)[C@@H]3NCCC[C@@H]3C(=O)N3CCCC3)C2)cc1 ; Cc1ccc(C[C@@H]2C[C@@H](C(=O)NCc3ccc4c(cnn4C)c3)N(C(=O)[C@@H]3NCCC[C@@H]3C(=O)N3CCC4(CC4)C3)C2)cc1]

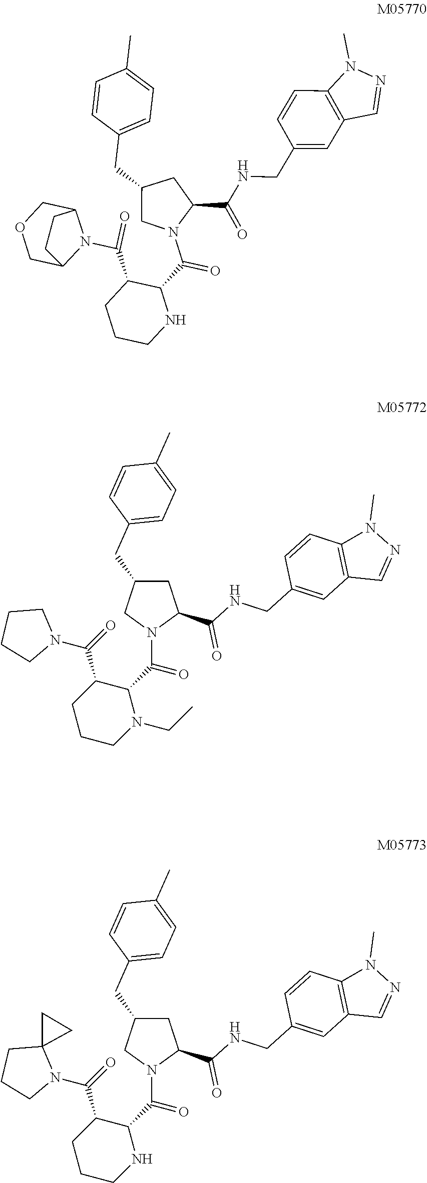 [SMILES: Cc1ccc(C[C@@H]2C[C@@H](C(=O)NCc3ccc4c(cnn4C)c3)N(C(=O)[C@@H]3NCCC[C@@H]3C(=O)N3C4CCC3COC4)C2)cc1 ; CCN1CCC[C@H](C(=O)N2CCCC2)[C@@H]1C(=O)N1C[C@H](Cc2ccc(C)cc2)C[C@H]1C(=O)NCc1ccc2c(cnn2C)c1 ; Cc1ccc(C[C@@H]2C[C@@H](C(=O)NCc3ccc4c(cnn4C)c3)N(C(=O)[C@@H]3NCCC[C@@H]3C(=O)N3CCCC34CC4)C2)cc1]

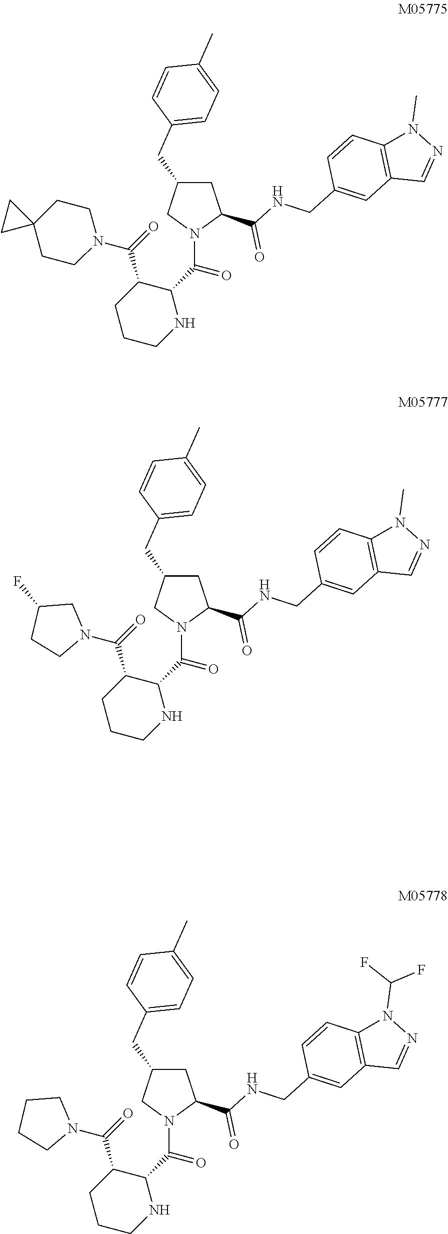 [SMILES: Cc1ccc(C[C@@H]2C[C@@H](C(=O)NCc3ccc4c(cnn4C)c3)N(C(=O)[C@@H]3NCCC[C@@H]3C(=O)N3CCC4(CC3)CC4)C2)cc1 ; Cc1ccc(C[C@@H]2C[C@@H](C(=O)NCc3ccc4c(cnn4C)c3)N(C(=O)[C@@H]3NCCC[C@@H]3C(=O)N3CC[C@H](F)C3)C2)cc1 ; Cc1ccc(C[C@@H]2C[C@@H](C(=O)NCc3ccc4c(cnn4C(F)F)c3)N(C(=O)[C@@H]3NCCC[C@@H]3C(=O)N3CCCC3)C2)cc1]

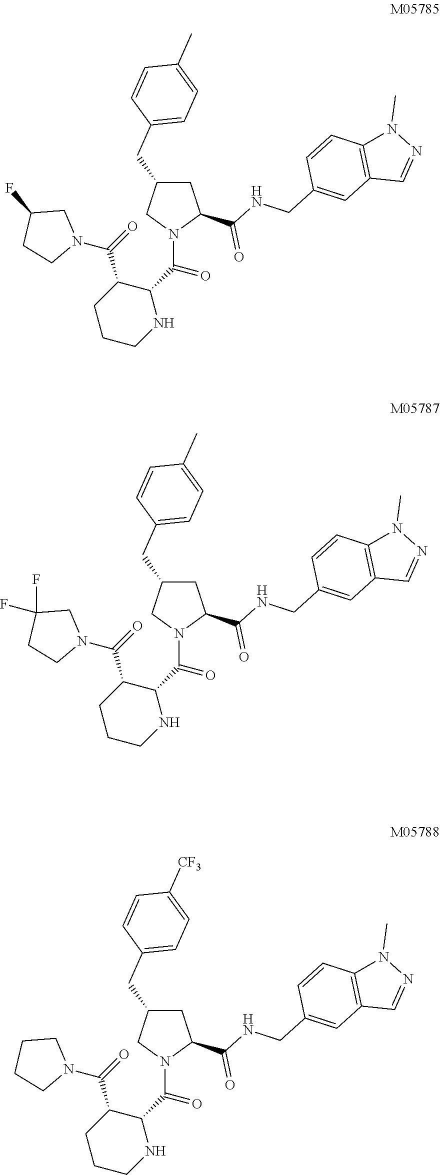 [SMILES: Cc1ccc(C[C@@H]2C[C@@H](C(=O)NCc3ccc4c(cnn4C)c3)N(C(=O)[C@@H]3NCCC[C@@H]3C(=O)N3CC[C@@H](F)C3)C2)cc1 ; Cc1ccc(C[C@@H]2C[C@@H](C(=O)NCc3ccc4c(cnn4C)c3)N(C(=O)[C@@H]3NCCC[C@@H]3C(=O)N3CCC(F)(F)C3)C2)cc1 ; Cn1ncc2cc(CNC(=O)[C@@H]3C[C@@H](Cc4ccc(C(F)(F)F)cc4)CN3C(=O)[C@@H]3NCCC[C@@H]3C(=O)N3CCCC3)ccc21]

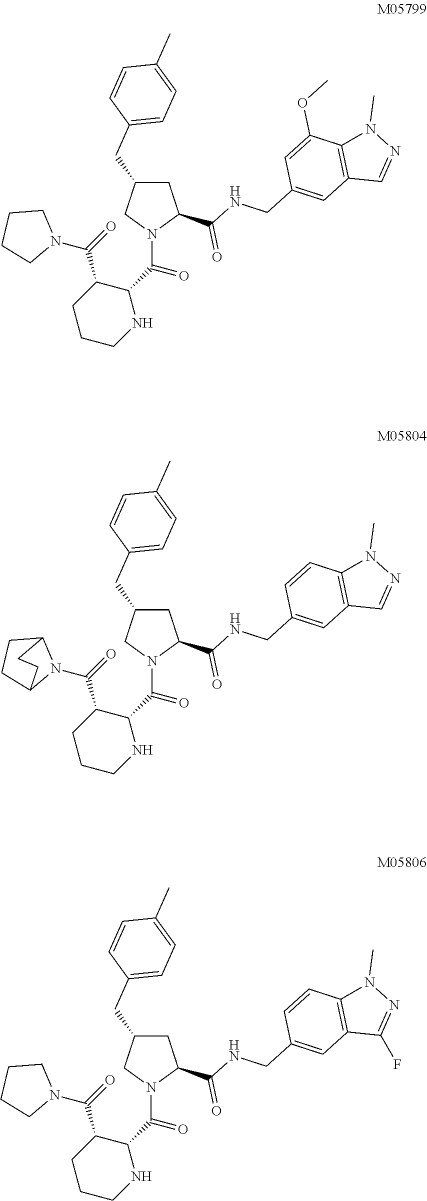 [SMILES: COc1cc(CNC(=O)[C@@H]2C[C@@H](Cc3ccc(C)cc3)CN2C(=O)[C@@H]2NCCC[C@@H]2C(=O)N2CCCC2)cc2cnn(C)c12 ; Cc1ccc(C[C@@H]2C[C@@H](C(=O)NCc3ccc4c(cnn4C)c3)N(C(=O)[C@@H]3NCCC[C@@H]3C(=O)N3C4CCC3CC4)C2)cc1 ; Cc1ccc(C[C@@H]2C[C@@H](C(=O)NCc3ccc4c(c3)c(F)nn4C)N(C(=O)[C@@H]3NCCC[C@@H]3C(=O)N3CCCC3)C2)cc1]

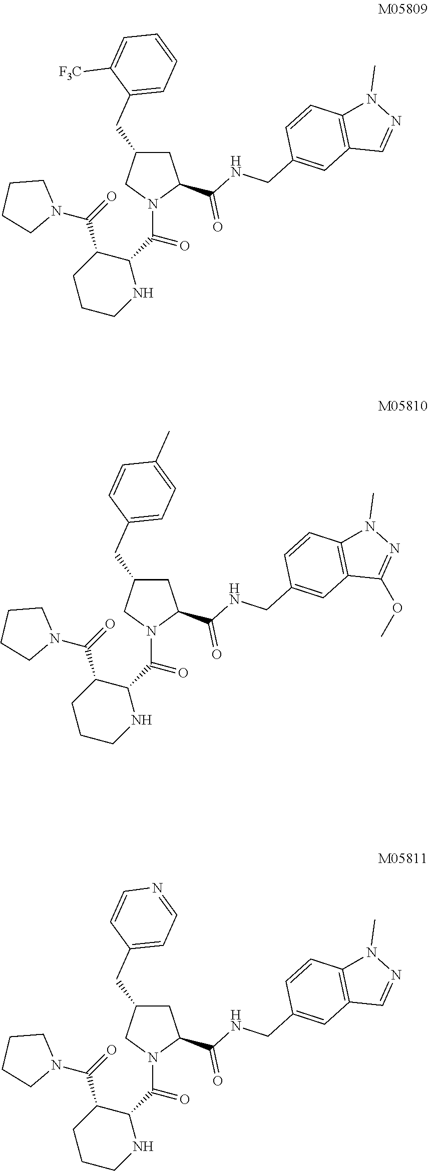 [SMILES: Cn1ncc2cc(CNC(=O)[C@@H]3C[C@@H](Cc4ccccc4C(F)(F)F)CN3C(=O)[C@@H]3NCCC[C@@H]3C(=O)N3CCCC3)ccc21 ; COc1nn(C)c2ccc(CNC(=O)[C@@H]3C[C@@H](Cc4ccc(C)cc4)CN3C(=O)[C@@H]3NCCC[C@@H]3C(=O)N3CCCC3)cc12 ; Cn1ncc2cc(CNC(=O)[C@@H]3C[C@@H](Cc4ccncc4)CN3C(=O)[C@@H]3NCCC[C@@H]3C(=O)N3CCCC3)ccc21]

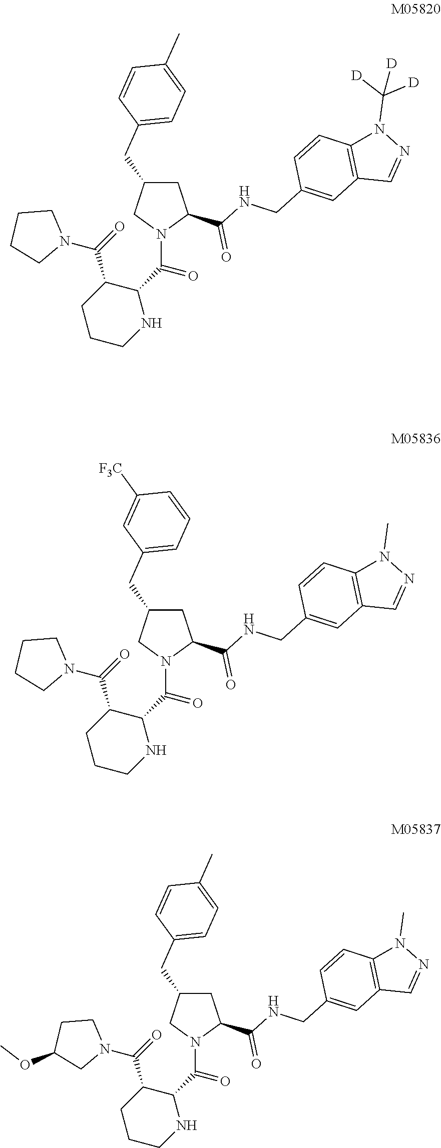 [SMILES: [2H]C([2H])([2H])n1ncc2cc(CNC(=O)[C@@H]3C[C@@H](Cc4ccc(C)cc4)CN3C(=O)[C@@H]3NCCC[C@@H]3C(=O)N3CCCC3)ccc21 ; Cn1ncc2cc(CNC(=O)[C@@H]3C[C@@H](Cc4cccc(C(F)(F)F)c4)CN3C(=O)[C@@H]3NCCC[C@@H]3C(=O)N3CCCC3)ccc21 ; CO[C@H]1CCN(C(=O)[C@H]2CCCN[C@H]2C(=O)N2C[C@H](Cc3ccc(C)cc3)C[C@H]2C(=O)NCc2ccc3c(cnn3C)c2)C1]

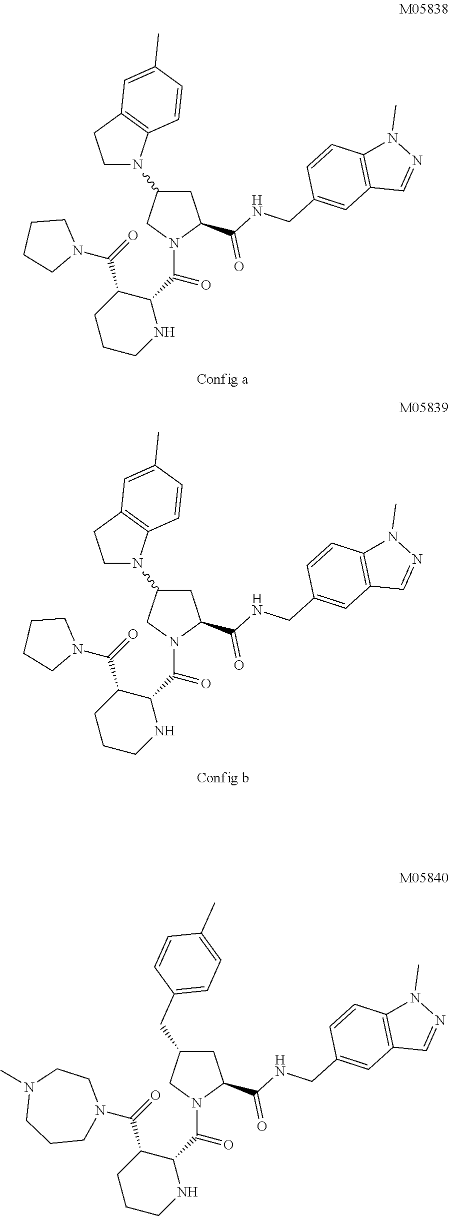 [SMILES: Cc1ccc2c(c1)CCN2C1C[C@@H](C(=O)NCc2ccc3c(cnn3C)c2)N(C(=O)[C@@H]2NCCC[C@@H]2C(=O)N2CCCC2)C1 ; Cc1ccc2c(c1)CCN2C1C[C@@H](C(=O)NCc2ccc3c(cnn3C)c2)N(C(=O)[C@@H]2NCCC[C@@H]2C(=O)N2CCCC2)C1 ; Cc1ccc(C[C@@H]2C[C@@H](C(=O)NCc3ccc4c(cnn4C)c3)N(C(=O)[C@@H]3NCCC[C@@H]3C(=O)N3CCCN(C)CC3)C2)cc1]

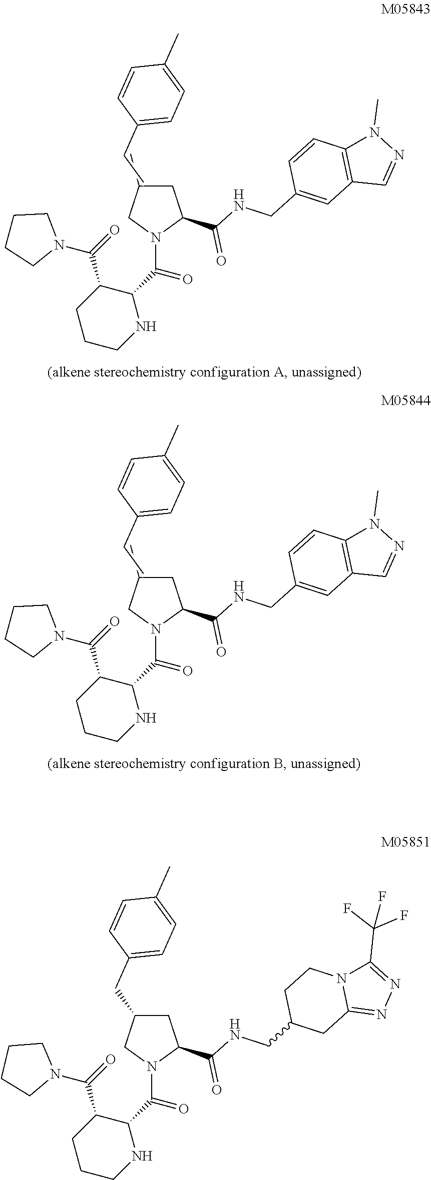

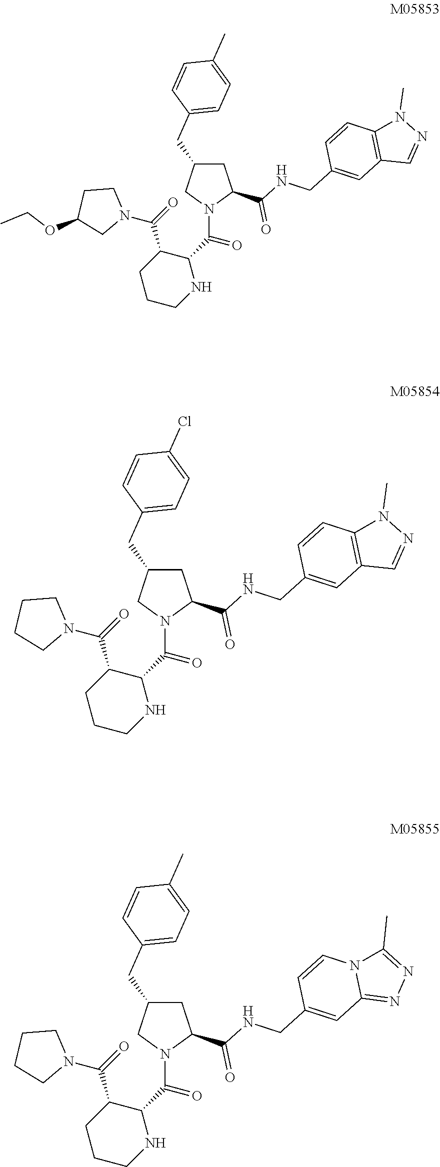 [SMILES: CCO[C@H]1CCN(C(=O)[C@H]2CCCN[C@H]2C(=O)N2C[C@H](Cc3ccc(C)cc3)C[C@H]2C(=O)NCc2ccc3c(cnn3C)c2)C1 ; Cn1ncc2cc(CNC(=O)[C@@H]3C[C@@H](Cc4ccc(Cl)cc4)CN3C(=O)[C@@H]3NCCC[C@@H]3C(=O)N3CCCC3)ccc21 ; Cc1ccc(C[C@@H]2C[C@@H](C(=O)NCc3ccn4c(C)nnc4c3)N(C(=O)[C@@H]3NCCC[C@@H]3C(=O)N3CCCC3)C2)cc1]

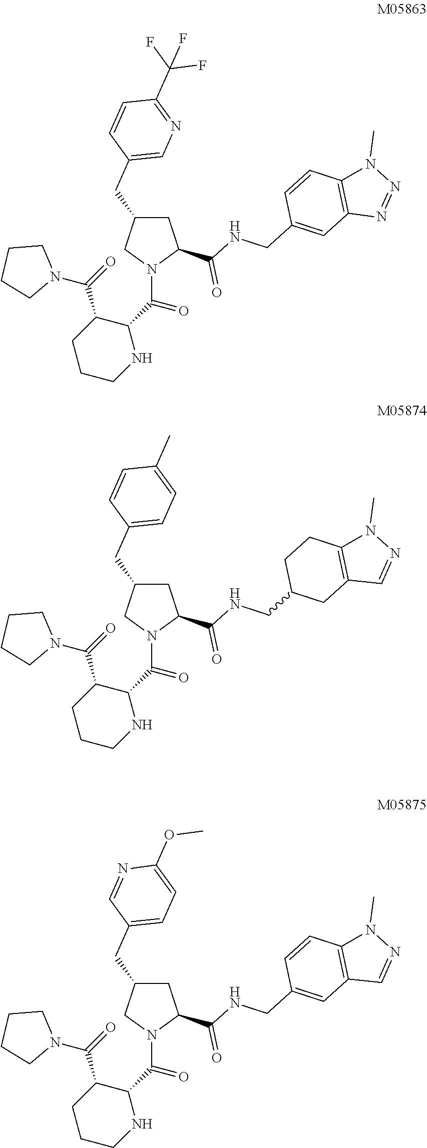 [SMILES: Cn1nnc2cc(CNC(=O)[C@@H]3C[C@@H](Cc4ccc(C(F)(F)F)nc4)CN3C(=O)[C@@H]3NCCC[C@@H]3C(=O)N3CCCC3)ccc21 ; Cc1ccc(C[C@@H]2C[C@@H](C(=O)NCC3CCc4c(cnn4C)C3)N(C(=O)[C@@H]3NCCC[C@@H]3C(=O)N3CCCC3)C2)cc1 ; COc1ccc(C[C@@H]2C[C@@H](C(=O)NCc3ccc4c(cnn4C)c3)N(C(=O)[C@@H]3NCCC[C@@H]3C(=O)N3CCCC3)C2)cn1]

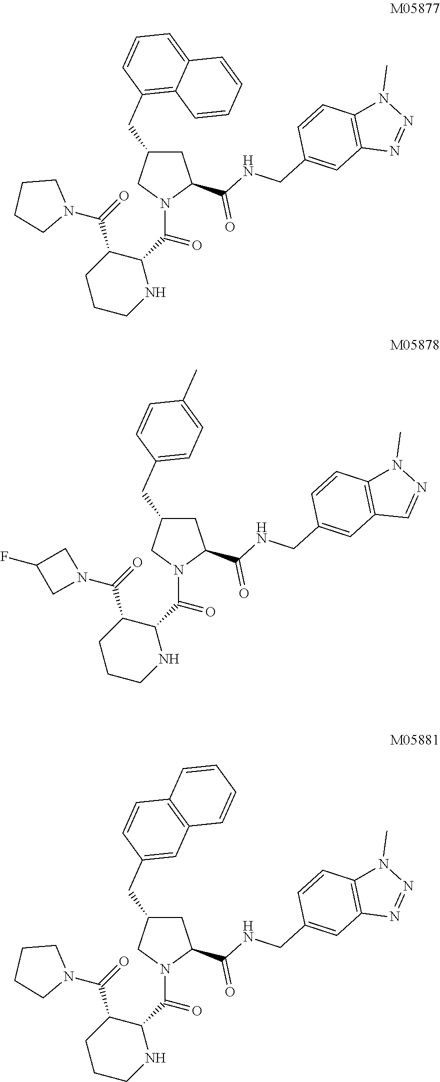 [SMILES: Cn1nnc2cc(CNC(=O)[C@@H]3C[C@@H](Cc4cccc5ccccc45)CN3C(=O)[C@@H]3NCCC[C@@H]3C(=O)N3CCCC3)ccc21 ; Cc1ccc(C[C@@H]2C[C@@H](C(=O)NCc3ccc4c(cnn4C)c3)N(C(=O)[C@@H]3NCCC[C@@H]3C(=O)N3CC(F)C3)C2)cc1 ; Cn1nnc2cc(CNC(=O)[C@@H]3C[C@@H](Cc4ccc5ccccc5c4)CN3C(=O)[C@@H]3NCCC[C@@H]3C(=O)N3CCCC3)ccc21]

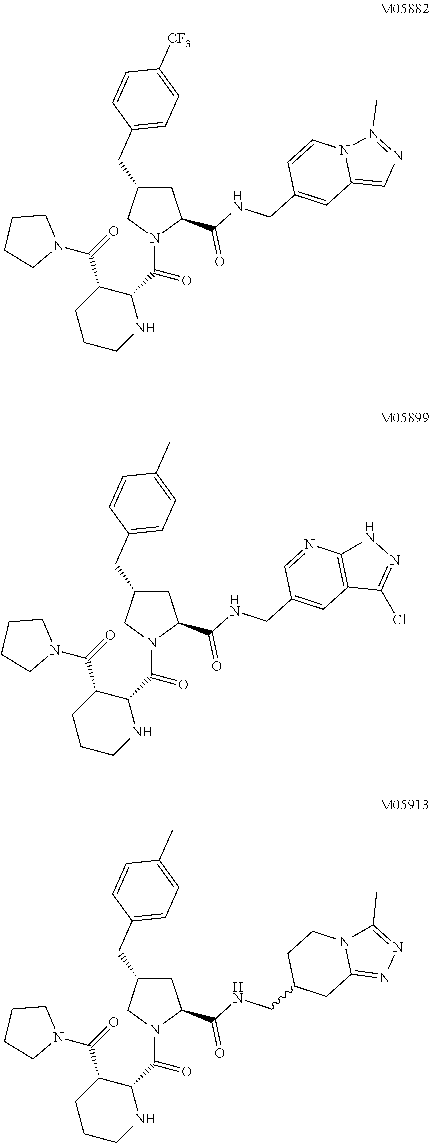

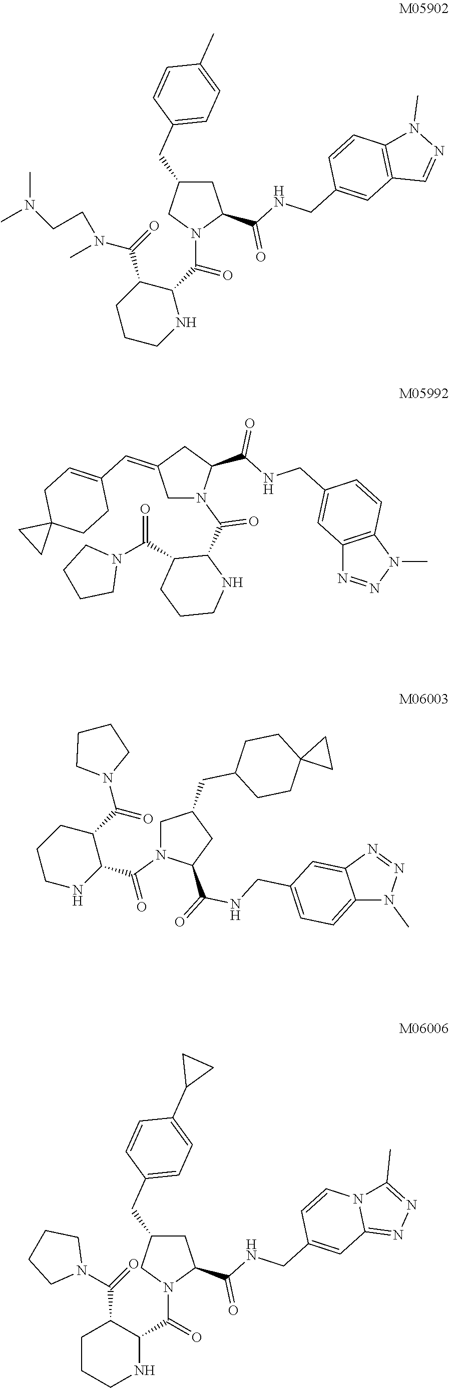 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN(C)C)C2)cc1 ; Cn1nnc2cc(CNC(=O)[C@@H]3C/C(=C/C4=CCC5(CC4)CC5)CN3C(=O)[C@@H]3NCCC[C@@H]3C(=O)N3CCCC3)ccc21 ; Cn1nnc2cc(CNC(=O)[C@@H]3C[C@@H](CC4CCC5(CC4)CC5)CN3C(=O)[C@@H]3NCCC[C@@H]3C(=O)N3CCCC3)ccc21 ; Cc1nnc2cc(CNC(=O)[C@@H]3C[C@@H](Cc4ccc(C5CC5)cc4)CN3C(=O)[C@@H]3NCCC[C@@H]3C(=O)N3CCCC3)ccn12]

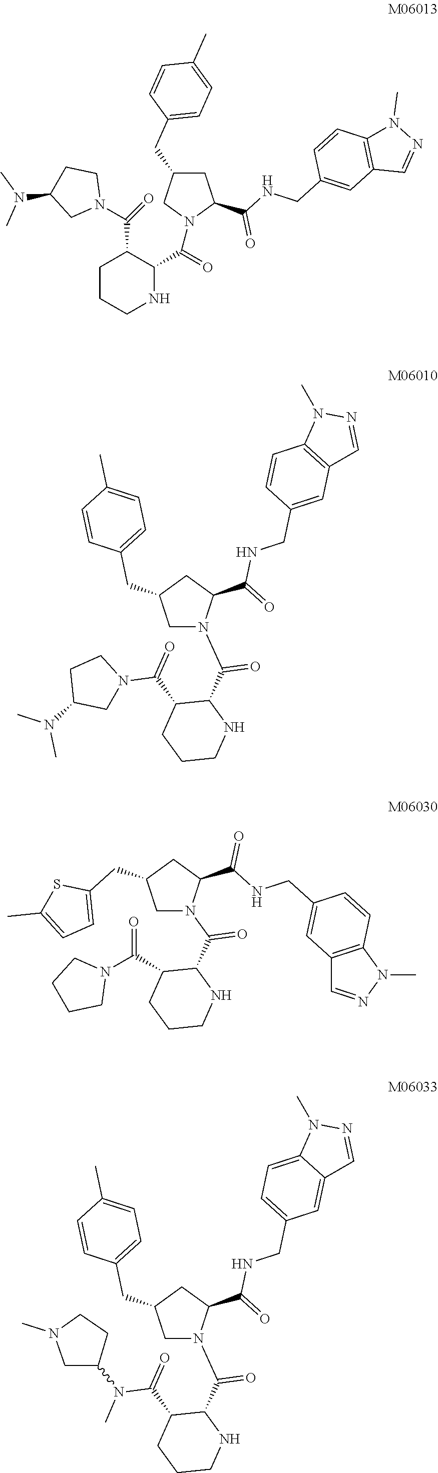

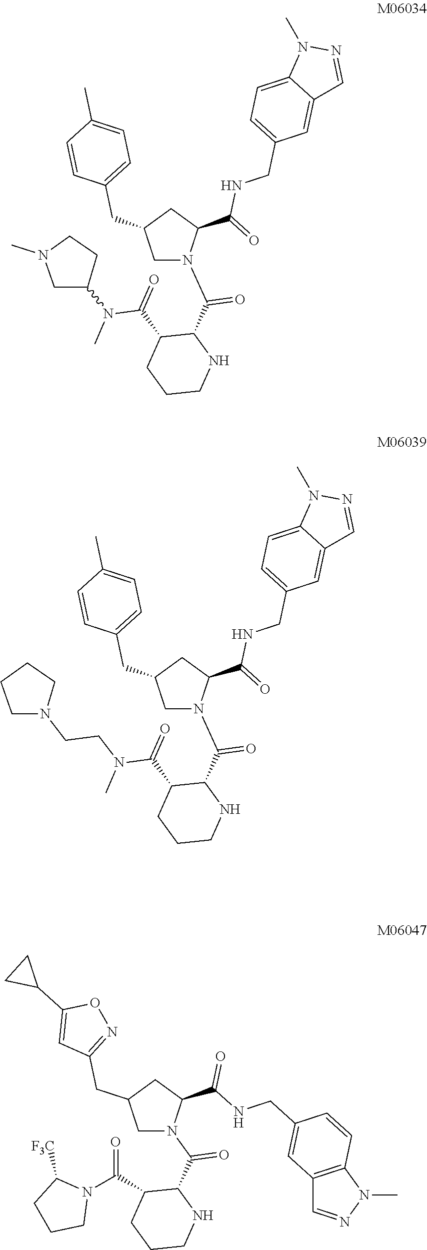 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CCN(C)C3)C2)cc1 ; Cc1ccc(C[C@@H]2C[C@@H](C(=O)NCc3ccc4c(cnn4C)c3)N(C(=O)[C@@H]3NCCC[C@@H]3C(=O)N(C)CCN3CCCC3)C2)cc1 ; Cn1ncc2cc(CNC(=O)[C@@H]3CC(Cc4cc(C5CC5)on4)CN3C(=O)[C@@H]3NCCC[C@@H]3C(=O)N3CCC[C@@H]3C(F)(F)F)ccc21]

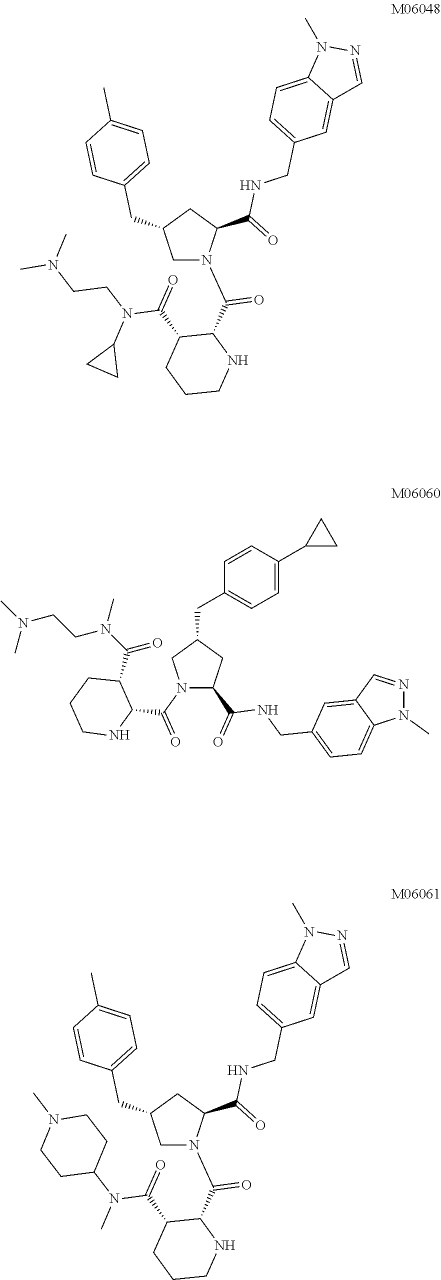

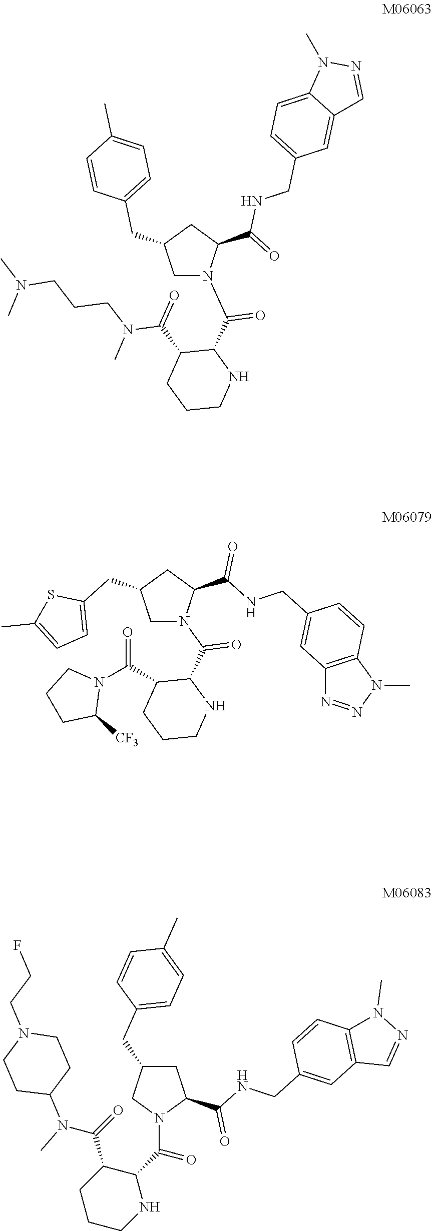

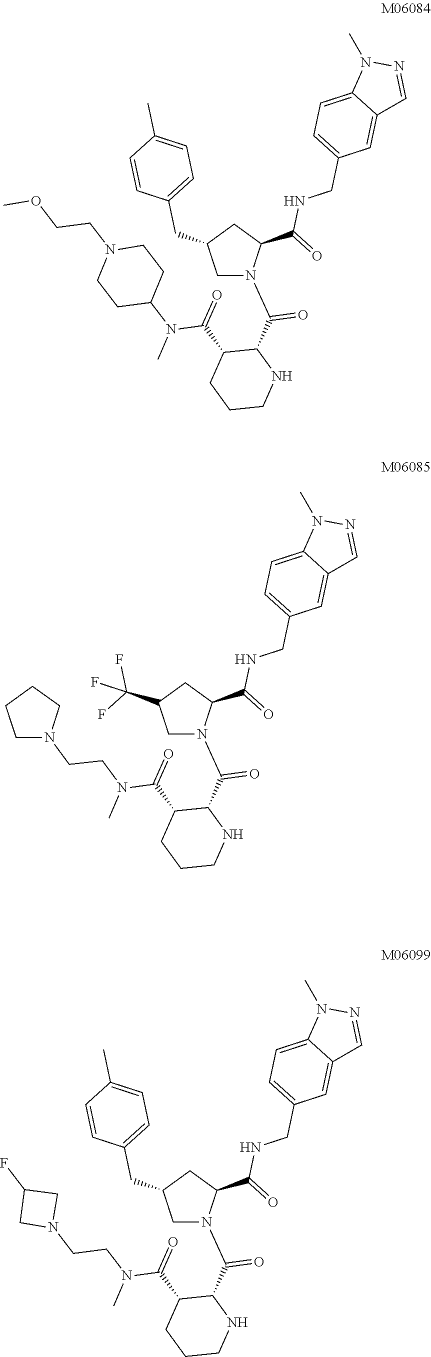

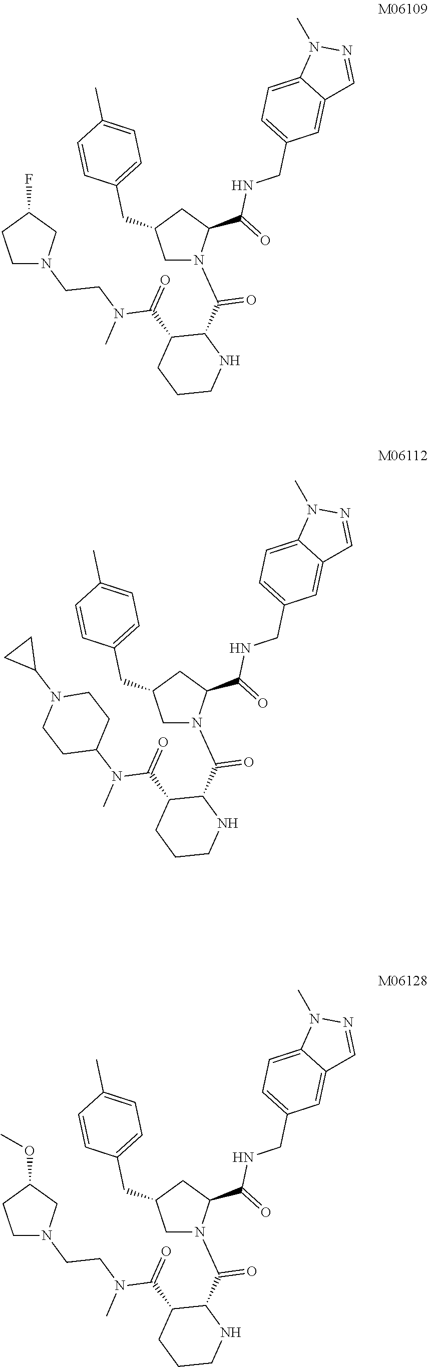 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN3CC[C@H](F)C3)C2)cc1 ; Cc1ccc(C[C@@H]2C[C@@H](C(=O)NCc3ccc4c(cnn4C)c3)N(C(=O)[C@@H]3NCCC[C@@H]3C(=O)N(C)C3CCN(C4CC4)CC3)C2)cc1 ; CO[C@H]1CCN(CCN(C)C(=O)[C@H]2CCCN[C@H]2C(=O)N2C[C@H](Cc3ccc(C)cc3)C[C@H]2C(=O)NCc2ccc3c(cnn3C)c2)C1]

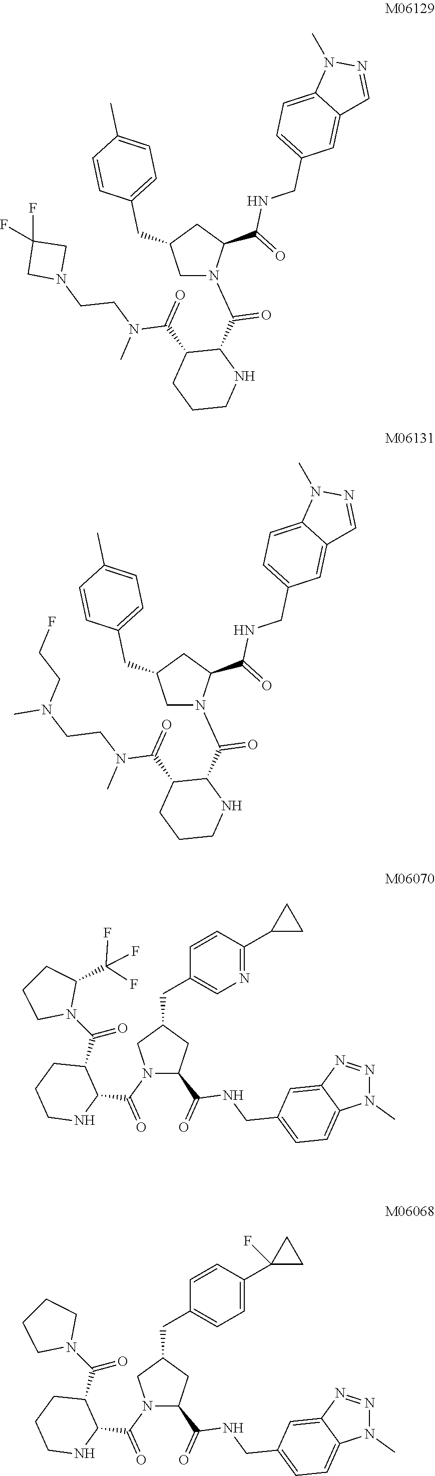 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN3CC(F)(F)C3)C2)cc1 ; Cc1ccc(C[C@@H]2C[C@@H](C(=O)NCc3ccc4c(cnn4C)c3)N(C(=O)[C@@H]3NCCC[C@@H]3C(=O)N(C)CCN(C)CCF)C2)cc1 ; Cn1nnc2cc(CNC(=O)[C@@H]3C[C@@H](Cc4ccc(C5CC5)nc4)CN3C(=O)[C@@H]3NCCC[C@@H]3C(=O)N3CCC[C@@H]3C(F)(F)F)ccc21 ; Cn1nnc2cc(CNC(=O)[C@@H]3C[C@@H](Cc4ccc(C5(F)CC5)cc4)CN3C(=O)[C@@H]3NCCC[C@@H]3C(=O)N3CCCC3)ccc21]

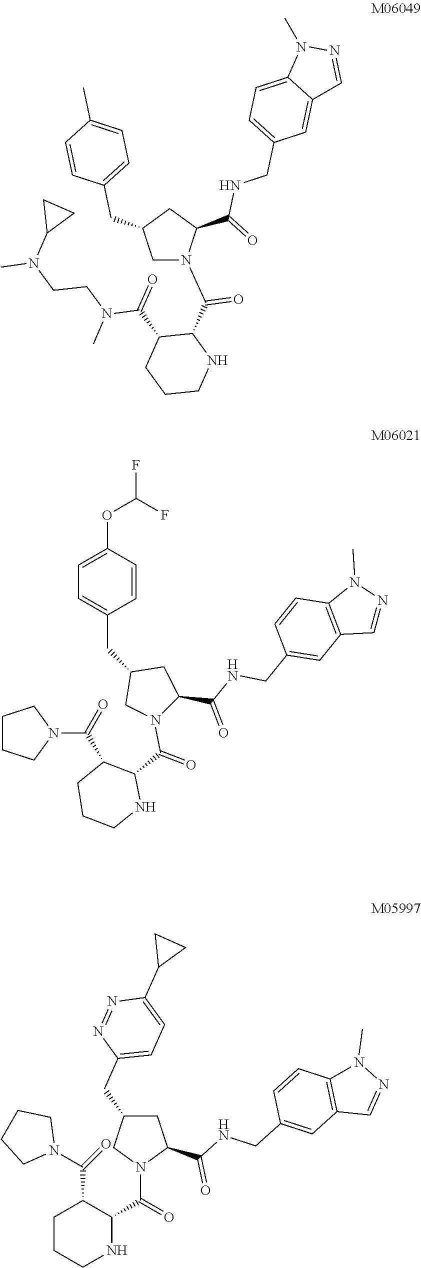 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN(C)C3CC3)C2)cc1 ; Cn1ncc2cc(CNC(=O)[C@@H]3C[C@@H](Cc4ccc(OC(F)F)cc4)CN3C(=O)[C@@H]3NCCC[C@@H]3C(=O)N3CCCC3)ccc21 ; Cn1ncc2cc(CNC(=O)[C@@H]3C[C@@H](Cc4ccc(C5CC5)nn4)CN3C(=O)[C@@H]3NCCC[C@@H]3C(=O)N3CCCC3)ccc21]

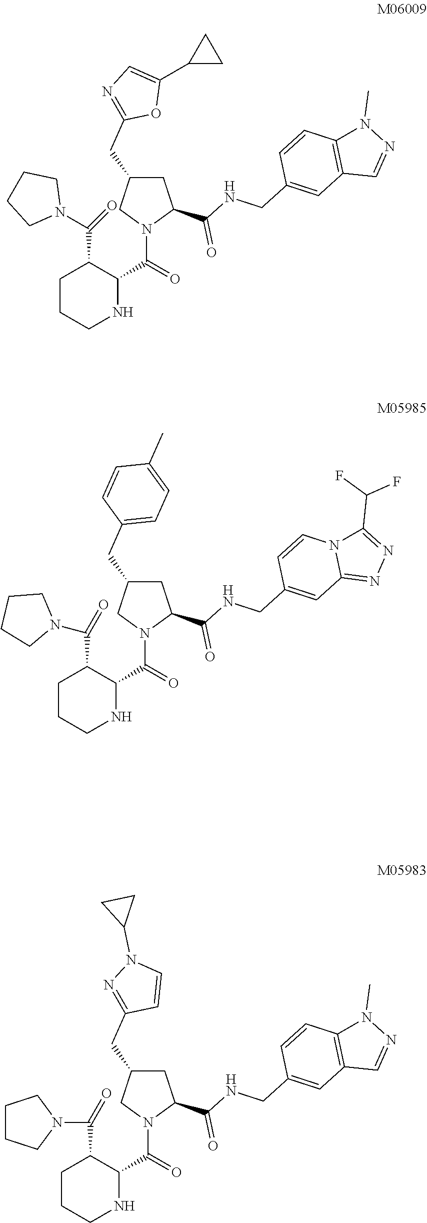 [SMILES: Cn1ncc2cc(CNC(=O)[C@@H]3C[C@@H](Cc4ncc(C5CC5)o4)CN3C(=O)[C@@H]3NCCC[C@@H]3C(=O)N3CCCC3)ccc21 ; Cc1ccc(C[C@@H]2C[C@@H](C(=O)NCc3ccn4c(C(F)F)nnc4c3)N(C(=O)[C@@H]3NCCC[C@@H]3C(=O)N3CCCC3)C2)cc1 ; Cn1ncc2cc(CNC(=O)[C@@H]3C[C@@H](Cc4ccn(C5CC5)n4)CN3C(=O)[C@@H]3NCCC[C@@H]3C(=O)N3CCCC3)ccc21]

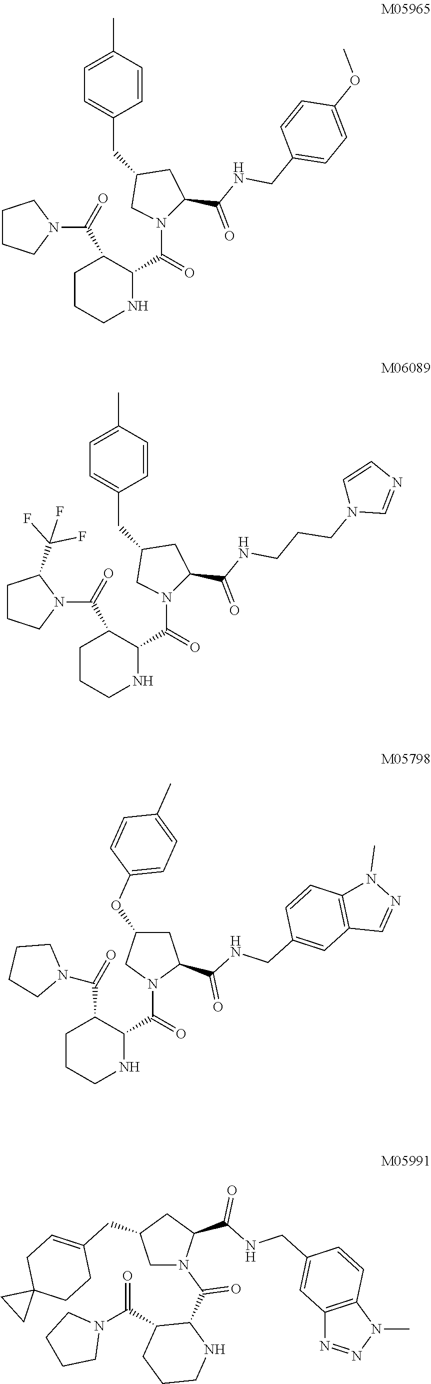 [SMILES: COc1ccc(CNC(=O)[C@@H]2C[C@@H](Cc3ccc(C)cc3)CN2C(=O)[C@@H]2NCCC[C@@H]2C(=O)N2CCCC2)cc1 ; Cc1ccc(C[C@@H]2C[C@@H](C(=O)NCCCn3ccnc3)N(C(=O)[C@@H]3NCCC[C@@H]3C(=O)N3CCC[C@@H]3C(F)(F)F)C2)cc1 ; Cc1ccc(O[C@@H]2C[C@@H](C(=O)NCc3ccc4c(cnn4C)c3)N(C(=O)[C@@H]3NCCC[C@@H]3C(=O)N3CCCC3)C2)cc1 ; Cn1nnc2cc(CNC(=O)[C@@H]3C[C@@H](CC4=CCC5(CC4)CC5)CN3C(=O)[C@@H]3NCCC[C@@H]3C(=O)N3CCCC3)ccc21]

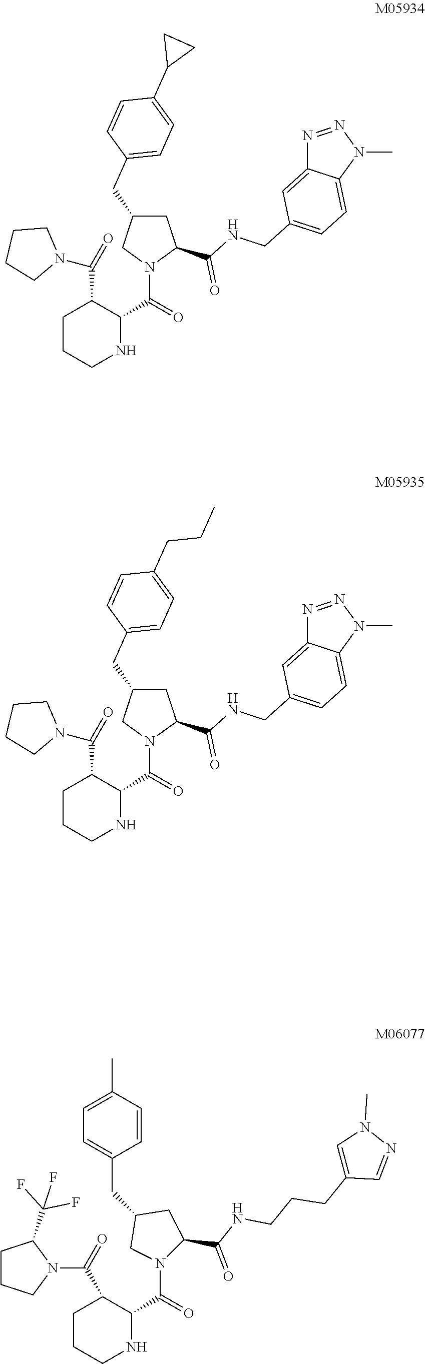 [SMILES: Cn1nnc2cc(CNC(=O)[C@@H]3C[C@@H](Cc4ccc(C5CC5)cc4)CN3C(=O)[C@@H]3NCCC[C@@H]3C(=O)N3CCCC3)ccc21 ; CCCc1ccc(C[C@@H]2C[C@@H](C(=O)NCc3ccc4c(c3)nnn4C)N(C(=O)[C@@H]3NCCC[C@@H]3C(=O)N3CCCC3)C2)cc1 ; Cc1ccc(C[C@@H]2C[C@@H](C(=O)NCCCc3cnn(C)c3)N(C(=O)[C@@H]3NCCC[C@@H]3C(=O)N3CCC[C@@H]3C(F)(F)F)C2)cc1]

In an aspect of the invention there is provided the compounds of the present invention for use as a medicament.

In accordance with another aspect, the present invention provides a pharmaceutical formulation comprising a compound of the present invention and a pharmaceutically acceptable excipient.

In an embodiment the pharmaceutical composition may be a combination product comprising an additional pharmaceutically active agent.

In a preferred aspect of the invention, the compounds are selective FXIIa inhibitors. By the term “selective FXIIa inhibitors” is meant compounds that selectively inhibit FXIIa over thrombin and FXa. Generally, a compound of the present invention may have a selectivity for FXIIa over thrombin of at least >10 fold, preferably at least >100 fold.

In accordance with another aspect of the invention, there is provided a compound of the present invention for use in the prevention or treatment of a condition which is modulated by Factor XIIa. Conditions preventable or treatable by modulation of Factor XIIa would ordinarily be conditions that are preventable or treatable by the inhibition of Factor XIIa. Accordingly, the compounds of the present invention may be for use in the prevention or treatment of a condition preventable or treatable by the inhibition of Factor XIIa.

In another aspect, the present invention provides the compounds of the present invention for use in the prevention or treatment of a condition, or as a co-therapy in a treatment or prevention of a condition, selected from:
thrombosis; deep venous thrombosis; thrombosis related to pregnancy; congenital pro-thrombotic disorders; thrombosis resulting from autoimmune conditions; transitory ischaemic attacks; myocardial infarction; peripheral arterial occlusion disorders; pulmonary embolisms; deep venousmicrovascular disease; stroke, including patients with atrial fibrillation with or without chronic kidney disease; disseminated intravascular coagulation (DIC); other conditions where inhibition of FXIIa could be beneficial such as arthritis, neurological inflammatory disorders, Alzheimer's disease, vascular dementia, macular degeneration, diabetic retinopathy, diabetic macular oedema, cerebral oedema in stroke, other causes of oedema, hereditary angioedema or acquired angioedema;
or for use in reducing the risk of a venous and/or arterial thrombosis in patients with an indication selected from:
   viral or bacterial infections, reperfusion injury (also know as ischaemia-reperfusion injury), renal insufficiency, liver diseases, myocardial infarction, angina pectoris (including unstable angina), atherosclerosis, stroke, cancer, silent brain ischaemia, and neurotraumatic disorder;
or for use in reducing the risk of a venous and/or arterial thrombosis occurring during a medical procedure selected from:
   complex left-sided ablation (pulmonary vein isolation; VT ablation), transcatheter aortic valve replacement (TAVR) (also known as transcatheter aortic valve implantation (TAVI)), spinal or epidural anaesthesia, lumbar diagnostic puncture, thoracic surgery, abdominal surgery, major orthopaedic surgery, liver biopsy, transurethral prostate resection, kidney biopsy, endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, prosthetic valve implantation, left ventricular assist device (LVAD), reocclusions and restenoses after angioplasty or aortocoronary bypass, extra corporeal membrane oxygenation (ECMO), extra corporeal circulation such as coronary artert bypass grafting (CABG), and a medical procedure comprising contact with artificial surfaces including renal dialysis;
or for use in reducing the risk of a venous and/or arterial thrombosis occurring in a patient who has undergone a medical procedure selected from:
   transcatheter aortic valve replacement (TAVR) (also known as transcatheter aortic valve implantation (TAVI)), major orthopaedic surgery, pacemaker or implantable cardioverter defibrillator (ICD) implantation (unless complex anatomical setting, e.g. congenital heart disease), mechanical valve implantation, prosthetic valve implantation, left ventricular assist device (LVAD), reocclusions and restenoses after angioplasty or aortocoronary bypass, extra corporeal membrane oxygenation (ECMO), and extra corporeal circulation such as coronary artert bypass grafting (CABG).

The compounds of the present invention may be for use in the prevention or treatment of a condition selected from the following or as a co-therapy in a treatment or prevention of a condition selected from: thrombosis, deep venous thrombosis, thrombosis related to pregnancy, congenital pro-thrombotic disorders, thrombosis resulting from autoimmune conditions, venous and arterial thrombosis as a result of viral or bacterial infections, sepsis, complex left-sided ablation (pulmonary vein isolation; VT ablation), reperfusion injury also know as ischaemia-reperfusion injury, transcatheter aortic valve replacement (TAVR) also known as transcatheter aortic valve implantation (TAVI), spinal or epidural anaesthesia, lumbar diagnostic puncture, thoracic surgery, abdominal surgery, major orthopaedic surgery, liver biopsy, transurethral prostate resection, kidney biopsy, renal insufficiency, liver diseases, endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, prosthetic valve implantation, myocardial infarction, angina pectoris (including unstable angina), reocclusions and restenoses after angioplasty or aortocoronary bypass, stroke, patients with atrial fibrillation to reduce their risk of stroke, patients with atrial fibriliation and chronic kidney disease, transitory ischaemic attacks, peripheral arterial occlusion disorders, pulmonary embolisms, deep venousmicrovascular disease, patients requiring extra corporeal membrane oxygenation (ECMO), patients requiring extra corporeal circulation such as coronary artert bypass grafting (CABG), disseminated intravascular coagulation (DIC), atherosclerosis, arthritis, thrombosis in patients with cancer, silent brain ischaemia, stroke, neurotraumatic disorder, neurological inflammatory disorders, medical procedures comprising contact with artificial surfaces including renal dialysis, other conditions where inhibition of FXIIa could be beneficial such as Alzheimer's disease, vascular dementia, macular degeneration, diabetic retinopathy, diabetic macular oedema, cerebral oedema in stroke, other causes of oedema, hereditary angioedema or acquired angioedema.

The condition preventable or treatable by the inhibition of Factor XIIa may be a condition associated with blood thickening, blood coagulation, or blood clot formulation for example the condition may be thrombosis.

In embodiments of the invention, compounds are provided for use in the prevention or treatment of or as a co-therapy for conditions associated with a high risk of bleeding, a low risk of bleeding, or thromboembolic disorders.

In embodiments of the invention, compounds are provided for use in the prevention or treatment of or as a co-therapy for conditions associated with a high risk of bleeding.

In embodiments of the invention, compounds are provided for use in the prevention or treatment of or as a co-therapy for conditions associated with a low risk of bleeding.

In embodiments of the invention, compounds are provided for use in the prevention or treatment of or as a co-therapy for conditions associated with thromboembolic disorders.

In embodiments of the invention, the compound of the invention is for use as part of a prevention or treatment for a condition associated with a high risk of bleeding, wherein the treatment is selected from complex left-sided ablation (pulmonary vein isolation; VT ablation), spinal or epidural anaesthesia, lumbar diagnostic puncture, thoracic surgery, abdominal surgery, major orthopaedic surgery, liver biopsy, transurethral prostate resection, kidney biopsy, liver diseases or renal insufficiency.

In embodiments of the invention, the compound of the invention is for use as part of a prevention or treatment for a condition associated with a low risk of bleeding, wherein the treatment is selected from endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, or prosthetic valve implantation.

In an embodiment, compounds of the present invention are for use to avoid or mitigate the contraindications of existing anticoagulant therapies, such as Dabigatran, Rivaroxaban, Apixaban, warfarin, Edoxaban and Betrixaban.

In an aspect of the invention there is provided a use of a compound of the invention to avoid or mitigate the contraindications of existing anticoagulant therapies, such as Dabigatran, Rivaroxaban, Apixaban, warfarin, Edoxaban and Betrixaban.

In an embodiment, the compounds of the present invention are for use in mitigating the contraindications of therapies using Rivaroxaban; wherein the contraindication may include: an estimated Glomerular Filtration Rate (eGFR) of less than 15 mL/minute/1.73 m², active bleeding, a significant risk of major bleeding from: current or recent gastro-intestinal ulcer, oesophageal varices, recent brain or spinal injury, recent brain, spine, or ophthalmic surgery, recent intracranial haemorrhage, malignant neoplasm, vascular aneurysm, prosthetic heart valve, liver disease associated with coagulopathy and clinically relevant bleeding risk, as well as people who have cirrhosis with Child Pugh B and C or people who are taking any other anticoagulants, except when switching to or from warfarin treatment; and people who are taking strong inhibitors of cytochrome P 3A4 enzyme and P-glycoprotein, such as ketoconazole, or HIV protease inhibitors such as ritonavir.

In an embodiment, the compounds of the present invention are for use in mitigating the contraindications of therapies using Apixaban; wherein the contraindication may include: creatinine clearance (CrCl) of less than 15 mL/min, or eGFR <15 mL/minute/1.73 m², active bleeding, a significant risk of major bleeding such as: current or recent gastro-intestinal ulcer, oesophageal varices, recent brain or spinal injury, recent brain, spine, or ophthalmic surgery, recent intracranial haemorrhage, malignant neoplasm, vascular aneurysm, liver disease associated with coagulopathy and clinically relevant bleeding risk, a prosthetic heart valve, people who are taking any other anticoagulants, except when switching to or from warfarin treatment, or people who are taking strong inhibitors of cytochrome P3A4 enzyme and P-glycoprotein, such as ketoconazole, or HIV protease inhibitors such as ritonavir.

In an embodiment, the compounds of the present invention are for use in mitigating the contraindications of therapies using Edoxaban; wherein the contraindication includes Edoxaban not being used in NVAF patients with CrCl >95 mL/minute because of an increased risk of ischemic stroke compared to warfarin.

In an embodiment, the compounds of the present invention are for use in mitigating the contraindications of therapies using Dabigatran; wherein the contraindication includes stroke prophylaxis with atrial fibrillation (Prevention of stroke and systemic embolism associated with nonvalvular atrial fibrillation), renal impairment CrCl <15 mL/min or dialysis, DVT or PE treatment (Indicated for treatment of deep vein thrombosis (DVT) and pulmonary embolus (PE) in patients who have been treated with a parenteral anticoagulant for 5-10 days) CrCl <30 mL/min or on dialysis, DVT or PE prophylaxis (Indicated for the prophylaxis of deep vein thrombosis (DVT) and pulmonary embolism (PE) following hip replacement surgery), Dabigatran is contraindicated with defibrotide, mifepristone and human prothrombin complex concentrate, dabigatran should not be used with the following: antithrombin alfa, antithrombin iii, apixaban, carbamazepine, dalteparin, dexamethasone, doxorubicin, doxorubicin liposomal, dronedarone, edoxaban, enoxaparin, fondaparinux, fosphenytoin, heparin, ketoconazole, lepirudin, nefazodone, phenobarbital, phenytoin, primidone, rifampin, st john's wort, tenofovir df, tipranavir, vinblastine and warfarin.

In an embodiment, the compounds of the present invention are for use in mitigating the contraindications of therapies using Dabigatran; wherein the contraindication includes: renal impairment (CrCl <15 mL/min), hemodialysis, hypersensitivity, ctive pathologic bleeding, impairment of hemostasis, mechanical or prosthetic heart valves, thromboembolic events (eg, valve thrombosis, stroke, TIAs, MI), excessive major bleeding (predominantly postoperative pericardial effusions requiring intervention for hemodynamic compromise), increased bleeding risk during labor and delivery, anticoagulants for active bleeding, elective surgery, or invasive procedures, patients at an increased risk of stroke, additive risk of bleeding when coadministered with antiplatelet agents, warfarin, heparin, fibrinolytic therapy, and long-term NSAIDs or aspirin, congenital or acquired coagulation disorders, ulcerative GI diseases and other gastritis like symptoms, recent haemorrhage, recent brain, spinal, or ophthalmic surgery, patients undergoing neuraxial anesthesia (spinal/epidural anesthesia), patients undergoing spinal puncture at risk of developing an epidural or spinal hematoma which can result in long-term or permanent paralysis, coadministration with P-gp inducers and inhibitors, P-gp inducers (eg, rifampicinn) or any combination thereof.

In an embodiment, the compounds of the present invention are for use in mitigating the contraindications of therapies using Betrixaban; wherein the contraindication includes: patients taking P-gp inhibitor, pateints who have severe renal impairment, patients with hepatic impairment, patients with intrinsic coagulation abnormalities, or patients with prosthetic heart valves, coadministration with drugs affecting hemostasis (thereby increasing bleeding risk), coadministration with aspirin, coadministration with other antiplatelet agents, coadministration with other anticoagulants, coadministration with heparin, coadministration with thrombolytic agents, coadministration with selective serotonin reuptake inhibitors (SSRIs), coadministration with serotonin-norepinephrine reuptake inhibitors (SNRIs), and coadministration with non-steroidal anti-inflammatory drugs (NSAIDs).

In an embodiment, compounds of the invention may be used as anticoagulants for the prophylaxis and/or therapy of thromboembolic disorders; wherein the disorder is one of: myocardial infarction, angina pectoris (including unstable angina), reocclusions and restenoses after angioplasty or aortocoronary bypass, stroke, patients with atrial fibrillation to reduce their risk of stroke, patients with atrial fibriliation and chronic kidney disease, transitory ischaemic attacks, peripheral arterial occlusion disorders, reperfusion injury also know as ischaemia-reperfusion injury, transcatheter aortic valve replacement (TAVR) also known as transcatheter aortic valve implantation (TAVI), pulmonary embolisms, deep venousmicrovascular disease or patients requiring extra corporeal membrane oxygenation (ECMO).

In an embodiment, compounds according to the invention may be suitable for preventing and/or treating disseminated intravascular coagulation (DIC).

In an embodiment, the compounds of the invention are also suitable for the prophylaxis and/or treatment of atherosclerosis and arthritis, and additionally also for the prophylaxis and/or treatment of thrombosis in patients with cancer.

In an embodiment the compounds of the present invention is for use in a method of preventing and/or treating thrombosis.

In an aspect of the invention the compound disclosed herein may be for use as an anticoagulant.

In another aspect, the present invention provides a method for the prevention or treatment of a condition selected from:
thrombosis; deep venous thrombosis; thrombosis related to pregnancy; congenital pro-thrombotic disorders; thrombosis resulting from autoimmune conditions; transitory ischaemic attacks; myocardial infarction; peripheral arterial occlusion disorders; pulmonary embolisms; deep venousmicrovascular disease; stroke, including patients with atrial fibrillation with or without chronic kidney disease; disseminated intravascular coagulation (DIC); other conditions where inhibition of FXIIa could be beneficial such as arthritis, neurological inflammatory disorders, Alzheimer's disease, vascular dementia, macular degeneration, diabetic retinopathy, diabetic macular oedema, cerebral oedema in stroke, other causes of oedema, hereditary angioedema or acquired angioedema;
or a method for reducing the risk of a venous and/or arterial thrombosis in patients with an indication selected from:
   viral or bacterial infections, reperfusion injury (also know as ischaemia-reperfusion injury), renal insufficiency, liver diseases, myocardial infarction, angina pectoris (including unstable angina), atherosclerosis, stroke, cancer, silent brain ischaemia, and neurotraumatic disorder;
or a method for reducing the risk of a venous and/or arterial thrombosis occurring during a medical procedure selected from:
   complex left-sided ablation (pulmonary vein isolation; VT ablation), transcatheter aortic valve replacement (TAVR) (also known as transcatheter aortic valve implantation (TAVI)), spinal or epidural anaesthesia, lumbar diagnostic puncture, thoracic surgery, abdominal surgery, major orthopaedic surgery, liver biopsy, transurethral prostate resection, kidney biopsy, endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, prosthetic valve implantation, left ventricular assist device (LVAD), reocclusions and restenoses after angioplasty or aortocoronary bypass, extra corporeal membrane oxygenation (ECMO), extra corporeal circulation such as coronary artert bypass grafting (CABG), and a medical procedure comprising contact with artificial surfaces including renal dialysis;
or a method for reducing the risk of a venous and/or arterial thrombosis occurring in a patient who has undergone a medical procedure selected from:
   transcatheter aortic valve replacement (TAVR) (also known as transcatheter aortic valve implantation (TAVI)), major orthopaedic surgery, pacemaker or implantable cardioverter defibrillator (ICD) implantation (unless complex anatomical setting, e.g. congenital heart disease), mechanical valve implantation, prosthetic valve implantation, left ventricular assist device (LVAD), reocclusions and restenoses after angioplasty or aortocoronary bypass, extra corporeal membrane oxygenation (ECMO), and extra corporeal circulation such as coronary artert bypass grafting (CABG);
wherein the method comprises administering a therapeutically effective amount of a compound of the invention or administering a therapeutically effective amount of a compound of the present invention as a co-therapy to a patient in need thereof.

In an aspect of the invention there is provided a method for prevention of thrombosis or deep venous thrombosis, prevention and/or treatment of a condition selected from: thrombosis, thrombosis related to pregnancy, congenital pro-thrombotic disorders, thrombosis resulting from autoimmune conditions, venous and arterial thrombosis as a result of viral or bacterial infections, sepsis, complex left-sided ablation (pulmonary vein isolation; VT ablation), spinal or epidural anaesthesia, lumbar diagnostic puncture, thoracic surgery, abdominal surgery, major orthopaedic surgery, liver biopsy, transurethral prostate resection, kidney biopsy, renal insufficiency, liver diseases, endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, prosthetic valve implantation, myocardial infarction, angina pectoris (including unstable angina), reocclusions and restenoses after angioplasty or aortocoronary bypass, stroke, patients with atrial fibrillation to reduce their risk of stroke, patients with atrial fibriliation and chronic kidney disease, transitory ischaemic attacks, peripheral arterial occlusion disorders, pulmonary embolisms, deep venousmicrovascular disease, patients requiring extra corporeal membrane oxygenation (ECMO), patients requiring extra corporeal circulation such as coronary artert bypass grafting (CABG), disseminated intravascular coagulation (DIC), atherosclerosis, arthritis, thrombosis in patients with cancer, silent brain ischaemia, stroke, neurotraumatic disorder, neurological inflammatory disorders, medical procedures comprising contact with artificial surfaces including renal dialysis, other conditions where inhibition of FXIIa could be beneficial such as Alzheimer's disease, vascular dementia, macular degeneration, diabetic retinopathy, diabetic macular oedema, cerebral oedema in stroke, other causes of oedema, hereditary angioedema or acquired angioedema, wherein the method comprises administering a therapeutically effective amount of a compound of the invention or administering a therapeutically effective amount of a compound of the present invention as a co-therapy.

In an aspect of the invention there is provided a method of preventing coagulation, wherein the method comprises the administration of a therapeutically effective amount of a compound of the invention.

In an aspect of the invention there is provided a method of preventing and/or treating thrombosis, wherein the method comprises the administration of a therapeutically effective amount of a compound of the invention.

In an aspect of the invention there is provided a use of a compound of the invention in the manufacture of a medicament for use in the prevention and/or treatment of conditions preventable and/or treatable by the inhibition of Factor XII (optionally Factor XIIa), for example the condition may be thrombosis.

In another aspect of the invention there is provided a pharmaceutical composition, wherein the composition comprises a compound of the invention and pharmaceutically acceptable excipients.

In an embodiment the pharmaceutical composition may be a combination product comprising an additional pharmaceutically active agent. The additional pharmaceutically active agent may be one disclosed elsewhere herein.

The compounds of the present invention may be used for the prevention and/or treatment of any of the conditions disclosed above. Alternatively, the compounds of the present invention may be used as a co-therapy in a prevention and/or treatment of a condition disclosed above. Where the compound of the present invention is used as a co-therapy with respect to a particular condition, it is meant that the compound of the invention could be used in combination with another art known therapy for the condition. For example a FXII(a) inhibitor may be used in combination with anti-platelet therapy with the aim of providing enhanced anti-thrombotic efficacy without incurring an increased risk of bleeding compared with the anti-platelet therapy alone. Furthermore, a FXII(a) inhibitor is likely to be used in combination with other treatments.

### DETAILED DESCRIPTION

Given below are definitions of terms used in this application. Any term not defined herein takes the normal meaning as the skilled person would understand the term.

The term “halo” or “halogen” refers to one of the halogens, group 17 of the periodic table. In particular, the term refers to fluorine, chlorine, bromine and iodine. Preferably, the term refers to fluorine or chlorine.

The term “alkyl” refers to a linear or branched hydrocarbon chain. For example, the term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl.

Alkylene groups may likewise be linear or branched and may have two places of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “alkoxy” refers to an alkyl group which is attached to a molecule via oxygen. For example, the term “C₁₋₆ alkoxy” refers to a group where the alkyl part may be linear or branched and may contain 1, 2, 3, 4, 5 or 6 carbon atoms, for example methyl, ethyl, n-propyl, iso-propyl, n-butyl, sec-butyl, tert-butyl, n-pentyl and n-hexyl. Therefore, the alkoxy group may be methoxy, ethoxy, n-propoxy, iso-propoxy, n-butoxy, sec-butoxy, tert-butoxy, n-pentoxy and n-hexoxy. The alkyl part of the alkoxy group may be unsubstituted or substituted by one or more substituents. Possible substituents are described below. Substituents for the alkyl group may be halogen, e.g. fluorine, chlorine, bromine and iodine, OH, C₁₋₆ alkoxy.

The term “haloalkyl” refers to a hydrocarbon chain substituted with at least one halogen atom independently chosen at each occurrence, for example fluorine, chlorine, bromine and iodine. For example, the term “C₁₋₆ haloalkyl” refers to a linear or branched hydrocarbon chain containing 1, 2, 3, 4, 5 or 6 carbon atoms substituted with at least one halogen. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

The term “alkenyl” refers to a branched or linear hydrocarbon chain containing at least one double bond. For example, the term “C₂₋₆ alkenyl” refers to a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl.

The term “alkynyl” refers to a branched or linear hydrocarbon chain containing at least one triple bond. For example, the term “C₂₋₆ alkynyl” refers to a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl.

The term “carbocyclic” refers to a saturated or unsaturated carbon containing ring system. A “carbocyclic” system may be monocyclic or a fused polycyclic ring system, for example, bicyclic or tricyclic. A “carbocyclic” moiety may contain from 3 to 14 carbon atoms, for example, 3 to 8 carbon atoms in a monocyclic system and 7 to 14 carbon atoms in a polycyclic system. “Carbocyclic” encompasses cycloalkyl moieties, cycloalkenyl moieties, aryl ring systems and fused ring systems including an aromatic portion.

The term “heterocyclic” refers to a saturated or unsaturated ring system containing at least one heteroatom selected from N, O or S. A “heterocyclic” system may contain 1, 2, 3 or 4 heteroatoms, for example 1 or 2. A “heterocyclic” system may be monocyclic or a fused polycyclic ring system, for example, bicyclic or tricyclic. A “heterocyclic” moiety may contain from 3 to 14 carbon atoms, for example, 3 to 8 carbon atoms in a monocyclic system and 7 to 14 carbon atoms in a polycyclic system. “Heterocyclic” encompasses heterocycloalkyl moieties, heterocycloalkenyl moieties and heteroaromatic moieties. For example, the heterocyclic group may be: oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term cycloalkyl refers to a saturated hydrocarbon ring system. For example “C₃₋₈ cycloalkyl” refers to a ring system containing 3, 4, 5, 6, 7 or 8 carbon atoms. For example, the “C₃₋₈ cycloalkyl” may be cyclopropyl, cyclobutyl, cyclopentyl, cyclohexyl, cycloheptyl and cyclooctyl.

The term “C₃₋₈ cycloalkenyl” refers to an unsaturated hydrocarbon ring system containing 3, 4, 5, 6, 7 or 8 carbon atoms that is not aromatic. The ring may contain more than one double bond provided that the ring system is not aromatic. For example, the “C₃₋₈ cycloalkyl” may be cyclopropenyl, cyclobutenyl, cyclopentenyl, cyclopentadienyl, cyclohexenyl, cyclohexadienly, cycloheptenyl, cycloheptadiene, cyclooctenyl and cycloatadienyl.

The term “heterocycloalkyl” refers to a saturated hydrocarbon ring system containing carbon atoms and at least one heteroatom within the ring selected from N, O and S. For example there may be 1, 2 or 3 heteroatoms, optionally 1 or 2. The “heterocycloalkyl” may be bonded to the rest of the molecule through any carbon atom or heteroatom. The “heterocycloalkyl” may have one or more, e.g. one or two, bonds to the rest of the molecule: these bonds may be through any of the atoms in the ring. For example, the “heterocycloalkyl” may be a “C₃₋₈ heterocycloalkyl” or “C₃₋₈ heterocyclic ring”.

The term “C₃₋₈ heterocycloalkyl” or “C₃₋₈ heterocyclic ring” refers to a saturated hydrocarbon ring system containing 3, 4, 5, 6, 7 or 8 atoms at least one of the atoms being a heteroatom within the ring selected from N, O and S. The “heterocycloalkyl” may be oxirane, aziridine, azetidine, oxetane, tetrahydrofuran, pyrrolidine, imidazolidine, succinimide, pyrazolidine, oxazolidine, isoxazolidine, thiazolidine, isothiazolidine, piperidine, morpholine, thiomorpholine, piperazine, and tetrahydropyran.

The term “heterocycloalkenyl” refers to an unsaturated hydrocarbon ring system, that is not aromatic, containing carbon atoms and at least one heteroatom within the ring selected from N, O and S. For example there may be 1, 2 or 3 heteroatoms, optionally 1 or 2. The “heterocycloalkenyl” may be bonded to the rest of the molecule through any carbon atom or heteroatom. The “heterocycloalkenyl” may have one or more, e.g. one or two, bonds to the rest of the molecule: these bonds may be through any of the atoms in the ring. For example, the “heterocycloalkenyl” may be a “C₃₋₈ heterocycloalkenyl”. The term “C₃₋₈ heterocycloalkenyl” refers to a saturated hydrocarbon ring system containing 3, 4, 5, 6, 7 or 8 atoms at least one of the atoms being a heteroatom within the ring selected from N, O and S. The “heterocycloalkenyl” may be tetrahydropyridine, dihydropyran, dihydrofuran, pyrroline.

The term “aromatic” when applied to a substituent as a whole means a single ring or polycyclic ring system with 4n+2 electrons in a conjugated π system within the ring or ring system where all atoms contributing to the conjugated π system are in the same plane.

The term “aryl” refers to an aromatic hydrocarbon ring system. The ring system has 4n+2 electrons in a conjugated π system within a ring where all atoms contributing to the conjugated π system are in the same plane. For example, the “aryl” may be phenyl and naphthyl. The aryl system itself may be substituted with other groups. The term “aryl” also includes bicyclic or tricyclic ring systems that are not completely aromatic but contain an aromatic ring within the ring system, for example, indane or tetralin.

The term “heteroaryl” refers to an aromatic hydrocarbon ring system with at least one heteroatom within a single ring or within a fused ring system, selected from O, N and S. The ring or ring system has 4n+2 electrons in a conjugated π system where all atoms contributing to the conjugated π system are in the same plane. For example, the “heteroaryl” may be imidazole, thiene, furane, thianthrene, pyrrol, benzimidazole, pyrazole, pyrazine, pyridine, pyrimidine and indole. The term “heteroaryl” also includes bicyclic or tricyclic ring systems that are not completely aromatic but contain an aromatic ring. The heteroatoms may be present within the ring system in the aromatic ring or in a non-aromatic ring. For example heteroaryl also encompasses chromene, chromane, indoline, tetrahydroquinoline,

A bond terminating in a “ [symbol] ” represents that the bond is connected to another atom that is not shown in the structure. A bond terminating inside a cyclic structure and not terminating at an atom of the ring structure represents that the bond may be connected to any of the atoms in the ring structure where allowed by valency.

A bond drawn as a solid line and a dotted line represents a bond which can be either a single bond or a double bond, where chemically possible. For example, the bond drawn below could be a single bond or a double bond.

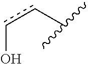

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different. The substituent(s) may be selected from: OH, NHR, amidino, guanidino, hydroxyguanidino, formamidino, isothioureido, ureido, mercapto, C(O)H, acyl, acyloxy, carboxy, sulfo, sulfamoyl, carbamoyl, cyano, azo, nitro, halo, C₁₋₆ alkyl, C₁₋₆ alkoxy, C₁₋₆ haloalkyl, C₃₋₈ cycloalkyl, C₂₋₆ alkenyl, C₂₋₆ alkynyl, aryl, heteroaryl or alkaryl. Where the group to be substituted is an alkyl group the substituent may be ═O. R may be selected from H, C₁₋₆ alkyl, C₃₋₈ cycloalkyl, phenyl, benzyl or phenethyl group, e.g. R is H or C₁₋₃ alkyl. Where the moiety is substituted with two or more substituents and two of the substituents are adjacent the adjacent substituents may form a C₄₋₈ ring along with the atoms of the moiety on which the substituents are substituted, wherein the C₄₋₈ ring is a saturated or unsaturated hydrocarbon ring with 4, 5, 6, 7, or 8 carbon atoms or a saturated or unsaturated hydrocarbon ring with 4, 5, 6, 7, or 8 carbon atoms and 1, 2 or 3 heteroatoms.

Substituents are only present at positions where they are chemically possible, the person skilled in the art being able to decide (either experimentally or theoretically) without inappropriate effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

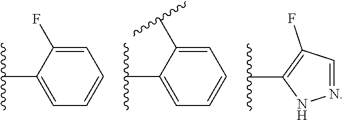

“Meta” substitution is a substitution pattern where two substituents are on carbons one carbon removed from each other, i.e with a single carbon atom between the substituted carbons. In other words there is a substituent on the second atom away from the atom with another substituent. For example the groups below are meta substituted.

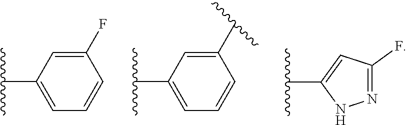

“Para” substitution is a substitution pattern where two substituents are on carbons two carbons removed from each other, i.e with two carbon atoms between the substituted carbons. In other words there is a substituent on the third atom away from the atom with another substituent. For example the groups below are para substituted.

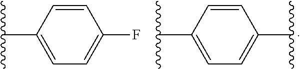

By “acyl” is meant an organic radical derived from, for example, an organic acid by the removal of the hydroxyl group, e.g. a radical having the formula R—C(O)—, where R may be selected from H, C₁₋₆ alkyl, C₃₋₈ cycloalkyl, phenyl, benzyl or phenethyl group, eg R is H or C₁₋₃ alkyl. In one embodiment acyl is alkyl-carbonyl. Examples of acyl groups include, but are not limited to, formyl, acetyl, propionyl and butyryl. A particular acyl group is acetyl.

Throughout the description the disclosure of a compound also encompasses pharmaceutically acceptable salts, solvates and stereoisomers thereof. Where a compound has a stereocentre, both (R) and (S) stereoisomers are contemplated by the invention, equally mixtures of stereoisomers or a racemic mixture are completed by the present application. Where a compound of the invention has two or more stereocenters any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, at least 60% or less. For example, the e.e. or d.e. may be 90% or more, 90% or more, 80% or more, 70% or more, 60% or more, 50% or more, 40% or more, 30% or more, 20% or more, or 10% or more.

The invention contemplates pharmaceutically acceptable salts of the compounds of the invention. These may include the acid addition and base salts of the compounds. These may be acid addition and base salts of the compounds. In addition the invention contemplates solvates of the compounds. These may be hydrates or other solvated forms of the compound.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (1) may be prepared by one or more of three methods:
(i) by reacting the compound of the invention with the desired acid or base:
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of any formula include references to salts, solvates and complexes thereof and to solvates and complexes of salts thereof.

The compounds of the invention include compounds of a number of formula as herein defined, including all polymorphs and crystal habits thereof, prodrugs and isomers thereof (including optical, geometric and tautomeric isomers) as hereinafter defined and isotopically-labelled compounds of the invention.

The present invention also includes all pharmaceutically acceptable isotopically-labelled compounds of the invention wherein one or more atoms are replaced by atoms having the same atomic number, but an atomic mass or mass number different from the atomic mass or mass number most commonly found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Before purification, the compounds of the present invention may exist as a mixture of enantiomers depending on the synthetic procedure used. The enantiomers can be separated by conventional techniques known in the art. Thus the invention covers individual enantiomers as well as mixtures thereof.

For some of the steps of the process of preparation of the compounds of the invention, it may be necessary to protect potential reactive functions that are not wished to react, and to cleave said protecting groups in consequence. In such a case, any compatible protecting radical can be used. In particular methods of protection and deprotection such as those described by T.W. GREENE (Protective Groups in Organic Synthesis, A. Wiley-Interscience Publication, 1981) or by P. J. Kocienski (Protecting groups, Georg Thieme Verlag, 1994), can be used. All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of the present invention as well as intermediates for the preparation thereof can be purified according to various well-known methods, such as for example crystallization or chromatography.

One or more compounds of the invention may be combined with one or more pharmaceutical agents, for example anti-inflammatory agents, anti-fibrotic agents, chemotherapeutics, anti cancer agents, immunosuppressants, anti-tumour vaccines, cytokine therapy, or tyrosine kinase inhibitors, for the treatment of conditions modulated by the inhibition of ROCK, for example fibrotic diseases, auto-immune, inflammatory-fibrotic conditions, inflammatory conditions, central nervous system disorders, or cancer.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

The compounds of the invention can be administered in vivo either alone or in combination with other pharmaceutically active agents, e.g. agents effective in particular for the treatment and/or prophylaxis of the aforementioned diseases. A suitable combination consists of a compound of the present invention with one or more active substances which may be mentioned by way of example and preferably are: lipid-lowering agents, in particular HMG-CoA-(3-hydroxy-3-methylglutaryl-coenzyme A)-reductase inhibitors; coronary therapeutics/vasodilators, in particular ACE (angiotensin converting enzyme) inhibitors; All (angiotensin II) receptor antagonists; β-adrenoceptor antagonists; alpha-1-adrenoceptor antagonists; diuretics; calcium channel blockers; substances which bring about an increase in cyclic guanosine monophosphate (cOMP), such as, for example, stimulators of soluble guanylate cyclase; plasminogen activators (thrombolytics/fibrinolytics) and thrombolysis/fibrinolysis-increasing compounds such as inhibitors of the plasminogen activator inhibitor (PAI inhibitors) or inhibitors of the thrombin-activated fibrinolysis inhibitor (TAFI); substances having anticoagulatory activity (anticoagulants); substances inhibiting platelet aggregation (platelet aggregation inhibitors, thrombocyte aggregation inhibitors); and fibrinogen receptor antagonists (glycoprotein IIb/IIIa antagonists).

The compounds of the invention may be advantageous in the treatment of cancer since cancer patients have a pro-thrombotic state and may need anticoagulants. This normally has to be balanced with risk of bleeding, therefore, the compounds described herein offer a safer anticoagulant in cancer patients because of the reduced risk of bleeding. For the treatment of cancer the compounds of the invention may be administered in combination with known cancer treating therapies.

Compounds of the invention may exist in a single crystal form or in a mixture of crystal forms or they may be amorphous. Thus, compounds of the invention intended for pharmaceutical use may be administered as crystalline or amorphous products. They may be obtained, for example, as solid plugs, powders, or films by methods such as precipitation, crystallization, freeze drying, or spray drying, or evaporative drying. Microwave or radio frequency drying may be used for this purpose.

For the above-mentioned compounds of the invention the dosage administered will, of course, vary with the compound employed, the mode of administration, the treatment desired and the disorder indicated. The skilled person within the field of drug dosage would readily identify a suitable dosage, for example the dosage may be a standard dosage amount.

A compound of the invention, or pharmaceutically acceptable salt thereof, may be used on their own but will generally be administered in the form of a pharmaceutical composition in which the compounds of the invention, or pharmaceutically acceptable salt thereof, is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration of the compounds of the invention, the pharmaceutical composition which is used to administer the compounds of the invention will preferably comprise from 0.05 to 99% w (percent by weight) compounds of the invention, more preferably from 0.05 to 80% w compounds of the invention, still more preferably from 0.10 to 70% w compounds of the invention, and even more preferably from 0.10 to 50% w compounds of the invention, all percentages by weight being based on total composition.

The pharmaceutical compositions may be administered topically (e.g. to the skin) in the form, e.g., of creams, gels, lotions, solutions, suspensions, or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the compounds of the invention may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the compounds of the invention may be admixed with, for example, a vegetable oil or polyethylene glycol. Hard gelatine capsules may contain granules of the compound using either the above-mentioned excipients for tablets. Also liquid or semisolid formulations of the compound of the invention may be filled into hard gelatine capsules. Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

For intravenous (parenteral) administration the compounds of the invention may be administered as a sterile aqueous or oily solution.

The size of the dose for therapeutic purposes of compounds of the invention will naturally vary according to the nature and severity of the conditions, the age and sex of the animal or patient and the route of administration, according to well-known principles of medicine.

Dosage levels, dose frequency, and treatment durations of compounds of the invention are expected to differ depending on the formulation and clinical indication, age, and co-morbid medical conditions of the patient. The standard duration of treatment with compounds of the invention may be any length of time. For example, the treatment duration may be days, weeks, months or years. The treatment may be indefinite. It may be that the treatment may be for between one and seven months for most clinical indications. It may be necessary to extend the duration of treatment beyond seven days in instances of recurrent infections or infections associated with tissues or implanted materials to which there is poor blood supply including bones/joints, respiratory tract, endocardium, and dental tissues.

The invention may also be defined according to the following clauses:
1. A compound according to formula (1) and pharmaceutically acceptable salts thereof:

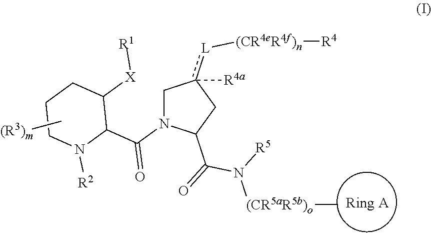

wherein
—X— is selected from: a bond, —C(O)—, C₁₋₃ alkylenyl and C₂ alkenylenyl;
R¹ is selected from: —NR^{1a}R^{1b}, a substituted or unsubstituted 5 to 10 membered monocyclic or bicyclic heteroaryl group having 1, 2 or 3 heteroatoms selected from O, N or S, a substituted or unsubstituted 6 to 10 membered monocyclic or bicyclic aryl group, a substituted or unsubstituted 3 to 10 membered monocyclic or bicyclic (fused, bridged or spiro) cycloalkyl group and a substituted or unsubstituted 3 to 10 membered monocyclic or bicyclic (fused, bridged or spiro) heterocyclic group having 1, 2 or 3 heteroatoms selected from O, N or S; wherein R^{1a} and R^{1b} are each independently selected from: substituted or unsubstituted C₁₋₆ alkyl, and substituted or unsubstituted C₃₋₆ cycloalkyl;
   wherein, when substituted, the substituent of R¹ is selected from: halo, ═O, —CN, —OH, C₁₋₆ alkyl, C₃₋₆ cycloalkyl, C₁₋₆ haloalkyl, —O—C₁₋₆ alkyl, —O—C₃₋₆ cycloalkyl, —O—C₁₋₆ haloalkyl, —NR^{1c}R^{1d}, —NR^{1c}(SO₂)R^{1d}, —NR^{1c}(C(O))R^{1d}, —C(O)NR^{1c}R^{1d}, and —SO₂NR^{1c}R^{1d}, a 5 to 10 membered heteroaryl group having 1, 2 or 3 heteroatoms selected from O, N or S, and a 6 to 10 membered aryl group; wherein R^{1c} and R^{1d} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
   wherein, when substituted, the substituent of R^{1a} and R^{1b} is selected from: halo, —CN, —OH, C₁₋₆ alkyl, C₃₋₆ cycloalkyl, C₁₋₆ haloalkyl, —O—C₁₋₆ alkyl, —O—C₃₋₆ cycloalkyl, —O—C₁₋₆ haloalkyl, —NR^{1e}R^{1f}, —NR^{1e}(SO₂)R^{1f}, —NR^{1e}(C(O))R^{1f}, —C(O)NR^{1e}R^{1f}, and —SO₂NR^{1e}R^{1f}; R^{1e} and R^{1f} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
R² is selected from: H, C₁₋₆ alkyl, C₁₋₆ haloalkyl, C₃₋₆ cycloalkyl, and 3 to 6 membered heterocyloalkyl;
R³ is selected from: halo, —CN, —OH, C₁₋₆ alkyl, C₁₋₆ haloalkyl, —O—C₁₋₆ alkyl, —O—C₁₋₆ haloalkyl, —NR^{3a}R^{3b}, —NR^{3a}(C(O))R^{3b}, and —C(O)NR^{3a}R^{3b}; wherein R^{3a} and R^{3b} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group; m is selected from 0, 1, 2, or 3;
wherein the residue

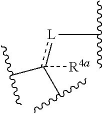

is selected from:

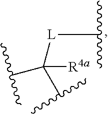

wherein L is selected from: bond, —O—, —NR^{4b}—, and —NR^{4c}C(O)—; and

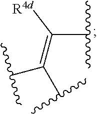

R^{4a} is selected from: H, —OH, halo, C₁₋₄ alkyl, or C₁₋₄ haloalkyl;
R^{4b} is H, C₁₋₆ alkyl or —C(O)C₁₋₆ alkyl;
R^{4c} is H or C₁₋₆ alkyl;
R^{4d} is H or C₁₋₆ alkyl;
R^{4e} and R^{4f} are independently at each occurrence selected from: H, —CN, halo, C₁₋₄ alkyl, C₁₋₄ haloalkyl, —OR^{4g}, —NR^{4g}R^{4h}, C₃₋₈ cycloalkyl, 3 to 6 membered heterocyclic ring, 6 to 10 membered aryl, 5 to 10 membered heteroaryl, wherein the C₃₋₈ cycloalkyl, 3 to 6 membered heterocyclic ring, 6 to 10 membered aryl or 5 to 10 membered heteroaryl group is unsubstituted or substituted with 1, 2 or 3 R⁴ⁱ groups; wherein R^{4g} and R^{4h} are independently at each occurrence selected from: H and C₁₋₄ alkyl; and wherein R⁴ⁱ is independently at each occurrence selected from: halo, C₁₋₄ alkyl, C₁₋₄ haloalkyl, —OR^{4j}, —NR^{4k}R^{4l}, —NR^{4k}(C(O))R^{4l}, —C(O)NR^{4k}R^{4l}, —CN, —C(O)R^{4g}, ═O, —SO₂R^{4g}, benzyl, phenyl, unsubstituted 5 or 6 membered heteroaryl, or methyl substituted 5 or 6 membered heteroaryl; R⁴ⁱ is selected from: H, C₁₋₄ alkyl, C₁₋₄ haloalkyl, phenyl or benzyl; R^{4k} and R^{4l} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
n is selected from 0, 1, 2, 3, or 4;
R⁴ is selected from: H, halo, —CN, C₁₋₄ alkyl, C₁₋₄ haloalkyl, —OR^{4g}, —NR^{4g}R^{4h}, a monocyclic or bicyclic 6 to 10 membered aryl, C₃₋₈ cycloalkyl, 3 to 6 membered heterocyclic ring, a monocyclic or bicyclic 5 to 10 membered heteroaryl, a bicyclic (fused, bridged, or spiro) 6 to 10 membered cycloalkyl ring system and a monocyclic or bicyclic (fused, bridged, or spiro) 6 to 10 membered heterocyclic ring system comprising 1, 2, or 3 heteroatoms selected from O, N or S, wherein the C₃₋₈ cycloalkyl, 3 to 6 membered heterocyclic ring, 6 to 10 membered aryl, 5 to 10 membered heteroaryl group, or 6 to 10 membered heterocyclic ring system is unsubstituted or substituted with 1, 2 or 3 R⁴ⁱ;
R⁵ is H or C₁₋₆ alkyl;
   is selected from 1 or 2;
R^{5a} and R^{5b} are independently at each occurrence selected from: H, substituted or unsubstituted C₁₋₆ alkyl, substituted or unsubstituted C₃₋₆ cycloalkyl, and substituted or unsubstituted C₁₋₆ haloalkyl, wherein each substituent is independently selected from halo, —OH, and —CN;
Ring A is selected from a substituted or unsubstituted 5 to 10 membered monocyclic or bicyclic heteroaryl group having 1, 2 or 3 heteroatoms selected from O, N or S, a substituted or unsubstituted 6 to 10 membered monocyclic or bicyclic aryl group, and a substituted or unsubstituted monocyclic or bicyclic (fused, bridged, or spiro) 6 to 10 membered heterocyclic ring system comprising 1, 2, or 3 heteroatoms selected from O, N or S, wherein, when substituted, the heteroaryl group, aryl group, or heterocyclic ring system are substituted with 1, 2, or 3 substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d};
R^{5c} and R^{5d} are independently at each occurrence selected from: H, substituted or unsubstituted C₁₋₆ alkyl, substituted or unsubstituted C₃₋₆ cycloalkyl, and substituted or unsubstituted C₁₋₆ haloalkyl, wherein each substituent is independently selected from halo, —OH, and —CN.
2. The compound of clause 1, wherein —X— is —C(O)—.
3. The compound of clause 1 or clause 2, wherein R¹ is selected from —NR^{1a}R^{1b}, a substituted or unsubstituted 5 or 6 membered monocyclic heteroaryl group having 1, 2 or 3 heteroatoms selected from O, N or S, and a substituted or unsubstituted 3 to 8 membered monocyclic or bicyclic (fused, bridged or spiro) heterocyclic group having 1, 2 or 3 heteroatoms selected from O, N or S.
4. The compound of clause 3, wherein R¹ is a substituted or unsubstituted 4 to 7 membered monocyclic or bicyclic (fused, bridged, or spiro) heterocyclic ring system comprising a nitrogen atom and 0 or 1 additional heteroatoms selected from O, N or S, wherein the heterocyclic ring system is connected to —X— via a nitrogen atom.
5. The compound of clause 3, wherein R¹ is selected from a substituted or unsubstituted:

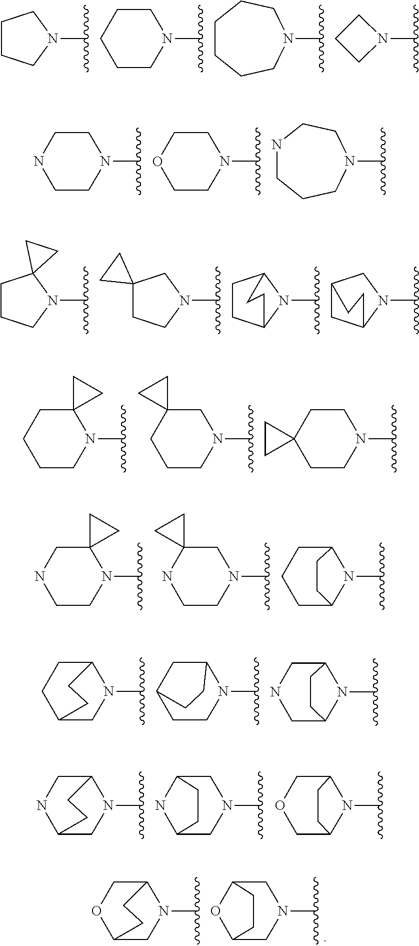

6. The compound of any of clauses 3 to 5, wherein, when substituted, the substituent of R¹ is selected from halo, C₁₋₃ alkyl, C₁₋₃ haloalkyl, and —O—C₁₋₃ alkyl.
7. The compound of clause 3, wherein R¹ is —NR^{1a}R^{1b}.
8. The compound of clause 7, wherein R^{1a} and R^{1b} are independently at each occurrence selected from: substituted or unsubstituted C₁₋₃ alkyl and substituted or unsubstituted C₃ cycloalkyl.
9. The compound of any of clauses 7 to 8, wherein, when R¹ and R^{1b} are substituted, each substituent is independently selected from: halo, C₁₋₃ alkyl, C₁₋₃ haloalkyl, —O—C₁₋₃ alkyl, —O—C₁₋₃ haloalkyl, C₃₋₆ cycloalkyl, and —NR^{1e}R^{1f}.
10. The compound of any preceding clause, wherein R² is selected from H, and C₁₋₃ alkyl.
11. The compound of any preceding clause, wherein m is 0.
12. The compound of any preceding clause, wherein the residue

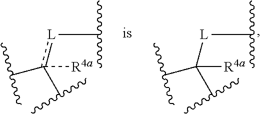

wherein L is a bond.
13. The compound of any of clauses 1 to 11, wherein the residue

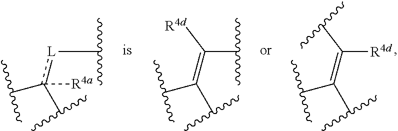

wherein R^{4d} is H.
14. The compound of any preceding clause, wherein R^{4e} and R^{4f} are H.
15. The compound of any preceding clause, wherein n is 1.
16. The compound of any of clauses 1 to 13, wherein n is 0.
17. The compound of any preceding clause, wherein R⁴ is selected from: a monocyclic or bicyclic 6 to 10 membered aryl, a monocyclic or bicyclic 5 to 10 membered heteroaryl, and a monocyclic or bicyclic (fused, bridged, or spiro) 6 to 10 membered heterocyclic ring system comprising 1, 2, or 3 heteroatoms selected from O, N or S, wherein the monocyclic or bicyclic 6 to 10 membered aryl, the monocyclic or bicyclic 5 to 10 membered heteroaryl, or the monocyclic or bicyclic (fused, bridged, or spiro) 6 to 10 membered heterocyclic ring system is unsubstituted or substituted with 1, 2 or 3 R⁴ⁱ.
18. The compound of any preceding clause, wherein R⁴ⁱ is independently at each occurrence selected from: halo, C, alkyl, C, haloalkyl, and —OR^{4j}.
19. The compound of any of clauses 1 to 16, wherein R⁴ is H.
20. The compound of any preceding clause, wherein R⁵ is H.
21. The compound of any preceding clause, wherein o is 1.
22. The compound of any preceding clause, wherein R^{5a} and R^{5b} are H.
23. The compound of any preceding clause, wherein Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}.
24. The compound of any preceding clause, wherein the bicyclic heteroaryl group or the bicyclic heterocyclic ring system are optionally substituted with 1, 2, or 3 substituents independently selected from: halo, C₁₋₃ alkyl, C₁₋₃ haloalkyl, deuterated C₁₋₃ alkyl, or —OR^{5c}.
25. The compound of any of clauses 1 to 22, wherein Ring A is selected from:

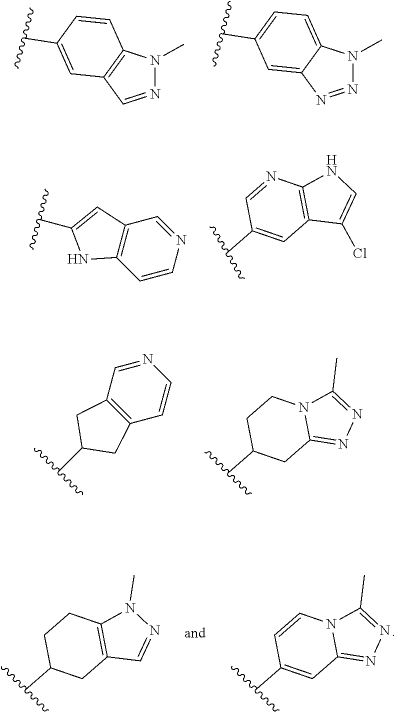

26. A pharmaceutical formulation comprising a compound of any of clauses 1 to 25 and a pharmaceutically acceptable excipient.
27. A compound of any of clauses 1 to 25, for use as a medicament.
28. A compound of any of clauses 1 to 25, for use the prevention or treatment of a condition selected from the following or as a co-therapy in a treatment or prevention of a condition selected from: thrombosis, deep venous thrombosis, thrombosis related to pregnancy, congenital pro-thrombotic disorders, thrombosis resulting from autoimmune conditions, venous and arterial thrombosis as a result of viral or bacterial infections, complex left-sided ablation (pulmonary vein isolation; VT ablation), reperfusion injury also know as ischaemia-reperfusion injury, transcatheter aortic valve replacement (TAVR) also known as transcatheter aortic valve implantation (TAVI), spinal or epidural anaesthesia, lumbar diagnostic puncture, thoracic surgery, abdominal surgery, major orthopaedic surgery, liver biopsy, transurethral prostate resection, kidney biopsy, renal insufficiency, liver diseases, endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, prosthetic valve implantation, myocardial infarction, angina pectoris (including unstable angina), reocclusions and restenoses after angioplasty or aortocoronary bypass, stroke, patients with atrial fibrillation to reduce their risk of stroke, patients with atrial fibriliation and chronic kidney disease, transitory ischaemic attacks, peripheral arterial occlusion disorders, pulmonary embolisms, deep venousmicrovascular disease, patients requiring extra corporeal membrane oxygenation (ECMO), patients requiring extra corporeal circulation such as coronary artert bypass grafting (CABG), disseminated intravascular coagulation (DIC), atherosclerosis, arthritis, thrombosis in patients with cancer, silent brain ischaemia, stroke, neurotraumatic disorder, neurological inflammatory disorders, medical procedures comprising contact with artificial surfaces including renal dialysis, other conditions where inhibition of FXIIa could be beneficial such as Alzheimer's disease, vascular dementia, macular degeneration, diabetic retinopathy, diabetic macular oedema, cerebral oedema in stroke, other causes of oedema, hereditary angioedema or acquired angioedema.

### Examples and Synthesis

¹H-NMR: Spectra are obtained on a Bruker DRX 400 MHz or Jeol ECS 400 MHz spectrometer. Spectra are measured at 294K (unless otherwise stated) and chemical shifts (δ-values) are reported in parts per million (ppm), referenced to either TMS (0.0 ppm), DMSO-d₆ (2.50 ppm), CDCl₃ (7.26 ppm), acetonitrile-d₃ (1.94 ppm), CD₂Cl₂ (5.32 ppm). Coupling constants (J) are reported in Hertz (Hz), spectra splitting pattern are designated as singlet (s), doublet (d), triplet (t), quadruplet (q), multiplet or more overlapping signals (m), broad signal (br); solvent is given in parentheses. ¹H-NMR integrations have been quantified when visible. When integrals are missing it is assumed that some proton signals are (partially) obscured by residual DMSO and/or water peak. Rotamers were observed for many compounds. Where investigated, the ¹H-NMR signals for such compounds were observed to coalesce at high temperature.

### Abbreviations

The following abbreviations are used in the Examples and other parts of the description. Ac: acetate; Boc: tert-butyloxycarbonyl; Cbz: carbobenzyloxy; CV: Column volumes; DCM: dichloromethane; dioxane: 1,4-dioxane; DIPEA: Diisopropyl ethylamine; DMAP: 4-(dimethylamino)pyridine; DMF: N,N-dimethylformamide; DMSO: dimethylsulfoxide; EtOAc: ethyl acetate; h: hour(s); HATU: 2-(7-aza-1H-benzotriazole-1-yl)-1,1,3,3-tetramethyluronium hexafluorophosphate; HPLC: High-performance liquid chromatography; min: minute(s); LCMS: Liquid chromatography-mass spectrometry; LiHMDS: Lithium hexamethyldisilazide; M: Molar; MS: mass spectroscopy; MW: Microwave; Prep: preparative; quant.: quantitative (conversion); Rt: retention time; rt or RT: room temperature; SCX: strong cation exchange; TEA: triethylamine; TFA: trifluoroacetic acid; THF: tetrahydrofuran; SPhos: 2-Dicyclohexylphosphino-2′,6′-dimethoxybiphenyl; UPLC: Ultra-performance liquid chromatography.

### Analytical Methods

Analysis of products and intermediates has been carried out using reverse phase analytical HPLC-MS using the parameters set out below.

HPLC Analytical Methods (run on an Agilent 1100 LC machine with Waters ZQ mass spectrometry):

AnaIpH2_MeOH_4 min: Phenomenex Luna C18 (2) 3 μm, 50×4.6 mm; A=water+0.1% formic acid; B=MeOH+0.1% formic acid; 45° C.; % B: 0.0 min 5%, 1.0 min 37.5%, 3.0 min 95%, 3.5 min 95%, 3.51 min 5%, 4.0 min 5%; 2.25 mL/min. With PDA 210-400 nm and MS detection 120-950 Da, ESi+/−(Waters ZQ LCMS)

AnalpH9_MeOH_4 min: Phenomenex Luna C18 (2) 3 μm, 50×4.6 mm; A=water pH 9 (Ammonium Bicarbonate 10 mM); B=MeOH+0.1% formic acid; 45° C.; % B: 0.0 min 5%, 1.0 min 37.5%, 3.0 min 95%, 3.5 min 95%, 3.51 5%, 4.0 min 5%; 2.25 mL/min. With PDA 210-400 nm and MS detection 120-950 Da, ESi+/−(Waters ZQ LCMS)

AnalpH2_MeCN_4 min: Waters Xbridge C18 3.5 μm, 50×4.6 mm; A=water pH 9 (Ammonium Bicarbonate 10 mM); B=MeCN; 45° C.; % B: 0.0 min 5%, 1.0 min 37.5%, 3.0 min 95%, 3.5 min 95%, 3.51 5%, 4.0 min 5%; 2.25 mL/min. With PDA 210-40 nm and MS detection 120-950 Da, ESi+/−.

AnalpH9_MeCN_4 min: Waters Xbridge C18 3.5 μm, 50×4.6 mm; A=water pH 9 (Ammonium Bicarbonate 10 mM); B=MeCN; 45° C.; % B: 0.0 min 5%, 1.0 min 37.5%, 3.0 min 95%, 3.5 min 95%, 3.51 5%, 4.0 min 5%; 2.25 mL/min. With PDA 210-40 nm and MS detection 120-950 Da, ESi+/−.

AnalpH2_MeOH_QC_V1: Phenomenex Gemini NX C18 5 μm, 150×4.6 mm; A=water+0.1% formic acid; B=MeOH+0.1% formic acid; 40° C.; % B: 0.0 min 5%, 0.5 min, 5%, 7.5 min 95%, 10.0 min 95%, 10.1 min 5%, 13.0 min 5%; 1.5 mL/min. With PDA 210-400 nm and MS detection 120-950 Da, ESi+/−(Waters ZQ LCMS)

AnalpH9_MeOH_QC_V1: Phenomenex Gemini NX C18 5 μm, 150×4.6 mm; A=water+pH 9 (Ammonium Bicarbonate 10 mM); B=MeOH; 40° C.; % B: 0.0 min 5%, 0.50 min 5%, 7.5 min 95%, 10.0 min 95%, 10.1 min 5%, 13.0 min 5%; 1.5 mL/min. With PDA 210-400 nm and MS detection 120-950 Da, ESi+/−(Waters ZQ LCMS)

AnalpH2_MeCN_QC_V2: Phenomenex Gemini NX C18 5 μm, 150×4.6 mm; A=water+0.1% formic acid; B=MeCN+0.1% formic acid; 40° C.; % B: 0.0 min 5%, 0.5 min 5%, 7.5 min 95%, 10.0 min 95%, 10.1 min 5%, 13.0 min 5%; 1.5 mL/min. With PDA 210-400 nm and MS detection 120-950 Da, ESi+/−. (Waters ZQ LCMS)

AnalpH9_MeCN_QC_V2: Phenomenex Gemini NX C18 5 μm, 150×4.6 mm; A=pH 9 Ammonium Bicarbonate 10 mM aqueous solution; B=MeCN; 40° C.; % B: 0.0 min 5%, 0.50 min 5%, 7.5 min 95%, 10.0 min 95%, 10.1 min 5%, 13.0 min 5%; 1.5 mL/min. With PDA 210-400 nm and MS detection 120-950 Da, ESi+/−(Waters ZQ LCMS) HPLC Analytical Methods (run on Waters Acuity UPLC with Waters QDA mass spectrometry):

UPLC_pH2_MeCN_QC_V1: Waters BEH C18 1.7 μm, 100×2.1 mm; A=water+0.1% formic acid; B=MeCN+0.1% formic acid; 40° C.; % B: 0.05 min 5%, 5.00 min 95%, 6.60 min 5% 0.35 mL/min. With PDA 210-400 nm and MS detection 120-1000 Da, ESi+/−. (Waters QDA LCMS)

UPLC_pH10_MeCN_QC_V1: Waters BEH C18 1.7 μm, 100×2.1 mm; A=pH 10 water+0.1% ammonia; B=MeCN; 40° C.; % B: 0.05 min 5%, 5 min 95%, 6.6 min 5%, 0.35 mL/min. With PDA 210-400 nm and MS detection 120-1000 Da, ESi+/−(Waters QDA LCMS)

Thermo_MeOH_UHPLC_1.2 min: Phenomenex Kinetex, 2.6 uM, 50×2.1 mm, A=water+0.1% formic acid; B=MeOH+0.1% formic acid; 2-95% B 0-1.0 min; 1.3 mL/min

### GENERAL METHODS

### General Method 1 (GM1): Amide Coupling

A mixture of carboxylic acid (1.0 eq), amine (1.0-1.5 eq), N,N-diisopropylethylamine or triethylamine (3.0-6.0 eq) and HATU (1.0-1.2 eq) in anhydrous solvents such as DMF and/or DCM was stirred at room temperature or 0° C. for 1-72 h.

The solvent was removed in vacuo and the residue dissolved in EtOAc or DCM. (Optionally the reaction mixture was directly diluted). A mixture of water and/or brine and/or aqueous saturated NaHCO₃ solution was added and the product extracted with EtOAc or DCM. The organic phase was optionally washed with brine (up to 3 times) and/or 10% aqueous citric acid. The organic phase was dried over Na2SO4 or MgSO4 or passed through a hydrophobic frit (phase separator, Biotage) and concentrated in vacuo. Crude material was either used without further purification, filtered through a silica plug, purified by flash column chromatography or preparative HPLC.

Optionally the reaction mixture was directly purified by preparative HPLC without aqueous work-up.

Optionally the starting amine reactant may be used as the TFA or HCl salt.

### General Method 2 (GM2): Boc Deprotection

Method Boc deprotection 2A: Method Boc deprotection 2A: Boc-protected amine was stirred in a mixture of DCM:TFA (in a ratio from 10:1 to 1:1) for 1-18 h.

Method Boc deprotection 2B: Boc-protected amine was dissolved in EtOAc or dioxane and either 4M HCl in dioxane or 1 M HCl in Et₂O added. The reaction mixture was stirred at room temperature for 1-18 h.

The reaction mixture was concentrated in vacuo to yield the crude material which was either used directly in the subsequent reaction, or purified by one of the following methods:
a) SCX-2 optionally followed by preparative HPLC
b) Dissolved in water and aqueous NH3 solution (35%) was added until pH=10. The resulting suspension was extracted with EtOAc and then with DCM. The two organic fractions were combined and evaporated to dryness.
c) The reaction was diluted with DCM and washed with saturated ammonium bicarbonate or sodium bicarbonate and optionally brine. The organic layer was dried over MgSO₄ and filtered. Optionally followed by preparative HPLC.
d) Crude triturated with Et₂O or DCM.
e) Crude directly purified by preparative HPLC.

### General Method 3 (GM3): Hydrogenation

The Cbz-protected amine (1 eq) was dissolved in either EtOH, MeOH or MeOH:DCM (1:1), placed under N₂ atmosphere, and Pd/C (10 wt %) was added. A H₂ atmosphere was introduced and the reaction mixture stirred at room temperature for 1-72 h. The mixture was filtered through celite and the filtrate concentrated to give the crude product which was either used with no further purification, or purified by SCX-2 or preparative HPLC.

Optionally additional aliquots of Pd/C may be added during the course of the reaction.

### Synthesis

### S1 Reagents

### Synthesis of (7-fluoro-1H-pyrrolo[3,2-c]pyridin-2-yl)methanamine (M05712-int)

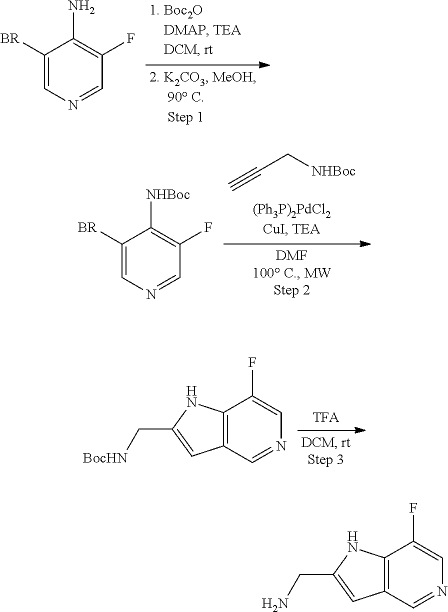

Step 1: 3-Bromo-5-fluoropyridin-4-amine (2 g, 9.95 mmol), 4-(dimethylamino)pyridine (DMAP) (608 mg, 4.97 mmol) and triethylamine (4.2 mL, 29.8 mmol) were dissolved in DCM (30 mL). Di-tert-butyl dicarbonate (4.6 g, 20.9 mmol) dissolved in DCM (10 mL) was added to the reaction mixture and was stirred at room temperature overnight. The mixture was washed with water (2×100 mL) and 10% aqueous citric acid (100 mL) to remove DMAP. The organic fraction was passed through a phase separator cartridge (Biotage), evaporated to dryness and redissolved in methyl alcohol (50 mL). Potassium carbonate (4.1 g, 29.8 mmol) was added and the reaction mixture was heated to reflux (90° C. outside temperature) for 4 hours. The reaction mixture was filtered, the filtrate was evaporated to dryness and purified by flash column chromatography (SiO₂, Biotage Isolera, 25 g Sfär column, eluting with 100% isohexane to 25% EtOAc in isohexane; target material eluted at 15% EtOAc in isohexane). The fractions containing target material were combined and evaporated to afford tert-butyl N-(3-bromo-5-fluoro-4-pyridyl)carbamate (2.2 g, 77%) as a yellow oil.

AnalpH9_MeCN_4 min, Rt: 2.20 min, m/z 291.0 and 293.0 [M+H]⁺ bromide splitting

Step 2: A solution of N-Boc-prop-2-ynylamine (1.2 g, 7.56 mmol), tert-butyl N-(3-bromo-5-fluoro-4-pyridyl)carbamate (1.1 g, 3.78 mmol), bis(triphenylphosphine)palladium(II) dichloride (133 mg, 0.189 mmol), copper(I) iodide (22 mg, 0.113 mmol) and triethylamine (2.1 mL, 15.1 mmol) in DMF (25 mL) was degassed by bubbling N₂ through for a few minutes, and was then heated to 100° C. for 1.5 hours in a microwave reactor. The reaction mixture was evaporated to dryness, was redissolved in DCM (100 mL) and washed with water (50 mL) and brine (50 mL). The organic fraction was passed through a phase separator cartridge (Biotage) and evaporated to dryness. The crude product was redissolved in DMF (40 mL) and the solution was heated to 100° C. overnight. The mixture was evaporated to dryness and the crude was purified by flash column chromatography (SiO₂, Biotage Isolera, 100 g Sfär column, eluting with 25% EtOAc in isohexane to 100% EtOAc; target material elutes at 65-80% EtOAc in isohexane). The fractions containing the product were purified again by flash column chromatography (SiO₂, Biotage Isolera, 100 g Sfär column, eluting with 50% EtOAc in isohexane to 100% EtOAc) affording tert-butyl N-[(7-fluoro-1H-pyrrolo[3,2-c]pyridin-2-yl)methyl]carbamate (701 mg, 70%) as a yellow solid.

AnalpH9_MeCN_4 min, Rt: 1.94 min, m/z 266.2 [M+H]⁺

Step 3: tert-Butyl N-[(7-fluoro-1H-pyrrolo[3,2-c]pyridin-2-yl)methyl]carbamate (320 mg, 1.21 mmol) was dissolved in DCM (20 mL) and trifluoroacetic acid (5.0 mL) was added. The reaction mixture was stirred at room temperature for 1 h. The mixture was evaporated to dryness, was redissolved in MeOH and was loaded onto an SCX-2 cartridge (Biotage, 5 g). The column was washed several times with MeOH, then the bound target material was eluted with 3.5 M NH₃ in MeOH. The solvent was evaporated to afford (7-fluoro-1H-pyrrolo[3,2-c]pyridin-2-yl)methanamine (178 mg, 89%) as a brown gum.

AnalpH9_MeCN_4 min, Rt: 0.98 min, m/z 166.2 [M+H]⁺

### Synthesis of (3-methyl-[1,2,4]triazolo[4,3-a]pyridin-7-yl)methanamine (M05855-int)

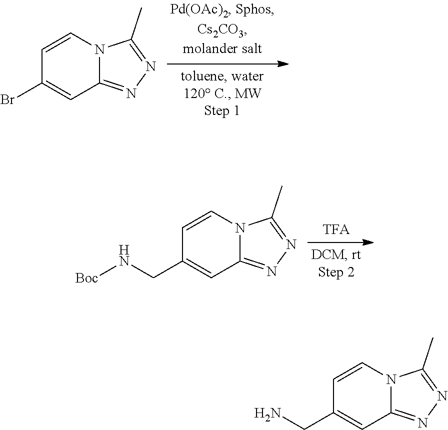 [SMILES: Cc1nnc2cc(Br)ccn12 ; Cc1nnc2cc(CNC(=O)OC(C)(C)C)ccn12 ; Cc1nnc2cc(CN)ccn12]

Step 1: To a stirred solution of 7-bromo-3-methyl-[1,2 4]triazolo[4.3-a]pyridine (250 mg, 1.18 mmol) in 1,4-dioxane (10 mL) was added potassium N-boc-aminomethyltrifluoroborate (559 mg, 2.36 mmol), palladium (II) acetate (53 mg, 0.24 mmol) and Sphos (194 mg, 0.47 mmol). To this was added caesium carbonate (1.15 g, 3.54 mmol) in water (2.5 mL). The reaction mixture was sparged with N₂ for 5 minutes. The solution was then heated at 120° C. in a microwave reactor for 2 hours. The reaction mixture was passed through a celite cartridge (2.5 g, Biotage), flushed with methanol and the combined filtrate concentrated under reduced pressure. Water (10 mL) was added, and the product was extracted into DCM (3×20 mL). The combined organic layers were washed with aqueous saturated NH₄Cl (10 mL) followed by brine (10 mL) and then passed through a phase separator cartridge (Biotage) and concentrated in vacuo to give tert-butyl ((3-methyl-[1,2,4]triazolo[4,3-a]pyridin-7-yl)methyl)carbamate (387 mg of crude, quantitative). The crude product was used directly in the next step without further purification, assuming 100% yield (307 mg).

AnalpH9_MeCN_4 min, Rt: 1.63 min, m/z 263.2 [M+H]⁺

Step 2: tert-Butyl N-[(3-methyl-[1,2,4]triazolo[4,3-a]pyridin-7-yl)methyl]carbamate (307 mg, 1.18 mmol) was dissolved in DCM (12 mL), the reaction mixture was cooled to 0° C. and trifluoroacetic acid (4 mL) was added dropwise over 5 mins under stirring. The solution was allowed to reach room temperature and stirred for 2 hours. The reaction mixture was concentrated in vacuo and the crude residue was redissolved in MeOH (2 mL) before being loaded onto an SCX-2 cartridge (2 g, Biotage). The cartridge was then eluted with MeOH (3 CV) and then the bound amine was eluted with 3.5M NH₃ in MeOH. The solvent was removed in vacuo and the solid was triturated with DCM to give (3-methyl-[1,2,4]triazolo[4,3-a]pyridin-7-yl)methanamine (102 mg, 53%) as a white solid.

AnalpH9_MeCN_4 min, Rt: 0.54 min, m/z 163.2 [M+H]⁺

### Synthesis of (3-methoxy-1-methyl-1H-indazol-5-yl)methanamine (M05810-int)

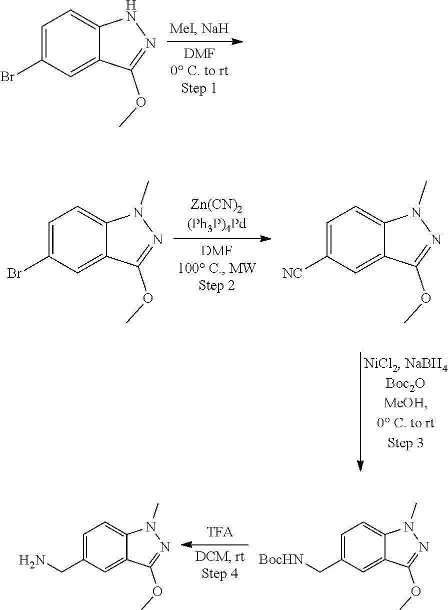 [SMILES: COc1n[nH]c2ccc(Br)cc12 ; COc1nn(C)c2ccc(Br)cc12 ; COc1nn(C)c2ccc(C#N)cc12 ; COc1nn(C)c2ccc(CN)cc12 ; COc1nn(C)c2ccc(CNC(=O)OC(C)(C)C)cc12]

Step 1: To a solution of 5-bromo-3-methoxy-1H-indazole (250 mg, 1.10 mmol) in N,N-dimethylformamide (10 mL) was added sodium hydride (60% dispersion in oil) (53 mg, 1.32 mmol) at 0° C. The solution was allowed to warm to room temperature and was stirred for 30 minutes. Iodomethane (0.14 mL, 2.20 mmol) was added, and the solution was stirred at room temperature for 3 hours. LCMS indicated the formation of two regioisomers in a 5:95 ratio. The reaction mixture was diluted with water (50 mL) and was extracted with EtOAc (3×30 mL). The combined organic fractions were washed with brine (3×30 mL), passed through a phase separator cartridge (Biotage) and evaporated to dryness. The crude material was then purified by preparative HPLC to afford the two isomeric target materials. The desired isomer 5-bromo-3-methoxy-1-methyl-indazole (197 mg, 74%, white solid) was formed preferentially.

AnalpH9_MeCN_4 min, Rt:2.58 min, m/z 241.0 and 243.0 [M+H]⁺

¹H-NMR (400 MHz, DMSO-D₆) δ 7.76 (s, 1H); 7.50 (s, 1H), 7.49 (s, 1H), 3.99 (s, 3H), 3.86 (s, 3H)

Step 2: To a solution of 5-bromo-3-methoxy-1-methyl-indazole (197 mg, 0.82 mmol) in N,N-dimethylformamide (4.0 mL) was added tetrakis(triphenylphosphine)palladium(0) (94 mg, 0.082 mmol) followed by zinc cyanide (115 mg, 0.981 mmol). The reaction mixture was degassed by bubbling N₂ through it for five minutes, then was heated to 100° C. for 1.5 hours in a microwave reactor. The reaction mixture was diluted with ethyl acetate (50 mL) and washed with an aqueous saturated solution of NaHCO₃ (3×30 mL). The organic extract was passed through a phase separator cartridge (Biotage) and concentrated in vacuo. The crude material was then purified by flash column chromatography (SiO₂, Biotage Isolera, 25 g SNAP column, eluting from 100% isohexane to 50% EtOAc in isohexane; target material elutes at 30% EtOAc in isohexane). Fractions containing target material were combined and evaporated to afford 3-methoxy-1-methyl-indazole-5-carbonitrile (81 mg, 53%) as a white solid.

AnalpH9_MeCN_4 min. Rt: 2.11 min, m/z 188.2 [M+H]⁺

Step 3: To a solution of 3-methoxy-1-methyl-indazole-5-carbonitrile (81 mg, 0.43 mmol) in methanol (10 mL) was added di-tert-butyl dicarbonate (189 mg, 0.865 mmol) followed by nickel (II) chloride (5.7 mg, 0.043 mmol). The solution was cooled to 0° C. and sodium borohydride (115 mg, 3.03 mmol) was added portion-wise over 10 minutes. The solution turned black upon addition and effervescence was observed. Once all the sodium borohydride was added, the solution was allowed to warm to room temperature and was stirred for 3 hours. The reaction was quenched with water (3 mL), then the solvent was removed in vacuo and the crude residue was redissolved in EtOAc (20 mL). The organic solution was washed with a saturated aqueous solution of NaHCO₃ (20 mL), followed by 1% aqueous solution of diethylenetriamine (20 mL), a saturated solution of NaHCO₃ (20 mL), brine (10 mL) and finally water (10 mL). The organic phase was passed through a phase separator cartridge (Biotage) and the solvent removed under vacuum to afford tert-butyl N-[(3-methoxy-1-methyl-indazol-5-yl)methyl]carbamate (113 mg, 90%) as a colourless oil.

AnalpH9_MeCN_4 min, Rt: 2.36 min, m/z 292.2 [M+H]⁺

Step 4: tert-Butyl N-[(3-methoxy-1-methyl-indazol-5-yl)methyl]carbamate (113.mg, 0.39 mmol) was dissolved in DCM (15 mL). Trifluoroacetic acid (5.0 mL) was added and the reaction mixture was stirred at room temperature for 1 hour. The reaction mixture was evaporated to dryness, was redissolved in MeOH and was loaded onto a SCX-2 column (Biotage, 2 g). The column was washed several times with MeOH, then the bound target material was eluted with 3.5 NH₃ in MeOH. The solvent was evaporated to afford (3-methoxy-1-methyl-indazol-5-yl)methanamine (37 mg, 50%) as a colourless gum.

AnalpH9_MeCN_4 min, Rt: 1.31 min, m/z 193.2 [M+H]⁺

Alternative N-alkylation methodology: Step 1—method B

### Synthesis of 1-(difluoromethyl)indazole-5-carbonitrile (M05778-int)

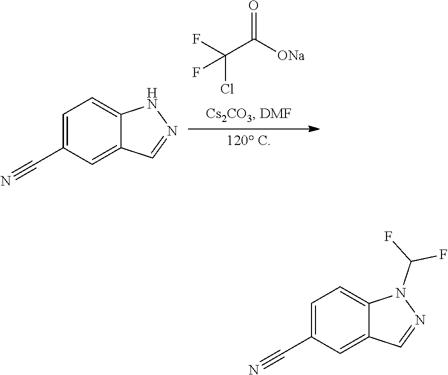

1H-indazole-5-carbonitrile (100 mg, 0.70 mmol) was dissolved in dry N,N-dimethylformamide (5 mL). Cs₂CO₃ (228 mg, 0.70 mmol) was added and the reaction was heated to 120° C. and stirred vigorously for 5 mins. Sodium chlorodifluoroacetate (213 mg, 1.4 mmol) was added to the reaction mixture and then stirred at 120° C. for 1 hour. A further portion of sodium chlorodifluoroacetate (106 mg, 0.70 mmol) was added and the reaction mixture was stirred for an additional 1 hour. The solvent was removed under vacuum and the residue was dissolved in DCM (10 mL). The organic solution was washed with water (10 mL), followed by aqueous saturated NH4Cl (10 mL) and brine (10 mL) before being passed through a phase separator (Biotage) and the solvent was removed under vacuum. The crude product was purified by flash column chromatography (SiO₂, Biotage isolera, 10 g, Sfär column, eluting from 5 to 50% EtOAc in isohexane) to give 1-(difluoromethyl)indazole-5-carbonitrile (73 mg, 54%) as an off-white solid.

AnalpH9_MeCN_4 min, Rt: 1.88, m/z 192.1 [M+H]⁺

¹H-NMR (400 MHz, CDCl₃) δ 8.18-8.20 (2H, m), 7.88-7.90 (1H, m), 7.74 (1H, dd, J=8.1, 1.4 Hz), 7.50 (1H, t, J=59.2 Hz)

The following compounds were made by analogous methods:

| | | | Mass, % |
|---|---|---|---|
| Example No. | Structure & conditions | Analytical Data | yield, state |
| M05806-int (3-fluoro-1-methyl- indazol-5- yl)methanamine | 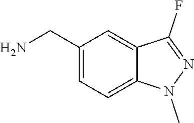 From 5-bromo-3-fluoro-1- methyl-1H-indazole Step 1 not required | AnalpH9_MeCN_4 min, Rt: 1.34 mins, m/z 163.2 [M − NH₃]⁺ ¹H-NMR (400 MHz, DMSO-D₆) δ 7.54-7.59 (2H, m), 7.45-7.49 (1H, m), 3.89 (3H, s), 3.80 (2H, s) | 96 mg, quantitative, Off-white solid |
| M05778-int [1-(difluoromethyl) indazol-5-yl] methanamine | 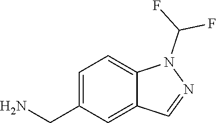 [SMILES: NCc1ccc2c(cnn2C(F)F)c1] From 1H-indazole-5-carbonitrile using Step 1 - method B Step 2: not needed | AnalpH9_MeCN_4 min, Rt: 1.42, m/z 198.2 [M + H]⁺ | 35 mg, 66%, colourless gum |
| M05820-int (1-(methyl-d3)-1H- indazol-5-yl) methanamine | 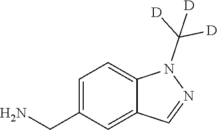 [SMILES: [2H]C([2H])([2H])n1ncc2cc(CN)ccc21] From 1H-indazole-5-carbonitrile Step 2: not needed | AnalpH9_MeCN_4 min, Rt: 1.06, m/z 165.2 [M + H]⁺ | 149 mg, 99%, colourless gum |
| M05851-int (3-(trifluoromethyl)- 5,6,7,8-tetrahydro- [1,2,4]triazolo[4,3- a]pyridin-7-yl) methanamine | 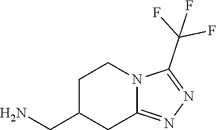 [SMILES: NCC1CCn2c(nnc2C(F)(F)F)C1] From 7-bromo-3-(trifluoromethyl)- [1,2,4] triazolo[4,3-a] pyridine Step 1: not required Step 3: reduction of nitrile also gives concommitment reduction of 6-membered ring to afford tert-butyl ((3-(trifluoromethyl)- 5,6,7,8-tetrahydro-[1,2,4]triazolo [4,3-a]pyridin-7-yl) methyl)carbamate | AnalpH9_MeCN_4 min, Rt: 1.09, m/z 221.2 [M + H]⁺ | 94 mg, 63%, yellow oil |

### Synthesis of (7-methoxy-1-methyl-indazol-5-yl)methanamine (M05799-int)

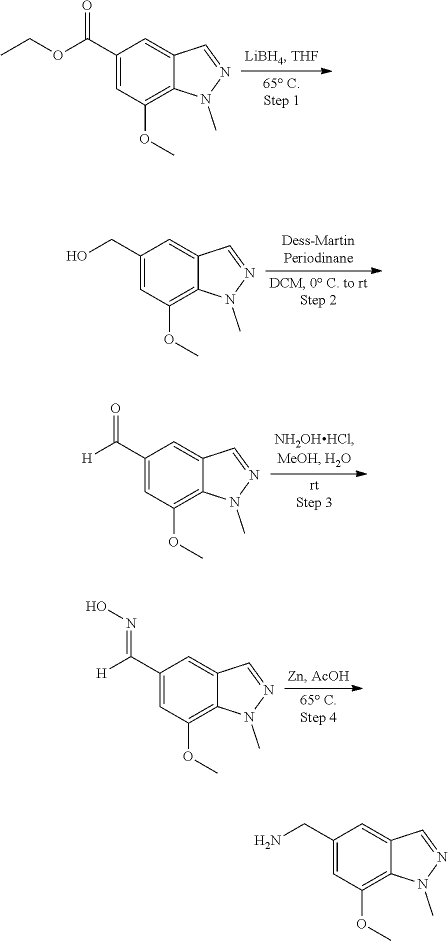 [SMILES: CCOC(=O)c1cc(OC)c2c(cnn2C)c1 ; COc1cc(CO)cc2cnn(C)c12 ; COc1cc(C=O)cc2cnn(C)c12 ; COc1cc(/C=N/O)cc2cnn(C)c12 ; COc1cc(CN)cc2cnn(C)c12]

Step 1: To a stirred solution of ethyl 7-methoxy-1-methyl-1H-indazole-5-carboxylate (500 mg, 2.13 mmol) in tetrahydrofuran (10 mL) was added lithium borohydride (139 mg, 6.40 mmol) and the mixture was stirred for 16 hours at 65° C. Once complete, the mixture was allowed to cool to room temperature and then carefully quenched with 10% aqueous citric acid (20 mL). The solution was then concentrated in vacuo to remove the organic solvent and the resulting mixture was extracted with DCM (3×30 mL). The combined organic layers were then dried over MgSO₄, filtered and concentrated to dryness, affording (7-methoxy-1-methyl-indazol-5-yl)methanol (400 mg, 98%), as a yellow oil.

AnalpH2_MeOH_4 min, Rt: 1.93 mins, m/z 193.2 [M+H]⁺

Step 2: To a stirred solution of (7-methoxy-1-methyl-indazol-5-yl)methanol (400 mg, 2.08 mmol) in anhydrous DCM (12 mL) at 0° C. was added Dess-Martin periodinane (1.3 g, 3.12 mmol). The mixture was stirred for 3 hours and was allowed to warm to rt during this period. The reaction was quenched by addition of 20% aqueous sodium thiosulfate (20 mL) and then aqueous saturated NaHCO₃ solution (10 mL) added. The mixture was then stirred for a further 30 minutes. The mixture was extracted with DCM (3×20 mL), and the combined organic layers dried over MgSO₄, filtered, and concentrated to dryness in vacuo to afford 7-methoxy-1-methyl-indazole-5-carbaldehyde (330 mg, 83%), as a light yellow solid.

AnalpH2_MeOH_4 min, Rt: 2.55 min, m/z 191.2 [M+H]⁺

Step 3: To a stirred solution of 7-methoxy-1-methyl-indazole-5-carbaldehyde (330 mg, 1.74 mmol) in methanol (10 mL) at rt was added a solution of hydroxylamine hydrochloride (265 mg, 3.82 mmol) in water (10 mL) and the mixture stirred at rt for 3 hours. The reaction mixture was then concentrated in vacuo to remove the organic solvent. The resultant aqueous suspension was extracted with DCM (3×30 mL), dried over MgSO₄, filtered and concentrated in vacuo to afford (5E)-7-methoxy-1-methyl-indazole-5-carbaldehyde oxime (340 mg, 96%) as an orange solid.

AnalpH2_MeOH_4 min, Rt: 2.49 min, m/z 206.2 [M+H]⁺

Step 4: To a stirred solution of (5E)-7-methoxy-1-methyl-indazole-5-carbaldehyde oxime (340 mg, 1.66 mmol) in acetic acid (17 mL) was added zinc dust (1.00 g, 15.3 mmol) carefully and the mixture stirred for 16 hours at 75° C. The mixture was cooled to room temperature and filtered through a pad of celite. The filtrate was concentrated to dryness in vacuo, and the residue re-dissolved in MeOH (5 mL). The solution was purified by SCX-2 (5 g, Biotage) washing with MeOH (5 CV) and eluting with 1M NH₃-MeOH (5 CV). Fractions containing the desired product were combined and the solvent evaporated in vacuo. The residue was lyophilised, affording (7-methoxy-1-methyl-indazol-5-yl)methanamine (210 mg, 66%) as an orange-brown gum.

AnalpH2_MeOH_4 min, Rt: 2.01 min, m/z 192.2 [M+H]⁺

### Synthesis of (3-chloro-1H-pyrrolo[2,3-b]pyridin-5-yl)methanamine (M05899-int)

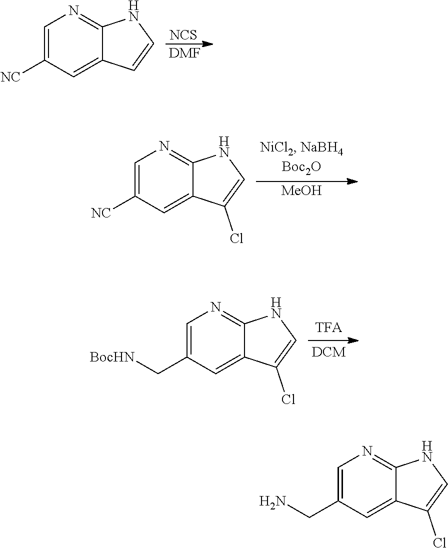

Step 1: 1 H-Pyrrolo[2,3-b]pyridine-5-carbonitrile (1 g, 6.64 mmol) was dissolved in N,N-dimethylformamide (10 mL). N-Chlorosuccinimide (886 mg, 6.64 mmol) was added and the reaction mixture was heated to 55° C. for 6 hours. A further aliquot of N-chlorosuccinimide (300 mg, 2.25 mmol) was added and the reaction mixture was heated at 55° C. for a further 2 hours, then allowed to cool to room temperature and stirred overnight.mA white precipitate was observed in the reaction mixture. The reaction mixture was filtered and the solid was washed with MeOH (1 mL). The precipitate was dried on under reduced pressure (6mbar at 50° C.) to afford 3-chloro-1H-pyrrolo[2,3-b]pyridine-5-carbonitrile (931 mg, 5.24 mmol, 79%) as a white solid.

AnalpH9_MeCN_4MIN: Rt=1.94 min; m/z=176.0; 178.0; [M+H]1; 98% pure.

Step 2: To a solution of 3-chloro-1H-pyrrolo[2,3-b]pyridine-5-carbonitrile (931 mg, 5.24 mmol) in methanol (150 mL) (material is not completely in solution) was added di-tert-butyl dicarbonate (2288 mg, 10.5 mmol) followed by nickel (II) chloride (69 mg, 0.52 mmol). The solution was cooled to 0° C. and sodium borohydride (1388 mg, 36.7 mmol) was added portionwise over 10 minutes. The solution turned black on addition and effervescence was observed. Once the addition of sodium borohydride was complete, the solution was allowed to warm to RT and was stirred overnight.

The reaction mixture was filtered and the residue washed with MeOH. The combined filtrate and washings were evaporated under reduced pressure and the residue was redissolved in EtOAc. The crude product was washed with a saturated solution of NaHCO₃, followed by a 1% solution of diethylenetriamine (20 mL), a saturated solution of NaHCO₃, brine and finally water. The organic phase was passed through a phase separator cartridge (Biotage) and the solvent removed. The crude product was purified by flash column chromatography (Biotage Isolera Four. 25 g Sfar column, DCM->5% MeOH/DCM). The target material co-elutes with the nitrile starting material. The product-containing fractions were combined and evaporated under reduced pressure to afford a mixture of nitrile starting material and tert-butyl ((3-chloro-1H-pyrrolo[2,3-b]pyridin-5-yl)methyl)carbamate as a yellow solid (419 mg). The crude product was used directly in the subsequent step.

Step 3: The crude product containing tert-butyl N-[(3-chloro-1H-pyrrolo[2,3-b]pyridin-5-yl)methyl]carbamate (419 mg, 1.49 mmol) was dissolved in trifluoroacetic acid (6.0 mL) and the reaction mixture was stirred at room temperature for 2 hours. The reaction mixture was evaporated and resuspended in 50 mL MeOH and filtered. The filtrate was loaded onto an SCX-2 column (Biotage, 5 g), the column was washed several times with MeOH, then the bound target material was eluted with NH3/MeOH. The solvent was evaporated to afford (3-chloro-1H-pyrrolo[2,3-b]pyridin-5-yl)methanamine (201 mg, 1.11 mmol, 74.4%) as a yellow solid.

AnalpH9_MeCN_4MIN Rt=1.37 min; m/z+=182.1; 85% pure.

### Synthesis of (3-methyl-5,6,7,8-tetrahydro-[1,2,4]triazolo[4,3-a]pyridin-7-yl)methanamine (M05913-int)

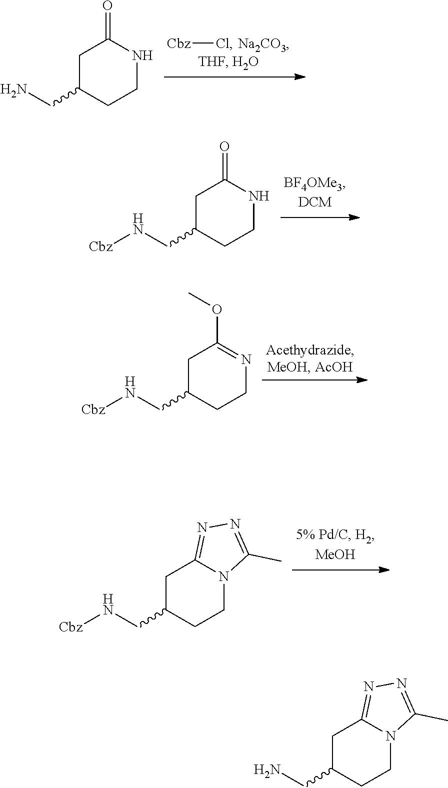 [SMILES: NCC1CCNC(=O)C1 ; O=C(Cl)OCc1ccccc1 ; O=C1CC(CNC(=O)OCc2ccccc2)CCN1 ; COC1=NCCC(CNC(=O)OCc2ccccc2)C1 ; Cc1nnc2n1CCC(CNC(=O)OCc1ccccc1)C2 ; Cc1nnc2n1CCC(CN)C2]

### Step 1: Synthesis of benzyl N-[(2-oxo-4-pipe ridyl)meth yl]carbamate

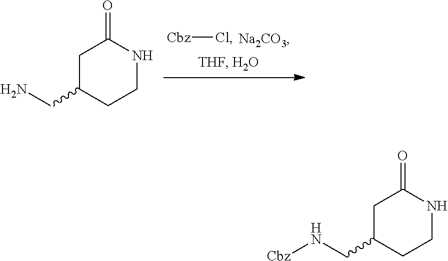 [SMILES: NCC1CCNC(=O)C1 ; O=C(Cl)OCc1ccccc1 ; O=C1CC(CNC(=O)OCc2ccccc2)CCN1]

To a solution of 4-(aminomethyl)piperidin-2-one (500 mg, 3.90 mmol) in tetrahydrofuran (10 mL) was added sodium carbonate (827 mg, 7.80 mmol) in water (5.0 mL). The solution was cooled to 0° C. and benzyl chloroformate (0.82 mL, 5.85 mmol) was added dropwise while stirring at this temperature. The reaction was stirred at 0° C. for 15 mins before being warmed to RT, and stirring continued for 4 hours. The reaction was quenched by addition of saturated aq. NH4Cl (10 mL) and the mixture stirred for 20 mins. THE was removed under vacuum and the product was extracted into DCM (3×30 mL). The combined organic extracts were passed through a phase separator (Biotage) and the product was purified by flash column chromatography (SiO₂, Biotage Isolera, 25 g Sfär column; eluting with 100% DCM to 10% MeOH in DCM) affording benzyl N-[(2-oxo-4-piperidyl)methyl]carbamate (905 mg, 3.45 mmol, 88%) as a colourless oil.

AnalpH9_MeCN_4 min, RT: 1.70, m/z 263.2 [M+H]⁺

### Step 2: Synthesis of benzyl ((6-methoxy-2,3,4,5-tetrahydropyridin-4-yl)methyl)carbamate

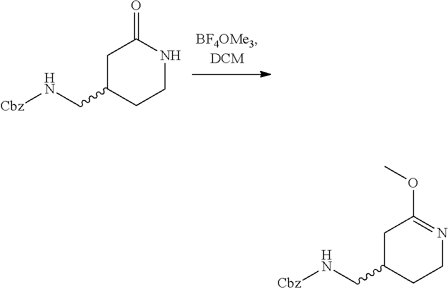 [SMILES: O=C1CC(CNC(=O)OCc2ccccc2)CCN1 ; COC1=NCCC(CNC(=O)OCc2ccccc2)C1]

To a stirred solution of benzyl N-[(2-oxo-4-piperidyl)methyl]carbamate (905 mg, 3.45 mmol) in dichloromethane (20 mL) at 0° C. was added trimethyloxonium tetrafluoroborate (914 mg, 5.87 mmol) portion-wise. The reaction mixture was stirred at 0° C. for 30 mins before being allowed to warm to room temperature and was stirred for a further 3 hours. The reaction was quenched by the addition of sat. aq. NaHCO₃ (15 mL) and the organic layer was separated. The aqueous layer was extracted with DCM (2×15 mL) and the combined organic extracts were washed with brine (15 mL), passed through a phase separator (Biotage) and the product was purified by flash column chromatography (SiO₂, Biotage Isolera, 25 g Sfär column; eluting with 100% DCM to 15% MeOH in DCM) to afford benzyl ((6-methoxy-2,3,4,5-tetrahydropyridin-4-yl)methyl)carbamate (185 mg, 19%) as a white solid.

AnalpH9_MeCN_4 min, RT: 2.08, m/z 277.2 [M+H]⁺

### Step 3: Synthesis of benzyl N-[(3-methyl-5,6,7,8-tetrahydro-[1,2,4]triazolo[4,3-a]pyridin-7-yl)methyl]carbamate

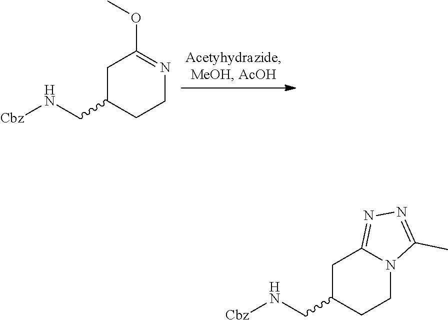 [SMILES: COC1=NCCC(CNC(=O)OCc2ccccc2)C1 ; Cc1nnc2n1CCC(CNC(=O)OCc1ccccc1)C2]

To a solution of benzyl N-[(6-methoxy-2,3,4,5-tetrahydropyridin-4-yl)methyl]carbamate (185 mg, 0.67 mmol) in methanol (5.0 mL) was added acethydrazide (52 mg, 0.67 mmol). The reaction was heated to 65° C. and stirred overnight. The reaction was cooled, and the solvent removed under reduced pressure. Acetic acid (3.0 mL) was added and the reaction mixture was heated to reflux and stirred for 2 hours. The solvent was removed under reduced pressure and the residue was redissolved in water (5 mL), basified with saturated aq. NaHCO₃ and the product extracted into DCM (3×10 mL). The combined organic extracts were passed through a phase separator (Biotage) and purified by flash column chromatography (SiO₂, Biotage Isolera, 10 g Sfär column; eluting with 100% DCM to 10% MeOH in DCM) to afford benzyl N-[(3-methyl-5,6,7,8-tetrahydro-[1,2,4]triazolo[4,3-a]pyridin-7-yl)methyl]carbamate (87 mg, 43%) as a colourless gum.

AnalpH9_MeCN_4 min, RT: 1.73, m/z 301.2 [M+H]⁺

### Step 4: Synthesis of (3-methyl-5,6,7,8-tetrahydro-[1,2,4]triazolo[4,3-a]pyridin-7-yl)methanamine

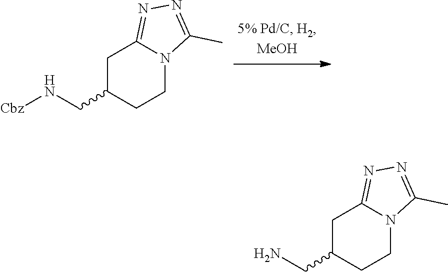 [SMILES: Cc1nnc2n1CCC(CNC(=O)OCc1ccccc1)C2 ; Cc1nnc2n1CCC(CN)C2]

To a stirred solution of benzyl N-[(3-methyl-5,6,7,8-tetrahydro-[1,2,4]triazolo[4,3-a]pyridin-7-yl)methyl]carbamate (87 mg, 0.29 mmol) in methanol (3.0 mL) was added under N₂ atmosphere Pd/C (10 wt %) (3.0 mg, 0.03 mmol). A hydrogen atmosphere was introduced and the reaction mixture was stirred for 3 hours. The reaction mixture was filtered through celite (Isolute, 2.5 g) and was loaded onto an SCX cartridge (2 g, Biotage). The cartridge was then eluted with MeOH (3 CV) and then the amine was eluted with NH₃ (3.5M in MeOH) to give (3-methyl-5,6,7,8-tetrahydro-[1,2,4]triazolo[4,3-a]pyridin-7-yl)methanamine (43 mg, 89%) as a colourless gum.

AnalpH9_MeCN_2 min, RT: 0.39, m/z 167.2 [M+H]⁺

### Synthesis of [3-(difluoromethyl)-[1,2,4]triazolo[4,3-a]pyridin-7-yl]methanamine

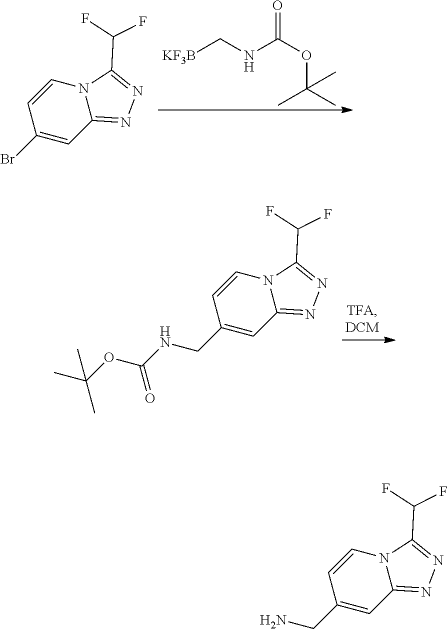

### Step 1: Synthesis of tert-butyl N-[[3-(difluoromethyl)-[1,2,4]triazolo[4,3-a]pyridin-7-yl]methyl]carbamate

To a solution of 7-bromo-3-(difluoromethyl)-[1,2,4]triazolo[4,3-a]pyridine (500 mg, 2.02 mmol) in 1,4-dioxane (10 mL) was added potassium N-Boc-aminomethyltrifluoroborate (956 mg, 4.03 mmol), Dicyclohexyl(2′,6′-dimethoxy-[1,1′-biphenyl]-2-yl)phosphine (SPhos) (331 mg, 0.806 mmol) and Palladium (II) acetate (91 mg, 0.403 mmol). To this was added cesium carbonate (1970 mg, 6.05 mmol) in water (2.5 mL). The mixture was stirred and degassed with N2 for 10 mins. The solution was then heated to 95° C. and left to stir overnight. The dioxane was removed in vacuo and the reaction mixture was diluted with water (10 mL) and DCM (10 mL). The layers were partitioned and the aqueous was further extracted with DCM (2×10 mL). The combined organics were washed with 1 M HCl (10 mL), sat soln of NaHCO₃(10 mL) followed by brine (10 mL) before being passed through a phase separator (Biotage) and concentrated in vacuo to give tert-butyl N-[[3-(difluoromethyl)-[1,2,4]triazolo[4,3-a]pyridin-7-yl]methyl]carbamate (601 mg, 2.01 mmol, 100%,) as a yellow oil, which was used crude in the next step.

AnalpH9_MeCN_4 min: Rt: 1.84 min, m/z 299.2 [M+H]⁺

### Step 2: Synthesis of [3-(difluoromethyl)-[1,2,4]triazolo[4,3-a]pyridin-7-yl]methanamine

To a solution of tert-butyl N-[[3-(difluoromethyl)-[1,2,4]triazolo[4,3-a]pyridin-7-yl]methyl]carbamate (601 mg, 2.01 mmol) in dichloromethane (16 mL) was added trifluoroacetic acid (4.0 mL). The reaction was stirred at RT for 3 hours. The solvent was removed under vacuum and the crude was redissolved in MeOH (3 mL) and loaded onto an SCX cartridge (5 g, Biotage). The cartridge was then eluted with MeOH and then the amine was eluted with NH3 3.5 M MeOH to give [3-(difluoromethyl)-[1,2,4]triazolo[4,3-a]pyridin-7-yl]methanamine (346 mg, 1.75 mmol, 87%) as an orange solid.

AnalpH9_MeCN_4 min: Rt: 0.87 min, m/z 199.1 [M+H]⁺

### S2 Reagents

### Synthesis of (2S)-1-tert-butoxycarbonyl-4-(5-methylindolin-1-yl)pyrrolidine-2-carboxylic acid (M05838-int and M05839-int)

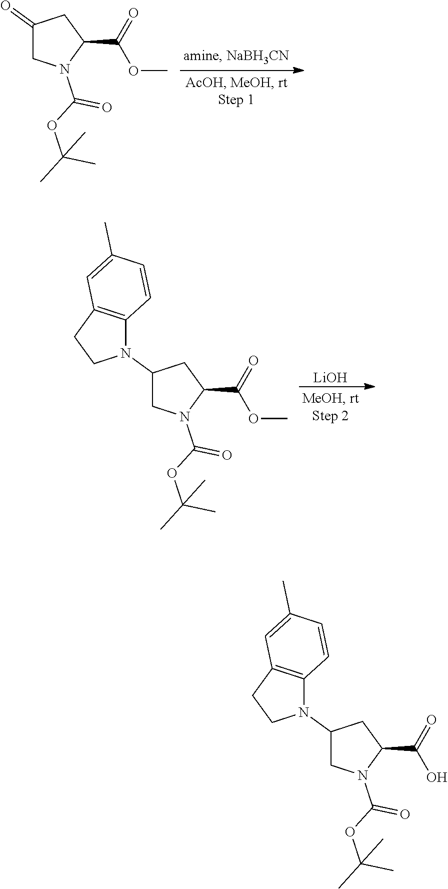

Step 1: 5-Methylindoline (0.80 mL, 6.17 mmol) was dissolved in methanol (10 mL) and (2S)-1-boc-4-oxo-proline methyl ester (500 mg, 2.06 mmol) was added. The reaction was stirred for 15 minutes and sodium cyanoborohydride (258.33 mg, 4.11 mmol) was added. The reaction was stirred at rt overnight. 20 mg of NaBH₄ was added and the reaction stirred at rt for 2 h. To the reaction mixture was added water (20 mL) and brine (20 mL) and the crude product extracted with EtOAc (2×30 mL). The combined organics were dried (MgSO₄), filtered and solvent removed in vacuo. The residue was partially purified by flash column chromatography (SiO₂, Biotage, 25 g Sfär column, eluting from 0 to 80% EtOAc in isohexane) to give a mixture containing O1-tert-butyl O2-methyl (2S)-4-(5-methylindolin-1-yl)pyrrolidine-1,2-dicarboxylate (1.40 g, quantitative) as a brown oil, and 5-methylindoline as the major impurity, accounting for the excess material. The crude product was used directly in the subsequent reaction.

AnalpH9_MeCN_4 min, Rt: 2.92 min, m/z 361.3 [M+H]⁺ Major impurity is amine (46%, Rt 1.98 min)

Step 2: O1-tert-butyl O2-methyl (2S)-4-(5-methylindolin-1-yl)pyrrolidine-1,2-dicarboxylate (assumed 741 mg, 2.06 mmol from previous step) was dissolved in methanol (10 mL) and 2 M LiOH (10 mL, 20.0 mmol) added. The reaction was stirred at rt for 2 h. The solvent was removed and the residue partitioned between EtOAc (50 mL) and water (50 mL). The organic layer was discarded and the aqueous layer acidified to pH4 using 2 M HCl. The aqueous phase was then extracted with 2×50 mL EtOAc and the combined organic layers dried (MgSO4), filtered and solvent removed to give (2S)-1-tert-butoxycarbonyl-4-(5-methylindolin-1-yl)pyrrolidine-2-carboxylic acid (706 mg, 99%) as a brown oil which was used crude in the next step.

AnalpH9_MeCN_4 min, Rt: 1.71 min, m/z 347.3 [M+H]⁺

### Synthesis of O1-tert-butyl O2-methyl (2S)-4-[(6-methoxy-3-pyridyl)methyl]-5-oxo-pyrrolidine-1,2-dicarboxylate (M05875-int)

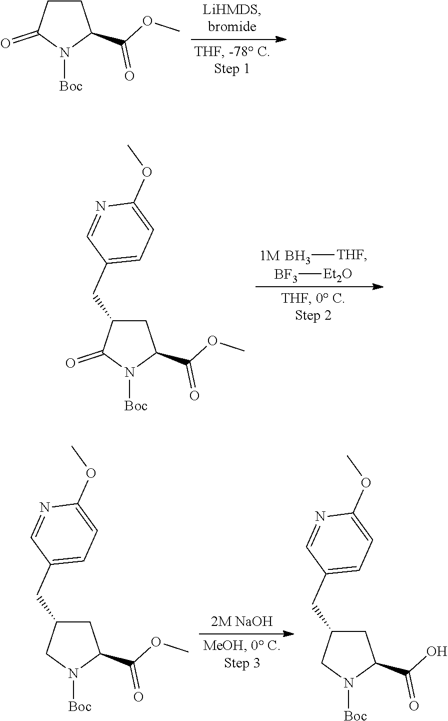

Step 1: Boc-L-Pyroglutamic acid methyl ester (160 mg, 0.66 mmol) was dissolved in dry THF (10 mL) and the solution was cooled to −78° C. LiHMDS (1 M in THF, 0.72 mL, 0.72 mmol) was added dropwise at −78° C. over 10 minutes. The solution was stirred at −78° C. for 1 hour before a solution of 5-(bromomethyl)-2-methoxy-pyridine (146 mg, 0.72 mmol) in dry THF (2 mL) was added dropwise over 10 minutes. The solution was stirred at −78° C. for 2 hours. Aqueous saturated NaHCO₃ was added, and the reaction mixture allowed to warm to room temperature. The reaction mixture was diluted with EtOAc (20 mL) and the layers were separated. The aqueous was extracted further with EtOAc (2×20 mL) and the combined organic layers were washed with brine (20 mL), passed through a phase separator, and concentrated in vacuo. The product was purified by flash column chromatography (SiO₂, Biotage, 10 g, Sfär column, eluting from 0 to 60% EtOAc in isohexane) to yield O1-tert-butyl O2-methyl (2S)-4-[(6-methoxy-3-pyridyl)methyl]-5-oxo-pyrrolidine-1,2-dicarboxylate (138 mg, 58%) as a colourless oil.

AnalpH9_MeCN_4 min, Rt: 2.54 min, m/z 351.2 [M+H]⁺.

¹H-NMR (400 MHz, CDCl₃) δ 7.88-7.96 (1H, m), 7.37-7.43 (1H, m), 6.64-6.71 (1H, m), 4.56-4.65 (1H, m), 3.89 (3H, s), 3.77 (3H, s), 3.11-3.16 (1H, m), 2.57-2.66 (2H, m), 2.25-2.52 (2H, m), 1.48 (9H, s).

Step 2: O1-tert-butyl O2-methyl (2S)-4-[(6-methoxy-3-pyridyl)methyl]-5-oxo-pyrrolidine-1,2-dicarboxylate (230 mg, 0.63 mmol) was dissolved in dry THE (5 mL). The mixture was cooled to 0° C. and 1 M BH₃-THF (6.32 mL, 6.32 mmol) was added dropwise over 5 mins under N₂ followed by addition of BF₃-Et₂O (234 μL, 1.9 mmol) dropwise over 5 mins. The reaction mixture was stirred at 0° C. for 2 hours before being warmed to room temperature and stirred for a further 2 hours. Aqueous saturated NaHCO₃ (5 mL) was added at 0° C. dropwise and the solution was stirred for 15 mins. The solution was diluted with water (10 mL) and EtOAc (20 mL). The layers were partitioned and the aqueous was extracted further with EtOAc (2×20 ml). The combined organics were washed with brine (20 mL) and passed through a phase separator (Biotage) and concentrated in vacuo. The crude residue was purified by flash column chromatography (SiO₂, Biotage isolera, 10 g, Sfär column, eluting from 0 to 60% of EtOAc in isohexane) to give O1-tert-butyl O2-methyl (2S,4R)-4-[(6-methoxy-3-pyridyl)methyl]pyrrolidine-1,2-dicarboxylate (185 mg, 83%) as a colourless oil.

AnalpH9_MeCN_4 min, Rt: 2.54 min, m/z 351.2 [M+H]⁺.

Step 3: 01-tert-butyl O2-methyl (2S,4R)-4-[(6-methoxy-3-pyridyl)methyl]pyrrolidine-1,2-dicarboxylate (185 mg, 0.53 mmol) was dissolved in methanol (5 mL) and NaOH (2 M in H₂O, 5 mL, 10 mmol) was added dropwise at 0° C. The reaction mixture was stirred for 2 hours. Methanol was removed in vacuo and the resulting aqueous solution was acidified to pH 4 with 2M HCl. The product was extracted into EtOAc (3×10 mL) and the combined organic layers were passed through a phase separator (Biotage) and concentrated in vacuo to give (2S,4R)-1-tert-butoxycarbonyl-4-[[6-(trifluoromethyl)-3-pyridyl]methyl]pyrrolidine-2-carboxylic acid (172 mg, 97%) as a colourless oil.

AnalpH9_MeCN_4 min, Rt: 1.68 min, m/z 337.2 [M+H]⁺.

The following compounds were made by analogous methods:

| | | | Mass, % |
|---|---|---|---|
| Example No. | Structure & conditions | Analytical Data | yield, state |
| (2S,4R)-1-(tert- butoxycarbonyl)-4-((6- (trifluoromethyl)pyridin-3- yl)methyl)pyrrolidine-2- carboxylic acid (M05863-int) | 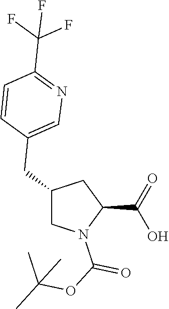 [SMILES: CC(C)(C)OC(=O)N1C[C@H](Cc2ccc(C(F)(F)F)nc2)C[C@H]1C(=O)O] Using 5-(bromomethyl)-2- (trifluoromethyl)pyridine | AnalpH9_MeCN_4 min: Rt: 1.58 min, m/z 375.2 [M + H]⁺ ¹H-NMR (400 MHz, CDCl₃) δ 8.58 (d, J = 9.2 Hz, 1H), 7.75-7.62 (m, 2H), 4.43 (d, J = 8.2 Hz, 0.6H), 4.32 (d, J = 7.8 Hz, 0.4H), 3.75- 3.67 (m, 0.4H), 3.54- 3.45 (m, 0.6H), 3.14 (t, J = 9.2 Hz, 0.4H), 3.04 (t, J = 9.8 Hz, 0.6H), 2.90-2.73 (m, 2H), 2.68- 2.55 (m, 1H), 2.46 (q, J = 6.3 Hz, 0.5H), 2.06-1.94 (m, 1H), 1.81 (dd, J = 21.1, 11.4 Hz, 0.5H), 1.48-1.36 (m, 9H) | 439 mg, 97%, colourless oil |
| (2S,4R)-1-tert- butoxycarbonyl-4-(4- cyclopropylphenyl)methyl pyrrolidine-2-carboxylic acid (M06006-int, M05934-int, M05935-int) | 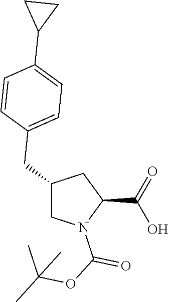 [SMILES: CC(C)(C)OC(=O)N1C[C@H](Cc2ccc(C3CC3)cc2)C[C@H]1C(=O)O] Using 4-(cyclopropyl) benzyl bromide | AnalpH9_MeCN_4 min: Rt: 1.90 min, m/z 346.2 [M + H]⁺ | 135 mg, 100%, orange oil |
| (2S,4S)-1-tert- butoxycarbonyl-4-[(5- cyclopropyloxazol-2- yl)methyl]pyrrolidine-2- carboxylic acid (M06009-int) | 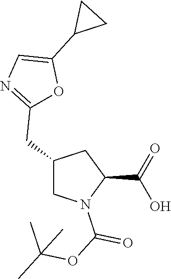 [SMILES: CC(C)(C)OC(=O)N1C[C@H](Cc2ncc(C3CC3)o2)C[C@H]1C(=O)O] Using 2-(bromomethyl)-5- cyclopropyl-oxazole | AnalpH9_MeCN_4 min: Rt: 1.53 min, m/z 237.3 [M + H-boc]⁺ | 52 mg, 90%, colourless gum |
| (2S,4S)-1-(tert- butoxycarbonyl)-4-((1- cyclopropyl-1H-pyrazol-3- yl)methyl)pyrrolidine-2- carboxylic acid | 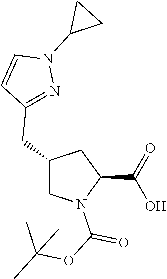 [SMILES: CC(C)(C)OC(=O)N1C[C@H](Cc2ccn(C3CC3)n2)C[C@H]1C(=O)O] Using 3-(bromomethyl)-1- cyclopropyl-1H-pyrazole (Synthesis ref: WO2020/ 95177, Page 82) | AnalpH9_MeCN_4 min: Rt: 2.13 min, m/z 336.3 [M + H]⁺ | 43 mg, 100%, white solid |
| (2S,4S)-1-tert- butoxycarbonyl-4-[(5- cyclopropylisoxazol-3- yl)methyl]pyrrolidine-2- carboxylic acid (M06047-int) | 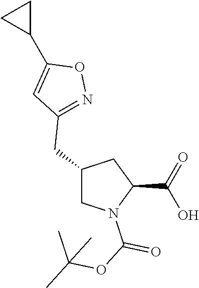 [SMILES: CC(C)(C)OC(=O)N1C[C@H](Cc2cc(C3CC3)on2)C[C@H]1C(=O)O] Using 3-(bromomethyl)-5- cyclopropyl-isoxazole | AnalpH9_MeCN_4 min: Rt: 1.52 min, m/z 337.3 [M + H]⁺ | 650 mg, 96%, colourless oil |

### Synthesis of (S)-1-(tert-butoxycarbonyl)-4-(4-methylbenzylidene)pyrrolidine-2-carboxylic acid (M05843-int)

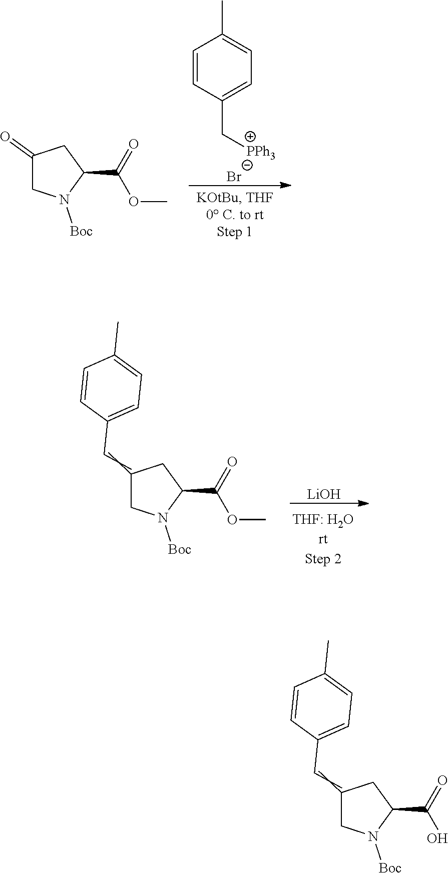

Step 1: To a stirred solution of (4-methylbenzyl)(triphenyl) phosphoniumbromide (2.5 g, 5.6 mmol) in DCM (25 mL) was added a solution of KOtBu (1 M in THE, 5.3 ml, 5.35 mmol) at 0° C. and the solution was stirred for 30 minutes and allowed to reach room temperature. Then, N-boc-4-oxo-L-proline methyl ester (700 mg, 2.55 mmol) was added and the reaction mixture stirred at room temperature overnight. The solvent was removed in vacuo and the residue was dissolved in ethyl acetate (50 mL) and washed with 10% aqueous citric acid (50 mL), aqueous saturated NaHCO₃ (50 mL) and brine (3×50 mL). The combined organic extracts were dried (MgSO₄), the solvent removed and the residue was purified by flash column chromatography (SiO₂, Biotage, 25 g Sfär column, eluting from 0 to 40% EtOAc in isohexane) to give 1-(tert-butyl) 2-methyl (S)-4-(4-methylbenzylidene)pyrrolidine-1,2-dicarboxylate (705 mg, 74%) as a pale yellow oil (as a mixture of E and Z stereoisomers).

AnalpH2_MeCN_4 min, Rt: 3.18 min, m/z 354.2 [M+Na]⁺

Step 2: 1-(tert-Butyl) 2-methyl (S)-4-(4-methylbenzylidene)pyrrolidine-1,2-dicarboxylate (900 mg, 2.72 mmol) in THE at rt was added to a solution of lithium hydroxide monohydrate (250 mg, 5.96 mmol) in water (6 mL) and the mixture was stirred for 3 hours. The solvents were evaporated in vacuo.

The residue was suspended in aqueous citric acid 10 mol % (20 mL) and extracted with ethyl acetate (2×25 mL). The combined organic extracts were dried over magnesium sulphate and filtered. The solvent was removed in vacuo and (S)-1-(tert-butoxycarbonyl)-4-(4-methylbenzylidene)pyrrolidine-2-carboxylic acid (710 mg, 82%) was obtained as a white solid (as a mixture of E and Z stereoisomers).

AnalpH9_MeCN_4 min, Rt: 1.86 min, m/z 316.1 [M−H]⁻

¹H-NMR (400 MHz, CD₂Cl₂) δ 7.30-6.92 (m, 4H), 6.37 (d, J=17.9 Hz, 1H), 4.65-4.35 (1H), 4.36-4.01 (m, 2H), 3.33-2.73 (m, 2H), 2.31 (s, 3H), 1.62-1.27 (9H) Synthesis of (2S,4R)-1-tert-butoxycarbonyl-4-(spiro[2.5]oct-6-en-6-ylmethyl)pyrrolidine-2-carboxylic acid (M05992-int, M05991-int)

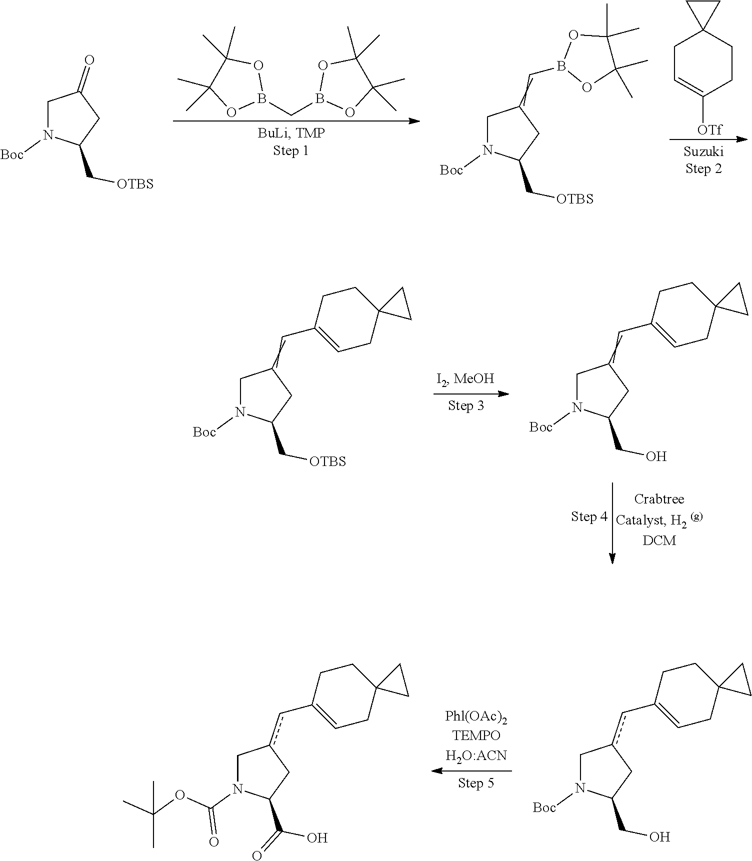

### Step 1: Synthesis of tert-butyl (S)-2-(((tert-butyldimethylsilyl)oxy)methyl)-4-((4,4,5,5-tetramethyl-1,3,2-dioxaborolan-2-yl)methylene)pyrrolidine-1-carboxylate

To a flask under a nitrogen atmosphere, 2,2,6,6-tetramethylpiperidine (8.62 mL, 51.05 mmol) and tetrahydrofuran (60 mL) were added and the solution cooled to −40° C. Then n-Butyllithium (2.5M solution in hexane) (20 mL, 51.1 mmol) was added dropwise. The reaction was kept at −40° C. for 1 h and then the bath was replaced with an acetone/dry ice bath (−78° C.). Then a solution of bis[(pinacolato)boryl]methane (11.4 g, 42.54 mmol) in THE was added at −78° C. and the reaction was stirred at the same temperature for 1 hour. A solution of tert-butyl (2S)-2-[[tert-butyl(dimethyl)silyl]oxymethyl]-4-oxo-pyrrolidine-1-carboxylate (18.22 g, 55.3 mmol) in tetrahydrofuran (60 mL) was added dropwise at −78° C. and the reaction was stirred at −78° C. for 2 hours and then overnight at room temperature. The reaction was quenched with ammonium chloride solution (50 mL) and the solvent removed. The residue was dissolved in DCM (300 mL) and washed with brine (2×100 mL), dried over magnesium sulfate, filtered and the solvent was removed in vacuo. The residue was purified by flash column chromatography (SiO₂, Biotage 330 g SFAR) 0-80% Et₂O/ihexane, the solvent removed to give tert-butyl (S)-2-(((tert-butyldimethylsilyl)oxy)methyl)-4-((4,4,5,5-tetramethyl-1,3,2-dioxaborolan-2-yl)methylene)pyrrolidine-1-carboxylate (14.3 g, 29.6 mmol, 70%) as a pale yellow oil.

1H-NMR (400 MHz, CHLOROFORM-D) δ 5.29 (s, 1H), 4.27-3.90 (m, 3H), 3.66-3.45 (m, 2H), 2.80-2.59 (m, 2H), 1.45 (s, 9H), 1.21 (s, 12H), 0.83 (s, 9H), −0.00 (s, 6H)

### Step 2: Synthesis of tert-butyl (S)-2-(((tert-butyldimethylsilyl)oxy)methyl)-4-(spiro[2.5]oct-5-en-6-ylmethylene)pyrrolidine-1-carboxylate

A flask with a teflon screw-cap was loaded with spiro[2.5]oct-6-en-6-yl trifluoromethanesulfonate (1.56 g, 6.09 mmol) [see for reference; AMBYS MEDICINES—WO2021/76938, 2021, A1], potassium carbonate (3.99 g, 12.18 mmol), tert-butyl (2S,4Z)-2-[[tert-butyl(dimethyl)silyl]oxymethyl]-4-[(4,4,5,5-tetramethyl-1,3,2-dioxaborolan-2-yl)methylene]pyrrolidine-1-carboxylate (5 g, 11.03 mmol), [1,1′-bis(diphenylphosphino)ferrocene]-dichloropalladium(II), complex with dichloromethane (499.6 mg, 0.61 mmol). The flask was sealed, placed under vacuum and then refilled with nitrogen. Then 1,4-dioxane (50 mL) and water (15 mL) were added and the suspension was degassed with a nitrogen balloon for 30 minutes. Then the flask was sealed under nitrogen and the reaction was stirred for 16 h at 90° C. The reaction was cooled down and the solvent was removed. The residue was purified by column chromatography (SiO₂, Biotage 100 g SFAR) 0-50% Et₂O/ihexane, the solvent removed to give and provided tert-butyl (S)-2-(((tert-butyldimethylsilyl)oxy)methyl)-4-(spiro[2.5]oct-5-en-6-ylmethylene)pyrrolidine-1-carboxylate (2440 mg, 5.63 mmol, 92%) as a pale yellow oil.

1H-NMR (400 MHz, CHLOROFORM-D) δ 5.80 (s, 1H), 5.54 (s, 1H), 4.23-3.79 (m, 4H), 3.59 (s, 2H), 3.35 (d, J=17.4 Hz, 1H), 2.65 (d, J=61.4 Hz, 2H), 2.31-2.17 (m, 2H), 2.00 (d, J=23.4 Hz, 2H), 1.46-1.33 (m, 9H), 0.86 (s, 9H), 0.27 (s, 4H), 0.05 (s, 6H).

### Step 3: Synthesis of tert-butyl (S)-2-(hydroxymethyl)-4-(spiro[2.5]oct-5-en-6-ylmethylene)pyrrolidine-1-carboxylate

To a solution of tert-butyl (2S,4Z)-2-[[tert-butyl(dimethyl)silyl]oxymethyl]-4-(spiro[2.5]oct-6-en-6-ylmethylene)pyrrolidine-1-carboxylate (2.44 g, 5.63 mmol) in methanol (20 mL) was added iodine (71.4 mg, 0.28 mmol) and the reaction was stirred overnight. Then saturated sodium thiosulfate was added until the solution became colourless and finally acetonitrile. The solvent was removed, then dichloromethane and magnesium sulfate were added. The suspension was filtered, the solvent was evaporated and the residue was purified by column chromatography (SiO₂, Biotage 50 g SFAR) 0-100% Et₂O/ihexane, the solvent removed to give tert-butyl (2S,4Z)-2-(hydroxymethyl)-4-(spiro[2.5]oct-6-en-6-ylmethylene)pyrrolidine-1-carboxylate (1.69 g, 5.29 mmol, 94%) as a colourless oil.

1H-NMR (400 MHz, CHLOROFORM-D) δ 5.79 (d, J=18.8 Hz, 1H), 5.56 (s, 1H), 4.19-3.89 (m, 4H), 3.58 (s, 2H), 2.78 (s, 1H), 2.38-1.99 (m, 5H), 1.47 (s, 11H), 0.29 (s, 4H)

### Step 4: Synthesis of tert-butyl (2S,4R)-2-(hydroxymethyl)-4-(spiro[2.5]oct-6-en-6-ylmethyl)pyrrolidine-1-carboxylate

A 50 mL flask was loaded with tert-butyl (2S,4Z)-2-(hydroxymethyl)-4-(spiro[2.5]oct-6-en-6-ylmethylene)pyrrolidine-1-carboxylate (1690 mg, 5.29 mmol) and (1Z;5Z)-cycloocta-1,5-diene; dimethyl-pyridin-1-ium-1-yl-(tricyclohexyl-A5-phosphanyl)iridium; hexafluorophosphate (133 mg, 0.159 mmol), evacuated and backfilled with nitrogen. Then dichloromethane (20 mL) was added by syringe and the resulting orange solution was cooled down to 0° C. A hydrogen balloon was bubbled inside the solution for 15 minutes×3 times and then the reaction was stirred with a hydrogen balloon for 2 hours. The solvent was removed and the residue was purified by column chromatography (SiO₂, Biotage 50 g SFAR) 0-100% Et₂O/ihexane, the solvent removed to give a mixture of tert-butyl (2S,4R)-2-(hydroxymethyl)-4-(spiro[2.5]oct-6-en-6-ylmethyl)pyrrolidine-1-carboxylate and tert-butyl (S,E)-2-(hydroxymethyl)-4-(spiro[2.5]oct-5-en-6-ylmethylene)pyrrolidine-1-carboxylate (1280 mg, 3.98 mmol, 75%) as a pale yellow oil.

1H-NMR (400 MHz, CHLOROFORM-D) δ 5.40 (s, 1H), 4.50 (s, 1H), 3.96 (d, J=48.1 Hz, 1H), 3.61-3.36 (m, 3H), 3.06 (d, J=17.9 Hz, 1H), 2.33 (t, J=19.9 Hz, 1H), 1.91 (d, J=46.7 Hz, 7H), 1.69-1.61 (m, 2H), 1.45 (s, 9H), 0.77-0.95 (1H), 0.27 (s, 4H)

The presence of the diene impurity was not identified until synthesis of the final compounds (General scheme 1).

### Step 5: Synthesis of (2S,4R)-1-tert-butoxycarbonyl-4-(spiro[2.5]oct-6-en-6-ylmethyl)pyrrolidine-2-carboxylic acid (M05992-int)

Tert-butyl (2S,4Z)-2-(hydroxymethyl)-4-(spiro[2.5]oct-6-en-6-ylmethylene)pyrrolidine-1-carboxylate (562 mg, 1.76 mmol) was dissolved in acetonitrile (5.0 mL) and water (5.0 mL). Then iodobenzene diacetate, (1247 mg, 3.87 mmol) and 2,2,6,6-tetramethylpiperidinoxy free radical (TEMPO) (55 mg, 0.352 mmol) were added. Then the mixture was stirred for 4 hours at room temperature.

Dichloromethane (20 ml) and brine (20 ml) were added, and the mixture was extracted, the aqueous layer was washed with DCM (20 ml) and the organic layers were dried over magnesium sulfate and filtered. The solvent was remove to give a crude mixture of (2S,4R)-1-tert-butoxycarbonyl-4-(spiro[2.5]oct-6-en-6-ylmethyl)pyrrolidine-2-carboxylic acid and (S,E)-1-(tert-butoxycarbonyl)-4-(spiro[2.5]oct-5-en-6-ylmethylene)pyrrolidine-2-carboxylic acid (450 mg, 1.34 mmol, 76%).

UPLC_pH9_MeCN_2 min, Rt: 1.90 min, m/z 236.3 [M+H-Boc]⁺ (mono-alkene), 234.2 [M+H-Boc]⁺ (diene)

The following compounds were made by analogous methods:

| | | | Mass, |
|---|---|---|---|
| | | | yield, |
| Example No. | Structure & conditions | Analytical Data | state |
| M05997-int | 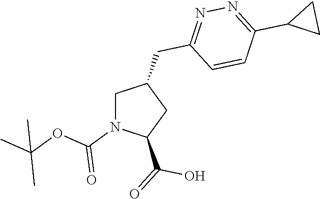 [SMILES: CC(C)(C)OC(=O)N1C[C@H](Cc2ccc(C3CC3)nn2)C[C@H]1C(=O)O] Using 3-bromo-6- cyclopropyl-pyridazine | AnalpH9_MeCN_4 min: Rt: 1.35 min, m/z 348.3 [M + H]⁺ | 60 mg, 39%. |
| M06068-int | 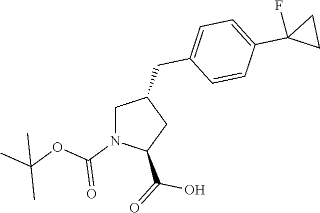 [SMILES: CC(C)(C)OC(=O)N1C[C@H](Cc2ccc(C3(F)CC3)cc2)C[C@H]1C(=O)O] Using 1-bromo-4-(1- fluorocyclopropyl)benzene | UPLC_pH9_MeCN_2 min, Rt: 1.43 min, m/z 264.3 [M + H]⁺ | 150 mg, 65%, orange oil |

### Synthesis of (2S,4R)-1-tert-butoxycarbonyl-4-(4-methylphenoxy)pyrrolidine-2-carboxylic acid (M05798-int)

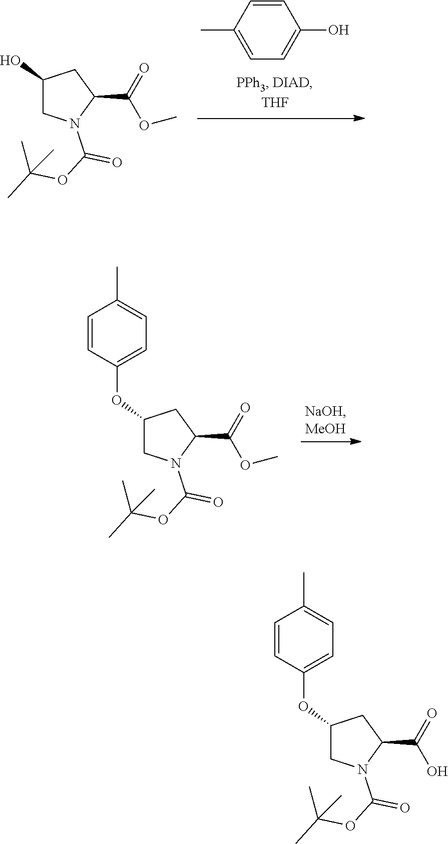

### Step 1: Synthesis of O1-tert-butyl O2-methyl (2S,4R)-4-(4-methylphenoxy)pyrrolidine-1,2-dicarboxylate

Boc-cis-hydroxyproline methyl ester (500.mg, 2.04 mmol) was dissolved in tetrahydrofuran (10 mL) and p-Cresol (441 mg, 4.08 mmol) followed by triphenylphosphine (1069 mg, 4.08 mmol) added. The reaction was stirred for 15 minutes and diisopropyl azodicarboxylate (0.75 mL, 4.08 mmol) was added. The reaction was stirred at rt overnight. The solvent was evaporated and the residue was purified by column chromatography (SiO₂, Biotage 25 g SFAR) 0-20% EtOAc/ihexane over 10 CV. The solvent was evaporated to give O1-tert-butyl O2-methyl (2S,4R)-4-(4-methylphenoxy)pyrrolidine-1.2-dicarboxylate (587 mg, 1.75 mmol, 86%) as a colourless oil.

AnalpH9_MeCN_4 min, Rt: 2.87 min, m/z 236.3 [M+H]⁺

### Step 2: Synthesis of (2S,4R)-1-tert-butoxycarbonyl-4-(4-methylphenoxy)pyrrolidine-2-carboxylic acid

O1-tert-butyl O2-methyl (2S,4R)-4-(4-methylphenoxy)pyrrolidine-1,2-dicarboxylate (587.mg, 1.75 mmol) was dissolved in methanol (10 mL) and 2 M NaOH (8.8 mL, 17.5 mmol) added. The reaction was stirred at rt for 2 h. The solvent was evaporated and the residue partitioned between EtOAc (20 mL) and Water (20 mL) and organic discarded. The aqueous was acidified to pH4 (using 2 M HCl) and extracted with 2×20 mL EtOAc. The combined organics were dried (MgSO₄), filtered and solvent evaporated to give (2S,4R)-1-tert-butoxycarbonyl-4-(4-methylphenoxy)pyrrolidine-2-carboxylic acid (519 mg, 1.61 mmol, 92%) as a colourless oil.

AnalpH9_MeCN_4 min, Rt: 1.67 min, m/z 490.3 [M+H]⁺

### Synthesis of S4 Reagents:

### Synthesis of (2R,3S)-1-tert-butoxycarbonyl-3-(pyrrolidine-1-carbonyl)piperidine-2-carboxylic acid

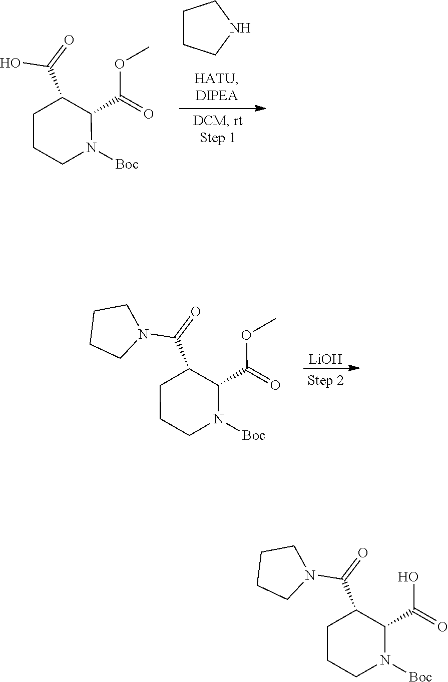

Step 1: To a stirred solution of (2R,3S)-1-tert-butoxycarbonyl-2-methoxycarbonyl-piperidine-3-carboxylic acid (1.00 g, 3.48 mmol) in DCM (23 mL) was added pyrrolidine (0.30 mL, 3.65 mmol) and N,N-diisopropylethylamine (1.8 mL, 10.4 mmol). The reaction mixture was stirred at rt for 15 min and then HATU (1323 mg, 3.48 mmol) was added. The reaction mixture was stirred at rt for 3 hours. A saturated aqueous solution of NaHCO₃ (20 mL) was added. The phases were separated, and the aqueous phase was extracted with DCM (3×30 mL). The organic phases were combined and passed through a phase separator. The organic phase was concentrated under vacuum to afford 2.6 g of crude product. The residue was redissolved in the minimum amount of EtOAc and filtered through a silica plug eluting with EtOAc. Fractions containing the product were combined, affording 2.1 g of 01-tert-butyl O2-methyl (2R,3S)-3-(pyrrolidine-1-carbonyl)piperidine-1,2-dicarboxylate (Assumed 100% yield, 1.2 g) as a yellow gum.

AnalpH9_MeCN_4 min, Rt: 2.23 min, m/z 341.2 [M+H]⁺

Optionally the amine may be used as the HCl salt.

Optionally the crude material may be used without silica filtration or may be purified by flash column chromatography or preparative HPLC.

Step 2: To a stirred solution of O1-tert-butyl O2-methyl (2R,3S)-3-(pyrrolidine-1-carbonyl)piperidine-1,2-dicarboxylate (1.18 g, 3.48 mmol) in methyl alcohol (58 mL) was added 17 mL of LiOH 1 M (17.4 mL, 17.4 mmol) at 0° C. The reaction mixture was stirred overnight at rt. The reaction mixture was neutralised to pH6 carefully by addition of 1 M HCl. The MeOH was concentrated under vacuum and the aqueous phase was carefully adjusted to pH9 with 1 M KOH (aq). The aqueous phase was washed with DCM (20 mL). The organic phase was discarded and the aqueous phase was acidified to pH4. The aqueous phase was extracted with DCM (3×20 mL). The organic phases were combined and passed through a phase separator (Biotage) before concentrating under vacuum, furnishing (2R,3S)-1-tert-butoxycarbonyl-3-(pyrrolidine-1-carbonyl)piperidine-2-carboxylic acid (545 mg, 48%) as a light yellow gum.

AnalpH9_MeCN_4 min, Rt: 1.47 min, m/z 325.2 [M−H]⁻

¹H-NMR (400 MHz, CDCl₃) δ 5.22 (br s, 0.5H), 4.95 (br s, 0.5H), 4.04 (dd, J=14.0, 3.3 Hz, 0.5H), 3.94 (dd, J=13.5, 4.8 Hz, 0.5H), 3.41-3.64 (m, 4H), 3.20 (td, J=13.5, 3.3 Hz, 0.5H), 3.06 (td, J=13.1, 3.3 Hz, 0.5H), 2.85-2.93 (m, 1H), 1.45-2.10 (m, 8H), 1.48 and 1.45 (2S, 9H) (Observed as mixture of rotamers).

Optionally the crude material may be purified by preparative HPLC (pH9 MeCN)

The following compounds were made by analogous methods:

| | | | Mass, |
|---|---|---|---|
| | | | yield, |
| Example No. | Structure & conditions | Analytical Data | state |
| M05733-S4 (2R,3S)-1-(tert- butoxycarbonyl)-3- (piperidine-1- carbonyl)piperidine-2- carboxylic acid | 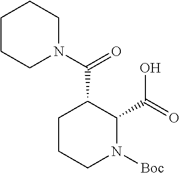 [SMILES: CC(C)(C)OC(=O)N1CCC[C@H](C(=O)N2CCCCC2)[C@@H]1C(=O)O] From piperidine Step 1: purified by preparative HPLC Step 2: used crude without further purification | AnalpH9_MeCN_4 min, Rt: 1.58 min, m/z 341.4 [M − Boc + H]⁺ | 30.0 mg, 49%, colourless gum |
| M05747-S4 (2R,3S)-1-(tert- butoxycarbonyl)-3- ((R)-2-(trifluoromethyl) pyrrolidine-1- carbonyl)piperidine-2- carboxylic acid | 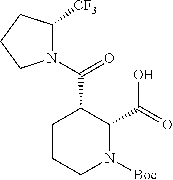 [SMILES: CC(C)(C)OC(=O)N1CCC[C@H](C(=O)N2CCC[C@@H]2C(F)(F)F)[C@@H]1C(=O)O] From 2-(R)-2- trifluoromethylpyrrolidine Step 1: purified by fltration through silica plug Step 2: purified by prepartive HPLC | AnalpH9_MeCN_4 min, Rt: 1.77 min, m/z 295.3 [M − Boc + H]⁺ | 32.6 mg, 23%, colourless oil |
| M05751-S4 (2R,3S)-1-(tert- butoxycarbonyl)-3- (dimethylcarbamoyl) piperidine-2- carboxylic acid | 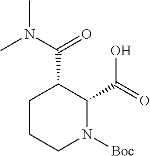 [SMILES: CN(C)C(=O)[C@H]1CCCN(C(=O)OC(C)(C)C)[C@H]1C(=O)O] From dimethylamine hydrochloride Step 1: purified by fltration through silica plug Step 2: purified by prepartive HPLC | AnalpH9_MeCN_4 min, Rt: 1.42 min, m/z 301.3 [M + H]⁺ | 36.3 mg, 35%, colourless oil |
| M05752-S4 (2R,3S)-1-(tert- butoxycarbonyl)-3- (cyclopropyl(methyl) carbamoyl)piperidine- 2-carboxylic acid | 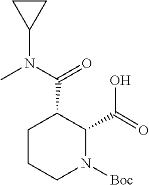 [SMILES: CN(C(=O)[C@H]1CCCN(C(=O)OC(C)(C)C)[C@H]1C(=O)O)C1CC1] From N-Methylcyclo- propanamine hydrochloride Step 1: Crude used without further purification Step 2: Crude used without further purification | AnalpH9_MeCN_4 min, Rt: 1.58 min, m/z 325.5 [M − H]⁻ | 91.0 mg, 67%, yellow gum |
| M05753-S4 (2R,3S)-1-(tert- butoxycarbonyl)-3- (morpholine-4- carbonyl)piperidine-2- carboxylic acid | 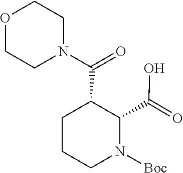 [SMILES: CC(C)(C)OC(=O)N1CCC[C@H](C(=O)N2CCOCC2)[C@@H]1C(=O)O] From morpholine Step 1: Purified by preparative HPLC Step 2: Crude used without further purification | AnalpH9_MeCN_4 min, Rt: 1.41 min, m/z 341.2 [M − H]⁻ | 96.0 mg, 67%, yellow gum |
| M05754-S4 (2R,3S)-1-(tert- butoxycarbonyl)-3- (4-(tert- butoxycarbonyl)piperazine-1- carbonyl)piperidine-2- carboxylic acid | 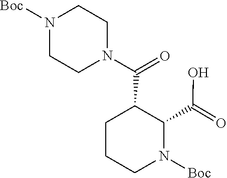 [SMILES: CC(C)(C)OC(=O)N1CCN(C(=O)[C@H]2CCCN(C(=O)OC(C)(C)C)[C@H]2C(=O)O)CC1] From 1-Boc-piperazine Step 1: purified by fltration through silica plug Step 2: purified by prepartive HPLC | AnalpH9_MeCN_4 min, Rt: 1.80 min, m/z 442.3 [M + H]⁺ | 58.0 mg, 31%, colourless oil |
| M05773-S4 (2R,3S)-1-(tert- butoxycarbonyl)-3- (4-azaspiro[2.4]heptane-4- carbonyl)piperidine-2- carboxylic acid | 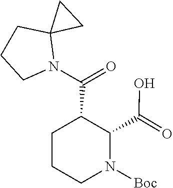 [SMILES: CC(C)(C)OC(=O)N1CCC[C@H](C(=O)N2CCCC23CC3)[C@@H]1C(=O)O] From 4-azaspiro[2.4] heptane hydrocloride Step 1: used crude without further purification Step 2: purified by prepartive HPLC | AnalpH9_MeCN_4 min, Rt: 1.67 min, m/z 253.2 [M − Boc + H]⁺ | 46.9 mg, 31% colourless gum |
| M06049-S4 (2R,3S)-1- tert-butoxycarbonyl-3- [2-[cyclopropyl(methyl) amino]ethyl-methyl- carbamoyl]piperidine-2- carboxylic acid | 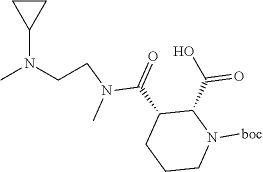 From N-methyl-N-[2- (methylamino)ethyl] cyclopropanamine Step 1: purified by fltration through silica plug Step 2: purified by prepartive HPLC | AnalpH9_MeCN_4 min, Rt: 1.62 min, m/z 384.3 [M + H]⁺ | 123 mg, 51%, colourless gum |
| M06010-S4 (2R,3S)-1- tert-butoxycarbonyl-3- [(3R)-3- (dimethylamino) pyrrolidine-1- carbonyl]piperidine- 2-carboxylic acid | 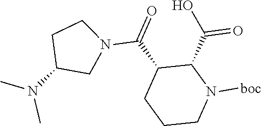 Using (3R)-3- (Dimethylamino)pyrrolidine | AnalpH9_MeCN_4 min, Rt: 1.48 min, m/z 370.4 [M + H]⁺ | 67.4 mg, 73%, clear film |
| M06013-S4 (2R,3S)-1-tert- butoxycarbonyl-3- [(3S)-3-(dimethylamino) pyrrolidine-1-carbonyl] piperidine- 2-carboxylic acid | 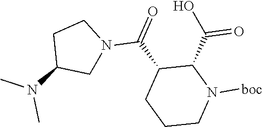 Using (3S)-3- (Dimetylamino)pyrrolidine | AnalpH9_MeCN_4 min, Rt: 1.40 min, m/z 370.4 [M + H]⁺ | 105 mg, 68%, colourless gum |
| M06033-S4; M06034-S4; (2R,3S)-1-tert- butoxycarbonyl-3-[methyl-(1- methylpyrrolidin-3- yl)carbamoyl] piperidine-2- carboxylic acid Double check which diastereomer for which final compound | 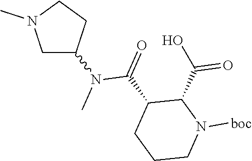 Using N,N′-Dimethyl- 3-aminopyrrolidine Diastereomers separated by Preparative HPLC | Peak 1: AnalpH9_MeCN_4 min, Rt: 1.28 min, m/z 370.4 [M + H]⁺ Peak 2: AnalpH9_MeCN_4 min, Rt: 1.29 min, m/z 370.4 [M + H]⁺ | Peak 1: 43 mg, 38%, white solid Peak 1: 31 mg, 27%, white solid |
| M06039-S4; M06085-S4 (2R,3S)-1-tert- butoxycarbonyl-3-[methyl(2- pyrrolidin-1-ylethyl) carbamoyl]piperidine- 2-carboxylic acid | 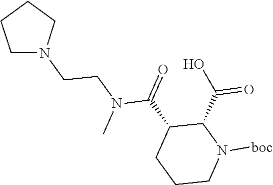 Using methyl[2- (pyrrolidin-1- yl)ethyl]amine | AnalpH9_MeCN_4 min, Rt: 1.51 min, m/z 384.5 [M + H]⁺ | 149 mg, 93%, white solid |
| M06048-S4 (2R,3S)-1-tert- butoxycarbonyl-3- [cyclopropyl-[2- (dimethylamino)ethyl] carbamoyl]piperidine- 2-carboxylic acid | 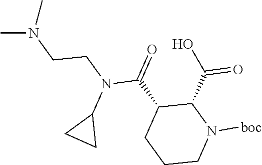 Using [2-(cyclopropylamino) ethyl]dimethylamine dihydrochloride | AnalpH9_MeCN_4 min, Rt: 1.66 min, m/z 384.4 [M + H]⁺ | 119 mg, 100%, white solid |
| M06061-S4 (2R,3S)-1- tert-butoxycarbonyl-3- [methyl-(1-methyl-4- piperidyl)carbamoyl] piperidine-2- carboxylic acid | 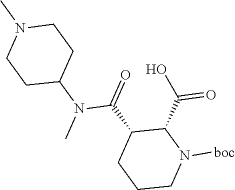 Using 1-methyl-4- (methylamino)piperidine | AnalpH9_MeCN_4 min, Rt: 1.44 min, m/z not recorded | 150 mg, 100%, off-white solid |
| M06063-S4 (2R,3S)-1- tert-butoxycarbonyl-3- [3-(dimethylamino)propyl- methyl-carbamoyl] piperidine-2- carboxylic acid | 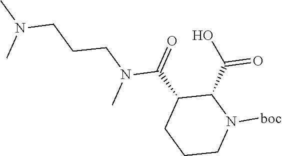 Using N,N,N′-trimethyl- 1,3-propnediamine | AnalpH9_MeCN_4 min, Rt: 1.44 min, m/z 372.3 [M + H]⁺ | 88 mg, 59%, colour- less gum |
| M06112-S4 (2R,3S)-1- tert-butoxycarbonyl-3- [(1-cyclopropyl-4- piperidyl)-methyl- carbamoyl]piperidine- 2-carboxylic acid | 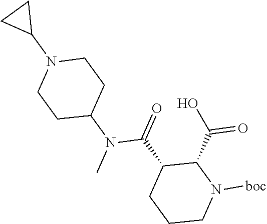 Using 1-cyclopropyl- N-methylpiperidin-4-amine | UPLC_pH9_MeCN_2 min, Rt: 1.33 min, m/z 410.3 [M + H]⁺ | 115 mg, 70%, clear film |

### Synthesis of (2R,3S)-1-benzyloxycarbonyl-3-(pyrrolidine-1-carbonyl)piperidine-2-carboxylic acid

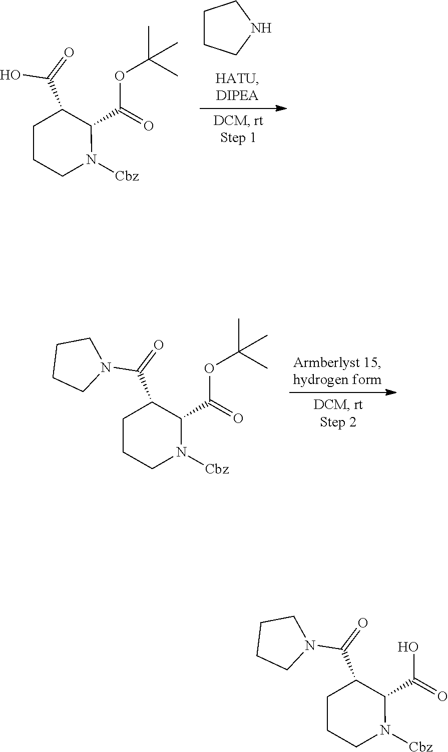

Step 1: To a stirred solution of pyrrolidine (0.4 mL, 4.91 mmol) and (2R,3S)-1-((benzyloxy)carbonyl)-2-(tert-butoxycarbonyl)piperidine-3-carboxylic acid (1.7 g, 4.68 mmol) in DCM (47 mL) at 0° C. was added N,N-diisopropylethylamine (2.4 mL, 14.0 mmol). The reaction mixture was stirred for 10 minutes and then HATU (2.13 g, 5.61 mmol) was added. The reaction mixture was stirred at 0° C. for 1 hour, then 20 mL of a saturated aqueous NaHCO₃ solution were added. The phases were separated and the aqueous phase was extracted with DCM (2×30 mL). The organic phases were combined and passed through a phase separator (Biotage). The organic phase was concentrated under vacuum and the residue was purified by flash column chromatography (SiO₂, Biotage, 50 g, Sfär column, eluting from 100% isohexane to 100% EtOAc), affording 01-benzyl O2-tert-butyl (2R,3S)-3-(pyrrolidine-1-carbonyl)piperidine-1,2-dicarboxylate (1.90 g, 98%) as a white solid.

AnalpH9_MeCN_4 min, Rt: 2.64 min, m/z 417.3 [M+H]⁺

¹H-NMR (400 MHz, CDCl₃) δ 7.33-7.37 (m, 5H), 5.11-5.22 (m, 2.7H), 4.93 (d, J=5.5 Hz, 0.3H), 3.98-4.16 (m, 1H), 3.33-3.70 (m, 5H), 2.60-2.70 (m, 1H), 1.71-2.05 (m, 8H), 1.42 and 1.39 (2s, 9H). (Observed as mixture of rotamers)

Optionally the amine may be used as the HCl salt.

Step 2: To a stirred solution of 01-benzyl O2-tert-butyl (2R,3S)-3-(pyrrolidine-1-carbonyl)piperidine-1,2-dicarboxylate (1.90 g, 4.56 mmol) in DCM (44 mL) was added Amberlyst 15, hydrogen form (2.9 g). The reaction mixture was stirred at rt for 6 hours, and then the solid resin was removed by filtration. The filtrate was concentrated under vacuum and the residue was dissolved in a 1:1 MeCN:water mixture and lyophilised affording (2R,3S)-1-benzyloxycarbonyl-3-(pyrrolidine-1-carbonyl)piperidine-2-carboxylic acid (1.00 g, 61%) as a white solid.

AnalpH9_MeCN_4 min, Rt: 1.62 min, m/z 361.2 [M+H]⁺

¹H-NMR (400 MHz, CDCl₃) δ 7.29-7.40 (m, 5H), 5.26 (br s, 0.6H), 5.10-5.22 (m, 2H), 5.06 (br s, 0.4H), 4.04-4.16 (m, 1H), 3.41-3.63 (m, 4H), 3.28 (td, J=13.5, 3.7 Hz, 0.6H), 3.17 (td, J=13.5, 3.7 Hz, 0.4H), 2.88-2.92 (m, 1H), 1.38-2.10 (m, 8H). (Observed as mixture of rotamers)

Optionally the crude material may be purified by preparative HPLC (pH9 MeCN)

The following compounds were made by analogous methods:

| | | | Mass, |
|---|---|---|---|
| Example No. | Structure & conditions | Analytical Data | state |
| M05766-S4 (2R,3S)-1- ((benzyloxy)carbonyl)-3- (5-azaspiro[2.4]heptane-5- carbonyl)piperidine-2- carboxylic acid | 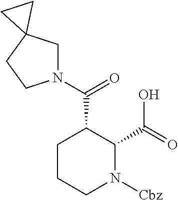 [SMILES: O=C(O)[C@H]1[C@@H](C(=O)N2CCC3(CC3)C2)CCCN1C(=O)OCc1ccccc1] From 5-azaspiro[2.4]heptane Step 1: Purified by flash column chromatography Step 2: TFA used instead of Aberlyst-15 hydrogen form. Crude used without further purification | AnalpH9_MeCN_4 min, Rt: 1.75 min, m/z 387.3 +M + H]⁺ | 52.0 mg, 41%, colourless gum |
| M05770-S4 (2R,3S)-1- ((benzyloxy)carbonyl)-3- (3-oxa-8-azabicyclo[3.2.1] octane-8-carbonyl)piperidine- 2-carboxylic acid | 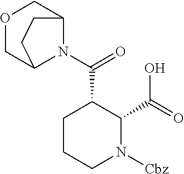 [SMILES: O=C(O)[C@H]1[C@@H](C(=O)N2C3CCC2COC3)CCCN1C(=O)OCc1ccccc1] From 3-oxa-8- azabicyclo[3.2.1]octane hydrochloride Step 1: Purified by flash column chromtography Step 2: Crude used without further purification | AnalpH9_MeCN_4 min: Rt: 1.57 min, m/z 403.3 [M + H]⁺ | 47.0 mg, 73%, colourless gum |
| M05775-S4 (2R,3S)-1- ((benzyloxy)carbonyl)-3- (3-oxa-8-azabicyclo[3.2.1] octane-8-carbonyl) piperidine-2-carboxylic acid | 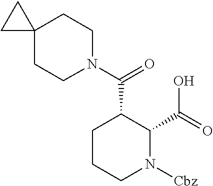 [SMILES: O=C(O)[C@H]1[C@@H](C(=O)N2CCC3(CC2)CC3)CCCN1C(=O)OCc1ccccc1] From 6-Azaspiro[2.5]octane hydrochloride Step 1: Purified by flash column chromtography Step 2: Crude used without further purification | AnalpH9_MeCN_4 min, Rt: 1.84 min, m/z 401.3 [M + H]⁺ | 83.9 mg, 58%, colourless gum |
| M05777-S4 (2R,3S)-1- ((benzyloxy)carbonyl)-3- ((S)-3-fluoropyrrolidine-1- carbonyl)piperidine- 2-carboxylic acid | 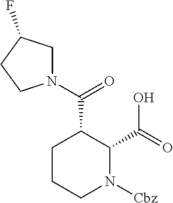 [SMILES: O=C(O)[C@H]1[C@@H](C(=O)N2CC[C@H](F)C2)CCCN1C(=O)OCc1ccccc1] From (S)-(+)-3- fluoropyrrolidine hydrochloride Step 1: Crude used without further purification Step 2: Crude used without further purification | AnalpH9_MeCN_4 min, Rt: 1.58 min, m/z 379.2 [M + H]⁺ | 89.0 mg, 71%, white solid |
| M05785-S4 (2R,3S)-1- ((benzyloxy)carbonyl)-3- ((R)-3-fluoropyrrolidine-1- carbonyl)piperidine- 2-carboxylic acid | 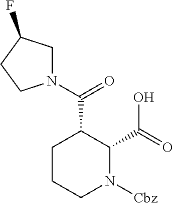 [SMILES: O=C(O)[C@H]1[C@@H](C(=O)N2CC[C@@H](F)C2)CCCN1C(=O)OCc1ccccc1] From (R)-(−)-3- fluoropyrrolidine hydrochloride Step 1: Purified by flash column chromatography Step 2: Crude used without further purification | AnalpH9_MeCN_4 min, Rt: 1.54 min, m/z 379.2 [M + H]⁺ | 70.7 mg, 62%, colourless gum |
| M05787-S4 (2R,3S)-1- ((benzyloxy)carbonyl)-3- (3,3-difluoropyrrolidine-1- carbonyl)piperidine- 2-carboxylic acid | 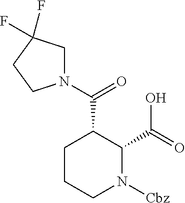 [SMILES: O=C(O)[C@H]1[C@@H](C(=O)N2CCC(F)(F)C2)CCCN1C(=O)OCc1ccccc1] From 3,3-difluoro- pyrrolidine hydrochloride Step 1: Crude purified by flash column chromatography Step 2: Crude used without further purification | AnalpH9_MeCN_4 min, Rt: 1.61 min, m/z 397.2 [M + H]⁺ | 91.0 mg, 70% colourless gum |
| M05804-S4 (2R,3S)-1- ((benzyloxy)carbonyl)-3- (7-azabicyclo[2.2.1]heptane- 7-carbonyl)piperidine- 2-carboxylic acid | 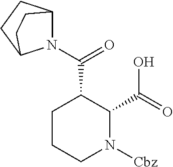 [SMILES: O=C(O)[C@H]1[C@@H](C(=O)N2C3CCC2CC3)CCCN1C(=O)OCc1ccccc1] From 7-azabicyclo[2,2,1] heptane hydrochloride Step 1: Purified by flash column chromtography Step 2: Purified by preparative HPLC | AnalpH9_MeCN_4 min, Rt: 1.78 min, m/z 387.3 [M + H]⁺ | 33.1 mg, 28%, colourless solid |
| M05837-S4 (2R,3S)-1- ((benzyloxy)carbonyl)-3- ((S)-3-methoxypyrrolidine-1- carbonyl)piperidine- 2-carboxylic acid | 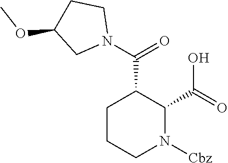 [SMILES: CO[C@H]1CCN(C(=O)[C@H]2CCCN(C(=O)OCc3ccccc3)[C@H]2C(=O)O)C1] From (3S)-3- methoxypyrollidine hydrochloride Step 1: Purified by flash column chromtography Step 2: Crude used without further purification | AnalpH9_MeCN_4 min, Rt: 1.59 min, m/z 391.2 [M + H]⁺ | 41.4 mg, 32%, off-white gum |
| M05840-int (2R,3S)-1- ((benzyloxy)carbonyl)-3- (4-methyl-1,4-diazepane-1- carbonyl)piperidine- 2-carboxylic acid | 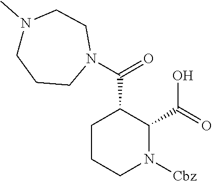 [SMILES: CN1CCCN(C(=O)[C@H]2CCCN(C(=O)OCc3ccccc3)[C@H]2C(=O)O)CC1] From 1-methylhomopiperazine Step 1: Crude used without further purification Step 2: purified by preparative HPLC | AnalpH9_MeCN_4 min, Rt: 1.55 min, m/z 404.2 [M + H]⁺ | 14.0 mg, 11%, colourless gum |
| M05853-S4 (2R,3S)-1- ((benzyloxy)carbonyl)-3- ((S)-3-ethoxypyrrolidine-1- carbonyl)piperidine- 2-carboxylic acid | 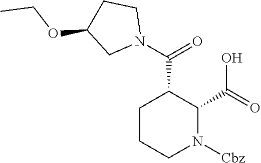 [SMILES: CCO[C@H]1CCN(C(=O)[C@H]2CCCN(C(=O)OCc3ccccc3)[C@H]2C(=O)O)C1] From (S)-3- ethoxypyrrolidine Step 1: Crude used without further purification Step 2: Crude used without further purification | AnalpH9_MeCN_4 min, Rt: 1.69 min, m/z 405.2 [M + H]⁺ | 73.6 mg, 55%, colourless gum |
| M05878-int (2R,3S)-1- ((benzyloxy)carbonyl)-3- (3-fluoroazetidine-1- carbonyl)piperidine-2- carboxylic acid | 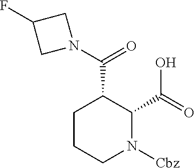 [SMILES: O=C(O)[C@H]1[C@@H](C(=O)N2CC(F)C2)CCCN1C(=O)OCc1ccccc1] From 3-fluoroazetidine hydrochloride Step 1: Purified by flash column chromtography Step 2: Purified by preparative HPLC | AnalpH9_MeCN_4 min, Rt: 1.58 min, m/z 365.1 [M + H]⁺ | 17.0 mg, 11%, colourless gum |

### Synthesis of (2R,3S)-1-((benzyloxy)carbonyl)-2-(tert-butoxycarbonyl)piperidine-3-carboxylic acid

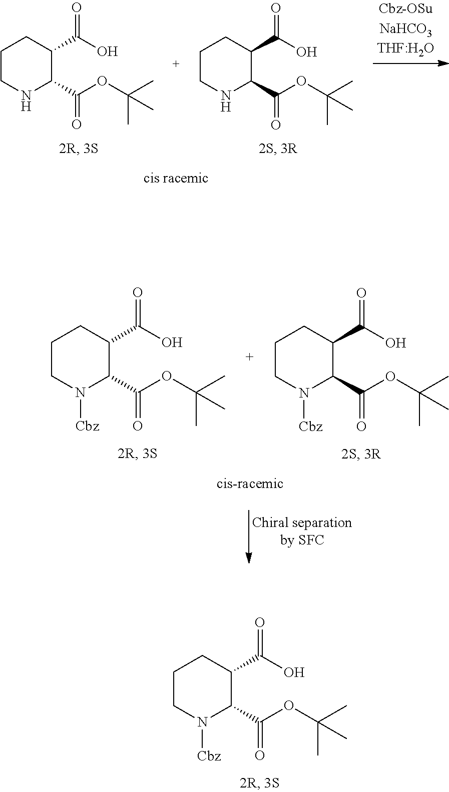 [SMILES: CC(C)(C)OC(=O)[C@@H]1NCCC[C@@H]1C(=O)O ; CC(C)(C)OC(=O)[C@H]1NCCC[C@H]1C(=O)O ; CC(C)(C)OC(=O)[C@H]1[C@@H](C(=O)O)CCCN1C(=O)OCc1ccccc1 ; CC(C)(C)OC(=O)[C@@H]1[C@H](C(=O)O)CCCN1C(=O)OCc1ccccc1 ; CC(C)(C)OC(=O)[C@H]1[C@@H](C(=O)O)CCCN1C(=O)OCc1ccccc1]

To a stirred solution of (2R,3S)-2-(tert-butoxycarbonyl)piperidine-3-carboxylic acid and (2S,3R)-2-5 (tert-butoxycarbonyl) pipe rid in e-3-carboxylic acid (cis racemic) (12 g, 0.052 mol) in THE (72 mL) and water (72 mL) was added NaHCO₃ (13.2 g, 0.157 mol) and stirred for 15 minutes. The reaction mixture was cooled and Cbz-OSu (13.05 g, 0.052 mol) was added portion-wise. The reaction mixture was allowed to warm to rt and stirred for 3 hours. Water (100 mL) was added and the organic phase was extracted with ethyl acetate (3×50 mL). The combined organic layer was washed with water (100 mL) and concentrated under vacuum to get crude compound which was purified by column chromatography (230-400 silica gel) using 0-40% ethyl acetate in pet-ether. Fractions containing the desired product were concentrated and the residue was further purified by SFC to get (2R,3S)-1-((benzyloxy)carbonyl)-2-(tert-butoxycarbonyl)piperidine-3-carboxylic acid (1.3 g, 6.8%) as a white solid.

AnalpH9_MeCN_4 min, Rt: 1.77 min, m/z 362.2 [M−H]⁻

¹H-NMR (400 MHz, DMSO-D₆) δ 7.30-7.39 (m, 5H), 5.02-5.21 (m, 3H), 3.91 (d, J=11.9 Hz, 1H), 2.79-2.84 (m, 0.5H), 2.56-2.70 (m, 1.5H), 1.89 (d, J=9.2 Hz, 1H), 1.64-1.70 (m, 1H), 1.36 (s) and 1.34 (s, 9H).

### Synthesis of (2R,3S)-1-tert-butoxycarbonyl-3-[2-[(3S)-3-fluoropyrrolidin-1-yl]ethyl-methyl-carbamoyl]piperidine-2-carboxylic acid (M06109-S4)

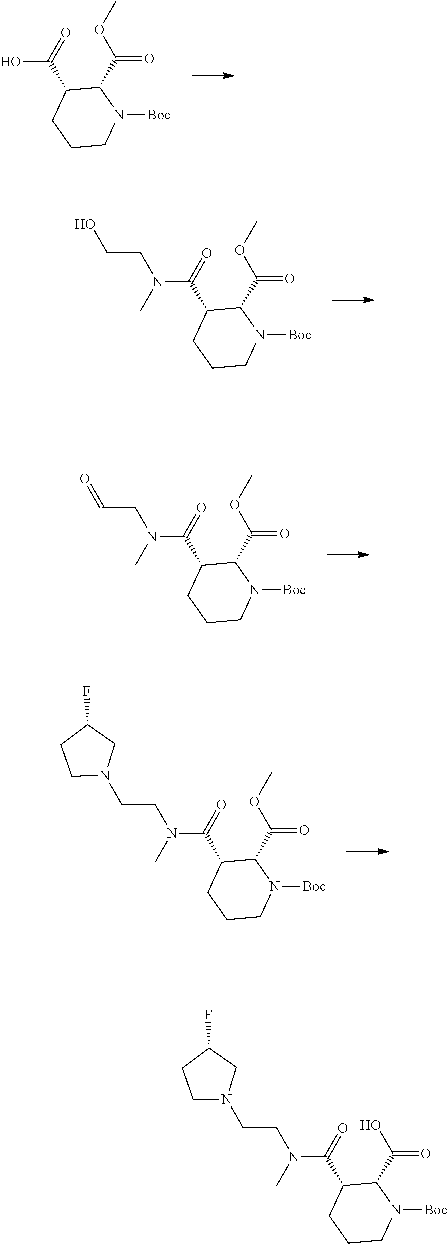 [SMILES: COC(=O)[C@H]1[C@@H](C(=O)O)CCCN1C(=O)OC(C)(C)C ; COC(=O)[C@H]1[C@@H](C(=O)N(C)CCO)CCCN1C(=O)OC(C)(C)C ; COC(=O)[C@H]1[C@@H](C(=O)N(C)CC=O)CCCN1C(=O)OC(C)(C)C ; COC(=O)[C@H]1[C@@H](C(=O)N(C)CCN2CC[C@H](F)C2)CCCN1C(=O)OC(C)(C)C ; CN(CCN1CC[C@H](F)C1)C(=O)[C@H]1CCCN(C(=O)OC(C)(C)C)[C@H]1C(=O)O]

### Step 1: Synthesis of O1-tert-butyl O2-methyl (2R,3S)-3-[2-hydroxyethyl(methyl)carbamoyl]piperidine-1,2-dicarboxylate

(2R,3S)-1-(tert-butoxycarbonyl)-2-(methoxycarbonyl)piperidine-3-carboxylic acid (750 mg, 2.61 mmol) was dissolved in anhydrous DCM (20.9 mL), added 2-(methylamino)ethanol (0.25 mL, 3.13 mmol) followed by N,N-Diisopropylethylamine (1.4 mL, 7.83 mmol) and finally HATU (1191 mg, 3.13 mmol). The reaction mixture was left to stir at room temperature overnight. The reaction was quenched with sat. NaHCO₃, the aqueous phase was then extracted with DCM (×3). All DCM layers were passed through a phase separator, combined, and concentrated in vacuo. The crude was purified by flash column chromatography (25 g Sfar, 0-25% DCM/DCM w/20% MeOH) and solvent removed to give O1-tert-butyl O2-methyl (2R,3S)-3-[2-hydroxyethyl(methyl)carbamoyl]piperidine-1,2-dicarboxylate (899 mg, 2.61 mmol, 100%) as a colourless oil.

UPLC_pH9_MeCN_2 min, Rt: 1.43 min, m/z 345.1 [M+H]⁺

### Step 2: Synthesis of O1-tert-butyl O2-methyl (2R,3S)-3-[methyl(2-oxoethyl)carbamoyl]piperidine-1.2-dicarboxylate

O1-tert-butyl O2-methyl (2R,3S)-3-[2-hydroxyethyl(methyl)carbamoyl]piperidine-1,2-dicarboxylate (899 mg, 2.61 mmol) was dissolved in DCM (26 mL), cooled to 0° C. then Dess-Martin periodinane (1.66 g, 3.92 mmol) was added portion-wise. The reaction was left to stir at 0° C. for 2.5 hours, ice removed after 1 hour. The reaction was quenched with sat. sodium thiosulfate solution, stirring for 10 minutes. The aqueous phase was then extracted with DCM (×3), all DCM layers were passed through a phase separator, combined and concentrated in vacuo to give O1-tert-butyl O2-methyl (2R,3S)-3-[methyl(2-oxoethyl)carbamoyl]piperidine-1,2-dicarboxylate (894 mg, 2.61 mmol, quant) as a yellow oil.

UPLC_pH9_MeCN_2 min, Rt: 1.43-1.57 min, m/z 243.2 [M+H−boc]⁺

### Step 3: Synthesis of O1-tert-butyl O2-methyl (2R,3S)-3-[2-[(3S)-3-fluoropyrrolidin-1-yl]ethyl-methyl-carbamoyl]piperidine-1,2-dicarboxylate

O1-tert-butyl O2-methyl (2R,3S)-3-[methyl(2-oxoethyl)carbamoyl]piperidine-1,2-dicarboxylate (149 mg, 0.435 mmol) was dissolved in MeOH (5.9 mL). (S)-(+)-3-Fluoropyrrolidine hydrochloride (67 mg, 0.522 mmol) was added followed by AcOH (0.3 mL). The reaction was left to stir for 1.5 hours after which Sodium cyanoborohydride (55 mg, 0.87 mmol) was added. The reaction was left to stir overnight at room temperature. The reaction was quenched with sat. NaHCO₃, the aqueous phase was then extracted with DCM (×3). All DCM layers were passed through a phase separator, combined and concentrated in vacuo. The crude was purified via SCX (2 g) to give O1-tert-butyl O2-methyl (2R,3S)-3-[2-[(3S)-3-fluoropyrrolidin-1-yl]ethyl-methyl-carbamoyl]piperidine-1,2-dicarboxylate (130 mg, 0.275 mmol, 63%) as a brown oil.

UPLC_pH9_MeCN_2 min, Rt: 1.63 min, m/z 416.2 [M+H]⁺

### Step 4: Synthesis of (2R,3S)-1-tert-butoxycarbonyl-3-[2-[(3S)-3-fluoropyrrolidin-1-yl]ethyl-methyl-carbamoyl]piperidine-2-carboxylic acid

O1-tert-butyl O2-methyl (2R,3S)-3-[2-[(3S)-3-fluoropyrrolidin-1-yl]ethyl-methyl-carbamoyl]piperidine-1,2-dicarboxylate (130 mg, 0.313 mmol) was dissolved in MeOH (5.2 mL), 1M hydroxylithium(aq) (1.6 mL, 1.56 mmol) was added dropwise and the reaction left to stir overnight at room temperature. Additional 1 M hydroxylithium(aq) (1.6 mL, 1.56 mmol) was added and the reaction stirred for 3 hours. 1 M HCl was added, the reaction was concentrated in vacuo, redissolved in DCM and filtered through a phase separator. The crude was purified via reverse phase flash, (30 g redisep gold rf cartridge, pH9/MeCN, 0-30% MeCN over 30CV, diastereomers eluted around 10CV between 10-15% MeCN). Fractions containing product were combined and concentrated to give (2R,3S)-1-tert-butoxycarbonyl-3-[2-[(3S)-3-fluoropyrrolidin-1-yl]ethyl-methyl-carbamoyl]piperidine-2-carboxylic acid (80.5 mg, 0.185 mmol, 59%) as a clear film.

UPLC_pH9_MeCN_2 min. Rt: 1.22 min. m/z 402.3 [M+H]⁺

The following compounds were made by analogous methods:

| | | | Mass, |
|---|---|---|---|
| | | | yield, |
| Example No. | Structure & conditions | Analytical Data | state |
| M06128-S4 (2R,3S)-1-tert- butoxycarbonyl-3-[2-[(3S)-3- methoxypyrrolidin-1-yl]ethyl- methyl-carbamoyl]piperidine- 2-carboxylic acid | 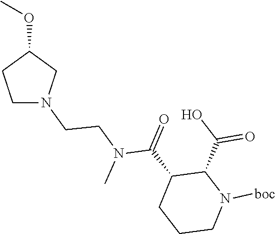 Using (3S)-3- methoxypyrollidine hydrochloride in step 3 | UPLC_pH9_MeCN_2 min, Rt: 1.21 min, m/z 414.3 [M + H]⁺ | 112 mg, 51%, colourless gum |
| M06129-S4 (2R,3S)-1-tert- butoxycarbonyl-3-[2-(3,3- difluoroazetidin-1-yl)ethyl- methyl-carbamoyl]piperidine- 2-carboxylic acid | 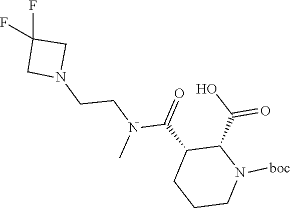 Using 3,3- Difluoroazetidine hydro- chloride in step 3 | UPLC_pH9_MeCN_2 min, Rt: 1.22 min, m/z 406.3 [M + H]⁺ | 51 mg, 41%, clear film |
| M06131-S4 (2R,3S)-1-tert- butoxycarbonyl-3-[2-[2- fluoroethyl (methyl)amino] ethyl-methyl-carbamoyl] piperidine-2-carboxylic acid | 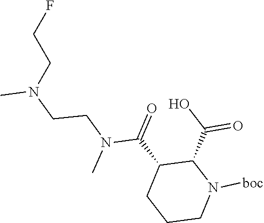 Using (2-fluoroethyl) (methyl)amine hydrochloride in step 3 | UPLC_pH9_MeCN_2 min, Rt: 1.23 min, m/z 390.2 [M + H]⁺ | 24.4 mg, 27%, colourless oil |

### Synthesis of (2R,3S)-1-tert-butoxycarbonyl-3-[2-(3-fluoroazetidin-1-yl)ethyl-methyl-carbamoyl]piperidine-2-carboxylic acid (M06099-int)

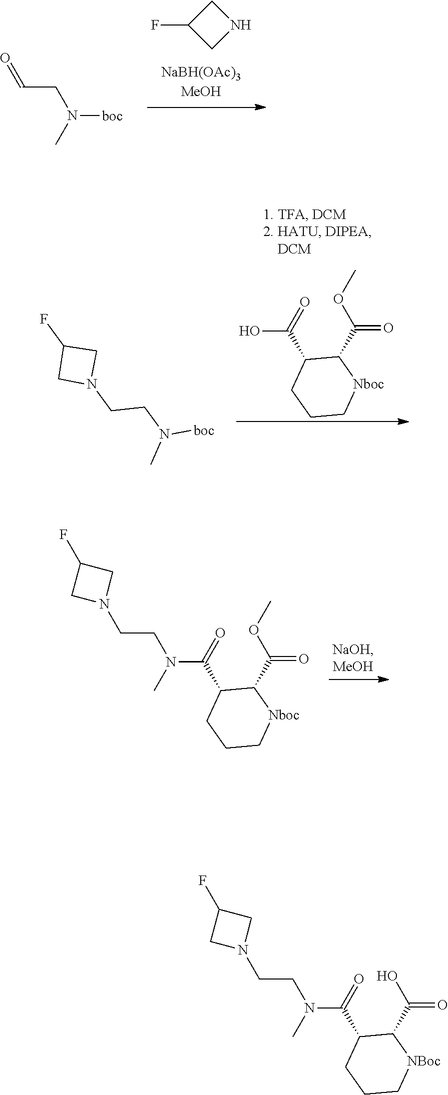

### Step 1: Synthesis of tert-butyl N-[2-(3-fluoroazetidin-1-yl)ethyl]-N-methyl-carbamate

N-Boc-(Methylamino)acetaldehyde (200 mg, 1.15 mmol) was dissolved in methanol (5.0 mL) and 3-fluoroazetidine hydrochloride (129 mg, 1.15 mmol) was added. After 20 minutes sodium triacetoxyborohydride (245 mg, 1.15 mmol) was added and the reaction stirred at rt overnight. To the mixture was added sat. aq. sodium bicarbonate (5 mL) and extracted with DCM (2×20 mL). The combined organics were passed through a phase separator and solvent removed. The residue was purified by SCX-2 (5 g), washing with MeOH and eluting with 0.5 M NH3/MeOH. The solvent was removed to give tert-butyl N-[2-(3-fluoroazetidin-1-yl)ethyl]-N-methyl-carbamate (100 mg, 0.430 mmol, 37%) as a pale yellow oil.

AnalpH9_MeCN_4 min, Rt: 2.02 min, m/z 233.4 [M+H]⁺

NMR (CDCl₃): 1H-NMR (400 MHz, CHLOROFORM-D) δ 5.20-5.01 (m, 1H), 3.72-3.64 (m, 2H), 3.19-3.07 (m, 4H), 2.94-2.86 (m, 3H), 2.68-2.55 (m, 2H), 1.53-1.37 (m, 9H)

### Step 2: Synthesis of O1-tert-butyl O2-methyl rac-(2R,3S)-3-[2-(3-fluoroazetidin-1-yl)ethyl-methyl-carbamoyl]piperidine-1,2-dicarboxylate

tert-butyl N-[2-(3-fluoroazetidin-1-yl)ethyl]-N-methyl-carbamate (100 mg, 0.43 mmol) was dissolved in dichloromethane (2.0 mL) and trifluoroacetic acid (0.20 mL) added. The reaction was stirred at rt overnight. The solvent was removed and the residue dissolved in dichloromethane (2.0 mL) and (2R,3S)-1-(tert-butoxycarbonyl)-2-(methoxycarbonyl)piperidine-3-carboxylic acid (124 mg, 0.43 mmol), N,N-Diisopropylethylamine (0.45 mL, 2.58 mmol) and HATU (164 mg, 0.430 mmol) were added sequentially. The reaction was stirred at rt for 4 hours. Water (20 mL) was added and the layers separated. The aqueous was extracted with 2×10 mL DCM and the combined organics passed through a phase separator and solvent removed. The residue was purified by SCX-2 (5 g), washing with MeOH and eluting with 0.5 M NH3/MeOH to give O1-tert-butyl O2-methyl rac-(2R,3S)-3-[2-(3-fluoroazetidin-1-yl)ethyl-methyl-carbamoyl]piperidine-1,2-dicarboxylate (133 mg, 0.331 mmol, 77%) as a brown oil.

AnalpH9_MeCN_4 min, Rt: 2.13 min, m/z 402.3 [M+H]⁺

### Step 3: Synthesis of (2R,3S)-1-tert-butoxycarbonyl-3-[2-(3-fluoroazetidin-1-yl)ethyl-methyl-carbamoyl]piperidine-2-carboxylic acid

O1-tert-butyl O2-methyl rac-(2R,3S)-3-[2-(3-fluoroazetidin-1-yl)ethyl-methyl-carbamoyl]piperidine-1,2-dicarboxylate (133 mg, 0.331 mmol) was dissolved in methanol (2.0 mL) and 1M hydroxylithium(aq) (1.66 mL, 1.66 mmol) was added. The reaction was stirred at rt overnight. The reaction was neutralised with 2M HCl and solvent removed. The residue was triturated with DCM, filtered and solvent removed to give crude compound as an orange oil. The product was purified by Preparative HPLC to give (2R,3S)-1-tert-butoxycarbonyl-3-[2-(3-fluoroazetidin-1-yl)ethyl-methyl-carbamoyl]piperidine-2-carboxylic acid (88 mg, 0.227 mmol, 69%) as a pale yellow oil.

AnalpH9_MeCN_4 min, Rt: 1.49 min, m/z 388.3 [M+H]⁺

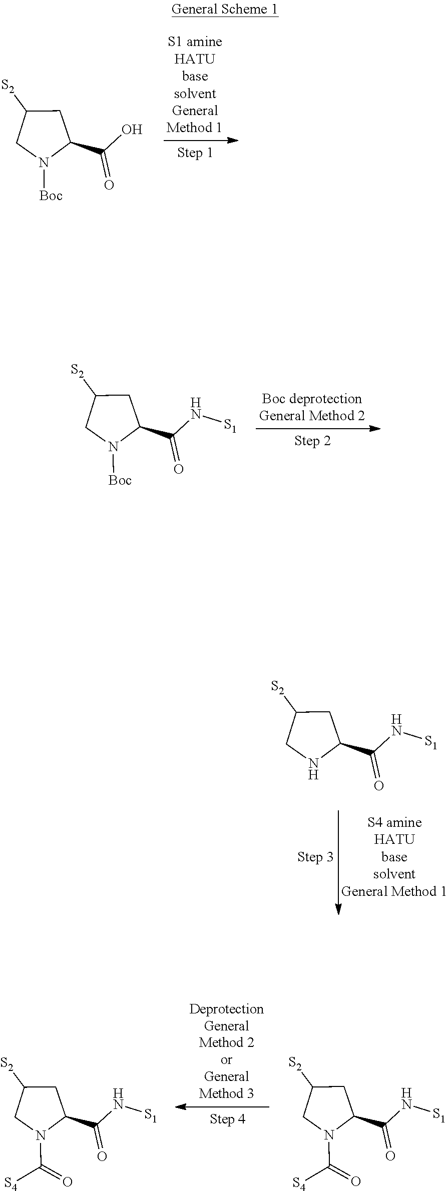

### Synthesis of (2S,4R)—N-((1-methyl-1H-indazol-5-yl)methyl)-4-(4-methylbenzyl)-1-((2R,3S)-3-(morpholine-4-carbonyl)piperidine-2-carbonyl)pyrrolidine-2-carboxamide (M05753)

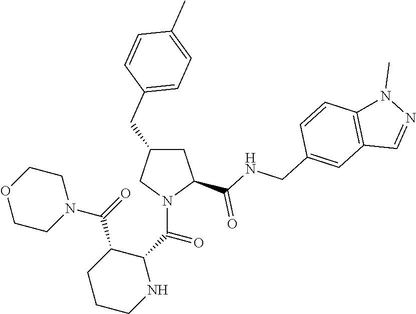 [SMILES: Cc1ccc(C[C@@H]2C[C@@H](C(=O)NCc3ccc4c(cnn4C)c3)N(C(=O)[C@@H]3NCCC[C@@H]3C(=O)N3CCOCC3)C2)cc1]

Step 1: To a stirred solution of (1-methylindazol-5-yl)methanamine (950 mg, 5.89 mmol) and Boc-(R)-4-(4-methylbenzyl)-L-proline (1.8 g, 5.6 mmol) in dichloromethane (28 mL) at room temperature was added N,N-diisopropylethylamine (2.90 mL, 16.8 mmol). The reaction mixture was stirred 10 minutes and then HATU (2.6 g, 6.74 mmol) was added in one portion. The reaction mixture was stirred 2 hours at room temperature. Aqueous saturated NaHCO₃ solution (30 mL) was added. The phases were separated, and the aqueous phase was extracted with DCM (2×50 mL). The organic phases were combined and passed through a phase separator (Biotage) before concentrating under vacuum. The residue was purified by flash column chromatography (SiO₂, Biotage Isolera, 100 g, Sfär column, eluting from 100% DCM to 10% MeOH in DCM) affording tert-butyl-(2S,4R)-2-[(1-methylindazol-5-yl)methylcarbamoyl]-4-(p-tolylmethyl)pyrrolidine-1-carboxylate (2.0 g, 77%) as a white solid.

AnalpH9_MeCN_4 min, rt: 2.46 min, m/z 363.3 [M+H−Boc]⁺

Step 2: tert-Butyl (2S,4R)-2-[(1-methylindazol-5-yl)methylcarbamoyl]-4-(p-tolylmethyl)pyrrolidine-1-carboxylate (2 g) was dissolved in DCM (50 mL) and TFA (22 mL) was added portionwise over 2 mins. The reaction mixture was left to stir at room temperature for 2.5 hours. The reaction mixture was then concentrated in vacuo. The residue was dissolved in MeOH and purified via SCX-2 cartridge (50 g, Biotage). The cartridge was washed through with 3 CV of MeOH. The bound compound was eluted with 3 CV of 3.5M NH₃ in MeOH. The solution was concentrated in vacuo affording (2S,4R)—N-((1-methyl-1H-indazol-5-yl)methyl)-4-(4-methylbenzyl)pyrrolidine-2-carboxamide (1.16 g, 74%) as an off-white solid.

AnalpH9_MeCN_4 min, Rt: 2.30 min, m/z 363.3 [M+H]⁺

Step 3: To a stirred solution of (2R,3S)-1-tert-butoxycarbonyl-3-(morpholine-4-carbonyl)piperidine-2-carboxylic acid (70 mg, 0.206 mmol) and (2S,4R)—N-[(1-methylindazol-5-yl)methyl]-4-(p-tolylmethyl)pyrrolidine-2-carboxamide (75 mg, 0.206 mmol) in dichloromethane (2.0 mL) at 0° C. was added N,N-diisopropylethylamine (0.072 mL, 0.411 mmol). The reaction mixture was stirred at room temperature for 10 min, and then HATU (94 mg, 0.247 mmol) was added. The reaction mixture was stirred at room temperature for 2 hours. Aqueous saturated NaHCO₃ solution (2 mL) was added. The phases were separated, and the aqueous phase was extracted with DCM (2×5 mL). The organic phases were combined and were passed through a phase separator before concentrating under vacuum. The residue was purified by preparative HPLC. The fractions containing the desired product were concentrated to afford tert-butyl (2R,3S)-2-[(2S,4R)-2-[(1-methylindazol-5-yl)methylcarbamoyl]-4-(p-tolylmethyl)pyrrolidine-1-carbonyl]-3-(morpholine-4-carbonyl)piperidine-1-carboxylate (70 mg, 0.102 mmol, 50%) as a white solid.

AnalpH9_MeCN_4 min, Rt: 2.64 min, m/z 687.5 [M+H]⁺

Step 4: To a stirred solution of tert-butyl (2R,3S)-2-[(2S,4R)-2-[(1-methylindazol-5-yl)methylcarbamoyl]-4-(p-tolylmethyl)pyrrolidine-1-carbonyl]-3-(morpholine-4-carbonyl)piperidine-1-carboxylate (70 mg, 0.102 mmol) in dichloromethane (5.3 mL) at room temperature was added trifluoroacetic acid (1.1 mL) dropwise. The reaction mixture was stirred at room temperature until consumption of starting material (6 hours). The reaction mixture was concentrated under vacuum. The residue was redissolved in MeOH (1 mL) and loaded onto a SCX-2 cartridge (Biotage, 1 g) eluting with MeOH (3 CV). The bound material was released by eluting with a 3.5M solution of NH₃ in MeOH (3 CV). The product-containing fractions were concentrated under vacuum and the residue was purified by preparative HPLC. Fractions containing the desired product were concentrated, to afford (2S,4R)—N-[(1-methylindazol-5-yl)methyl]-1-[(2R,3S)-3-(morpholine-4-carbonyl)piperidine-2-carbonyl]-4-(p-tolylmethyl)pyrrolidine-2-carboxamide (41 mg, 69%) as a white solid.

AnalpH9_MeOH_QC_v1, Rt: 7.85 mins, m/z 587.6 [M+H]⁺

AnalpH2_MeOH_QC_v1, Rt: 5.87 mins, m/z 587.6 [M+H]⁺

¹H-NMR (400 MHz, DMSO-D₆) δ 8.59 (t, J=6.2 Hz, 0.5H), 8.08 (t, J=6.0 Hz, 0.4H), 7.95 (d, J=11 Hz, 1H), 7.58-7.51 (m, 2H), 7.28 (dd, J=8.9, 1.1 Hz, 0.5H), 7.24 (dd, J=8.7, 1.4 Hz, 0.4H), 7.11-7.04 (m, 4H), 5.24-5.23 (m, 0.6H), 4.44-4.24 (m, 2.5H), 4.02-3.96 (m, 3.5H), 3.52-3.40 (m, 8H), 3.16 (t, J=8.9 Hz, 0.5H), 3.00-2.76 (m, 3H), 2.70-2.53 (m, 2H), 2.38-2.08 (m, 5H), 2.02-1.23 (m, 5H), 1.13-1.10 (m, 0.6H).

The following compounds were made by analogous methods:

| | | | Mass, % |
|---|---|---|---|
| Example | | | yield, |
| No | Structure & Conditions | Analytical Data | state |
| M04490 | 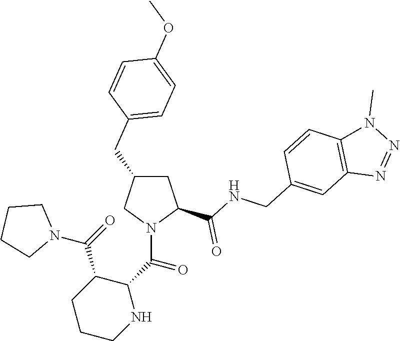 [SMILES: COc1ccc(C[C@@H]2C[C@@H](C(=O)NCc3ccc4c(c3)nnn4C)N(C(=O)[C@@H]3NCCC[C@@H]3C(=O)N3CCCC3)C2)cc1] | AnalpH2_MeOH_QC_v1, Rt: 5.45 min, m/z 588.5 [M + H]⁺ AnalpH9_MeOH_QC_v1, Rt: 7.42 min, m/z 588.5 [M + H]⁺ | 41 mg, 68%, white solid |
| | (2S,4R)-4-(4-methoxybenzyl)-N-((1-methyl-1H- | | |
| | benzo[d]1,2,3]triazol-5-yl)methyl)-1-((2R,3S)- | | |
| | 3-(pyrrolidine-1-carbonyl)piperidine-2- | | |
| | carbonyl)pyrrolidine-2-carboxamide | | |
| | S1: (1-methyl-1H-benzo[d][1,2,3]triazol-5- | | |
| | yl)methanamine | | |
| | S2: (2S,4R)-1-(tert-butoxycarbonyl)-4-(4- | | |
| | methoxybenzyl)pyrrolidine-2-carboxylic acid | | |
| | S4: (2R,3S)-1-(tert-butoxycarbonyl(-3- | | |
| | pyrrolidine-1-carbonyl)piperidine-2-carboxylic | | |
| | acid | | |
| | Step 1: GM1 with DMF and DIPEA. Amine | | |
| | used as HCl salt. Purified by flash column | | |
| | chromatography | | |
| | Step 2: GM2B. Purification d | | |
| | Step 3: GM1 with DMF and DIPEA. Purified | | |
| | by prep HPLC | | |
| | Step 4: GM2A. Final purification a (SCX-2 and | | |
| | prep HPLC) | | |
| M00466 | 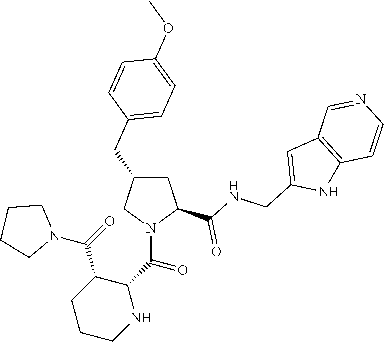 [SMILES: COc1ccc(C[C@@H]2C[C@@H](C(=O)NCc3cc4cnccc4[nH]3)N(C(=O)[C@@H]3NCCC[C@@H]3C(=O)N3CCCC3)C2)cc1] | AnalpH2_MeOH_QC_v1, Rt: 3.83 min, m/z 573.3 [M + H]⁺ AnalpH9_MeOH_QC_v1, Rt: 7.50 min, m/z 573.3 [M + H]⁺ 1H-NMR (400 MHz, DMSO-D₆) δ 11.17- 11.25 (m, 1H), 8.66 (s, 1H), 8.56 (t, J = 6.0 Hz, 0.5H), 8.14 (t, J = 5.7 Hz, 0.4H), 8.05-8.07 (m, 1H), 7.25-7.29 (m, 1H), 7.04-7.08 (m, 2H 6.77-6.82 (m, 2H), 6.28 (s, 1H), 5.18-5.19 (m, 0.5H), 4.28-4.44 (m, 3H), 3.97-4.01 (m, 0.5H), 3.68 (s, 3H), 3.52-3.53 (m, 0.4H), 3.41-3.46 (m, 0.4H), | 31.0 mg, 33%, white solid |
| | (2S,4R)-N-((1H-pyrrolo[3,2-c]pyridin-2- | 3.17-3.22 (m, 1H), | |
| | yl)methyl)-4-(4-mthoxybenzyl)-1-((2R,3S)-3- | 3.07-3.14 (m, 1H), | |
| | (pyrrolidine-1-carbonyl)piperidine-2- | 2.92-3.07 (m, 1H), | |
| | carbonyl)pyrrolidine-2-carboxamide | 2.83-2.86 (m, 0.5H), | |
| | S1: (1H-pyrrolo[3,2-c]pyridin-2-yl)methanamine | 2.74-2.75 (m, 0.4H), | |
| | S2: (2S,4R)-1-(tert-butoxycarbonyl)-4-(4- | 2.25-2.33 (m, 1H), | |
| | methoxybenzyl)pyrrolidine-2-carboxylic acid | 1.60-1.97 (m, 8H), | |
| | S4: (2R,3S)-1-(tert-butoxycarbonyl)-3- | 1.19-1.54 (m, 1H), | |
| | (pyrrolidine-1-carbonyl)piperidine-2-carboxylic | 0.98-1.06 (m, 0.5H). | |
| | acid | | |
| | Step 1: GM1 with DMF and DIPEA. Purified by | | |
| | column chromatography. | | |
| | Step 2: GM2A. Purification b. | | |
| | Step 3: GM1 with DMF/DCM and DIPEA. | | |
| | Purified by column chromatography | | |
| | Step 4: GM2A final purification a (SCX-2 and | | |
| | prep) | | |
| M00467 | 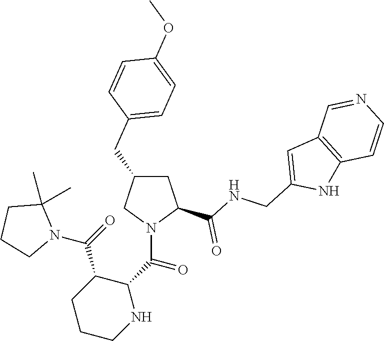 [SMILES: COc1ccc(C[C@@H]2C[C@@H](C(=O)NCc3cc4cnccc4[nH]3)N(C(=O)[C@@H]3NCCC[C@@H]3C(=O)N3CCCC3(C)C)C2)cc1] | Thermo_MeOH_UHPLC_ 1.2 min LCMS: Rₜ = 0.5 min m/z = 601.50 [M + H]⁺ | 21 mg, white solid |
| | S1: (1H-pyrrolo[3,2-c]pyridin-2-yl)methanamine | | |
| | S2: (2S, 4R)-1-(tert-butoxycarbonyl)-4-(4- | | |
| | methoxybenzyl)pyrrolidine-2-carboxylic acid | | |
| | S4: (2R,3S)-1-(tert-butoxycarbonyl)-3-(2,2- | | |
| | dimethylpyrrolidine-1-carbonyl)piperidine-2- carboxylic acid | | |
| | Step 1: GM1 with DMF and DIPEA. Purified by | | |
| | column chromatography. | | |
| | Step 2: GM2A. Purification b | | |
| | Step 3: GM1 with DMF/DCM and DIPEA. | | |
| | Purified by column chromatography | | |
| | Step 4: GM2A | | |
| M05675 | 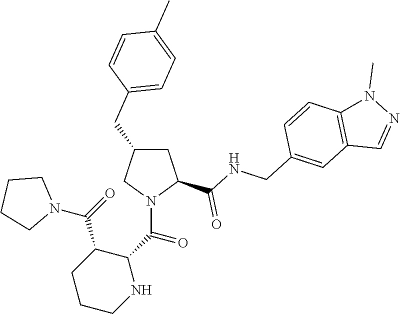 [SMILES: Cc1ccc(C[C@@H]2C[C@@H](C(=O)NCc3ccc4c(cnn4C)c3)N(C(=O)[C@@H]3NCCC[C@@H]3C(=O)N3CCCC3)C2)cc1] | AnalpH2_MeOH_QC_v1, Rt: 6.22 min, m/z 571.5 [M + H]⁺ AnalpH9_MeOH_QC_v1, Rt: 8.11 min, m/z 571.5 [M + H]⁺ ¹H-NMR (400 MHz, DMSO-D₆) δ 8.57 (t, J = 6.0 Hz, 0.6H), 8.08 (t, J = 6.0 Hz, 0.4H), 7.96 (s, 1H), 7.51-7.58 (m, 2H), 7.25 (ddd, J = 19.2, 8.7, 1.4 Hz, 1H), 7.04-7.11 (m, 4H), 5.19-5.22 (m, 1H), 4.25-4.43 (m, 2H), 3.07-3.97 (m, 1H), 4.02 (s, 3H), 3.42-3.54 (m, 1H), 3.11-3.25 (m, 3H), 3.02-3.08 (m, 0.4H), | 19 mg, 86% white solid |
| | (2S,4R)-N-((1-methyl-1H-indazol-5-yl)methyl)- | 2.84-2.99 (m, 2H), | |
| | 4-(4-methylbenzyl)-1-((2R,3S)-3-(pyrrolidine-1- | 2.77-2.75 (m, 0.4H), | |
| | carbonyl)piperidine-2-carbonyl)pyrrolidine-2- | 2.54-2.68 (m, 0.5H), | |
| | carboxamide | 2.29-2.37 (m, 0.4H), | |
| | S1: 1-methyl-1H-indazol-5-yl)methanamine | 2.26 (s, 3H), 2.13-2.23 | |
| | S2: (2S,4R)-1-(tert-butoxycarbonyl)-4-(4- | (m, 0.3H), 1.66-2.02 | |
| | methylbenzyl)pyrrolidine-2-carboxylic acid | (m, 8H), 1.48-1.60 (m, | |
| | S4: (2R,3S)-1-(tert-butoxycarbonyl)-3- | 0.6H), 1.24-1.36 (m, | |
| | (pyrrolidine-1-carbonyl)piperidine-2-carboxylic | 1H), 1.05-1.15 (m, | |
| | acid | 0.6H) | |
| | Step1: GM1 with DMF and DIPEA. Purified by | | |
| | column chromatogaphy. | | |
| | Step 2: GM2A. Purification a (SCX-2). | | |
| | Step 3: GM1 with DMF and DIPEA. Purified by | | |
| | prep HPLC | | |
| | Step 4: GM2A final purification a (SCX-2) | | |
| M05712 | 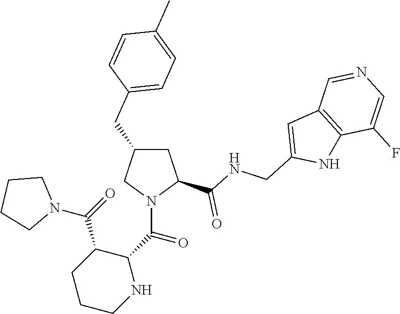 [SMILES: Cc1ccc(C[C@@H]2C[C@@H](C(=O)NCc3cc4cncc(F)c4[nH]3)N(C(=O)[C@@H]3NCCC[C@@H]3C(=O)N3CCCC3)C2)cc1] | AnalpH2_MeOH_QC_v1, Rt: 4.33 min, m/z 575.5 [M + H]⁺ AnalpH9_MeOH_QC_v1, Rt: 8.12 min, m/z 575.5 [M + H]⁺ ¹H-NMR (400 MHz, DMSO-D₆) δ 12.05 (s, 0.4H), 11.92 (s, 0.4H), 8.60 (t, J = 5.7 Hz, 0.4H), 8.56 (t, J = 2.3 Hz, 1H), 8.52 (d, J = 2.7 Hz, 0.1H), 8.16 (t, J = 5.7 Hz, 0.5H), 8.08 (dd, J = 8.0, 3.0 Hz, 1H), 8.02 (d, J = 3.2 Hz, 0.1H), 7.06-7.08 (m, 4H), 7.02 (d, J = 10.1 Hz, 0.1H), 6.97 (d, J = 7.8 Hz, 0.1H), 6.60 | 13.8 mg, 76%, white solid |
| | (2S,4R)-N-((7-fluoro-1H-pyrrolo[3,2-c]pyridin-2- | (s, 0.1H), 6.41 (t, J = | |
| | yl)methyl)-4-(4-methylbenzyl)-1-((2R,3S)-3- | 3.4 Hz, 1H), 5.22 (d, | |
| | (pyrrolidine-1-carbonyl)piperidine-2- | J = 7.3 Hz, 0.5H), 4.75 | |
| | carbonyl)pyrrolidine-2-carboxamide | (d, J = 5.5 Hz, 0.1H), | |
| | S1: (7-fluoro-1H-pyrrolo[3,2-c]pyridine-2- | 4.31-4.48 (m, 3H), | |
| | yl)methanamine | 4.25-4.18 (0.1H), 4.01- | |
| | S2: (2S,4R)-1-(tert-butoxycarbonyl)-4-(4- | 4.10 (m, 1H), 3.38-3.55 | |
| | methylbenzyl)pyrrolidine-2-carboxylic acid | (m, 1H), 3.30 (s, 1H), | |
| | S4: (2R,3S)-1-(tert-butoxycarbonyl)-3- | 3.10-3.27 (m, 3H), | |
| | (pyrrolidine-1-carbonyl)piperidine-2-carboxylic | 2.74-3.06 (m, 3H), | |
| | acid | 2.54-2.66 (m, 1H), | |
| | Step1: GM1 with DMF and DIPEA. Purified by | 2.30-2.33 (m, 0.3H), | |
| | column chromatogaphy. | 2.25 (s, 3H), 2.22 (s, | |
| | Step 2: GM2A. Purification a (SCX-2). | 0.3H), 1.68-1.98 (m, | |
| | Step 3: GM1 with DMF and DIPEA. Purified by | 9H), 1.53-1.56 (m, | |
| | prep HPLC | 0.3H), 1.37-1.44 (m, | |
| | Step 4: GM2A final purification a (SCX-2) and | 0.5H), 1.22-1.29 (m, | |
| | prep HPLC) | 0.5H), 1.07-1.13 (m, | |
| | | 0.5H) | |
| M05720 | 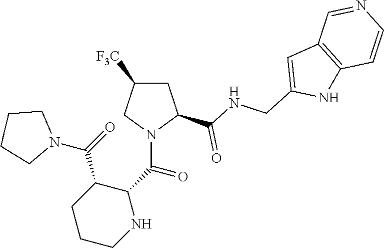 [SMILES: O=C(NCc1cc2cnccc2[nH]1)[C@@H]1C[C@H](C(F)(F)F)CN1C(=O)[C@@H]1NCCC[C@@H]1C(=O)N1CCCC1] | AnalpH2_MeOH_QC_v1, Rt: 1.29 min, m/z 521.4 [M + H]⁺ AnalpH9_MeOH_QC_v1, Rt: 7.09 min, m/z 521.3 [M + H]⁺ ¹H-NMR (400 MHz, DMSO-D₆) δ 11.31 (0.4H, s), 11.20 (0.5H, s), 8.70-8.73 (1.4H, m), 8.25-8.28 (0.5H, m), 8.08-8.11 (1H, m), 7.30-7.34 (1H, m), 6.39 (1H, d, J = 6.4 Hz), 5.26 (0.4H, t, J = 7.8 Hz), 4.32-4.52 (2.4H, | 19.3 mg, 56%, white solid |
| | (2S,4S)-N-((1H-pyrrolo[3,2-c]pyridin-2- | m), 4.01 (0.5H, dd, J = | |
| | yl)methyl)-1-((2R,3S)-3-(pyrrolidine-1- | 11.2, 8.0 Hz), 3.90-3.90 | |
| | carbonyl)piperidine-2-carbonyl)-4- | (0.4H, m), 3.71 (0.5H, t, | |
| | (trifluoromethyl)pyrrolidine-2-carboxamide | J = 11.0 Hz), 3.58-3.58 | |
| | S1: (1H-pyrrolo[3,2-c]pyridin-2-yl)methanamine | (0.5H, m), 3.39-3.44 | |
| | dihydrochloride | (1.5H, m), 3.18-3.26 | |
| | S2: (2S,4S)-1-(tert-butoxycarbonyl)-4- | (3.5H, m), 3.05-3.06 | |
| | (trifluoromethyl)pyrrolidine-2-carboxylic acid | (1H, m), 2.58-2.84 | |
| | S4: (2R,3S)-1-(tert-butoxycarbonyl)-3- | (3.3H, m), 2.47-2.36 | |
| | pyrrolidine-1-carbonyl)piperidine-2-carboxylic | (0.5H, m), 1.56-2.04 | |
| | acid | (7.4H, m), 1.30-1.40 | |
| | Step1: GM1 with DMF and DIPEA. Purified by | (1.4H, m), 0.97-0.97 | |
| | column chromatogaphy. | (0.4H, m) | |
| | Step 2: GM2A. Purification a (SCX-2). | | |
| | Step 3: GM1 with DMF and DIPEA. Purified by | | |
| | prep HPLC | | |
| | Step 4: GM2A final purification a (SCX-2) and | | |
| | prep HPLC) | | |
| M05733 | 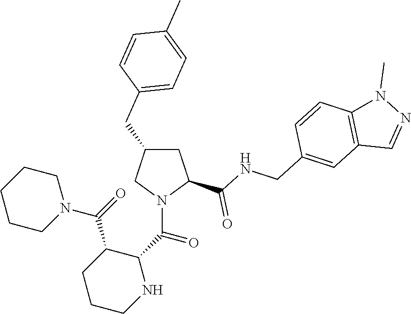 [SMILES: Cc1ccc(C[C@@H]2C[C@@H](C(=O)NCc3ccc4c(cnn4C)c3)N(C(=O)[C@@H]3NCCC[C@@H]3C(=O)N3CCCCC3)C2)cc1] | AnalpH2_MeOH_QC_v1, Rt: 6.25 min, m/z 585.6 [M + H]⁺ AnalpH9_MeOH_QC_v1, Rt: 8.27 min, m/z 585.6 [M + H]⁺ | 13.6 mg, 61%, white solid |
| | (2S,4R)-N-((1-methyl-1H-indazol-5-yl)methyl)- | | |
| | 4-(4-methylbenzyl)-1-((2R,3S)-3-(piperidine-1- | | |
| | carbonyl)piperidine-2-carbonyl)pyrrolidine-2- | | |
| | carboxamide | | |
| | S1: 1-methyl-1H-indazol-5-yl)methanamine | | |
| | S2: (2S,R)-1-(tert-butoxycarbonyl)-4-(4- | | |
| | methylbenzyl)pyrrolidine-carboxylic acid | | |
| | S4: (2R,3S)-1-tert-butoxycarbonyl)-3- | | |
| | (piperidine-1-carbonyl)piperidine-2-carboxylic | | |
| | acid | | |
| | Step1: GM1 with DMF and DIPEA. Purified by | | |
| | column chromatogaphy. | | |
| | Step 2: GM2A. Purification a (SCX-2). | | |
| | Step 3: GM1 with DMF and DIPEA. Purified by | | |
| | prep HPLC | | |
| | Step 4: GM2A final purification a (SCX-2) and | | |
| | prep) | | |
| M05747 | 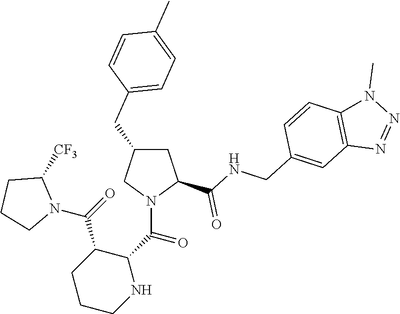 [SMILES: Cc1ccc(C[C@@H]2C[C@@H](C(=O)NCc3ccc4c(c3)nnn4C)N(C(=O)[C@@H]3NCCC[C@@H]3C(=O)N3CCC[C@@H]3C(F)(F)F)C2)cc1] | AnalpH2_MeOH_QC_v1, Rt: 6.39 min, m/z 639.6 [M + H]⁺ AnalpH9_MeOH_QC_v1, Rt: 8.45 min, m/z 639.6 [M + H]⁺ ¹H-NMR (400 MHz, DMSO-D₆) δ 8.59 (t, J = 6.0 Hz, 0.6H), 8.02 (t, J = 4.6 Hz, 0.3H), 7.96 (d, J = 4.6 Hz, 1H), 7.58-7.51 (m, 2H), 7.30-7.26 (dd, J = 8.7, 1.4 Hz, 0.6H), 7.24- 7.20 (dd, J = 8.7, 1.4 Hz, 0.4 H), 7.12-7.03 (m, 4H), 5.25 (t, J = 5.3 Hz, 0.6H), 4.73 (t, J = 7.8 Hz, 1H), 4.45-4.22 (m, 2H), 4.09 (s, 0.2H), | 14.7 mg, 78%, white solid |
| | (2S,4R)-N-((1-methyl-1H-indazol-5-yl)methyl)- | 4.01 (s, 3H), 3.64-3.39 | |
| | 4-(4-methylbenzyl)-1-((2R,3S)-3-((R)-2- | (m, 3H), 3.19-3.14 (m, | |
| | (trifluoromethyl)pyrrolidine-1- | 0.3H), 3.02-2.91 (m, | |
| | carbonyl)piperidine-2-carbonyl)pyrrolidine-2- | 2H), 2.81 (s, 0.3H), | |
| | carboxamide | 2.68-2.54 (m, 2H), | |
| | S1: (1-methyl-1H-indazol-5-yl)methanamine | 2.44-2.07 (m, 5H), | |
| | S2: (2S,4R)-1-(tert-butoxycarbonyl)-4-(4- | 1.95-1.89 (m, 5H), | |
| | methylbenzyl)pyrrolidine-2-carboxylic acid | 1.81-1.08 (m, 4H) | |
| | S4: (2R,3S)-1-(tert-butoxycarbonyl)-3-((R)-2- | | |
| | (trifluoromethyl)pyrroldine-1- | | |
| | carbonyl)piperidine-2-carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. | | |
| | Purification by filtration through silica plug | | |
| | Step 4: GM2A final purification a (SCX) | | |
| M05748 | 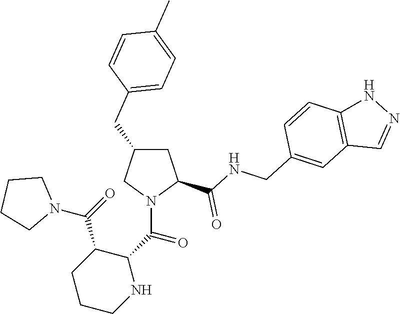 [SMILES: Cc1ccc(C[C@@H]2C[C@@H](C(=O)NCc3ccc4[nH]ncc4c3)N(C(=O)[C@@H]3NCCC[C@@H]3C(=O)N3CCCC3)C2)cc1] | AnalpH2_MeOH_QC_v1, Rt: 5.80 min, m/z 557.5 [M + H]⁺ AnalpH9_MeOH_QC_v1, Rt: 7.62 min, m/z 557.5 [M + H]⁺ ¹H-NMR (400 MHz, DMSO-D₆) δ 8.56 (t, J = 6.0 Hz, 0.5H), 8.05 (t, J = 5.7 Hz, 0.4H), 7.98 (s, 1H), 7.54 (d, J = 16.5 Hz, 1H), 7.44 (dd, J = 12.4, 8.7 Hz, 1H), 7.16-7.23 (m, 1H), 7.03-7.09 (m, 4H), 5.18-5.20 (m, 1H), 4.24-4.40 (m, 2H), 3.97-4.03 (m, 0.3H), 3.33-3.53 (m, 3H), 3.10-3.23 (m, 2H), | 26.5 mg, 83%, white solid |
| | (2S,4R)-N-((1H-indazol-5-yl)methyl)-4-(4- | 2.82-3.05 (m, 2H), | |
| | methylbenzyl)-1-((2R,3S)-3-(pyrrolidine-1- | 2.72-2.76 (m, 0.4H), | |
| | carbonyl)piperidine-2-carbonyl)pyrrolidine-2- | 2.53-2.66 (m, 2H), | |
| | carboxamide | 2.29-2.33 (m, 0.4H), | |
| | S1: (1H--indazol-5-yl)methanamine | 2.25 (s, 3H), 1.65-1.98 | |
| | S2: (2S,4F)-1-(tert-butoxycarbonyl)-4-(4- | (m, 8H), 1.53-1.56 (m, | |
| | methylbenzyl)pyrrolidine-2-carboxoylic acid | 0.3H), 1.29-1.36 (m, | |
| | S4: (2R,3S)-1-(tert-botoxycarbonyl)-3- | 0.6H), 1.06-1.15 (m, | |
| | (pyrrolidine-1-carbonyl)pperidine-2-carboxylic | 0.6H) | |
| | acid | | |
| | Step 1: GM1 with DMF and DIPEA. Purified by | | |
| | column chromatography | | |
| | Step 2: GM2A. Purification a (SCX-2). | | |
| | Step 3: GM1 with DMF and DIPEA. | | |
| | Purified by prep HPLC | | |
| | Step 4: GM2A final purification a (SCX-2) | | |
| M05751 | 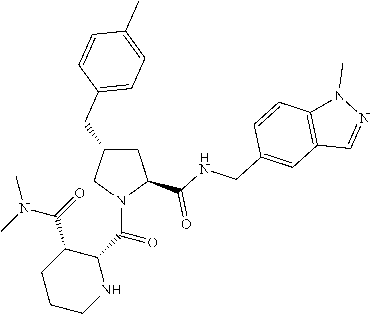 [SMILES: Cc1ccc(C[C@@H]2C[C@@H](C(=O)NCc3ccc4c(cnn4C)c3)N(C(=O)[C@@H]3NCCC[C@@H]3C(=O)N(C)C)C2)cc1] | AnalpH2_MeOH_QC_v1, Rt: 5.92 min, m/z 545.5 [M + H]⁺ AnalpH9_MeOH_QC_v1, Rt: 7.82 min, m/z 545.5 [M + H]⁺ ¹H-NMR (400 MHz, DMSO-D₆) δ 8.56 (t, J = 5.95 Hz, 0.5H), 8.05 (t, J = 5.95 Hz, 0.3H), 7.96-7.91 (m, 1H), 7.54 (dt, J = 16.8, 4.4 Hz, 2H), 7.30-7.26 (dd, J = 8.7, 1.4 Hz, 0.5H), 7.25-7.21 (dd, J = 8.7, 1.4 Hz, 0.3H), 7.10- 7.04 (m, 4H), 5.24 (s, 0.5H), 4.42-4.25 (m, 2H), 4.03-3.97 (m, 3H), 3.57-3.43 (m, 1H), | 13.6 mg, 67%, White solid |
| | (2R,3S)-N,N-dimethyl-2-((2S,4R)-2-(((1- | 3.26-2.54 (m, 15H), | |
| | methyl-1H-indazol-5-yl)methyl)carbamoyl)-4- | 2.35-2.26 (m, 3H), | |
| | (4-methylbenzyl)pyrrlidine-1- | 2.19-1.08 (m, 4H) | |
| | carbonyl)piperidine-3-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3- | | |
| | (dimethylcarbamoyl)piperidine-2-carboxylic | | |
| | acid | | |
| | Step 1: GM1 with DMF and DIPEA. Purified by | | |
| | column chromatography. | | |
| | Step 2: GM2A. Purification a (SCX-2) | | |
| | Step 3: GM1 with DMF and DIPEA. Purified by | | |
| | prep HPLC | | |
| | Step 4: GM2A final purification a (SCX) | | |
| M05752 | 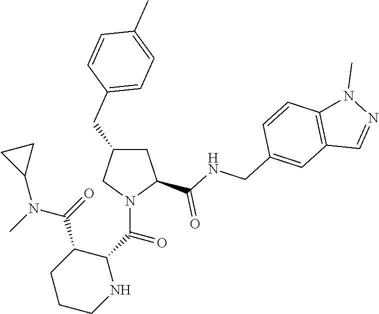 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CC3)C2)cc1] | AnalpH2_MeOH_QC_v1, Rt: 6.09 min, m/z 571.6 [M + H]⁺ AnalpH9_MeOH_QC_v1, Rt: 8.13 min, m/z 571.5 [M + H]⁺ ¹H-NMR (400 MHz, DMSO-D₆) δ 8.62-8.55 (t, J = 6.0 Hz, 0.6H), 8.10 (t, J = 6.0 Hz, 0.4H), 7.98-7.91 (m, 1H), 7.59-7.47 (m, 2H), 7.31-7.27 (dd, J = 8.7, 1.4 Hz, 0.5H), 7.24- 7.20 (dd, J = 8.7, 1.8 Hz, 0.4H) 7.11-7.04 (m, 4H), 5.26 (t, J = 5.0 Hz, 0.6H), 4.43-4.24 (m, 2H), 4.06-4.01 (m, 3H), 3.53-3.45 (m, 1H), | 33.3 mg, 82%, White solid |
| | (2R,3S)-N-cyclopropyl-N-methyl-2-((2S,4R)-2- | 3.13-2.71 (m, 7H), | |
| | (((1-methyl-1H-indazol-5-yl)methyl)carbamoyl)- | 2.67-2.54 (m, 3H), | |
| | 4-(4-methylbenyl)pyrrolidine-1- | 2.41-2.10 (m, 5H), | |
| | carbonyl)piperidine-3-carboxamide | 1.92-1.53 (m, 4H), | |
| | S1: (1-methyl-1H-indazol-5-yl)methnamine | 1.31-1.23 (m, 1H), | |
| | S2: (2S,4R)-1-tert butoxycabonyl)-4-(4- | 1.14-1.05 (m, 1H), | |
| | methylbenzyl)pyrrolidine-2-carboxylic acid | 0.77-0.63 (m, 3H) | |
| | S4: (2R,3S)-tert-butoxycarbonyl)-3- | | |
| | (cyclopropyl(methyl)carbamoyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DMF and DIPEA. Purified by | | |
| | prep HPLC | | |
| | Step 4: GM2A final purification a (SCX-2 | | |
| | followed by prep HPLC) | | |
| M05754 | 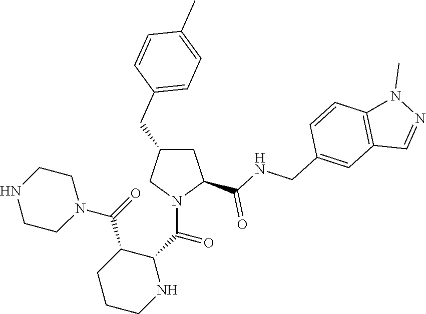 [SMILES: Cc1ccc(C[C@@H]2C[C@@H](C(=O)NCc3ccc4c(cnn4C)c3)N(C(=O)[C@@H]3NCCC[C@@H]3C(=O)N3CCNCC3)C2)cc1] | AnalpH2_MeOH_QC_v1, Rt: 4.57 min, m/z 586.6 [M + H]⁺ AnalpH9_MeOH_QC_v2, Rt: 7.68 mins, m/z 586.6 [M + H]⁺ | 19.7 mg, 64%, White solid |
| | (2S,4R)-N-((1-methyl-1H-indazol-5-yl)methyl)- | | |
| | 4-(4-methylbenzyl)-1-((2R,3S)-3-(piperazine-1- | | |
| | carbonyl)piperidine-2-carbonyl)pyrrolidine-2- | | |
| | carboxamide | | |
| | S1: (1-methyl-1H-indazol-5-yl)methnamine | | |
| | S2: (2S,4R)-1-tert butoxycabonyl)-4-(4- | | |
| | methylbenzyl)pyrrolidine-2-carboxylic acid | | |
| | S4: (2R,3S)-1-tert-butoxycarbonyl)-3- | | |
| | (piperazine-1-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DMF and DIPEA. Purified by | | |
| | prep HPLC | | |
| | Step 4: GM2A final purification a (SCX | | |
| | followed by prep HPLC) | | |
| M05764 | 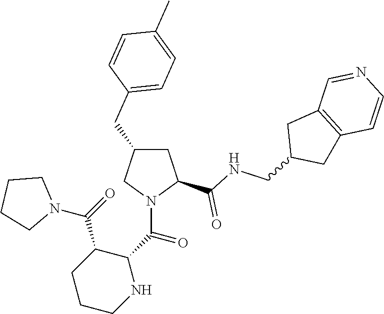 [SMILES: Cc1ccc(C[C@@H]2C[C@@H](C(=O)NCC3Cc4ccncc4C3)N(C(=O)[C@@H]3NCCC[C@@H]3C(=O)N3CCCC3)C2)cc1] | AnalpH2_MeOH_QC_v1, Rt: 4.27 min, m/z 558.5 [M + H]⁺ AnalpH9_MeOH_QC_v1, Rt: 8.05 min, m/z 558.5 [M + H]⁺ ¹H-NMR (400 MHz, ACETONITRILE-D₃) δ 8.38-8.34 (m, 1H), 8.29-8.27 (m, 1H), 7.26-6.97 (m, 5.5H), 6.93-6.82 (m, 0.5H), 5.18 (d, J = 7.3 Hz, 0.3H), 4.34 (dd, J = 8.7, 2.7 Hz, 0.7H), 4.08- 3.99 (m, 1H), 3.50-2.94 (m, 12H), 2.87-2.83 (m, 1H), 2.73-2.52 (m, 6H), 2.50-2.38 (m, 1H), 2.30 (s, 3H), 1.91-1.85 (m, | 12.0 mg, 47%, White solid |
| | (2S,4R)-N-((6,7-dihydro-5H- | 2H), 1.83-1.61 (m, 4H), | |
| | cyclopenta[c]pyridin-6-yl)methyl)-4-(4- | 1.41-1.28 (m, 1H) | |
| | methylbenzyl)-1-((2R,3S)-3-(pyrrolidine-1- | | |
| | carbonyl)piperidine-2-carbonyl)pyrrolidine-2- | | |
| | carboxamide | | |
| | S1: (6,7-dihydro-5H-cyclopenta[c]pyridin-6- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3- | | |
| | pyrrolidine-1-carbonyl)piperidine-2-carboxylic | | |
| | acid | | |
| | Step 1: GM1 with DMF and DIPEA. Crude | | |
| | used without further purification | | |
| | Step 2: GM2A. Purification a SCX-2 | | |
| | Step 3: GM1 with DMF and DIPEA. Purified by | | |
| | prep HPLC | | |
| | Step 4: GM2A final purification e (crude by | | |
| | prep HPLC). | | |
| | Obtained as a mixture of diastereoisomers | | |
| M05773 | 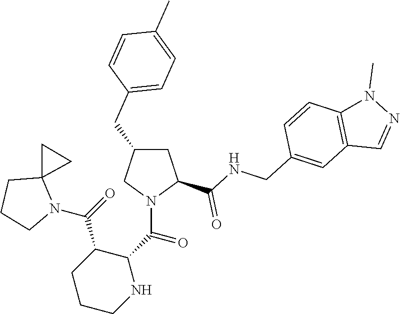 [SMILES: Cc1ccc(C[C@@H]2C[C@@H](C(=O)NCc3ccc4c(cnn4C)c3)N(C(=O)[C@@H]3NCCC[C@@H]3C(=O)N3CCCC34CC4)C2)cc1] | AnalpH2_MeCN_QC_V2, Rt: 5.06 min, m/z 597.4 [M + H]⁺ AnalpH9_MeCN_QC_V2, Rt: 7.17 min, m/z 597.4 [M + H]⁺ ¹H-NMR (400 MHz, DMSO-D₆) δ 8.55 (t, J = 5.7 Hz, 0.6H), 8.11 (t, J = 5.1 Hz, 0.4H), 7.95 (m, 1H), 7.58-7.51 (m, 2H), 7.29-7.22 (m, 1H), 7.11-7.04 (m, 4H), 5.24 (s, 0.6H), 4.42-4.17 (m, 2H), 4.05-3.95 (m, 3H), 3.58-3.38 (m, 3H), 3.17-2.86 (m, 3H), 2.67-2.54 (m, 2H), 2.46-2.42 (m, 1H), 2.35-2.26 (m, 4H), | 32.0 mg, 53%, White solid |
| | (2S,4R)-1-((2R,3S)-3-(4-azaspiro[2.4]heptane- | 2.14-2.07 (m, 1H), | |
| | 4-carbonyl)piperidine-2-carbonyl-N-((1- | 1.96-1.67 (m, 10H), | |
| | methyl-1H-indazol-5-yl)methyl)-4-(4- | 1.47 (m, 1H), 1.32-1.23 | |
| | methylbenzyl)pyrrolidine-2-carboxamide | (m, 1H), 1.15-1.06 (m, | |
| | S1: (1-methyl-1H-indazol-5-yl)methanamine | 1H), 0.35 (s, 2H) | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3-(4- | | |
| | azaspiro[2.4]heptane-4-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by | | |
| | prep HPLC | | |
| | Step 4: GM2A final purification a (SCX | | |
| | followed by prep HPLC | | |
| M05843 | 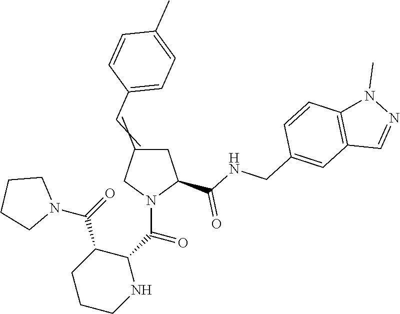 | UPLC_pH2_MeCN_QC_ V1, Rt: 3.24 min, m/z 569.3 [M + H]⁺ UPLC_pH10_MeCN_QC_ V1, Rt: 4.30 min, m/z 569.3 [M + H]⁺ 1H-NMR (400 MHz, ACETONITRILE-D₃) δ 7.79 (d, J = 7.3 Hz, 1H), 7.48 (d, J = 6.9 Hz, 1H), 7.32 (d, J = 8.2 Hz, 2H), 7.23-7.07 (m, 6H), 6.43 (s, 1H), 5.56-5.31 (0.8H), 4.76-4.64 (m, 1.2H), 4.51- 4.22 (m, 3H), 4.19-4.06 (m, 0.5H), 3.96 (s, 3H), 3.62 (d, J = 3.2 Hz, 1H), 3.46-2.91 (m, 9H), 2.73- 2.56 (m, 1.4H), 2.35 (d, J = 14.2 Hz, 4H), 1.89-1.60 | 2.5 mg, 5%, white solid |
| | (S)-N-((1-methyl-1H-indazol-5-yl)methyl)-4-(4- | (m, 7H), 1.56-1.16 (3H) | |
| | methylbenzylidene)-1-((2R,3S)-3-(pyrrolidine- | | |
| | 1-carbonyl)piperidine-2-carbonyl)pyrrolidine-2- | | |
| | carboxamide (alkene stereochemistry | | |
| | configuration A, unassigned) | | |
| | S1: (1-methyl-1H-indazol-5-yl)methanamine | | |
| | S2: (S)-1-(tert-butoxycarbonyl)-4-(4- | | |
| | methylbenzylidene)pyrrolidine-2-carboxylic | | |
| | acid (as mixture of E and Z stereisomers) | | |
| | S4: (2R,3S)-1-(tert-butoxycarbonyl)-3- | | |
| | (pyrrolidine-1-carbonyl)piperidine-2-carboxylic | | |
| | acid | | |
| | Step 1: GM1 with DMF and DIPEA. Purified by | | |
| | prep HPLC | | |
| | Step 2: GM2A. Purification b. | | |
| | Step 3: GM1 with DMF and DIPEA. Purified by | | |
| | prep HPLC and | | |
| | Step 4: GM2A. Purification c followed by | | |
| | preparative, chiral chromatography | | |
| M05844 | 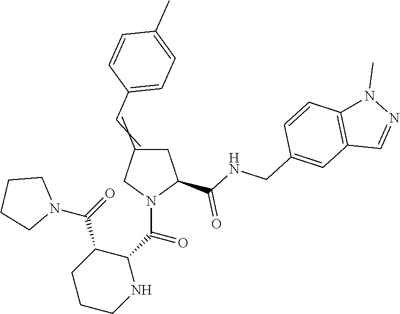 | | |
| | (S)-N-((1-methyl-1H-indazol-5-yl)methyl)-4-(4- | | |
| | methylbenzylidene)-1-((2R,3S)-3-(pyrrolidine- | | |
| | 1-carbonyl)piperidine-2-carbonyl)pyrrolidine-2- | | |
| | carboxamide (alkene stereochemistry | | |
| | configuration B, unassigned) | | |
| | S1: (1-methyl-1H-indazol-5-yl)methanamine | | |
| | S2: (S)-1-(tert-butoxycarbonyl)-4-(4- | | |
| | methylbenzylidene)pyrrolidine-2-carboxylic | | |
| | acid(as mixture of E and Z alkene | | |
| | stereoisomers) | | |
| | S4: (2R,3S)-1-(tert-butoxycarbonyl)-3- | | |
| | (pyrrolidine-1-carbonyl)piperidine-2-carboxylic | | |
| | acid | | |
| | Step 1: GM1 with DMF and DIPEA. Purified by | | |
| | prep HPLC | | |
| | Step 2: GM2A. Purification b. | | |
| | Step 3: GM1 with DMF and DIPEA. Purified by | | |
| | prep HPLC | | |
| | Step 4: GM2A. Purification c followed by | | |
| | preparative, chiral chromatography | | |
| M05851 | 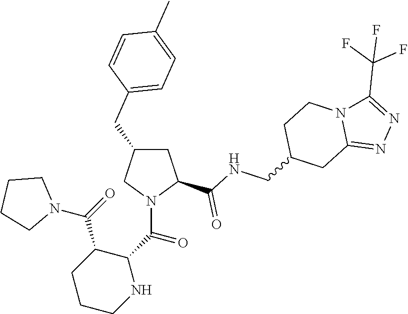 [SMILES: Cc1ccc(C[C@@H]2C[C@@H](C(=O)NCC3CCn4c(nnc4C(F)(F)F)C3)N(C(=O)[C@@H]3NCCC[C@@H]3C(=O)N3CCCC3)C2)cc1] | UPLC_pH2_MeCN_QC_ V1, Rt: 3.20 min, m/z 630.3 [M + H]⁺ UPLC_pH10_MeCN_QC V1, Rt: 4.29 min, m/z 630.3 [M + H]⁺ | 20.8 mg, 80%, white solid |
| | (2S,4R)-4-(4-methylbenzyl)-1-((2R,3S)-3- | | |
| | (pyrrolidine-1-carbonyl)piperidine-2-carbonyl)- | | |
| | N-((3-(trifluoromethyl)-5,6,7,8-tetrahydro- [1,2,4]triazolo[4,3-1]pyridin-7- | | |
| | yl)methyl)pyrrolidine-2-carboxamide | | |
| | S1: (3-(trifluoromethyl)-5,6,7,8-tetrahydro- | | |
| | [1,2,4]triazolo[4,3-a]pyridin-7-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3- | | |
| | pyrrolidine-1-carbonyl)piperidine-2-carboxylic | | |
| | acid | | |
| | Step 1: GM1 with DMF and DIPEA. Purified by | | |
| | prep HPLC. | | |
| | Step 2: GM2A. Purification a (SCX-2). | | |
| | Step 3: GM1 with DMF and DIPEA. Purified by | | |
| | prep HPLC. | | |
| | Step 4: GM2A final purification a (SCX-2) | | |
| M05854 | 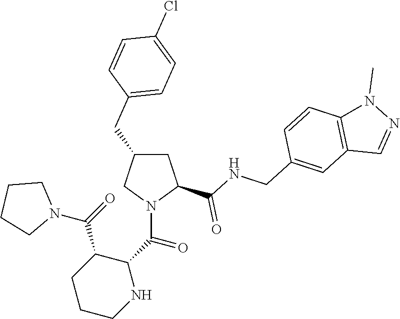 [SMILES: Cn1ncc2cc(CNC(=O)[C@@H]3C[C@@H](Cc4ccc(Cl)cc4)CN3C(=O)[C@@H]3NCCC[C@@H]3C(=O)N3CCCC3)ccc21] | UPLC_pH2_MeCN_QC_ V1, Rt: 3.52 min, m/z 591.3 [M + H]⁺ UPLC_pH10_MeCN_QC_ V1, Rt: 4.68 mins, m/z 591.2 [M + H]⁺ ¹H-NMR (400 MHz, DMSO-D₆) δ 8.60 (0.6H, t, J = 6.0 Hz), 8.14 (0.3H, t, J = 5.7 Hz), 7.97 (1H, s), 7.51-7.58 (2H, m), 7.16- 7.35 (5H, m), 5.21-5.23 (0.5H, m), 4.25-4.42 (2.4H, m), 4.01 (3.4H, s), 3.64-3.52 (0.4H, m), 3.37- 3.49 (1.3H, m), 2.90-3.26 (5.4H, m), 2.59-2.79 (3.9H, m), 2.18-2.37 (2.3H, m), 1.66-1.98 (7.7H, m), 1.54-1.57 | 22.0 mg, 83%, white solid |
| | (2S,4R)-4-(4-chlorobenzyl)-N-((1-methyl-1H- | (0.8H, m), 1.29-1.37 | |
| | indazol-5-yl)methyl)-1-((2R,3S)-3-(pyrrolidine- | (0.9H, m), 1.10 (0.5H, d, | |
| | 1-carbonyl)piperidine-2-carbonyl)pyrrolidine-2- | J = 12.8 Hz) | |
| | carboxamide | | |
| | S1: (1-methyl-1H-indazol-5-yl)methanamine | | |
| | S2: (2S,4R)-1-(tert-butoxycarbonyl)-4-(4- | | |
| | chlorobenzyl)pyrrolidine-2-carboxylic acid | | |
| | S4: (2R,3S)-1-(tert-butoxycarbonyl)-3- | | |
| | pyrrolidine-1-carbonyl)piperidine-2-carboxylic | | |
| | acid | | |
| | Step 1: GM1 with DMF and DIPEA. Purified by | | |
| | column chromatography | | |
| | Step 2: GM2A. Purification a (SCX-2). | | |
| | Step 3: GM1 with DMF and DIPEA. Purified by | | |
| | prep HPLC. | | |
| | Step 4: GM2A final purification a (SCX-2) | | |
| M05855 | 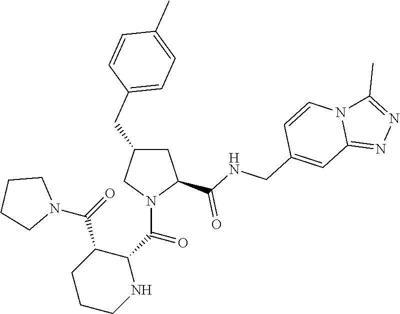 [SMILES: Cc1ccc(C[C@@H]2C[C@@H](C(=O)NCc3ccn4c(C)nnc4c3)N(C(=O)[C@@H]3NCCC[C@@H]3C(=O)N3CCCC3)C2)cc1] | UPLC_pH2_MeCN_QC_ V1, Rt: 3.08 min, m/z 572.3 [M + H]⁺ UPLC_pH10_MeCN_QC_ V1, Rt: 4.13 min, m/z 572.3 [M + H]⁺ 1H-NMR (400 MHz, DMSO-D₆) δ 8.69 (t, J = 6.0 Hz, 0.5H), 8.30 (d, J = 6.9 Hz, 0.4H), 8.21-8.25 (m, 1H), 7.41 (d, J = 6.4 Hz, 1H), 7.04-7.11 (m, 4H), 6.77-6.82 (m, 1H), 5.29 (s, 0.4H), 4.20-4.36 (m, 2H), 3.99 (dd, J = 9.6, 6.9 Hz, 0.4H), 3.56 (s, 0.3H), 3.35-3.47 (m, 2H), 3.14-3.21 (m, 2H), 2.96- 3.07 (m, 2H), 2.79 (s, 0.4H), 2.55-2.64 (m, 4H), | 17.3 mg, 76%, white solid |
| | (2S,4R)-N-((3-methyl-[1,2,4]triazolo[4,3- | 2.17-2.36 (m, 4H), 1.47- | |
| | a]pyridin-7-yl)methyl)-4-(4-methylbenzyl)-1- | 1.95 (m, 9H), 1.13-1.29 | |
| | ((2R,3S)-3-(pyrrolidine-1-carbonyl)piperidine-2 | (m, 1H). | |
| | carbonyl)pyrrolidine-2-carboxamide | | |
| | S1: (3-methyl-[1,2,4]trizolo[4,3-a]pyridin-7- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3- | | |
| | pyrrolidine-1-carbonyl)piperidine-2-carboxylic | | |
| | acid | | |
| | Step 1: GM1 with DMF and DIPEA. Purified by | | |
| | column chromatography. | | |
| | Step 2: GM2A. Purification a (SCX-2). | | |
| | Step 3: GM1 with DMF and DIPEA. Purified by | | |
| | prep HPLC. | | |
| | Step 4: GM2A final purification a (SCX-2) | | |
| M05899 | 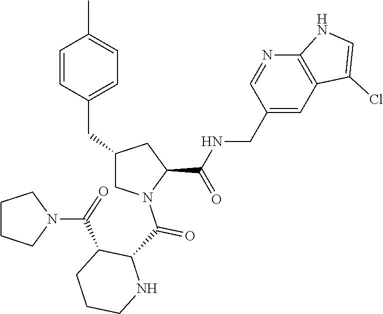 [SMILES: Cc1ccc(C[C@@H]2C[C@@H](C(=O)NCc3cnc4[nH]cc(Cl)c4c3)N(C(=O)[C@@H]3NCCC[C@@H]3C(=O)N3CCCC3)C2)cc1] | 1H-NMR (400 MHz, DMSO-d6) δ 11.99-11.85 (m, 1H), 8.64 (t, J = 5.5 Hz, 0.7H), 8.29-8.10 (m, 1.3H), 7.83-7.74 (m, 1H), 7.70-7.61 (m, 1H), 7.12- 7.03 (m, 4H), 5.20 (br s, 0.6H), 4.51-4.26 (m, 2.6H), 3.98 (t, J = 8.0 Hz, 0.3H), 3.65-3.44 (m, 0.8H), 3.28-3.10 (m, 2.5H), 3.09-2.74 (m, 2.3H), 2.70-2.55 (m, 2H), 2.44-2.31 (m, 1H), 2.26 (s, 3H), 2.21-2.06 (m, 0.8H), 2.05-1.39 (m, 9H), 1.38-1.13 (m, 1.2H), 1.10- 0.94 (m, 0.6H) 27 Hₐₗᵢₚₕ seen, 1H underneath the water | |
| | (2S,4R)-N-((3-chloro-1H-pyrrolo[2,3-b]pyridin- | peak and DMSO peak | |
| | 5-yl)methyl)-4-(4-methylbenzyl)-1-((2R,3S)-3- | | |
| | (pyrrolidine-1-carbonyl)piperidine-2- | | |
| | carbonyl)pyrrolidine-2-carboxamide | | |
| | S1: (3-chloro-1H-pyrrolo[2,3-b]pyridin-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3- | | |
| | pyrrolidine-1-carbonyl)piperidine-2-carboxylic | | |
| | acid | | |
| | Step 1: GM1 with DMF and DIPEA. Purified by | | |
| | column chromatography. | | |
| | Step 2: GM2A Purification a (SCX-2). | | |
| | Step 3: GM1 with DMF and DIPEA. Purified by | | |
| | prep HPLC. | | |
| | Step 4: GM2A final purification a (SCX-2) | | |
| M05913 | 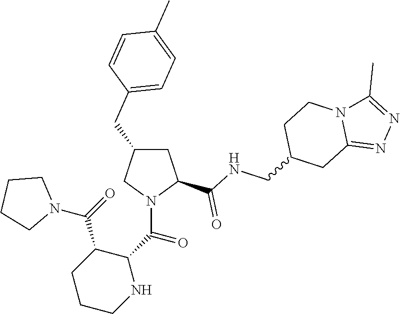 [SMILES: Cc1ccc(C[C@@H]2C[C@@H](C(=O)NCC3CCn4c(C)nnc4C3)N(C(=O)[C@@H]3NCCC[C@@H]3C(=O)N3CCCC3)C2)cc1] | UPLC_pH2_MeCN_QC_ V1, Rt: 2.76 min, m/z 576.3 [M + H]⁺ UPLC_pH9_MeCN_QC_ V1, Rt: 4.04 min, m/z 576.4 [M + H]⁺ ¹H-NMR (400 MHz, DMSO-D₆) δ 8.19 (d, J = 4.6 Hz, 0.3H), 7.89 (s, 0.5H), 7.00-7.08 (m, 4H), 5.06 (s, 0.1H), 4.20 (t, J = 5.5 Hz, 1H), 3.85- 3.95 (m, 2H), 3.57-3.67 (m, 1H), 3.34-3.45 (m, 1H), 3.18-3.21 (m, 2H), 2.95-3.13 (m, 2H), 2.83- 2.87 (m, 1H), 2.52-2.67_ (m, 2H), 2.50 (s, 1H), 2.27-2.38 (m, 1H), 2.22 (d, J = 4.1 Hz, 6H), 1.66- | 31.2 mg, 75%, white solid |
| | (2S,4R)-N-[(3-mthyl-5,6,7,8-tetrahydro- | 1.89 (m, 11H), 1.45-1.57 | |
| | [1,2,4]triazolo[4,3-a]pyidin-7-yl)methyl]-4-(p- | (m, 1H), 1.19- | |
| | tolylmethyl)-1-[(2R,3S)-3-(pyrrolidine-1- | 1.31 (m, 0.2H) | |
| | carbonyl)piperidine-2-carbonyl]pyrrolidine-2- | | |
| | carboxamide | | |
| | S1: (3-methyl-5,6,7,8-tetrahydro- | | |
| | [1,2,4]triazolo[4,3-a]pyridin-7-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-(tert-butoxycarbonyl)-3- | | |
| | pyrrolidine-1-carbonyl)piperidine-2-carboxylic | | |
| | acid | | |
| | Step 1: GM1 with DCM and DIPEA. Purified by | | |
| | column chromatography. | | |
| | Step 2: GM2A. Purification a (SCX-2) | | |
| | Step 3: GM1 with DMF and DIPEA. | | |
| | Purification by preparative HPLC | | |
| | Step 4: GM2A final purification a (SCX) | | |
| M05983 | 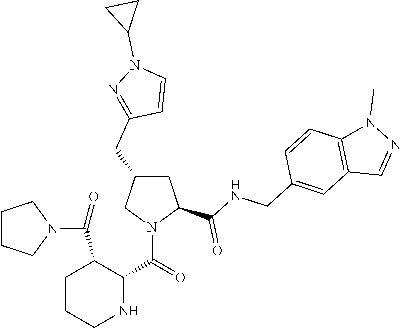 [SMILES: Cn1ncc2cc(CNC(=O)[C@@H]3C[C@@H](Cc4ccn(C5CC5)n4)CN3C(=O)[C@@H]3NCCC[C@@H]3C(=O)N3CCCC3)ccc21] | UPLC_pH9_MeCN_QC_ V1 Rt: 3.93 min, m/z 587.4 [M + H]+ UPLC_pH2_MeCN_QC_ V1Rt: 2.93 min, m/z 587.4 [M + H]+ 1H-NMR (400 MHz, DMSO-D6) δ 8.61 (t, J = 6.0 Hz, 0.5H), 8.12 (t, J = 6.0 Hz, 0.5H), 7.97 (d, J = 2.7 Hz, 1H), 7.63-7.53 (m, 3H), 7.32-7.24 (m, 1H), 5.99 (dd, J = 13.3, 2.3 Hz, 1H), 5.18 (d, J = 6.9 Hz, 0.5H), 4.44-4.25 (m, 2H), 4.06-3.97 (m, 3.5H), 3.64-3.52 (m, 2H), 3.42-3.36 (m, 1H) 3.27-3.06 (m, 3H), 3.00-2.75 (m, 2H), | 15.1 mg, 93%, White solid |
| | (2S,4S)-4-((1-cyclopropyl-1H-pyrazol-3- | 2.57-2.52 (m, 2H), | |
| | yl)methyl)-N-((1-methyl-1H-indazol-5- | 2.43-2.19 (m, 1H), | |
| | yl)methyl)-1-((2R,3S)-3-(pyrrolidine-1- | 2.07-1.68 (m, 8H), 1.55 | |
| | carbonyl)piperidine-2-carbonyl)pyrrolidine- | (q, J = 12.4 Hz, 0.5H), | |
| | 2-carboxamide | 1.34-1.24 (m, 1H), | |
| | S1: 5-(Aminomethyl)-1-methyl-1H-indazole | 1.15-1.03 (m, 0.5H), | |
| | S2: (2S,4S)-1-(tert-butoxycarbonyl)-4-((1- | 1.00-0.87 (m, 4H) | |
| | cyclopropyl-1H-pyrazol-3- | | |
| | yl)methyl)pyrrolidine-2-carboxylic acid | | |
| | S4: (2R,3S)-1-(tert-butoxycarbonyl)-3- | | |
| | pyrrolidine-1-carbonyl)piperidine-2- | | |
| | carboxylic acid | | |
| | Step 1: GM1 with DCM and DIPEA. Amine | | |
| | used as HCl salt. | | |
| | Step 2: GM2A. No purifiction | | |
| | Step 3: GM1 with DMF and DIPEA. | | |
| | Purification by prep HPLC | | |
| | Step 4: GM2A. Final purification a | | |
| M05985 | 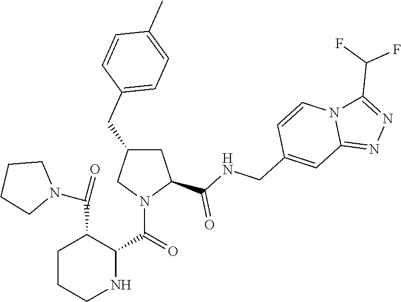 [SMILES: Cc1ccc(C[C@@H]2C[C@@H](C(=O)NCc3ccn4c(C(F)F)nnc4c3)N(C(=O)[C@@H]3NCCC[C@@H]3C(=O)N3CCCC3)C2)cc1] | UPLC_pH2_MeCN_QC_ V1Rt: 3.32 min, m/z 608.3 [M + H]+ UPLC_pH9_MeCN_QC_ V1 Rt: 4.52 min, m/z 608.3 [M + H]+ 1H-NMR (400 MHz, DMSO-D6) δ 8.73 (t, J = 6.2 Hz, 0.5H), 8.56 (dd, J = 26.6, 7.3 Hz, 1H), 8.28 (s, 0.5H), 7.87-7.60 (m, 2H), 7.12-7.01 (m, 5H), 5.30 (s, 0.5H), 4.43-4.28 (m, 2H), 4.03 (dd, J = 10.1, 6.0 Hz, 0.5H), 3.56- 3.35 (m, 2H), 3.25-3.14 (m, 2H), 3.09-2.97 (m, 2H), 2.79-2.54 (m, 4H), | 33.78 mg, 90%, Off- White Solid |
| | (2S,4R)-N-[[3-(difluoromethyl)- | 2.33-2.22 (m, 4H), | |
| | [1,2,4]triazolo[4,3-a]pyridin-7-yl]methyl]-4- | 1.99-1.66 (m, 7H), | |
| | (p-tolylmethyl)-1-[(2R,3S)-3-(pyrrolidine-1- | 1.59-1.11 (m, 2H) | |
| | carbonyl)piperidine-2-carbonyl]pyrrolidine- | | |
| | 2-carboxamide | | |
| | S1: [3-(difluoromethyl)-[1,2,4]triazolo[4,3- | | |
| | a]pyridin-7-yl]methanamine | | |
| | S2: Boc-(R)-y-(4-methylbenzyl)-L-proline | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DCM and DIPEA. | | |
| | Step 2: GM2A. | | |
| | Step 3: GM1 with DMF and DIPEA. | | |
| | Purification by prep HPLC | | |
| | Step 4: GM2A. Final purification a | | |
| M05997 | 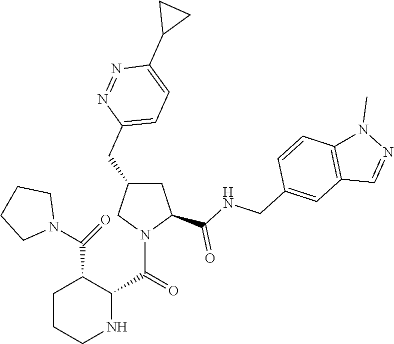 [SMILES: Cn1ncc2cc(CNC(=O)[C@@H]3C[C@@H](Cc4ccc(C5CC5)nn4)CN3C(=O)[C@@H]3NCCC[C@@H]3C(=O)N3CCCC3)ccc21] | UPLC_pH9_MeCN_QC_ V1 Rt: 3.73 min, m/z 599.4 [M + H]+ UPLC_pH2_MeCN_QC_ V1 Rt: 2.79 min, m/z 599.4 [M + H]+ 1H-NMR (400 MHz, DMSO-D6) δ 8.59 (t, J = 6.0 Hz, 0.5H), 8.10 (d, J = 5.5 Hz, 0.5H), 7.99 (d, J = 3.7 Hz, 1H), 7.64-7.53 (m, 2H), 7.38-7.28 (m, 3H), 5.04 (t, J = 7.8 Hz, 0.5H), 4.48-4.19 (m, 2.5H), 4.00 (d, J = 10.1 Hz, 3H), 3.84- 3.54 (m, 2H), 3.46-3.36 (m, 2H), 3.26-3.10 (m, 2H), 3.02-2.89 (m, 3H), 2.85-2.57 (m, 3H), 2.45- 2.32 (m, 1H), 2.24-2.15 (m, 1H), 2.07-1.91 (m, 1H), 1.86-1.47 (m, 7H), | 9 mg, 64%, Light Yellow Solid |
| | (2S,4S)-4-[(6-cyclopropylpyridazin-3- | 1.35-1.24 (m, 1H), 1.09- | |
| | yl)methyl]-N-[(1-methylindazol-5- | 0.99 (m, 5H) | |
| | yl)methyl]-1-[(2R,3S)-3-(pyrrolidine-1- | | |
| | carbonyl)piperidine-2-carbonyl]pyrrolidine- | | |
| | 2-carboxamide | | |
| | S1: 5-(Aminomethyl)-1-methyl-1H-indazole | | |
| | S2: (2S,4S)-1-tert-butoxycarbonyl-4-[(6- | | |
| | cyclopropylpyridazin-3- | | |
| | yl)methyl+pyrrolidine-2-carboxylic acid | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. | | |
| | Step 2: GM2A. | | |
| | Step 3: GM1 with DMF and DIPEA. | | |
| | Purification by prep HPLC | | |
| | Step 4: GM2A. Final purification a | | |
| M06009 | 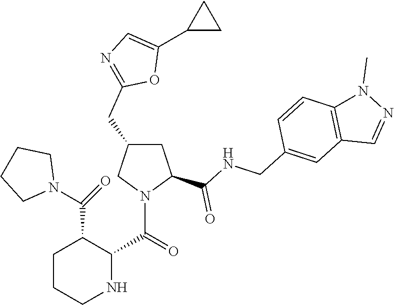 [SMILES: Cn1ncc2cc(CNC(=O)[C@@H]3C[C@@H](Cc4ncc(C5CC5)o4)CN3C(=O)[C@@H]3NCCC[C@@H]3C(=O)N3CCCC3)ccc21] | UPLC_pH2_MeCN_QC_ V1 Rt: 3.06 min, m/z 588.4 [M + H]+ 1H-NMR (400 MHz, DMSO-D6) δ 8.64 (t, J = 6.0 Hz, 0.5H), 8.16 (t, J = 6.2 Hz, 0.5H), 7.99-7.97 (m, 1H), 7.65-7.53 (m, 2H), 7.33-7.25 (m, 1H), 6.73-6.69 (m, 1H), 5.21 (d, J = 7.3 Hz, 0.5H), 4.46-4.26 (m, 3H), 4.14- 4.09 (m, 0.5H), 4.01 (s, 3H), 3.63 (dd, J = 11.4, 7.8 Hz, 0.5H), 3.43-3.35 (m, 2H), 3.26-2.85 (m, 4H), 2.78-2.53 (m, 4H), 2.21-1.66 (m, 9H), 1.57- 1.07 (m, 3H), 0.92-0.85 (m, 2H), 0.69-0.65 (m, 2H) | 6.4 mg, 94%, White Solid |
| | (2S,4S)-4-[(5-cyclopropyloxazol-2- | | |
| | yl)methyl]-N-9(1-methylindazol-5- | | |
| | yl(methyl]-1-[(2R,3S)-3-(pyrrolidine-1- | | |
| | carbonyl)piperidine-2-carbonyl]pyrrolidine- | | |
| | 2-carboxamide | | |
| | S1: 5-(Aminomethyl)-1-methyl-1H-indazole | | |
| | S2: (2S,4S)-1-tert-butoxycarbonyl-4-[(5- | | |
| | cyclopropyloxazol-2-yl)methyl]pyrrolidine- | | |
| | 2-carboxylic acid | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. | | |
| | Step 2: GM2A. purification a | | |
| | Step 3: GM1 with DMF and DIPEA. | | |
| | Purified by prep HPLC | | |
| | Step 4: GM2A. Final purification a | | |
| M06021 | 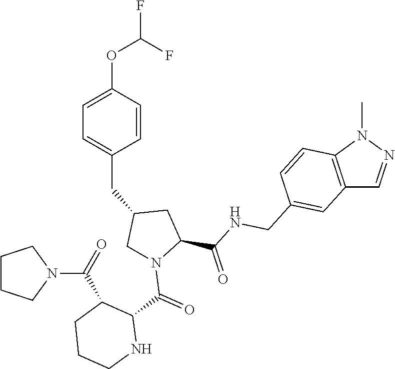 [SMILES: Cn1ncc2cc(CNC(=O)[C@@H]3C[C@@H](Cc4ccc(OC(F)F)cc4)CN3C(=O)[C@@H]3NCCC[C@@H]3C(=O)N3CCCC3)ccc21] | UPLC_pH9_MeCN_QC_ V1 Rt: 4.64 min, m/z 623.4 [M + H]+ UPLC_pH2_MeCN_QC_ V1Rt: 3.42 min, m/z 623.4 [M + H]+ 1H-NMR (400 MHz, DMSO-D6) δ 8.59 (t, J = 6.0 Hz, 0.5H), 8.09 (d, J = 6.0 Hz, 0.5H), 7.97 (s, 1H), 7.58-7.51 (m, 2H), 7.37-7.19 (m, 4H), 7.11- 7.00 (m, 2H), 5.21 (s, 0.5H), 4.42-4.19 (m, 2.5H), 4.03 (d, J = 11.0 Hz, 3H), 3.51-3.38 (m, 2H), 3.29-3.13 (m, 3H), 3.06-2.82 (m, 3H), 2.75- 2.53 (m, 4H), 2.36-2.17 (m, 2H), 1.99-1.64 (m, 8H), 1.55 (q, J = 12.4 Hz, 0.5H), 1.36-1.24 (m, 1H), 1.09 (d, J = 12.8 Hz, 0.5H) | 26.29 mg, 69%, White Solid |
| | (2S,4R)-4-[[4- | | |
| | difluoromethoxy)phenyl]methyl]-N-[(1- | | |
| | methylindazol-5-yl)methyl]-1-[(2R,3S)-3- | | |
| | (pyrrolidine-1-carbonyl)piperidine-2- | | |
| | carbonyl]pyrrolidine-2-carboxamide | | |
| | S1: 5-(Aminomethyl)-1-methyl-1H-indazole | | |
| | S2: (2S,4R)-1-tert-butoxycarbonyl)-4-(4- | | |
| | (difluoromethoxy)benzyl)pyrrolidine-2- | | |
| | carboxylic acid (Peptech PTC21316) | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. | | |
| | Step 2: GM2A. purification a | | |
| | Step 3: GM1 with DMF and DIPEA. | | |
| | Purified by prep HPLC | | |
| | Step 4: GM2A. Final purification a | | |
| M06049 | 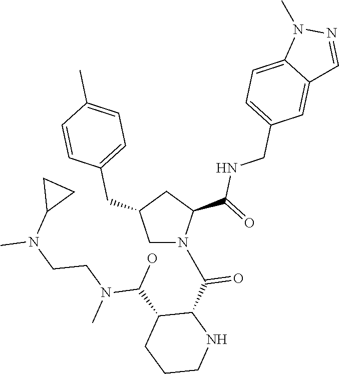 [SMILES: Cc1ccc(C[C@@H]2C[C@@H](C(=O)NCc3ccc4c(cnn4C)c3)N(C(=O)[C@@H]3NCCC[C@@H]3C(O)N(C)CCN(C)C3CC3)C2)cc1] | UPLC_pH9_MeCN_QC_ V1 Rt: 5.04 min, m/z 628.4 [M + H]+ UPLC_pH2_MeCN_QC_ V1 Rt: 2.66 min, m/z 628.4 [M + H]30 1H-NMR (400 MHz, DMSO-D₆) δ 8.55 (t, J = 6.2 Hz, 0.5H), 8.02 (s, 0.5H), 7.95 (d, J = 1.4 Hz, 1H), 7.54 (dt, J = 17.6, 4.4 Hz, 2H), 7.26 (dd, J = 19.7, 8.7 Hz, 1H), 7.10-7.04 (m, 4H), 5.27 (d, J = 4.1 Hz, 0.5H), 4.43-4.25 (m, 2.5H), 4.02-3.97 (m, 3H), 3.57-3.36 (m, 3H), 3.26-2.87 (m, 7H), 2.74 (d, J = 9.6 Hz, 1H), 2.67-2.54 (m, 4H), 2.33-2.06 (m, 7H), 1.92-1.07 (m, 6H), 0.43-0.19 (m, 4H) | 60.26 mg, 57%, White Solid |
| | (2R,3S)-N-[2- | | |
| | [cyclopropyl(methyl)amino]ethyl]N-methyl- | | |
| | 2-[(2S,4R)-2-[(1-methylindazol-5- | | |
| | yl)methylcarbamoyl]-4-(p- | | |
| | tolylmethyl)pyrrolidine-1- | | |
| | carbonyl]piperidine-3-carboxamide | | |
| | S1: 5-(Aminomethyl)-1-methyl-1H-indazole | | |
| | S2: Boc-(R)-y-(4-methylbenzyl)-L-proline | | |
| | S4: (2R,3S)-1-tert-butoxycarbonyl-3-[2- | | |
| | [cyclopropyl(methyl)amino]ethyl-methyl- | | |
| | carbamoyl]piperidine-2-carboxylic acid | | |
| | Step 1: GM1 with DMF and DIPEA. | | |
| | Step 2: GM2A. purification a | | |
| | Step 3: GM1 with DMF and DIPEA. | | |
| | Purified by prep HPLC | | |
| | Step 4: GM2A. Final purification a | | |
| M06068 | 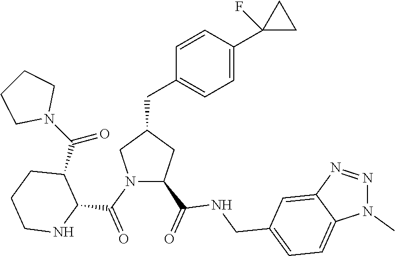 [SMILES: Cn1nnc2cc(CNC(=O)[C@@H]3C[C@@H](Cc4ccc(C5(F)CC5)cc4)CN3C(=O)[C@@H]3NCCC[C@@H]3C(=O)N3CCCC3)ccc21] | UPLC_pH9_MeCN_QC_ V1 Rt: 4.95 min, m/z 616.5 [M + H]30 UPLC_pH2_MeCN_QC_ V1 Rt: 3.46 min, m/z 616.5 [M + H]30 1H-NMR (400 MHz, DMSO-D₆) δ 8.69 (t, J = 6.0 Hz, 0.5H), 8.22 (t, J = 6.2 Hz, 0.5H), 7.82- 7.72 (m, 2H), 7.46-7.36 (m, 1H), 7.27-7.17 (m, 4H), 5.26 (s, 0.5H), 4.49-4.32 (m, 2H), 4.28 (d, J = 2.3 Hz, 3H), 4.02 (dd, J = 9.4, 6.6 Hz, 0.5H), 3.54-3.37 | 29 mg, 51%, White Solid |
| | (2S,4R)-4-[[4-(1- | (m, 2H), 3.28-3.12 (m, | |
| | fluorocyclopropyl)phenyl]methyl]-N-[(1- | 2H), 3.07-2.85 (m, 2H), | |
| | methylbenzotriazol-5-yl)methyl]-1- | 2.76-2.54 (m, 5H), | |
| | [(2R,3S)-3-(pyrrolidine-1- | 2.39-2.18 (m, 1H), | |
| | carbonyl)piperidine-2-carbonyl]pyrrolidine- | 2.01-1.64 (m, 7H), | |
| | 2-carboxamide | 1.60-1.23 (m, 3H), | |
| | S1: (1-methyl-1H-benzo[d][1,2,3]triazol-5- | 1.16-1.08 (m, 2H) | |
| | yl)methanamine | | |
| | S2: (2S,4R)-1-tert-butoxycarbonyl-4- | | |
| | [[4-(1-fluorocyclopropyl)phenyl]methyl] | | |
| | pyrrolidine-2-carboxylic acid | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. | | |
| | Amine used as HCl salt. | | |
| | Step 2: GM2A. No purification | | |
| | Step 3: GM1 with DMF and DIPEA. | | |
| | Purified by prep HPLC | | |
| | Step 4: GM2A. Final purification e | | |
| M06089 | 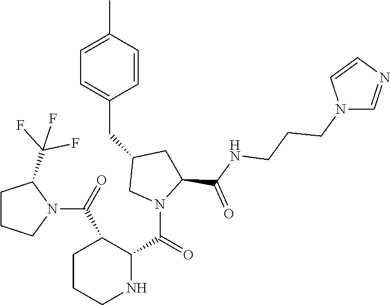 [SMILES: Cc1ccc(C[C@@H]2C[C@@H](C(=O)NCCCn3ccnc3)N(C(=O)[C@@H]3NCCC[C@@H]3C(=O)N3CCC[C@@H]3C(F)(F)F)C2)cc1] | UPLC_pH2_MeCN_QC_ V1 Rt: 2.75 min, m/z 603.3 [M + H]30 UPLC_pH9_MeCN_QC_ V1 Rt: 4.79 min, m/z 603.4 [M + H]30 1H-NMR (400 MHz, DMSO-D6) δ 8.11 (d, J = 5.5 Hz, 0.5H), 7.56 (d, J = 19.2 Hz, 1.5H), 7.14-7.04 (m, 5H), 6.88-6.85 (m, 1H), 5.23 (s, 0.5H), 4.52- 4.88 (1H), 4.03-4.34 (1H), 3.93 (t, J = 6.9 Hz, 1H), 3.87 (t, J = 7.3 Hz, 1H), 3.57-3.44 (m, 3H), 3.24- 2.94 (m, 5.5H), 2.74-2.54 (m, 2H), 2.43-2.31 (m, 2H), 2.26 (d, J = 4.1 Hz, 3H), 1.98-1.58 (m, 12H), | 58.1 mg, 85%, White Solid |
| | (2S,4R)-N-(3-imidazol-1-ylpropyl)-4-(p- | 1.28 (s, 1H) | |
| | tolylmethyl)-1-[(2R,3S)-3-[(2R)-2- | | |
| | trifluoromethyl)pyrrolidine-1- | | |
| | carbonyl]piperidine-2-carbonyl]pyrrolidine- | | |
| | 2-carboxamide | | |
| | S1: 1-(3-Aminopropyl)imidazole | | |
| | S2: Boc-(R)-y-(4-methylbenzyl)-L-proline | | |
| | S4: (2R,3S)-1-tert-butoxycarbonyl-3-[(2R)- | | |
| | 2-(trifluoromethyl)pyrrolidine-1- | | |
| | carbonyl]piperidine-2-carboxylic acid | | |
| | Step 1: GM1 with DMF and DIPEA. | | |
| | Amine used as HCl salt. | | |
| | Step 2: GM2A. Final purification a | | |
| | Step 3: GM1 with DMF and DIPEA. | | |
| | Purified by prep HPLC | | |
| | Step 4: GM2A. Final purification a | | |
| M06077 | 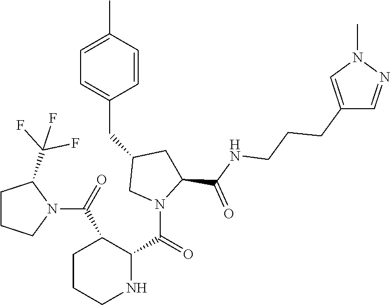 [SMILES: Cc1ccc(C[C@@H]2C[C@@H](C(=O)NCCCc3cnn(C)c3)N(C(=O)[C@@H]3NCCC[C@@H]3C(=O)N3CCC[C@@H]3C(F)(F)F)C2)cc1] | UPLC_pH2_MeCN_QC_ V1 Rt: 3.55 min, m/z 617.5 [M + H]30 UPLC_pH9_MeCN_QC_ V1 Rt: 5.01 min, m/z 617.5 [M + H]30 | 38 mg, 92.9%, White Solid |
| | (2S,4R)-N-(1-methylpyrazol-4-yl)-4-(p- | | |
| | tolylmethyl)-1-[(2R,3S)-3-[(2 | | |
| | tolylmethyl)-1-[(2R,3S)-3-[(2R)-2- | | |
| | (trifluoromethyl)pyrrolidine-1- | | |
| | carbonyl]piperidine-2-carbonyl]pyrrolidine- | | |
| | 2-carboxamide | | |
| | S1: 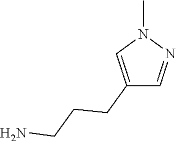 | | |
| | S2: Boc-(R(-y-(4-methylbenzyl)-L-proline | | |
| | S4: (2R,3S)-1-tert-butoxycarbonyl-3-[(2R)- | | |
| | 2-(trifluoromethyl)pyrrolidine-1- | | |
| | carbonyl]piperidine-2-carboxylic acid | | |
| | Step 1: GM1 with DMF and DIPEA. | | |
| | Step 2: GM2A. Final purification a | | |
| | Step 3: GM1 with DMF and DIPEA. | | |
| | Purified by prep HPLC | | |
| | Step 4: GM2A. Final purification a | | |
| M05991 | 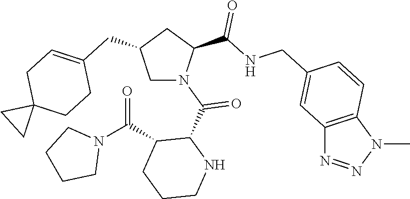 [SMILES: Cn1nnc2cc(CNC(=O)[C@@H]3C[C@@H](CC4=CCC5(CC4)CC5)CN3C(=O)[C@@H]3NCCC[C@@H]3C(=O)N3CCCC3)ccc21] | UPLC_pH9_MeCN_QC_ V1, Rt: 5.0, m/z 588.4 [M + H]30 UPLC_pH2_MeCN_QC_ V1, Rt: 3.58, m/z 588.4 [M + H]30 1H-NMR (400 MHz, ACETONITRILE-D3) δ 7.92 (s, 0.5H), 7.79 (d, J = 29.3 Hz, 1.5H), 7.56 (d, J = 11.9 Hz, 1H), 7.43 (d, J = 16.5 Hz, 1H), 6.98-7.14 (1H), | 2.1 mg, 30%, white solid |
| | (2S,4R)-N-[(1-methylbenzotriazol-5- | 5.41 (s, 1H), 4.50-4.31 | |
| | yl)methyl]-1-[(2R,3S)-3-(pyrrolidine-1- | (m, 4H), 4.20 (s, 5H), | |
| | carbonyl)piperidine-2-carbonyl]-4- | 4.01 (d, J = 36.2 Hz, | |
| | spiro[2.5]oct-6-en-6-ylmethyl)pyrrolidine- | 2H), 3.43-2.88 (m, | |
| | 2-carboxamide | 12H), 1.83-1.62 (m, | |
| | S1: (1-methyl-1H-benzo[d][1,2,3]triazol-5- | 1H), 1.37-1.22 (m, 9H), | |
| | yl)methanamine | 0.21 (t, J = 13.6 Hz, | |
| | S2: (2S,4R)-1-(tert-butoxycarbonyl)-4- | 4H) | |
| | (spiro[2.5]oct-5-en-6- | | |
| | ylmethylene)pyrrolidine-2-carboxylic acid | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. Amine | | |
| | used as HCl salt. Purified by Prep HPLC | | |
| | Step 2: GM2A. No purification | | |
| | Step 3: GM1 with DMF and DIPEA. | | |
| | Purified by prep HPLC | | |
| | Step 4: GM2A. Final purification e (Separated | | |
| | from diene M05992 at this stage) | | |
| M05992 | 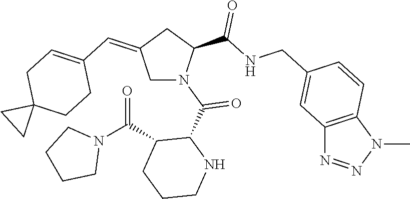 [SMILES: Cn1nnc2cc(CNC(=O)[C@@H]3C/C(=C/C4=CCC5(CC4)CC5)CN3C(=O)[C@@H]3NCCC[C@@H]3C(=O)N3CCCC3)ccc21] | UPLC_pH9_MeCN_QC_ V1, Rt: 4.82, m/z 568.4 [M + H]30 UPLC_pH2_MeCN_QC_ V1, Rt: 3.49, m/z 586.4 [M + H]30 1H-NMR (400 MHz, ACETONITRILE-D3) δ 7.81-7.73 (m, 1H), 7.57 (s, 1H), 7.37 (dd, J = 33.2, 9.4 Hz, 1H), 7.12-7.02 (m, 4H), 4.43-4.34 (m, 2H), 4.20 (d, J = 13.3 Hz, 3H), | 2.8 mg, 40%, white solid |
| | (S,Z)-N-((1-methyl-1H- | 4.02-3.92 (m, 1H), 3.47- | |
| | benzo[d][1,2,3]triazol-5-yl)methyl)-1- | 2.90 (m, 6H), 2.75-2.52 | |
| | ((2R,3S)-3-(pyrrolidine-1- | (m, 7H), 1.85-1.31 (m, | |
| | carbonyl)piperidine-2-carbonyl)-4- | 10H), 1.24-1.13 (m, 4H). | |
| | (spiro[2.5]oct-5-en-6- | Missing protons: 3 | |
| | ylmethylene)pyrrolidine-2-carboxamide | | |
| | S1: (1-methyl-1H-benzo[d][1,2,3]triazol-5- | | |
| | yl)methanamine | | |
| | S2: (S,Z)-1-(tert-butoxycarbonyl)-4- | | |
| | (spiro[2.5]oct-5-en-6- | | |
| | ylmethylene)pyrrolidine-2-carboxylic acid | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. Amine | | |
| | used as HCl salt. Purified by Prep HPLC | | |
| | Step 2: GM2A. No purification | | |
| | Step 3: GM1 with DMF and DIPEA. | | |
| | Purified by prep HPLC | | |
| | Step 4: GM2A. Final purification e (Separated | | |
| | from M05991 at this stage) | | |
| M06006 | 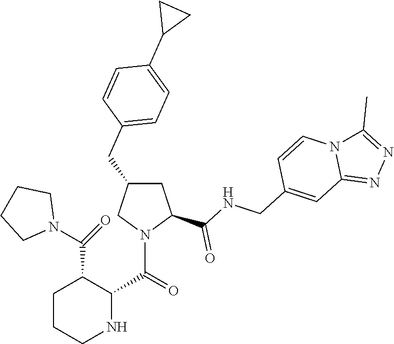 [SMILES: Cc1nnc2cc(CNC(=O)[C@@H]3C[C@@H](Cc4ccc(C5CC5)cc4)CN3C(=O)[C@@H]3NCCC[C@@H]3C(=O)N3CCCC3)ccn12] | UPLC_pH9_MeCN_QC_ V1, Rt: 4.45, m/z 598.4 [M + H]30 UPLC_pH2_MeCN_QC_ V1, Rt: 3.18, m/z 598.4 [M + H]30 1H-NMR (400 MHz, DMSO-D6) δ 8.64 (t, J = 5.7 Hz, 0.5H), 8.28-8.18 (m, 1.5H), 7.38 (d, J = 6.4 Hz, 1H), 7.02 (t, J = 8.7 Hz, 2H), 6.95 (dd, J = 8.0, 5.7 Hz, 2H), 6.77 (ddd, J = 12.5, 7.2, 1.5 Hz, 1H), 5.26 (d, J = 9.2 Hz, 0.5H), 4.33-4.17 (m, 3H), 3.95 (dd, J = 9.8, 6.2 Hz, 0.5H), 3.51-3.32 (m, 2H), 3.20-3.09 (m, 1H), 3.05- 2.93 (m, 1H), 2.64-2.53 (m, 7H), 2.31-2.25 (m, 2H), 1.97-1.44 (m, 10H), | 9.3 mg, 53%, White Solid |
| | (2S,4R)-4-(4-cyclopropylbenzyl)-N-((3- | 1.26-1.13 (m, 1H), 0.89- | |
| | methyl-[1,2,4]triazolo[4,3-a]pyridin-7- | 0.81 (m, 2H), 0.60-0.56 | |
| | yl)methyl)-1-((2R,3S)-3-(pyrrolidine-1- | (m, 2H). Missing protons: | |
| | carbonyl)piperidine-2-carbonyl)pyrrolidine- | 2 | |
| | 2-carboxamide | | |
| | S1: (3-methyl-[1,2,4]triazolo[4,3-a]pyridin- | | |
| | 7-yl)methanamine | | |
| | S2: (2S,4R)-1-tert-butoxycarbonyl-4-[(4- | | |
| | cyclopropylphenyl)methyl]pyrrolidine-2- | | |
| | carboxylic acid | | |
| | S4: (2R,3S)-1-(tert-butoxycarbonyl)-3- | | |
| | (pyrrolidine-1-carbonyl)piperidine-2- | | |
| | carboxylic acid | | |
| | Step 1: GM1 with DMF and DIPEA. | | |
| | Purified by Prep | | |
| | Step 2: GM2B. Purification a | | |
| | Step 3: GM1 with DMF and DIPEA. | | |
| | Purified by prep HPLC | | |
| | Step 4: GM2A. Final purification a | | |
| M06010 | 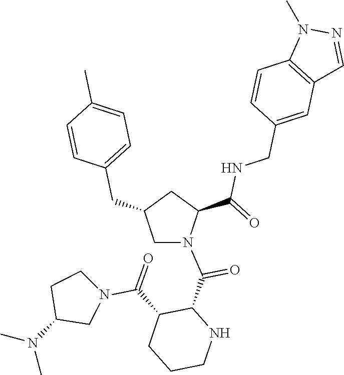 [SMILES: Cc1ccc(C[C@@H]2C[C@@H](C(=O)NCc3ccc4c(cnn4C)c3)N(C(=O)[C@@H]3NCCC[C@@H]3C(=O)N3CC[C@@H](N(C)C)C3)C2)cc1] | UPLC_pH9_MeCN_QC_ V1, Rt: 4.50, m/z 614.5 [M + H]30 UPLC_pH2_MeCN_QC_ V1, Rt: 2.54, m/z 614.5 [M + H]30 1H-NMR (400 MHz, DMSO-D6) δ 8.63-8.58 (m, 0.5H), 8.15-8.04 (m, 0.5H), 7.96 (d, J = 1.8 Hz, 1H), 7.55 (dd, J = 17.6, 8.0 Hz, 2H), 7.26 (dd, J = 19.0, 8.5 Hz, 1H), 7.09-7.04 (m, 4H), 5.21-5.08 (m, 1H), 4.38-4.27 (m, 3H), 4.04-4.01 (m, 4H), 3.66-3.38 (m, 4H), 3.29-2.54 (m, 7H), 2.33-1.10 (m, 17H). Missing protons: 1 | 62.9 mg, 73%, White Solid |
| | (2S,4R)-1-((2R,3S)-3-((R)-3- | | |
| | (dimethylamino)pyrrolidine-1- | | |
| | carbonyl)piperidine-2-carbonyl)-N-((1- | | |
| | methyl-1H-indazol-5-yl)methyl)-4-(4- | | |
| | methylbenzyl)pyrrolidine-2-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3-[(3R)- | | |
| | 3-(dimethylamino)pyrrolidine-1- | | |
| | carbonyl]piperidine-2-carboxylic acid | | |
| | Step 1: GM1 with DMF and DIPEA. | | |
| | Purified by column chromatography. | | |
| | Step2: GM2A. Purification a (SCX-2). | | |
| | Step 3: GM1 with DMF and DIPEA. | | |
| | Purified by prep HPLC | | |
| | Step 4: GM2A. Final purification a | | |
| M06013 | 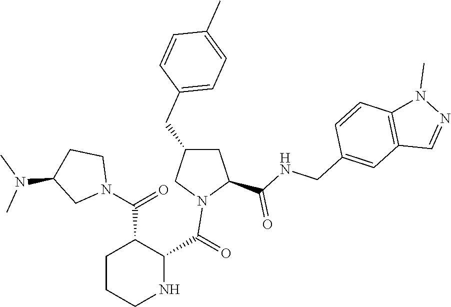 [SMILES: Cc1ccc(C[C@@H]2C[C@@H](C(=O)NCc3ccc4c(cnn4C)c3)N(C(=O)[C@@H]3NCCC[C@@H]3C(=O)N3CC[C@H](N(C)C)C3)C2)cc1] | UPLC_pH2_MeCN_QC_ V1Rt: 2.55, m/z 614.5 [M + H]30 UPLC_pH9_MeCN_QC_ V1Rt: 4.6, m/z [M + H]30 1H-NMR (400 MHz, DMSO-D6) δ 8.58 (dd, J = 15.3, 6.2 Hz, 0.5H), 8.17- 8.07 (m, 0.5H), 7.96 (s, 1H), 7.55 (dd, J = 18.1, 8.0 Hz, 2H), 7.29-7.22 (m, 1H), 7.11-7.04 (m, 4H), 5.22-5.19 (m, 0.5H), 4.43- 4.25 (m, 2.5H), 4.02 (s, 3H), 3.57-3.38 (m, 5H), 3.14-2.80 (m, 5H), 2.73- 2.55 (m, 5H), 2.33 (t, J = 3.7 Hz, 0.5H), 2.27 (s, 3H), 2.16-2.11 (m, 6H), 2.03-1.88 (m, 2H), 1.82- | 66 mg, 91%, White Solid |
| | (2S,4R)-1-((2R,3S)-3-((S)-3- | 1.46 (m, 3H), 1.30 (d, J = | |
| | (dimethylamino)pyrrolidine-1- | 25.2 Hz, 1H), 1.09 (d, J = | |
| | carbonyl)piperidine-2-carbonyl)-N-((1- | 12.8 Hz, 0.5H) | |
| | methyl-1H-indazol-5-yl)methyl)-4-(4- | | |
| | methylbenzyl)pyrrolidine-2-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3-[(3S)- | | |
| | 3-(dimethylamino)pyrrolidine-1- | | |
| | carbonyl]piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06033 | 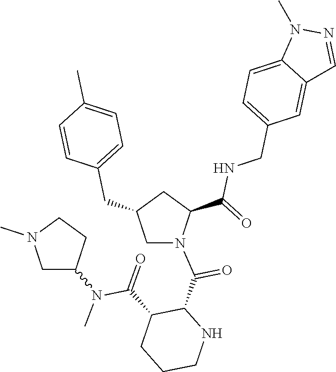 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CCN(C)C3)C2)cc1] | UPLC_pH2_MeCN_QC_ V1Rt: 2.61, m/z 614.4 [M + H]30 UPLC_pH9_MeCN_QC_ V1 Rt: 4.97, m/z 614.5 [M + H]30 1H-NMR (400 MHz, DMSO-D6) δ 8.58 (q, J = 5.8 Hz, 0.5H), 8.09- 8.05 (m, 0.5H), 7.95 (d, J = 8.2 Hz, 1H), 7.58- 7.51 (m, 2H), 7.29-7.22 (m, 1H), 7.10-7.04 (m, 4H), 5.30-5.21 (m, 0.5H), 5.06-4.96 (m, 0.5H), 4.43-4.24 (m, 2H), 4.05-3.97 (m, 3H), 3.54-3.42 (m, 1H), 3.15-2.53 (m, 11H), 2.37- 1.06 (m, 18H). Missing protons: 1 | 27.9 mg, 86%, Brown Solid |
| | (2R,3S)-N-methyl-2-((2S,4R)-2-(((1- | | |
| | methyl-1H-indazol-5-yl)methyl)carbmoyl)- | | |
| | 4-(4-methylbenzyl)pyrrolidine-1-carbonyl)- | | |
| | N-(1-methylpyrrolidin-3-yl)piperidine-3- | | |
| | carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-tert-butoxycarbonyl-3- | | |
| | [methyl-(1-methylpyrrolidin-3- | | |
| | yl)carbamoyl]piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06034 | 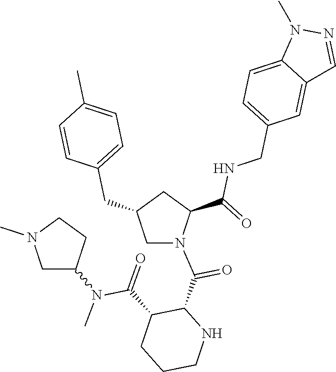 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CCN(C)C3)C2)cc1] | UPLC_pH2_MeCN_QC_ V1Rt: 2.6, m/z 614.5 [M + H]30 UPLC_pH9_MeCN_QC_ V1Rt: 4.97, m/z 614.4 [M + H]30 1H-NMR (400 MHz, DMSO-D6) δ 8.58 (t, J = 6.2 Hz, 0.5H), 8.15- 8.03 (m, 0.5H), 7.95 (d, J = 6.4 Hz, 1H), 7.55 (dd, J = 18.8, 8.2 Hz, 2H), 7.25 (dd, J = 19.7, 8.7 Hz, 1H), 7.08-7.04 (m, 4H), 5.28-5.22 (m, 0.5H), 5.07-4.91 (m, 0.5H), 4.41-4.24 (m, 3H), 4.07-3.92 (m, 3H), 3.52-3.42 (m, 1H), 3.15-2.55 (m, 10H), 2.35- 1.06 (m, 17H). Missing protons: 3 | 29.04 mg, 79%, Off- White Solid |
| | (2R,3S)-N-methyl-2-((2S,4R)-2-(((1- | | |
| | methyl-1H-indazol-5-yl)methyl)carbamoyl)- | | |
| | 4-(4-methylbenzyl)pyrrolidine-1-carbonyl)- | | |
| | N-(1-methylpyrrolidin-3-yl)piperidine-3- | | |
| | carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-tert-butoxycarbonyl-3- | | |
| | [methyl-(1-methylpyrrolidin-3- | | |
| | yl)carbamoyl]piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06039 | 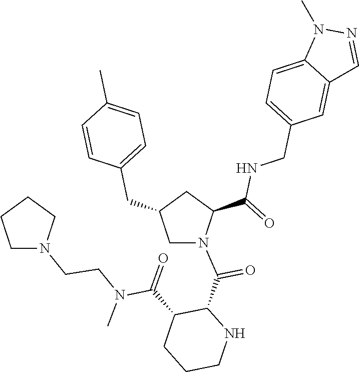 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN3CCCC3)C2)cc1] | UPLC_pH9_MeCN_QC_ V1, Rt: 5.74, m/z 628.4 [M + H]30 UPLC_pH2_MeCN_QC_ V1, Rt: 2.6, m/z 628.4 [M + H]30 1H-NMR (400 MHz, DMSO-D6) δ 8.57 (t, J = 6.0 Hz, 0.6H), 8.12-8.04 (m, 0.4H), 7.96 (d, J = 3.2 Hz, 1H), 7.55 (q, J = 8.4 Hz, 2H), 7.29-7.22 (m, 1H), 7.10-7.04 (m, 4H), 5.27 (s, 0.5H), 4.43-4.19 (m, 2.5H), 4.01 (t, J = 16.0 Hz, 3.5H), 3.56-3.41 (m, 1.5H), 3.17-2.87 (m, 6H), 2.76 (d, J = 8.7 Hz, 1H), 2.67-2.54 (m, 4H), 2.47-2.40 (m, 2H), 2.33- 2.26 (m, 4H), 2.13 (d, J = 24.3 Hz, 1H), 1.91-1.64 (m, 8H), 1.45-1.07 (m, 2H). Missing protons: 2 | 37 mg, 49%, White Gum |
| | (2R,3S)-N-methyl-2-((2S,4R)-2-(((1- | | |
| | methyl-1H-indazol-5-yl)methyl)carbamoyl)- | | |
| | 4-(4-methylbenzyl)pyrrolidine-1-carbonyl)- | | |
| | N-(2-(pyrrolidin-1-yl)ethyl)piperidine-3- | | |
| | carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | +methyl(2-pyrrolidin-1- | | |
| | ylethyl)carbamoyl+piperidine-2-carboxylic | | |
| | acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06048 | 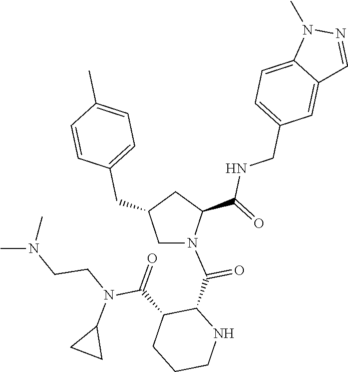 [SMILES: Cc1ccc(C[C@@H]2C[C@@H](C(=O)NCc3ccc4c(cnn4C)c3)N(C(=O)[C@@H]3NCCC[C@@H]3C(=O)N(CCN(C)C)C3CC3)C2)cc1] | UPLC_pH9_MeCN_QC_ V1, Rt: 5.74, m/z 628.4 [M + H UPLC_pH2_MeCN_QC_ V1, Rt: 2.7, m/z 628.4 [M + H]30 1H-NMR (400 MHz, DMSO-D6) δ 8.60 (t, J = 6.0 Hz, 0.5H), 8.07 (t, J = 5.7 Hz, 0.5H), 7.96-7.91 (m, 1H), 7.58-7.47 (m, 2H), 7.33-7.21 (m, 1H), 7.10-7.03 (m, 4H), 5.30 (t, J = 5.3 Hz, 0.5H), 4.43- 4.19 (m, 2H), 4.11-4.07 (m, 0.5H), 4.03-3.97 (m, 3H), 3.54-3.46 (m, 2H), 3.31 (s, 2H), 3.15-2.84 (m, 4H), 2.74-2.54 (m, 4H), 2.36-2.22 (m, 6H), 2.17-2.03 (m, 6H), 1.96- 1.67 (m, 3H), 1.51-1.06 (m, 2H), 0.91-0.63 (m, 4H) | 5 mg, 9%, Off- White Solid |
| | (2R,3S)-N-cyclopropyl-N-(2- | | |
| | (dimethylamino)ethyl)-2-((2S,4R)-2-(((1- | | |
| | methyl-1H-indazol-5-yl)methyl)carbamoyl)- | | |
| | 4-(4-methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | +cyclopropyl- | | |
| | (dimethylamino)ethyl+carbamoyl+piperidine- | | |
| | 2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06061 | 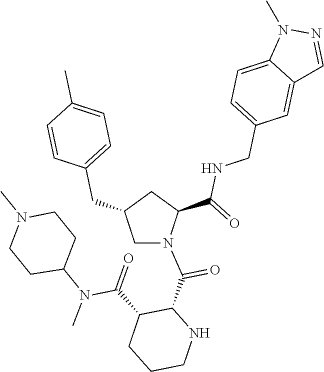 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CCN(C)CC3)C2)cc1] | UPLC_pH9_MeCN_QC_ V1, Rt: 5.06, m/z 628.4 [M + H]30 UPLC_pH2_MeCN_QC_ V1, Rt: 2.56, m/z 628.6 [M + H]30 1H-NMR (400 MHz, DMSO-D6) δ 8.59 (d, J = 6.0 Hz, 0.6H), 8.11-8.04 (m, 0.4H), 7.96 (d, J = 4.1 Hz, 1H), 7.57 (t, J = 4.1 Hz, 1H), 7.52 (d, J = 8.2 Hz, 1H), 7.30-7.21 (m, 1H), 7.10-7.04 (m, 4H), 5.30-5.24 (m, 1H), 4.44- 4.15 (m, 3H), 4.02 (s, 4H), 3.49-3.40 (m, 1H), 3.15 (t, J = 8.7 Hz, 0.5H), 3.03- 2.87 (m, 2.5H), 2.83-2.77 (m, 2H), 2.72-2.66 (m, 1H), 2.66-2.53 (m, 4H), 2.35-2.26 (m, 4H), 2.21- 2.11 (m, 3H), 1.95-1.23 (m, 12H), 1.14-1.08 (m, | 48 mg, 41%, White Solid |
| | (2R,3S)-N-methyl-2-((2S,4R)-2-(((1- | 1H) | |
| | methyl-1H-indazol-5-yl)methyl)carbamoyl)- | | |
| | 4-(4-methylbenzyl)pyrrolidine-1-carbonyl)- | | |
| | N-(1-methylpiperidin-4-yl)piperidine-3- | | |
| | carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | +methyl-(1-methyl-4- | | |
| | piperidyl)carbamoyl+piperidine-2-carboylic | | |
| | acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification e | | |
| M06063 | 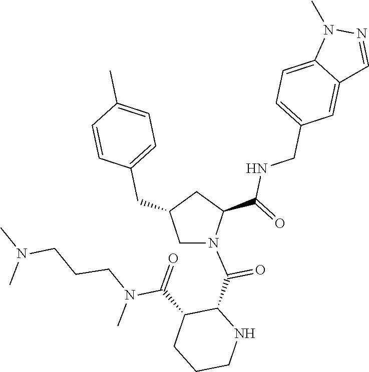 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCCN(C)C)C2)cc1] | UPLC_pH2_MeCN_QC_ V1, Rt: 2.62, m/z 616.6 [M + H]30 UPLC_pH9_MeCN_QC_ V1, Rt: 5.23, m/z 616.6 [M + H]30 1H-NMR (400 MHz, DMSO-D₆) δ 8.58 (t, J = 6.2 Hz, 0.5H), 8.09 (dt, J = 23.5, 5.8 Hz, 0.5H), 7.99-7.95 (m, 1H), 7.58-7.51 (m, 2H), 7.26 (dd, J = 21.5, 9.2 Hz, 1H), 7.10-7.03 (m, 4H), 5.27 (t, J = 5.0 Hz, 1H), 4.43-4.24 (m, 2H), 4.09-3.97 (m, 3H), 3.52-3.45 (m, 1H), 3.29-2.88 (m, 6H), 2.74-2.53 (m, 4H), 2.33-2.07 (m, 12H), 1.92-1.08 (m, 8H) Missing protons: 2 | 88.5 mg, 89%, Off- White Solid |
| | (2R,3S)-N-(3-(dimethylamino)propyl)-N- | | |
| | methyl-2-((2S,4R)-2-(((1-methyl-1H- | | |
| | indazol-5-yl)methyl)carbamoyl)-4-(4- | | |
| | methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | (dimethylamino)propyl-methyl- | | |
| | carbamoyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06085 | 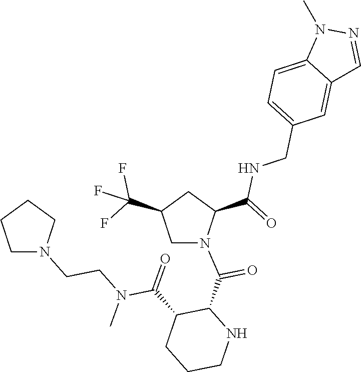 [SMILES: CN(CCN1CCCC1)C(=O)[C@H]1CCCN[C@H]1C(=O)N1C[C@@H](C(F)(F)F)C[C@H]1C(=O)NCc1ccc2c(cnn2C)c1] | UPLC_pH9_MeCN_QC_ V1, Rt: 4.35, m/z 592.4 [M + H]30 UPLC_pH2_MeCN_QC_ V1, Rt: 2.1, m/z 592.4 [M + H]30 1H-NMR (400 MHz, DMSO-D6) δ 8.67 (m, 0.5H), 8.20 (broad s, 0.5H), 7.99 (s, 1H), 7.63-7.55 (m, 2H), 7.3- 7.32 (d, J = 8.7 Hz, 0.5H), 7.30-7.27 (d, J = 8.7, 0.5H), 5.34-5.27 (m, 0.5H), 4.48-4.30 (m, 3H), 4.02 (s, 3.5H), 3.93-3.84 (m, 1H), 3.71-3.45 (m, 2H), 3.27-3.21 (m, 2H), 3.11-2.98 (m, 2H), 2.95 (s, 1H), 2.89-2.78 (broad m, 1H), 2.76 (s, 1H), 2.68-2.52 (m, 2H), 2.48-2.36 (m, 4H), | 21.2 mg, 89%, Light Yellow Solid |
| | (2R,3S)-N-methyl-2-((2S,4S)-2-(((1- | 2.12-1.99 (m, 1H), | |
| | methyl-1H-indazol-5-yl)methyl)carbamoyl)- | 1.99-1.83 (m, 2H), | |
| | 4-(trifluoromethyl)pyrrolidine-1-carbonyl)- | 1.82-1.72 (m, 1H), | |
| | N-(2-(pyrrolidin-1-yl)ethyl)piperidine-3- | 1.71-1.59 (m, 4H), | |
| | carboxamide | 1.39-1.31 (m, 1H), 1.26- | |
| | S1: (1-methyl-1H-indazol-5- | 1.21 (m, 1H). Missing | |
| | yl)methanamine | protons: 1 | |
| | S2: (2S,4S)-1-(tert-Butoxycarbonyl)-4- | | |
| | (trifluoromethyl)pyrrolidine-2-carboxylic | | |
| | acid | | |
| | S4: (2R,3S)-1-tert-butoxycarbonyl-3- | | |
| | +methyl(2-pyrrolidin-1- | | |
| | ylethyl)carbamoyl+piperidine-2-carboxylic | | |
| | acid | | |
| | Step 1: GM1 with DMF and DIPEA. | | |
| | Purified by column chromatography | | |
| | Step 2: GM2A. Purification a | | |
| | Step 3: GM1 with DMF and DIPEA. | | |
| | Purified by prep HPLC | | |
| | Step 4: GM2A. Final purification e | | |
| M06099 | 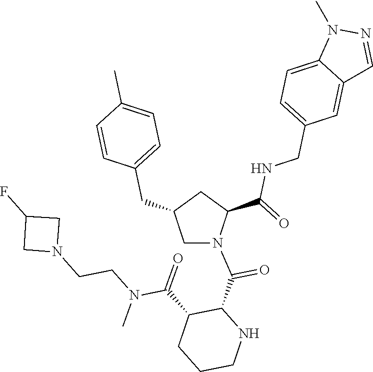 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN3CC(F)C3)C2)cc1] | UPLC_pH9_MeCN_QC_ V1, Rt: 4.65, m/z 632.5 [M + H]30 UPLC_pH2_MeCN_QC_ V1, Rt: 2.62, m/z 632.5 [M + H]30 1H-NMR (400 MHz, DMSO-D6) δ 8.52 (t, J = 6.0 Hz, 0.5H), 8.01 (s, 0.5H), 7.91 (d, J = 4.6 Hz, 1H), 7.54-7.47 (m, 2H), 7.25-7.18 (m, 1H), 7.06-7.00 (m, 4H), 5.22-5.12 (m, 1H), 4.99 (q, J = 5.0 Hz, 0.5H), 4.39-4.21 (m, 2.5H), 3.98 (t, J = 14.4 Hz, 4H), 3.53-3.41 (m, 3H), 3.16-2.84 (m, 8H), 2.70 (d, J = 8.7 Hz, 1H), 2.65-2.50 (m, 4H), 2.32-2.22 (m, 3.5H), 2.14-2.06 (m, 0.5H), 1.92-1.60 (m, 4H), 1.41 | 40 mg, 97%, Off- White Solid |
| | (2R,3S)-N-(2-(3-fluoroazetidin-1-yl)ethyl)- | (d, J = 12.8 Hz, 0.5H), | |
| | N-methyl-2-((2S,4R)-2-(((1-methyl-1H- | 1.31-1.19 (m, 1H), 1.06 | |
| | indazol-5-yl)methyl)carbamoyl)-4-(4- | (d, J = 9.6 Hz, 0.5H) | |
| | methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3-+2-(3- | | |
| | fluoroazetidin-1-yl)ethyl-methyl- | | |
| | carbamoyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06109 | 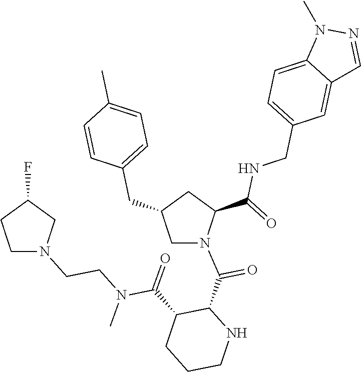 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN3CC[C@H](F)C3)C2)cc1] | UPLC_pH2_MeCN_QC_ V1, Rt: 2.64, m/z 646.6 [M + H]30 UPLC_pH9_MeCN_QC_ V1, Rt: 4.78, m/z 646.5 [M + H]30 1H-NMR (400 MHz, DMSO-D6) δ 8.64 (s, 0.5H), 8.37 (s, 0.5H), 8.00 (d, J = 4.1 Hz, 1H), 7.59 (dd, J = 15.1, 8.2 Hz, 2H), 7.30 (dd, J = 16.7, 9.4 Hz, 1H), 7.17-7.08 (m, 4H), 5.37-5.07 (m, 1H), 4.44- 4.30 (m, 2H), 4.07-3.97 (m, 4H), 3.54-3.45 (m, 1H), 3.20-2.58 (m, 12H), 2.37-2.30 (m, 5H), 2.13 (d, J = 14.2 Hz, 1H), 1.93- 1.27 (m, 8H). Missing protons: 4 | 87.5 mg, 82%, White Solid |
| | (2R,3S)-N-(2-((S)-3-fluoropyrrolidin-1- | | |
| | yl)ethyl)-N-methyl-2-((2S,4R)-2-(((1- | | |
| | methyl-1H-indazol-5-yl)methyl)carbamoyl)- | | |
| | 4-(4-methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | +(3S)-3-fluoropyrrolidin-1-yl+ethyl-methyl- | | |
| | carbamoyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06112 | 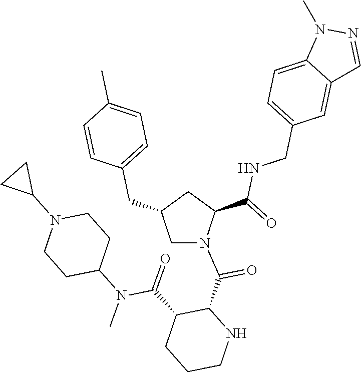 [SMILES: Cc1ccc(C[C@@H]2C[C@@H](C(=O)NCc3ccc4c(cnn4C)c3)N(C(=O)[C@@H]3NCCC[C@@H]3C(=O)N(C)C3CCN(C4CC4)CC3)C2)cc1] | UPLC_pH2_MeCN_QC_ V1, Rt: 2.63, m/z 654.6 [M + H]30 UPLC_pH9_MeCN_QC_ V1, Rt: 5.14, m/z 654.5 [M + H]30 1H-NMR (400 MHz, DMSO-D6) δ 8.60 (t, J = 5.5 Hz, 0.5H), 8.14- 8.04 (m, 0.5H), 8.00 (d, J = 4.1 Hz, 1H), 7.63- 7.55 (m, 2H), 7.34-7.26 (m, 1H), 7.15-7.08 (m, 4H), 5.38-5.25 (d, J = 7.8 Hz, 1H), 4.43-4.27 (m, 3H), 4.07-4.05 (m, 3H), 3.59-3.44 (m, 1H), 3.19-2.91 (m, 5H), 2.79-2.58 (m, 5H), 2.37-2.10 (m, 6H), 1.95-1.12 (m, 10H), 0.44-0.29 (m, 4H). Missing protons: 4 | 56.8 mg, 86%, White Solid |
| | (2R,3S)-N-(1-cyclopropylpiperidin-4-yl)-N- | | |
| | methyl-2-((2S,4R)-2-(((1-methyl-1H- | | |
| | indazol-5-yl)methyl)carbamoyl)-4-(4- | | |
| | methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3-+(1- | | |
| | cyclopropyl-4-piperidyl)-methyl- | | |
| | carbamoyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06128 | 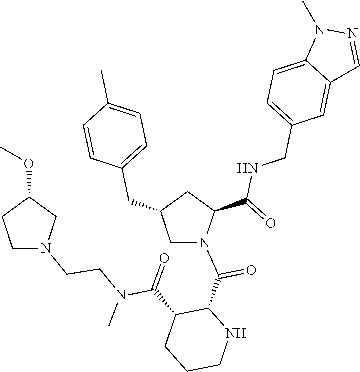 [SMILES: CO[C@H]1CCN(CCN(C)C(=O)[C@H]2CCCN[C@H]2C(=O)N2C[C@H](Cc3ccc(C)cc3)C[C@H]2C(=O)NCc2ccc3c(cnn3C)c2)C1] | UPLC_pH2_MeCN_QC_ V1, Rt: 2.63, m/z 658.6 [M + H]30 UPLC_pH9_MeCN_QC_ V1, Rt: 5.02, m/z 658.7 [M + H]30 1H-NMR (400 MHz, DMSO-D6) δ 8.57-8.50 (m, 0.5H), 8.11-8.01 (m, 0.5H), 7.95 (dd, J = 3.66, 1.14 Hz, 1H), 7.58-7.49 (m, 2H), 7.31-7.22 (m, 1H), 7.10-7.04 (m, 4H), 5.24 (t, J = 5.50 Hz, 1H), 4.48-4.24 (m, 2H), 4.02 (s, 3H), 3.88-3.77 (m, 1H), 3.54-3.39 (m, 2H), 3.24-2.88 (m, 8H), 2.77-2.53 (m, 6H), 2.45-1.22 (m, 15H), 1.10-1.02 (m, 1H). Missing protons: 2 | 39.0 mg, 100%, Brown Gum |
| | (2R,3S)-N-(2-((S)-3-methoxypyrrolidin-1- | | |
| | yl)ethyl)-N-methyl-2-((2S,4R)-2-(((1- | | |
| | methyl-1H-indazol-5-yl)methyl)carbamoyl)- | | |
| | 4-(4-methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | +(3S)-3-methoxypyrrolidin-1-yl+ethyl- | | |
| | methyl-carbamoyl+piperidine-2-carboxylic | | |
| | acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06129 | 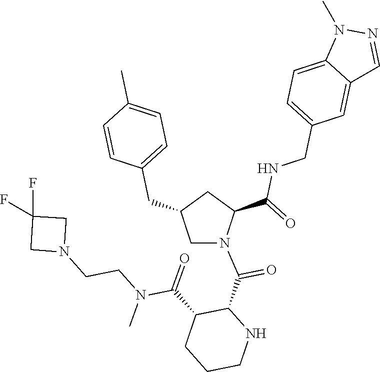 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN3CC(F)(F)C3)C2)cc1] | UPLC_pH2_MeCN_QC_ V1, Rt: 3.03, m/z 650.6 [M + H]30 UPLC_pH9_MeCN_QC_ V1, Rt: 4.85, m/z 650.6 [M + H]30 1H-NMR (400 MHz, DMSO-D6) δ 8.58 (t, J = 5.7 Hz, 0.5H), 8.09 (d, J = 6.0 Hz, 0.5H), 7.96 (d, J = 4.1 Hz, 1H), 7.55 (dd, J = 17.40, 8.9 Hz, 2H), 7.29-7.21 (m, 1H), 7.10-7.04 (m, 4H), 5.27 (d, J = 5.0 Hz, 1H), 4.44-4.24 (m, 2H), 4.02 (s, 3H), 3.64-3.45 (m, 5H), 3.24-2.54 (m, 11H), 2.37-1.23 (m, 9H), 1.09 (d, J = 9.2 Hz, 1H). Missing protons: 3 | 52.1 mg, 90%, White Solid |
| | (2R,3S)-N-(2-(3,3-difluoroazetidin-1- | | |
| | yl)ethyl)-N-methyl-2-((2S,4R)-2-(((1- | | |
| | methyl-1H-indazol-5-yl)methyl)carbamoyl)- | | |
| | 4-(4-methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3- | | |
| | (3,3-difluoroazetidin-1-yl)ethyl-methyl- | | |
| | carbamoyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06131 | 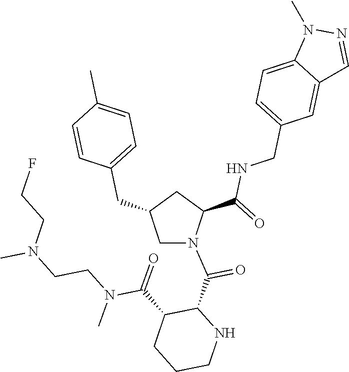 [SMILES: Cc1ccc(C[C@@H]2C[C@@H](C(=O)NCc3ccc4c(cnn4C)c3)N(C(=O)[C@@H]3NCCC[C@@H]3C(=O)N(C)CCN(C)CCF)C2)cc1] | UPLC_pH2_MeCN_QC_ V1, Rt: 2.61, m/z 634.6 [M + H]30 UPLC_pH9_MeCN_QC_ V1, Rt: 5.27, m/z 634.6 [M + H]30 1H-NMR (400 MHz, DMSO-D6) δ 8.58 (t, J = 6.0 Hz, 0.5H), 8.12 (s, 0.5H), 7.96 (d, J = 2.7 Hz, 1H), 7.55 (dd, J = 16.49, 8.2 Hz, 2H), 7.25 (dd, J = 20.1, 9.2 Hz, 1H), 7.11-7.04 (m, 4H), 5.27 (s, 1H), 4.56- 4.25 (m, 4H), 4.01 (s, 3H), 3.50-3.38 (m, 2H), 3.26-2.90 (m, 5H), 2.78-2.54 (m, 7H), 2.46-2.18 (m, 8H), 1.90-1.65 (m, 4H), 1.46-1.23 (m, 2H), 1.09 (d, J = 10.5 Hz, 1H). Missing protons: 2 | 5.01 mg, 73%, White Solid |
| | (2R,3S)-N-(2-((2- | | |
| | fluoroethyl)(methyl)amino)ethyl)-N-methyl- | | |
| | 2-((2S,4R)-2-(((1-methyl-1H-indazol-5- | | |
| | yl)methyl)carbamoyl)-4-(4- | | |
| | methylbenzyl)pyrrolidine-1- | | |
| | carbonyl)piperidine-3-carboxamide | | |
| | S1: (1-methyl-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-tert-butoxycarbonyl-3-+2- | | |
| | fluoroethyl(methyl)amino+ethyl-methyl- | | |
| | carbamoyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06079 | 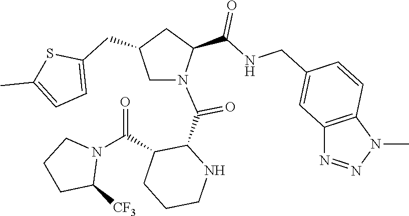 | UPLC_pH2_MeCN_QC_ V1, Rt: 3.51, m/z 646.4 [M + H]30 UPLC_pH9_MeCN_QC_ V1, Rt: 4.95, m/z 646.4 [M + H]30 1H-NMR (400 MHz, DMSO-D6) δ 8.70 (s, 0.5H), 8.15 (s, 0.5H), 7.81-7.71 (m, 2H), 7.39 (dd, J = 25.2, 7.3 Hz, 1H), 6.60 (s, 2H), 5.26 (d, J = 7.8 Hz, 1H), 4.80-4.72 (m, 1H), 4.54-4.17 (m, 6H), | 53 mg, 92%, White Solid |
| | (2S,4S)-N-+(1-methylbenzotriazol-5- | 3.60-3.38 (m, 3H), 3.01- | |
| | yl)methyl+-4-+(5-methyl-2-thienyl)methyl+-1- | 2.62 (m, 7H), 2.34-2.23 | |
| | +(2R,3S)-3-+(2R)-2- | (m, 3H), 1.99-1.11 (m, | |
| | (trifluoromethyl)pyrrolidine-1- | 10H) | |
| | carbonyl+piperidine-2-carbonyl+pyrrolidine- | | |
| | 2-carboxamide | | |
| | S1: (1-methyl-1H-benzo | | |
| | yl)methanamine | | |
| | S2: (2S,4S)-1-tert-butoxycarbonyl-4-+(5- | | |
| | methyl-2-thienyl)methyl+pyrrolidine-2- | | |
| | carboxylic acid | | |
| | S4: (2R,3S)-1-tert-butoxycarbonyl-3-+(2R)- | | |
| | 2-(trifluoromethyl)pyrrolidine-1- | | |
| | carbonyl+piperidine-2-carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06030 | 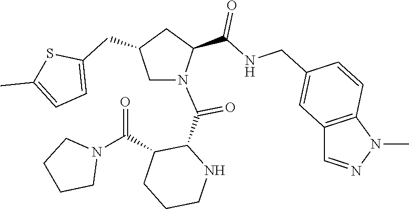 | UPLC_pH2_MeCN_QC_ V1, Rt: 3.36, m/z 577.3 [M + H]30 UPLC_pH9_MeCN_QC_ V1, Rt: 4.63, m/z 577.4 [M + H]30 1H-NMR (400 MHz, DMSO-D6) δ 8.61 (t, J = 6.2 Hz, 0.5H), 8.12 (t, J = 6.0 Hz, 0.5H), 7.97 (d, J = 0.9 Hz, 1H), 7.56 (q, J = 8.1 Hz, 2H), 7.31-7.24 (m, 1H), 6.63-6.60 (m, 2H), 5.22 (d, J = 7.8 Hz, 0.5H), | 52 mg, 96%, off- white Solid |
| | (2S,4S)-N-((1-methyl-1H-indazol-5- | 4.44-4.26 (m, 2H), 4.08- | |
| | yl)methyl)-4-((5-methylthiophen-2- | 3.98 (m, 3.5H), 3.58-3.35 | |
| | yl)methyl)-1-((2R,3S)-3-(pyrrolidine-1- | (m, 2H), 3.26-2.65 (m, | |
| | carbonyl)piperidine-2-carbonyl)pyrrolidine- | 5H), 2.38 (s, 3H), 2.33- | |
| | 2-carboxamide | 2.21 (m, 1H), 2.04-1.54 | |
| | S1: (1-methyl-1H-indazol-5- | (m, 8H), 1.37-1.08 (m, | |
| | yl)methanamine | 2H). Missing protons: 5 | |
| | S2: (2S,4S)-1-tert-butoxycarbonyl-4-+(5- | | |
| | methyl-2-thienyl)methyl+pyrrolidine-2- | | |
| | carboxylic acid | | |
| | S4: (2R,3S)-1-tert-butoxycarbonyl-3- | | |
| | (pyrrolidine-1-carbonyl)piperidine-2- | | |
| | carboxylic acid | | |
| | Step 1: GM1 with DMF and DIPEA. | | |
| | Purified by column chromatography. | | |
| | Step 2: GM2A. Purification a (SCX-2). | | |
| | Step 3: GM1 with DMF and DIPEA. | | |
| | Purified by prep HPLC | | |
| | Step 4: GM2A. Final purification a | | |
| M06047 | 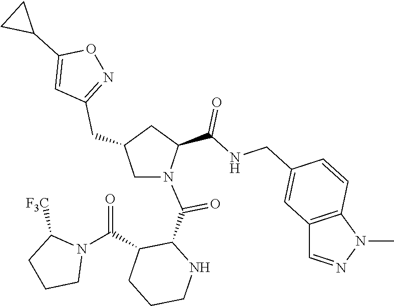 [SMILES: Cn1ncc2cc(CNC(=O)[C@@H]3C[C@@H](Cc4cc(C5CC5)on4)CN3C(=O)[C@@H]3NCCC[C@@H]3C(=O)N3CCC[C@@H]3C(F)(F)F)ccc21] | UPLC_pH9_MeCN_QC_ V1, Rt: 4.70 mins, m/z 656.3 [M + H]30 UPLC_pH2_MeCN_QC_ V1, Rt: 3.39 mins, m/z 656.3 [M + H]30 1H-NMR (400 MHz, DMSO-D6) δ 8.66 (t, J = 6.2 Hz, 0.6H), 8.10 (t, J = 5.7 Hz, 0.4H), 7.99- 7.96 (m, 1H), 7.59 (d, J = 8.7 Hz, 1H), 7.54 (t, J = 4.4 Hz, 1H), 7.28 (ddd, J = 25.1, 8.6, 1.5 Hz, 1H), 6.09-6.07 (m, 1H), 5.25 (d, J = 6.0 Hz, 0.5H), 4.76-4.67 (m, 1H), 4.47-4.23 (m, 2.5H), 4.02 (d, J = 1.4 Hz, 3H), 3.65-3.40 (m, | 102 mg, 89%, white solid |
| | (2S,4S)-4-+(5-cyclopropylisoxazol-3- | 3H), 3.04-2.90 (m, 2H), | |
| | yl)methyl+-N-+(1-methylindazol-5- | 2.67-2.56 (m, 4H), | |
| | yl)methyl+-1-+(2R,3S)-3-+(2R)-2- | 2.46-2.38 (m, 1H), | |
| | (trifluoromethyl)pyrrolidine-1- | 2.17-1.73 (m, 8H), | |
| | carbonyl+piperidine-2-carbonyl+pyrrolidine- | 1.64-1.29 (m, 2H), | |
| | 2-carboxamide | 1.14-0.96 (m, 3H), | |
| | S1: 5-(Aminomethyl)-1-methyl-1H-indazole | 0.85-0.81 (m, 2H). | |
| | S2: (2S,4S)-1-tert-butoxycarbonyl-4-+(5- | Missing protons: 1 | |
| | cyclopropylisoxazol-3- | | |
| | yl)methyl+pyrrolidine-2-carboxylic acid | | |
| | S4: (2R,3S)-1-tert-butoxycarbonyl-3-+(2R)- | | |
| | 2-(trifluoromethyl)pyrrolidine-1- | | |
| | carbonyl+piperidine-2-carboxylic acid | | |
| | Step 1: GM1 with DMF and DIPEA. Amine | | |
| | used as HCl salt. Purified by Prep HPLC | | |
| | Step 2: GM2A. No purification | | |
| | Step 3: GM1 with DMF and DIPEA. | | |
| | Purified by prep HPLC | | |
| | Step 4: GM2A. Final purification e | | |

### Synthesis of (2$,4R)-1-((2R,3S)-3-(5-azaspiro[2.4]heptane-5-carbonyl)piperidine-2-carbonyl)-N-((1-methyl-1H-indazol-5-yl)methyl)-4-(4-methylbenzyl)pyrrolidine-2-carboxamide (M05766)

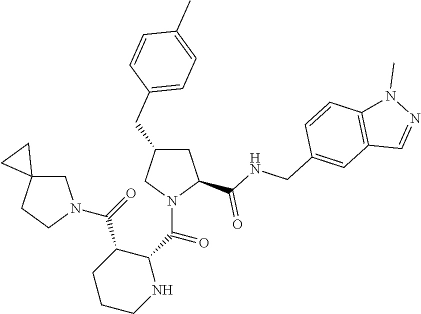 [SMILES: Cc1ccc(C[C@@H]2C[C@@H](C(=O)NCc3ccc4c(cnn4C)c3)N(C(=O)[C@@H]3NCCC[C@@H]3C(=O)N3CCC4(CC4)C3)C2)cc1]

Step 1: To a stirred solution of (1-methylindazol-5-yl)methanamine (950 mg, 5.89 mmol) and Boc-(R)-4-(4-methylbenzyl)-L-proline (1.8 g, 5.61 mmol) in dichloromethane (28 mL) at room temperature was added N,N-diisopropylethylamine (2.9 mL, 16.8 mmol). The reaction mixture was stirred 10 minutes and then HATU (2.6 g, 6.74 mmol) was added in one portion. The reaction mixture was stirred 2 hours at room temperature. Aqueous saturated NaHCO₃ solution (30 mL) was added. The phases were separated, and the aqueous phase was extracted with DCM (2×50 mL). The organic phases were combined and passed through a phase separator (Biotage) before concentrating under vacuum. The residue was purified by flash column chromatography (SiO₂, Biotage Isolera, 100 g, Sfär column, eluting from 0% to 10% MeOH in DCM) affording tert-butyl rac-(2S,4R)-2-[(1-methylindazol-5-yl)methylcarbamoyl]-4-(p-tolylmethyl)pyrrolidine-1-carboxylate (2.0 g, 77%) as a white solid.

AnalpH9_MeCN_4 min, Rt: 2.46 min, m/z 363.3 [M+H-Boc]⁺

Step 2: tert-Butyl (2S,4R)-2-[(1-methylindazol-5-yl)methylcarbamoyl]-4-(p-tolylmethyl)pyrrolidine-1-carboxylate (2 g) was dissolved in DCM (50 mL) forming a clear yellow solution. TFA (22 mL) was added dropwise over 2 mins. The reaction mixture was stirred at room temperature 2.5 hours. The reaction mixture was concentrated in vacuo. The residue was dissolved in MeOH and purified via SCX-2 cartridge (50 g, Biotage). The cartridge was washed through with 3 CV of MeOH. Bound compound was eluted with 3 CV of 3.5M NH₃ in MeOH. The basic MeOH solution was concentrated in vacuo affording (2S,4R)—N-((1-methyl-1H-indazol-5-yl)methyl)-4-(4-methylbenzyl)pyrrolidine-2-carboxamide (1.16 g, 74% yield) as an off-white solid.

AnalpH9_MeCN_4 min, Rt: 2.30 min, m/z 363.3 [M+H]⁺

Step 3: To a stirred solution of (2R,3S)-3-(5-azaspiro[2.4]heptane-5-carbonyl)-1-benzyloxycarbonyl-piperidine-2-carboxylic acid (52 mg, 0.14 mmol) and (2S,4R)—N-[(1-methylindazol-5-yl)methyl]-4-(p-tolylmethyl)pyrrolidine-2-carboxamide (51 mg, 0.14 mmol) in DCM (2 mL) was added N,N-diisopropylethylamine (0.047 mL, 0.27 mmol). The reaction mixture was stirred at 0° C. for 10 min, and then HATU (61 mg, 0.16 mmol) was added. The reaction mixture was stirred at 0° C. for 1 hour. The reaction mixture was diluted with DCM (10 mL) and saturated aqueous solution of NaHCO₃ (10 mL) was added. The aqueous phase was extracted with DCM (3×10 mL). The organic combined phases were passed through a phase separator and concentrated under vacuum. The residue was purified by preparative HPLC. The fractions containing the desired product were concentrated, redissolved in 1:1 MeCN:water and lyophilised affording (2R,3S)-3-(5-azaspiro[2.4]heptane-5-carbonyl)-2-[(2S,4R)-2-[(1-methylindazol-5-yl)methylcarbamoyl]-4-(p-tolylmethyl)pyrrolidine-1-carbonyl]piperidine-1-carboxylate (91 mg, 93%) as a white solid.

AnalpH9_MeCN_4 min, Rt: 2.93 min, m/z 731.6 [M+H]⁺

Step 4: A stirred solution of benzyl (2R,3S)-3-(5-azaspiro[2.4]heptane-5-carbonyl)-2-[(2S,4R)-2-[(1-methylindazol-5-yl)methylcarbamoyl]-4-(p-tolylmethyl)pyrrolidine-1-carbonyl]piperidine-1-carboxylate (91 mg, 0.12 mmol) in ethanol (2.5 mL) at room temperature was placed under reduced pressure, filled with nitrogen and the process repeated a further 2 times. Palladium 5% weight on Carbon (13 mg, 0.13 mmol) was added and the reaction mixture was placed under reduced pressure, filled with hydrogen and the process repeated a further 2 times. The reaction mixture was stirred at room temperature until starting material was consumed (6 hours). The reaction mixture was filtered through a pad of celite, eluting with DCM. The combined filtrate and washings were concentrated under reduced pressure, affording 70 mg of crude product as colourless gum. The residue was purified by preparative HPLC. Fractions containing the desired product were concentrated under vacuum. The residue was redissolved in a 1:1 MeCN:water and lyophilised affording (2S,4R)-1-[(2R,3S)-3-(5-azaspiro[2.4]heptane-5-carbonyl)piperidine-2-carbonyl]-N-[(1-methylindazol-5-yl)methyl]-4-(p-tolylmethyl)pyrrolidine-2-carboxamide (50 mg, 67%) as a white solid.

AnalpH9_MeOH_QC_v1, Rt: 8.30 min, m/z 597.6 [M+H]⁺

AnalpH2_MeOH_QC_v1, Rt: 6.26 min, m/z 597.6 [M+H]⁺

¹H-NMR (400 MHz, DMSO-D₆) δ 8.58 (q, J=6.1 Hz, 0.6H), 8.08-8.13 (m, 0.4H), 7.94-7.96 (m, 1H), 7.51-7.58 (m, 2H), 7.22-7.29 (m, 1H), 7.04-7.10 (m, 4H), 5.19-5.21 (m, 0.6H), 4.25-4.42 (m, 2.3H), 3.98-4.05 (m, 0.5H), 4.01 (s, 3H), 3.36-3.59 (m, 2H), 3.25-3.30 (m, 1H), 3.05-3.17 (m, 2H), 2.75-2.99 (m, 2H), 2.54-2.68 (m, 1H), 2.13-2.37 (m, 1.5H), 2.26 (s, 3H), 1.88-2.03 (m, 1.3H), 1.64-1.85 (m, 4H), 1.50-1.61 (m, 0.4H), 1.23-1.40 (m, 1H), 1.08-1.15 (m, 0.5H), 0.47-0.60 (m, 4H)

The following compounds were made by analogous methods:

| | | | Mass, % |
|---|---|---|---|
| Example | | | yield, |
| No. | Structure & Conditions | Analytical Data | state |
| M05770 | 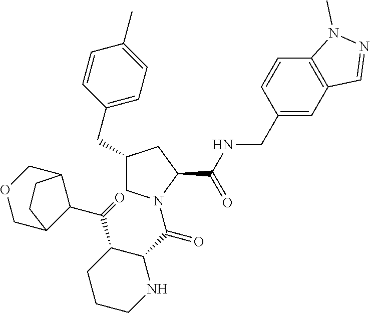 [SMILES: Cc1ccc(C[C@@H]2C[C@@H](C(=O)NCc3ccc4c(cnn4C)c3)N(C(=O)[C@@H]3NCCC[C@@H]3C(=O)C3C4CCC3COC4)C2)cc1] | AnalpH2_MeCN_QC_V 2, Rt: 4.60 min, m/z 613.4 [M + H]⁺ AnalpH9_MeCN_QC_V 2, Rt: 6.39 min, m/z 613.4 [M + H]⁺ ¹H-NMR (400 MHz, DMSO-D₆) δ 8.60 (t, J = 6.0 Hz, 0.6H), 8.06- 8.13 (s, 0.4H), 7.95-7.6 (m, 1H), 7.51-7.58 (m, 2H), 7.23-7.30 (m, 1H), 7.05-7.11 (m, 4H), 5.30 (t, J = 5.0 Hz, 0.4H), 5.18 (br d, J = 6.0 Hz, 0.2H), 4.22-4.46 (m, 3.4H), 3.98-4.18 (m, 1.3H), 4.01 (s, 3H), 3.83-3.89 (m, 0.3H), 3.72 (br d, J = 10.5 Hz, | 61.0 mg, 66%, off-white solid |
| | (2S,4R)-1-((2R,3S)-3-(3-oxa-8- | 0.2H), 3.35-3.60 (m, | |
| | azabicyclo[3.2.1]octane-8-carbonyl)piperidine- | 4H), 3.17 (br s, 0.4H), | |
| | 2-carbonyl)-N-((1-methyl-1H-indazol-5- | 2.75-3.01 (m, 3H), | |
| | yl)methyl)-4-(4-methylbenzyl)pyrrolidine-2- | 2.57-2.68 (m, 1H), | |
| | carboxamide | 2.11-2.41 (m, 1H), 2.26 | |
| | S1: (1-methyl-1H-indazol-5-yl)methanamine | (s, 3H), 1.18-1.93 (m, | |
| | S2: (2S,4R)-1-(tert-butoxycarbonyl)-4-(4- | 9H), 1.12 (m, 1H). | |
| | methylbenzyl)pyrrolidine-2-carboxylic acid | | |
| | S4: (2R,3S)-1-((benzyloxy)carbonyl)-3-(3-oxa- | | |
| | 8-azabicyclo[3.2.1]octane-8- | | |
| | carbonyl)piperidine-2-carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by prep HPLC | | |
| | Step 4: GM3. Final purification prep HPLC | | |
| M05775 | 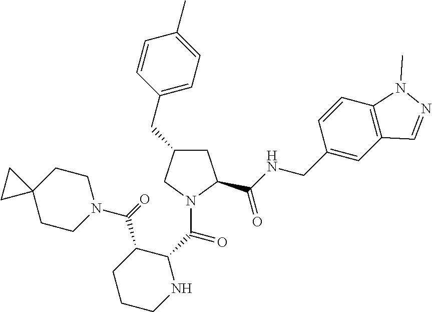 [SMILES: Cc1ccc(C[C@@H]2C[C@@H](C(=O)NCc3ccc4c(cnn4C)c3)N(C(=O)[C@@H]3NCCC[C@@H]3C(=O)N3CCC4(CC3)CC4)C2)cc1] | AnalpH2_MeCN_QC_V 2, Rt: 5.15 min, m/z 611.5 [M + H]⁺ AnalpH9_MeCN_QC_V 2, Rt: 7.32 min, m/z 611.4 [M + H]⁺ ¹H-NMR (400 MHz, DMSO-D₆) δ 8.55 (t, J = 6.0 Hz, 0.6H), 8.03 (t, J = 6.0 Hz, 0.4H), 7.94 (m, 1H), 7.57-7.51 (m, 2H), 7.29-7.22 (m, 1H), 7.08-7.04 (m, 4H), 5.27 (d, J = 6.0 Hz, 0.5H), 4.43-4.25 (m, 2H), 4.09-3.95 (m, 3H), 3.50-3.40 (m, 3H), 3.18 (t, J = 8.9 Hz, 0.4H), 3.04-2.87 (m, 3H), | 40.7 mg, 49%, white solid |
| | | 2.75-2.54 (m, 3H), | |
| | (2S,4R)-1-((2R,3S)-3-(6-azaspiro[2.5]octane-6- | 2.35-2.17 (m, 4H), | |
| | carbonyl)piperidine-2-carbonyl)-N-((1-methyl- | 2.07-1.11 (m, 10H), | |
| | 1H-indazol-5-yl)methyl)-4-(4- | 0.34 (s, 4H) | |
| | methylbenzyl)pyrrolidine-2-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-(6- | | |
| | azaspiro[2.5]octane-6-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by | | |
| | prep HPLC | | |
| | Step 4: GM3. Final purification prep HPLC. | | |
| M05777 | 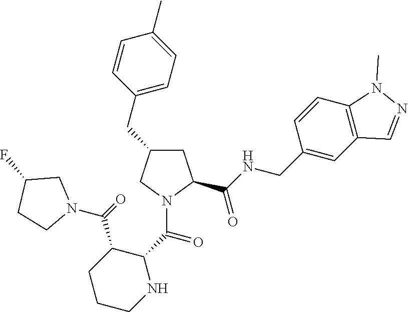 [SMILES: Cc1ccc(C[C@@H]2C[C@@H](C(=O)NCc3ccc4c(cnn4C)c3)N(C(=O)[C@@H]3NCCC[C@@H]3C(=O)N3CC[C@H](F)C3)C2)cc1] (2S,4R)-1-((2R,3S)-3-((S)-3-fluoropyrrolidine- 1-carbonyl)piperidine-2-carbonyl)-N-((1- methyl-1H-indazol-5-yl)methyl)-4-(4- methylbenzyl)pyrrolidine-2-carboxamide S1: (1-methyl-1H-indazol-5-yl)methanamine | AnalpH2_MeCN_QC_V 2, Rt: 4.70 min, m/z 589.4 [M + H]⁺ AnalpH9_MeCN_QC_V 2, Rt: 6.42 min, m/z 589.4 [M + H]⁺ ¹H-NMR (400 MHz, DMSO-D₆) δ 8.59 (t, J = 5.7 Hz, 0.6H), 8.02- 8.15 (m, 0.4H), 7.95- 7.96 (m, 1H), 7.51-7.57 (m, 2H), 7.22-7.30 (m, 1H), 7.02-7.10 (m, 4H), 5.14-5.43 (m, 1.6H), 4.23-4.43 (m, 2.3H), 3.93-4.09 (m, 0.5H), 4.01 (s, 3H), 3.42-3.77 (m, 4H), 2.90-3.28 (m, 2H), 2.55-2.80 (m, 1H), 1.89-2.40 (m, 4.3H), 2.26 (s, 3H), 1.44-1.71 (m, 2.4H), 1.21-1.44 (m, 1H), 1.02-1.16 (m, 0.6H) | 110.0 mg, 89%, white solid |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-((S)-3- | | |
| | fluoropyrrolidine-1-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by | | |
| | prep HPLC | | |
| | Step 4: GM3. Final purification prep HPLC. | | |
| M05778 | 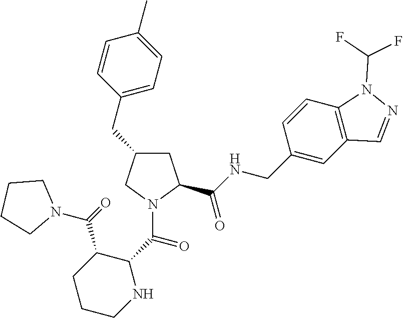 [SMILES: Cc1ccc(C[C@@H]2C[C@@H](C(=O)NCc3ccc4c(cnn4C(F)F)c3)N(C(=O)[C@@H]3NCCC[C@@H]3C(=O)N3CCCC3)C2)cc1] | AnalpH2_MeCN_QC_V 2, Rt: 5.06 min, m/z 607.5 [M + H]⁺ AnalpH9_MeCN_QC_V 2, Rt: 6.99 min, m/z 607.3 [M + H]⁺ ¹H-NMR (400 MHz, DMSO-D₆) δ 8.63-8.64 (m, 0.4H), 8.32-8.37 (m, 1H), 8.29-8.30 (m, 0.2H), 8.14-8.16 (m, 1H), 7.99-8.00 (m, 0.2H), 7.65-7.80 (m, 2H), 7.43 (dd, J = 22.7, 8.5 Hz, 1H), 7.02-7.07 (m, 4H), 5.19 (s, 0.5H), 4.28-4.42 (m, 2H), 4.00-4.03 (m, 0.4H), 3.43-3.55 (m, 2H), 3.11-3.25 (m, 3H), | 3.0 mg, 8%, off- white solid |
| | (2S,4R)-N-((1-(difluoromethyl)-1H-indazol-5- | 2.79-3.04 (m, 2H), | |
| | yl)methyl)-4-(4-methylbenzyl)-1-((2R,3S)-3- | 2.63-2.73 (m, 2H), | |
| | (pyrrolidine-1-carbonyl)piperidine-2- | 2.29-2.33 (m, 1H), 2.25 | |
| | carbonyl)pyrrolidine-2-carboxamide | (s, 3H), 1.65-2.14 (m, | |
| | S1: (1-(difluoromethyl)-1H-indazol-5- | 8H), 1.50-1.52 (m, | |
| | yl)methanamine | 0.5H), 1.13-1.28 (m, | |
| | S2: (2S,4R)-1-(tert-butoxycarbonyl)-4-(4- | 2H), 1.04 (d, J = 11.9 | |
| | methylbenzyl)pyrrolidine-2-carboxylic acid | Hz, 0.4H), 0.82-0.85 | |
| | S4: (2R,3S)-1-((benzyloxy)carbonyl)-3- | (m, 0.1H). | |
| | (pyrrolidine-1-carbonyl)piperidine-2-carboxylic | | |
| | acid | | |
| | Step 1: GM1 with DMF and DIPEA. Purified by | | |
| | column chromatography | | |
| | Step 2: GM2A. Purification a (SCX-2) | | |
| | Step 3: GM1 with DMF and DIPEA. Reaction | | |
| | mixture purified directly by prep HPLC | | |
| | Step 4: GM3. Final purification prep HPLC. | | |
| M05785 | 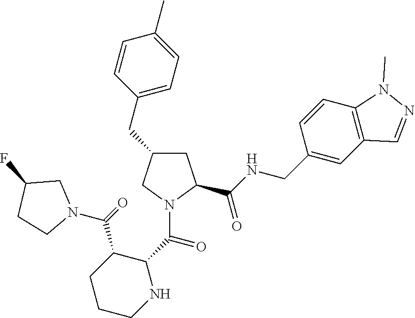 [SMILES: Cc1ccc(C[C@@H]2C[C@@H](C(=O)NCc3ccc4c(cnn4C)c3)N(C(=O)[C@@H]3NCCC[C@@H]3C(=O)N3CC[C@@H](F)C3)C2)cc1] (2S,4R)-1-((2R,3S)-3-((R)-3-fluoropyrrolidine- | AnalpH2_MeCN_QC_V 2, Rt: 4.84 min, m/z 589.4 [M + H]⁺ AnalpH9_MeCN_QC_V 2, Rt: 6.40 min, m/z 589.3 [M + H]⁺ ¹H-NMR (400 MHz, DMSO-D₆) δ 8.58 (m, 0.5H), 8.10 (dt, J = 27.3, 5.8 Hz, 0.3H), 7.96-7.90 (m, 1H), 7.58-7.51 (m, 2H), 7.26 (m, 1H), 7.08-6.96 (m, 4H), 5.41-5.20 (m, 2H), 4.43-4.19 (m, 2H), 4.04 (s, 3H), 3.73-3.39 (m, 4H), 3.24-2.77 (m, 3H), 2.69-2.54 (m, 2H), 2.42-2.31 (m, 1H), 2.26 (s, 3H), 2.20-1.24 (m, 7H), 1.12 (m, 1H) | 20.0 mg, 29%, white solid |
| | 1-carbonyl)piperidine-2-carbonyl)-N-((1- | | |
| | methyl-1H-indazol-5-yl)methyl)-4-(4- | | |
| | methylbenzyl)pyrrolidine-2-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-((R)-3- | | |
| | fluoropyrrolidine-1-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by | | |
| | prep HPLC | | |
| | Step 4: GM3. Final purification prep HPLC | | |
| M05787 | 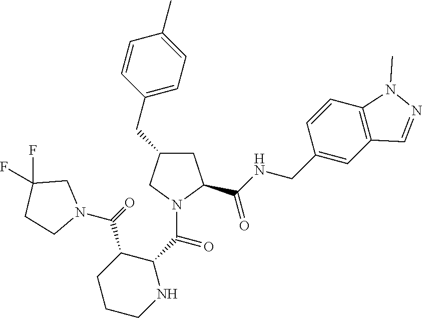 [SMILES: Cc1ccc(C[C@@H]2C[C@@H](C(=O)NCc3ccc4c(cnn4C)c3)N(C(=O)[C@@H]3NCCC[C@@H]3C(=O)N3CCC(F)(F)C3)C2)cc1] | AnalpH2_MeCN_QC_V 2, Rt: 5.00 min, m/z 607.4 [M + H]⁺ AnalpH9_MeCN_QC_V 2, Rt: 6.81 min, m/z 607.4 [M + H]⁺ ¹H-NMR (400 MHz, DMSO-D₆) δ 8.60-8.63 (m, 0.6H), 8.11 (t, J = 5.7 Hz, 0.4H), 7.86- 7.95 (m, 1H), 7.50-7.58 (m, 2H), 7.21-7.30 (m, 1H), 7.03-7.11 (m, 4H), 5.20 (t, J = 5.3 Hz, 1H), 4.25-4.44 (m, 2.4H), 3.34-4.08 (m, 4H), 4.01 (s, 3H), 2.90-3.27 (m, 3H), 2.54-2.71 (m, 2H), 2.13-2.44 (m, 2H), 2.25 | 70.0 mg, 62%, white solid |
| | | (s, 3H), 1.51-2.01 (m, | |
| | (2S,4R)-1-((2R,3S)-3-(3,3-difluoropyrrolidine-1- | 4.5H), 1.23-1.35 (m, | |
| | carbonyl)piperidine-2-carbonyl)-N-((1-methyl- | 1H), 1.07-1.15 (m, | |
| | 1H-indazol-5-yl)methyl)-4-(4- | 0.6H) | |
| | methylbenzyl)pyrrolidine-2-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-(3,3- | | |
| | difluoropyrrolidine-1-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by | | |
| | prep HPLC | | |
| | Step 4: GM3. Final purification prep HPLC | | |
| M05788 | 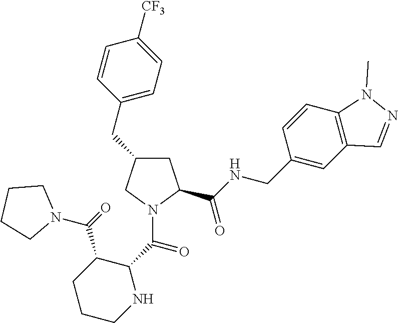 [SMILES: Cn1ncc2cc(CNC(=O)[C@@H]3C[C@@H](Cc4ccc(C(F)(F)F)cc4)CN3C(=O)[C@@H]3NCCC[C@@H]3C(=O)N3CCCC3)ccc21] (2S,4R)-N-((1-methyl-1H-indazol-5-yl)methyl)- 1-((2R,3S)-3-(pyrrolidine-1-carbonyl)piperidine- 2-carbonyl)-4-(4-(trifluoromethyl)benzyl)pyrrolidine-2-carboxamide S1: (1-methyl-1H-indazol-5-yl)methanamine S2: (2S,4R)-1-(tert-butoxycarbonyl)-4-(4- (trifluoromethyl)benzyl)pyrrolidine-2-carboxylic acid S4: (2R,3S)-1-((benzyloxy)carbonyl)-3- (pyrrolidine-1-carbonyl)piperidine-2-carboxylic acid Step 1: GM1 with DMF and TEA instead of DIPEA. Crude used without further purification | AnalpH2_MeCN_QC_V 2, Rt: 4.95 min, m/z 625.4 [M + H]⁺ AnalpH9_MeCN_QC_V 2, Rt: 6.71 min, m/z 625.4 [M + H]⁺ ¹H-NMR (400 MHz, DMSO-D₆) δ 8.60 (t, J = 6.0 Hz, 0.6H), 8.12 (t, J = 6.0 Hz, 0.4H), 7.97 (s, 1H), 7.66 (dd, J = 8.0, 5.3 Hz, 2H), 7.57 (t, J = 4.4 Hz, 1H), 7.53 (d, J = 8.2 Hz, 1H), 7.48-7.40 (m, 2H), 7.28 (dd, J = 8.9, 1.1 Hz, 0.6H), 7.23 (dd, J = 8.9, 1.1 Hz, 0.4H), 5.24 (t, J = 4.6 Hz, 0.6H), 4.44- 4.22 (m, 2.6H), 4.09- 3.99 (m, 3.8H), 3.58- 3.43 (m, 1H), 3.28-3.11 (m, 3H), 3.09-2.85 (m, 2H), 2.83-2.57 (m, 2H), 2.26-2.12 (m, 0.5H), 2.04-1.89 (m, 1.6H), 1.88-1.64 (m, 6H), 1.63-1.48 (m, 0.6H), 1.40-1.22 (m, 1H), 1.17-1.04 (m, 0.8H). | 35.0 mg, 35%, white solid |
| | Step 2: GM2A. Purification a (SCX-2). | | |
| | Step 3: GM1 with DMF and TEA. Crude used | | |
| | without further purification | | |
| | Step 4: GM3. Final purification prep HPLC | | |
| M05799 | 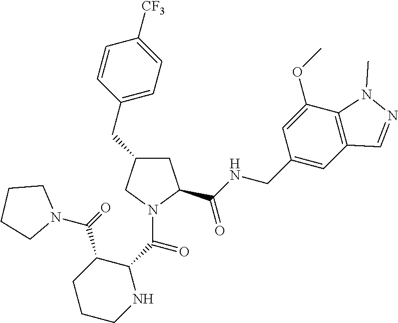 (2S,4R)-N-((7-methoxy-1-methyl-1H-indazol-5- yl)methyl)-4-(4-methylbenzyl)-1-((2R,3S)-3- (pyrrolidine-1-carbonyl)piperidine-2- | AnalpH2_MeOH_QC_V 1, Rt: 6.44 min, m/z 601.4 [M + H]⁺ AnalpH9_MeCN_QC_V 2, Rt: 6.70 min, m/z 601.4 [M + H]⁺ ¹H-NMR (400 MHz, DMSO-D₆) δ 8.52 (t, J = 6.2 Hz, 0.5H), 8.05 (t, J = 6.0 Hz, 0.5H), 7.83 (s, 1H), 7.07-6.99 (m, 5H), 6.71-6.54 (m, 1H), 5.21-5.13 (m, 0.5H), 4.33-4.16 (m, 2.3H), 4.13 (s, 3H), 4.02-3.92 (m, 0.5H), 3.83 (m, 3H), 3.54-3.34 (m, 1H), 3.24-3.06 (m, 2.3H), 3.04-2.82 (m, 2H), 2.74-2.49 (m, 3H), 2.22 (m, 3H), 1.98-1.44 (m, 8.8H), 1.31-1.17 (m, 1H), 1.13-0.98 (m, 0.8H). | 20.0 mg, 82%, white solid |
| | carbonyl)pyrrolidine-2-carboxamide | | |
| | S1: (7-methoxy-1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic acid | | |
| | Step 1: GM1 with with DMF and TEA instead | | |
| | of DIPEA. Purified by column chromatography | | |
| | Step 2: GM2A. Purification a (SCX-2) | | |
| | Step 3: GM1 with DMF and TEA. Crude used | | |
| | without further purification | | |
| | Step 4: GM3. Final purification prep HPLC | | |
| M05804 | 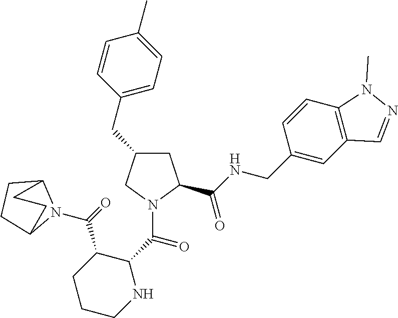 [SMILES: Cc1ccc(C[C@@H]2C[C@@H](C(=O)NCc3ccc4c(cnn4C)c3)N(C(=O)[C@@H]3NCCC[C@@H]3C(=O)N3C4CCC3CC4)C2)cc1] | AnalpH2_MeCN_QC_V 2, Rt: 4.89 min, m/z 597.4 [M + H]⁺ AnalpH9_MeCN_QC_V 2, Rt: 6.77 min, m/z 597.4 [M + H]⁺ ¹H-NMR (400 MHz, DMSO-D₆) δ 8.62-8.57 (t, J = 6.0 Hz, 0.5H), 8.11-8.06 (t, J = 0.40 Hz, 0.4H), 7.98-7.91 (m, 1H), 7.60-7.49 (m, 2H), 7.29-7.27 (dd, J = 8.7, 1.4 Hz, 0.5H), 7.25-7.22 (dd, J = 8.7, 1.4 Hz, 0.4H), 7.11-7.04 (m, 4H), 6.64 (s, 0.1H), 5.22-5.18 (m, 0.5H), 4.45-4.12 (m, 4H), 4.02 | 15.6 mg, 73%, white solid |
| | | (s, 3H), 3.95 (m, 0.3H), | |
| | (2S,4R)-1-((2R,3S)-3-(7- | 3.51-3.39 (m, 1H), 3.31 | |
| | azabicyclo[2.2.1]heptane-7- | (s, 1H), 3.28-3.14 (m, | |
| | carbonyl)piperidine-2-carbonyl)-N-((1-methyl- | 1H), 2.97-2.75 (m, 3H), | |
| | methylbenzyl)pyrrolidine-2-carboxamide | 2.67-2.54 (m, 2H), 2.37- | |
| | 1H-indazol-5-yl)methyl)-4-(4- | 2.29 (m, 1H), 2.26 (m, | |
| | S1: (1-methyl-1H-indazol-5-yl)methanamine | 3H), 2.21-2.00 (m, 1H), | |
| | S2: (2S,4R)-1-(tert-butoxycarbonyl)-4-(4- | 1.92-1.23 (m, 12H), | |
| | methylbenzyl)pyrrolidine-2-carboxylic acid | 1.13 (m, 1H) | |
| | S4: (2R,3S)-1-((benzyloxy)carbonyl)-3-(7- | | |
| | carbonyl)piperidine-2-carboxylic acid | | |
| | Step 1: GM1 with DMF and DIPEA. Purified by | | |
| | column chromatography. | | |
| | azabicyclo[2.2.1]heptane-7- | | |
| | Step 2: GM2A. Purified by SCX-2 | | |
| | Step 3: GM1 with DCM and DIPEA. Purified by | | |
| | prep HPLC | | |
| | Step 4: GM3. Final purification prep HPLC | | |
| M05806 | 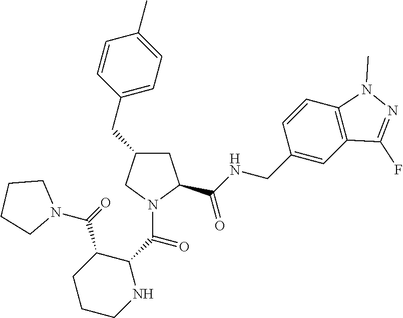 [SMILES: Cc1ccc(C[C@@H]2C[C@@H](C(=O)NCc3ccc4c(c3)c(F)nn4C)N(C(=O)[C@@H]3NCCC[C@@H]3C(=O)N3CCCC3)C2)cc1] | AnalpH2_MeCN_QC_V 2, Rt: 4.96 min, m/z 589.4 [M + H]⁺ AnalpH9_MeCN_QC_V 2, Rt: 6.99 min, m/z 589.4 [M + H]⁺ ¹H-NMR (400 MHz, DMSO-D₆) δ 8.62 (0.6H, t, J = 6.2 Hz), 8.13 (0.4H, t, J = 6.0 Hz), 7.52-7.60 (1H, m), 7.49 (1H, s), 7.31-7.38 (1H, m), 7.04-7.10 (4H, m), 5.23-5.25 (0.5H, m), 4.25-4.44 (2.4H, m), 3.96-4.00 (0.3H, m), 3.90 (3H, s), 3.37- 3.52 (1.5H, m), 3.04- 3.25 (3.3H, m), 2.88- 2.99 (2H, m), 2.54-2.77 | 13.0 mg, 32%, white solid |
| | (2S,4R)-N-((3-fluoro-1-methyl-1H-indazol-5- | (2.6H, m), 2.20-2.28 | |
| | yl)methyl)-4-(4-methylbenzyl)-1-((2R,3S)-3- | (4.3H, m), 1.69-2.02 | |
| | (pyrrolidine-1-carbonyl)piperidine-2- | (7.5H, m), 1.52-1.58 | |
| | carbonyl)pyrrolidine-2-carboxamide | (0.7H, m), 1.26-1.37 | |
| | S1: (3-fluoro-1-methyl-1H-indazol-5- | (1H, m), 1.09-1.14 | |
| | yl)methanamine | (0.7H, m) | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic acid | | |
| | Step 1: GM1 with DMF and DIPEA. Purified by | | |
| | column chromatography | | |
| | Step 2: GM2A. Purification a (SCX-2) | | |
| | Step 3: GM1 with DMF and DIPEA. Reaction | | |
| | mixture purified directly by prep HPLC | | |
| | Step 4: GM3. Final purification prep HPLC | | |
| M05809 | 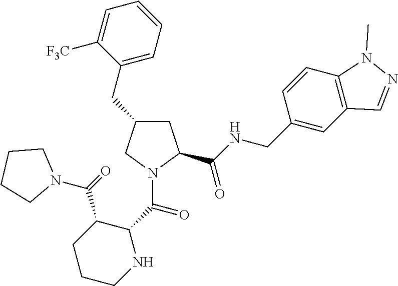 [SMILES: Cn1ncc2cc(CNC(=O)[C@@H]3C[C@@H](Cc4ccccc4C(F)(F)F)CN3C(=O)[C@@H]3NCCC[C@@H]3C(=O)N3CCCC3)ccc21] | AnalpH2_MeCN_QC_V 2, Rt: 4.86 min, m/z 625.5 [M + H]⁺ AnalpH9_MeCN_QC_V 2, Rt: 6.62 min, m/z 625.4 [M + H]⁺ | 6.2 mg, 39%, white solid |
| | (2S,4R)-N-((1-methyl-1H-indazol-5-yl)methyl)- | | |
| | 1-((2R,3S)-3-(pyrrolidine-1-carbonyl)piperidine- | | |
| | 2-carbonyl)-4-(2- | | |
| | (trifluoromethyl)benzyl)pyrrolidine-2- | | |
| | carboxamide | | |
| | S1: (1-methyl-1H-indazol-5-yl)methanamine | | |
| | S2: (2S,4R)-1-(tert-butoxycarbonyl)-4-(2- | | |
| | (trifluoromethyl)benzyl)pyrrolidine-2-carboxylic | | |
| | acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DMF and TEA. Crude used | | |
| | without further purification | | |
| | Step 2: GM2A. Purification a (SCX-2) | | |
| | Step 3: GM1 with DMF and TEA. Crude used | | |
| | without further purification | | |
| | Step 4: GM3. Final purification prep HPLC | | |
| M05810 | 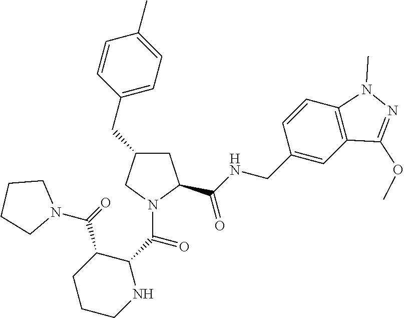 [SMILES: COc1nn(C)c2ccc(CNC(=O)[C@@H]3C[C@@H](Cc4ccc(C)cc4)CN3C(=O)[C@@H]3NCCC[C@@H]3C(=O)N3CCCC3)cc12] | AnalpH2_MeCN_QC_V 2, Rt: 5.00 min, m/z 601.4 [M + H]⁺ AnalpH9_MeCN_QC_V 2, Rt: 6.77 min, m/z 601.4 [M + H]⁺ ¹H-NMR (400 MHz, DMSO-D₆) δ 8.58 (t, J = 6.0 Hz, 0.7H), 8.08 (t, J = 5.7 Hz, 0.3H), 7.47- 7.35 (m, 2H), 7.25 (qd, J = 8.5, 1.4 Hz, 1H), 7.11-7.03 (m, 4H), 5.23 (q, J = 3.2 Hz, 0.6H), 4.45-4.18 (m, 2.4H), 4.02-3.93 (m, 3.4H), 3.83 (s, 3H), 3.56-3.40 (m, 0.9H), 3.29-3.11 (m, 2H), 3.08-2.80 (m, | 23.0 mg, 66%, white solid |
| | | 3H), 2.78-2.54 (m, 2H), | |
| | (2S,4R)-N-((3-methoxy-1-methyl-1H-indazol-5- | 2.47 (s, 0.6H), 2.37- | |
| | yl)methyl)-4-(4-methylbenzyl)-1-((2R,3S)-3- | 2.17 (m, 4.3H), 2.04- | |
| | (pyrrolidine-1-carbonyl)piperidine-2- | 1.65 (m, 7.4H), 1.63- | |
| | carbonyl)pyrrolidine-2-carboxamide | 1.49 (m, 0.5H), 1.42- | |
| | S1: (3-methoxy-1-methyl-1H-indazol-5- | 1.22 (m, 1H), 1.11 (d, | |
| | yl)methanamine | J = 10.5 Hz, 0.6H) | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic acid | | |
| | Step 1: GM1 with DMF and DIPEA. Purified by prep HPLC | | |
| | Step 2: GM2A. Purification a (SCX-2) | | |
| | Step 3: GM1 with DMF and DIPEA. Reaction | | |
| | mixture purified directly by prep HPLC | | |
| | Step 4: GM3. Final purification prep HPLC | | |
| M05811 | 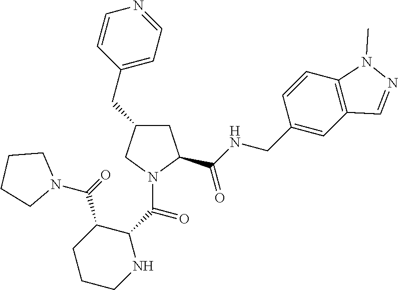 [SMILES: Cn1ncc2cc(CNC(=O)[C@@H]3C[C@@H](Cc4ccncc4)CN3C(=O)[C@@H]3NCCC[C@@H]3C(=O)N3CCCC3)ccc21] (2S,4R)-N-((1-methyl-1H-indazol-5-yl)methyl)- 4-(pyridin-4-ylmethyl)-1-((2R,3S)-3- (pyrrolidine-1-carbonyl)piperidine-2- carbonyl)pyrrolidine-2-carboxamide S1: (1-methyl-1H-indazol-5-yl)methanamine S2: (2S,4R)-1-(tert-butoxycarbonyl)-4-(pyridin- 4-ylmethyl)pyrrolidine-2-carboxylic acid S4: (2R,3S)-1-((benzyloxy)carbonyl)-3- (pyrrolidine-1-carbonyl)piperidine-2-carboxylic acid Step 1: GM1a with DMF and DIPEA. Purified | AnalpH2_MeCN_QC_V 2, Rt: 3.17 min, m/z 558.4 [M + H]⁺ AnalpH9_MeCN_QC_V 2, Rt: 5.10 min, m/z 558.4 [M + H]⁺ ¹H-NMR (400 MHz, DMSO-D₆) δ 8.60 (t, J = 6.0 Hz, 0.6H), 8.46 (2H, td, J = 4.5, 1.5 Hz), 8.12 (0.4H, t, J = 6.2 Hz), 7.97 (1H, s), 7.51- 7.58 (2H, m), 7.21-7.30 (3H, m), 5.24 (0.6H, t, J = 4.8 Hz), 4.24-4.42 (2.3H, m), 4.02-4.07 (3.3H, m), 3.47-3.51 (1H, m), 3.36-3.43 (0.7H, m), 3.12-3.27 (3H, m), 2.84-3.06 (2.5H, m), 2.57-2.74 (3.4H, m), 2.34-2.43 (0.7H, m), 2.15-2.24 (0.5H, m), 1.66-1.99 (7.6H, m), 1.50-1.57 (0.5H, m), 1.28-1.38 (1H, m), 1.08-1.16 (0.6H, m) | 25.8 mg, 59%, white solid |
| | by column chromatography | | |
| | Step 2: GM2A. Purification a (SCX-2). | | |
| | Step 3: GM1 with DMF and DIPEA. Purified by prep HPLC. | | |
| | Step 4: GM3. Final purification prep HPLC | | |
| M05820 | 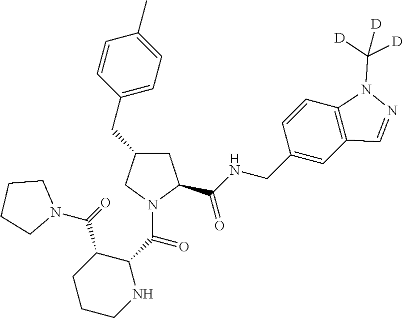 [SMILES: [2H]C([2H])([2H])n1ncc2cc(CNC(=O)[C@@H]3C[C@@H](Cc4ccc(C)cc4)CN3C(=O)[C@@H]3NCCC[C@@H]3C(=O)N3CCCC3)ccc21] | AnalpH2_MeCN_QC_V 2, Rt: 6.47 min, m/z 574.4 [M + H]⁺ AnalpH9_MeCN_QC_V 2, Rt: 4.72 min, m/z 574.4 [M + H]⁺ ¹H-NMR (400 MHz, DMSO-D₆) δ 8.58 (t, J = 6.0 Hz, 0.6H), 8.09 (t, J = 6.0 Hz, 0.4H), 7.95 (s, 1H), 7.51-7.57 (m, 2H), 7.20-7.27 (m, 1H), 7.03-7.10 (m, 4H), 5.19-5.21 (m, 0.5H), 4.23-4.41 (m, 2H), 3.97-4.01 (m, 0.4H), 3.43-3.54 (m, 1H), 3.35-3.41 (m, 1H), 3.10-3.26 (m, 2H), | 19.6 mg, 65%, white solid |
| | | 2.81-3.06 (m, 2H), | |
| | (2S,4R)-N-((1-(methyl-d3)-1H-indazol-5- | 2.72-2.74 (m, 0.4H), | |
| | yl)methyl)-4-(4-methylbenzyl)-1-((2R,3S)-3- | 2.53-2.67 (m, 2H), | |
| | (pyrrolidine-1-carbonyl)piperidine-2- | 2.28-2.32 (m, 0.4H), | |
| | carbonyl)pyrrolidine-2-carboxamide | 2.25 (s, 3H), 2.14-2.20 | |
| | S1: (1-(methyl-d3)-1H-indazol-5- | (m, 0.3H), 1.63-1.99 | |
| | yl)methanamine | (m, 8H), 1.49-1.59 (m, | |
| | S2: (2S,4R)-1-(tert-butoxycarbonyl)-4-(4- | 0.5H), 1.22-1.35 (m, | |
| | methylbenzyl)pyrrolidine-2-carboxylic acid | 1H), 1.06-1.13 (m, | |
| | S4: (2R,3S)-1-((benzyloxy)carbonyl)-3- | 0.6H) | |
| | (pyrrolidine-1-carbonyl)piperidine-2-carboxylic | | |
| | acid | | |
| | Step 1: GM1 with DMF and DIPEA. Purified by | | |
| | column chromatography. | | |
| | Step 2: GM2A. Purification a (SCX-2). | | |
| | Step 3: GM1 with DMF and DIPEA. Reaction | | |
| | mixture purified directly by prep HPLC | | |
| | Step 4: GM3. Final purification prep HPLC | | |
| M05836 | 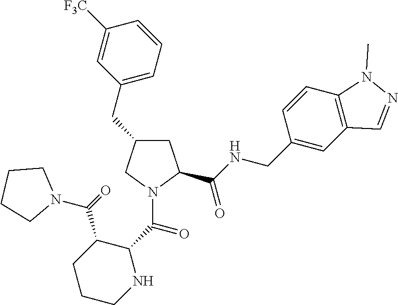 [SMILES: Cn1ncc2cc(CNC(=O)[C@@H]3C[C@@H](Cc4cccc(C(F)(F)F)c4)CN3C(=O)[C@@H]3NCCC[C@@H]3C(=O)N3CCCC3)ccc21] (2S,4R)-N-((1-methyl-1H-indazol-5-yl)methyl)- 1-((2R,3S)-3-(pyrrolidine-1-carbonyl)piperidine- 2-carbonyl)-4-(3- (trifluoromethyl)benzyl)pyrrolidine-2-carboxamide S1: (1-methyl-1H-indazol-5-yl)methanamine S2: (2S,4R)-1-(tert-butoxycarbonyl)-4-(3- (trifluoromethyl)benzyl)pyrrolidine-2-carboxylic acid | UPLC_pH2_MeCN_QC V1, Rt: 3.43 min, m/z 625.3 [M + H]⁺ UPLC_pH10_MeCN_QC_ V1, Rt: 4.48 min, m/z 625.3 [M + H]⁺ ¹H-NMR (400 MHz, DMSO-D₆) δ 8.57 (t, J = 6.0 Hz, 0.5H), 8.08 (t, J = 6.0 Hz, 0.4H), 7.91 (s, 1H), 7.55-7.46 (m, 6H), 7.25- 7.22 (dd, J = 8.7, 1.4 Hz, 0.5H), 7.20-7.16 (dd, J = 8.7, 1.4 Hz, 0.3H), 5.20 (t, J = 4.6 Hz, 0.5H), 4.38- 4.18 (m, 2H), 4.01-3.95 (m, 3H), 3.52-3.36 (m, 2H), 3.27 (s, 1H), 3.23- 3.07 (m, 3H), 3.01-2.66 (m, 4H), 2.63-2.51 (m, 1H), 2.50 (s, 0.2H), 2.37- 2.27 (m, 0.6H), 2.15 (m, 0.5H), 1.96-1.60 (m, 7H), 1.58-1.44 (m, 0.5H), 1.32- 1.19 (m, 1H), 1.11-1.04 (m, 1H) | 23.1 mg, 35%, white solid |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DCM and DIPEA. Crude | | |
| | used without further purification | | |
| | Step 2: GM2A. Purification a (SCX-2). | | |
| | Step 3: GM1 with DCM and DIPEA. Purified by | | |
| | prep HPLC | | |
| | Step 4: GM3. Final purification prep HPLC | | |
| M05837 | 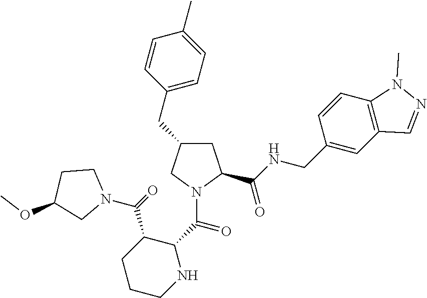 [SMILES: CO[C@H]1CCN(C(=O)[C@H]2CCCN[C@H]2C(=O)N2C[C@H](Cc3ccc(C)cc3)C[C@H]2C(=O)NCc2ccc3c(cnn3C)c2)C1] | UPLC_pH2_MeCN_QC_ V1, Rt: 3.23 min, m/z 601.3 [M + H]⁺ UPLC_pH10_MeCN_QC_ V1, Rt: 4.20 min, m/z 601.3 [M + H]⁺ ¹H-NMR (400 MHz, DMSO-D₆) δ 8.56 (m, 0.5H), 8.08 (m, 0.3H), 7.92-7.85 (m, 1H), 7.54- 7.42 (m, 2H), 7.31-7.18 (m, 1H), 7.07-6.90 (m, 4H), 5.15 (s, 0.5H), 4.40- 4.13 (m, 2H), 3.99-3.90 (m, 4H), 3.81 (s, 0.5H), 3.67 (m, 0.1H), 3.57-3.34 (m, 2H), 3.26 (s, 1H), 3.24- 3.16 (m, 4H), 3.12-2.70 | 15.0 mg, 43%, white solid |
| | | (m, 3H), 2.63-2.50 (m, | |
| | (2S,4R)-1-((2R,3S)-3-((S)-3- | 2H), 2.29 (m, 0.5H), 2.21 | |
| | methoxypyrrolidine-1-carbonyl)piperidine-2- | (m, 3H), 2.01-1.47 (m, | |
| | carbonyl)-N-((1-methyl-1H-indazol-5- | 6H), 1.32-1.19 (m, 1H), | |
| | yl)methyl)-4-(4-methylbenzyl)pyrrolidine-2- | 1.11-1.04 (m, 0.6H) | |
| | carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-((S)-3- | | |
| | methoxypyrrolidine-1-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Purified by | | |
| | prep HPLC | | |
| | Step 4: GM3. Final purification prep HPLC | | |
| M05838 Config a | 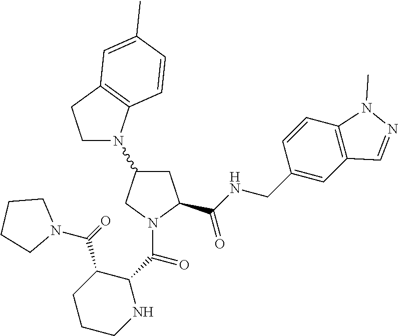 [SMILES: Cc1ccc2c(c1)CCN2C1C[C@@H](C(=O)NCc2ccc3c(cnn3C)c2)N(C(=O)[C@@H]2NCCC[C@@H]2C(=O)N2CCCC2)C1] (2S)-N-((1-methyl-1H-indazol-5-yl)methyl)-4- (5-methylindolin-1-yl)-1-((2R,3S)-3-(pyrrolidine- 1-carbonyl)piperidine-2-carbonyl)pyrrolidine-2- carboxamide (diastereomer A) S1: (1-methyl-1H-indazol-5-yl)methanamine | UPLC_pH2_MeCN_QC_ V1, Rt: 3.32 min, m/z 598.3 [M + H]⁺ UPLC_pH10_MeCN_QC V1, Rt: 4.37 min, m/z 598.4 [M + H]⁺ ¹H-NMR (400 MHz, DMSO-D₆) δ 8.62 (t, J = 6.0 Hz, 0.5H), 8.10 (t, J = 6.0 Hz, 0.5H), 7.96 (d, J = 11.4 Hz, 1H), 7.62-7.52 (m, 2H), 7.30 (qd, J = 9.0, 1.4 Hz, 1H), 6.84 (s, 1H), 6.78 (d, J = 7.8 Hz, 1H), 6.48 (dd, J = 10.8, 8.0 Hz, 1H), 5.17 (t, J = 8.0 Hz, 0.5H), 4.45-4.27 (m, 2.5H), 4.03 (t, J = 7.8 Hz, 4H), 3.95-3.82 (m, 1H), 3.65-3.52 (m, 1H), 3.46- 3.40 (m, 1H), 3.27-3.13 (m, 4H), 3.06-3.01 (m, 1H), 2.89-2.65 (m, 5H), 2.16-2.07 (m, 3H), 1.98- 1.61 (m, 7H), 1.51-1.23 (m, 2H) | 18.0 mg, 65%, white solid |
| | S2: (2S)-1-(tert-butoxycarbonyl)-4-(5- | | |
| | methylindolin-1-yl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1b with DMF and DIPEA. Purified | | |
| | by column chromatography and preparative | | |
| | HPLC | | |
| | Step 2: GM2A. No purification. Crude material | | |
| | used. | | |
| | Step 3: GM1 with DCM/DMF and DIPEA. | | |
| | Purified by prep HPLC. | | |
| | Step 4: GM3. Final purification prep HPLC | | |
| M05839 Config b | 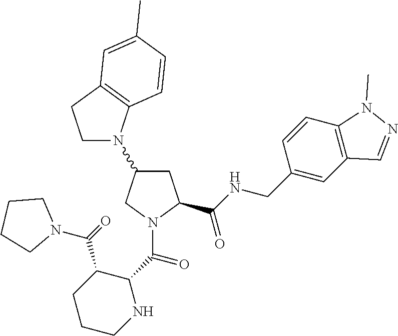 [SMILES: Cc1ccc2c(c1)CCN2C1C[C@@H](C(=O)NCc2ccc3c(cnn3C)c2)N(C(=O)[C@@H]2NCCC[C@@H]2C(=O)N2CCCC2)C1] | UPLC_pH2_MeCN_QC V1, Rt: 3.33 min, m/z 598.4 [M + H]⁺ UPLC_pH10_MeCN_QC_ V1, Rt: 4.36 min, m/z 598.3 [M + H]⁺ | 10.0 mg, quantitative white solid 9.0 mg, 46%, |
| | ((2S)-N-((1-methyl-1H-indazol-5-yl)methyl)-4- | | |
| | (5-methylindolin-1-yl)-1-((2R,3S)-3-(pyrrolidine- | | |
| | 1-carbonyl)piperidine-2-carbonyl)pyrrolidine-2- | | |
| | carboxamide (diastereoisomer B) | | |
| | S1: (1-methyl-1H-indazol-5-yl)methanamine | | |
| | S2: (2S)-1-(tert-butoxycarbonyl)-4-(5- | | |
| | methylindolin-1-yl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1b with DMF and DIPEA. Purified | | |
| | by column chromatography and preparative | | |
| | HPLC | | |
| | Step 2: GM2A. No purification. Crude material | | |
| | used | | |
| | Step 3: GM1 with DCM/DMF and DIPEA. | | |
| | Purified by prep HPLC | | |
| | Step 4: GM3. No final purification after celite | | |
| | filtration. | | |
| M05840 | 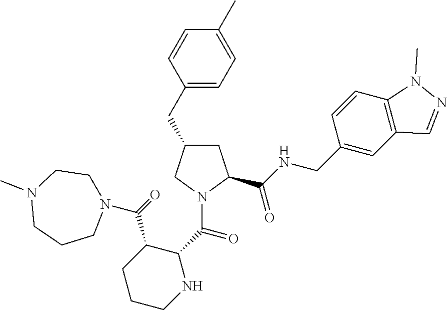 [SMILES: Cc1ccc(C[C@@H]2C[C@@H](C(=O)NCc3ccc4c(cnn4C)c3)N(C(=O)[C@@H]3NCCC[C@@H]3C(=O)N3CCCN(C)CC3)C2)cc1] (2S,4R)-1-((2R,3S)-3-(4-methyl-1,4-diazepane- 1-carbonyl)piperidine-2-carbonyl)-N-((1- methyl-1H-indazol-5-yl)methyl)-4-(4- | UPLC_pH2_MeCN_QC_ V1, Rt: 2.37 min, m/z 614.4 [M + H]⁺ UPLC_pH10_MeCN_QC_ V1, Rt: 4.15 min, m/z 614.4 [M + H]⁺ ¹H-NMR (400 MHz, DMSO-D₆) δ 8.56-8.61 (m, 0.6H), 8.07-8.12 (m, 0.4H), 7.95 (d, J = 5.0 Hz, 1H), 7.51-7.58 (m, 2H), 7.26 (ddd, J = 20.3, 8.8, 1.3 Hz, 1H), 7.04-7.10 (m, 4H), 5.25 (br s, 0.6H), 4.22-4.44 (m, 2.4H), 3.95- 4.05 (m, 0.5H), 4.01 (s, 3H), 3.38-3.54 (m, 2H), 2.82-3.24 (m, 3H), 2.55- 2.77 (m, 1H), 2.29-2.45 (1H), 2.09-2.29 (m, 6H), 1.59-1.98 (m, 5H), 1.05- 1.52 (m, 2H). | 9.0 mg, 46%, white solid |
| | methylbenzyl)pyrrolidine-2-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-(4- | | |
| | methyl-1,4-diazepane-1-carbonyl)piperidine-2- | | |
| | carboxylic acid | | |
| | Step 1: GM1 with DMF and DIPEA. Purified by | | |
| | column chromatography. | | |
| | Step 2: GM2A. Purification a (SCX-2) | | |
| | Step 3: GM1 with DCM and DIPEA. Crude | | |
| | used without further purification | | |
| | Step 4: GM3. Final purification prep HPLC | | |
| M05853 | 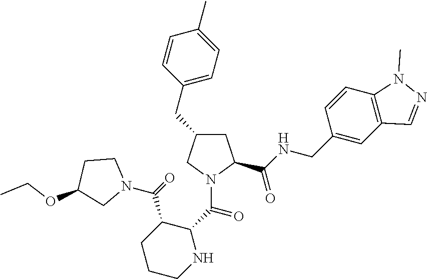 [SMILES: CCO[C@H]1CCN(C(=O)[C@H]2CCCN[C@H]2C(=O)N2C[C@H](Cc3ccc(C)cc3)C[C@H]2C(=O)NCc2ccc3c(cnn3C)c2)C1] | UPLC_pH2_MeCN_QC V1, Rt: 3.36 min, m/z 615.3 [M + H]⁺ UPLC_pH10_MeCN_QC V1, Rt: 4.22 min, m/z 615.3 [M + H]⁺ | 30.5 mg, 81%, white solid |
| | (2S,4R)-1-((2R,3S)-3-((S)-3-ethoxypyrrolidine- | | |
| | 1-carbonyl)piperidine-2-carbonyl)-N-((1- | | |
| | methyl-1H-indazol-5-yl)methyl)-4-(4- | | |
| | methylbenzyl)pyrrolidine-2-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3-((S)-3- | | |
| | ethoxypyrrolidine-1-carbonyl)piperidine-2- | | |
| | carboxylic acid | | |
| | Step 1: GM1 with DMF and DIPEA. Purified by | | |
| | column chromatography | | |
| | Step 2: GM2A. Purification a (SCX-2) | | |
| | Step 3: GM1 with DCM and DIPEA. Purified by | | |
| | prep HPLC | | |
| | Step 4: GM3. Final purification SCX-2 | | |
| M05863 | 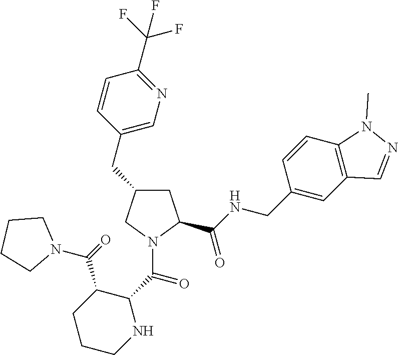 [SMILES: Cn1ncc2cc(CNC(=O)[C@@H]3C[C@@H](Cc4ccc(C(F)(F)F)nc4)CN3C(=O)[C@@H]3NCCC[C@@H]3C(=O)N3CCCC3)ccc21] (2S,4R)-N-((1-methyl-1H- benzo[d][1,2,3]triazol-5-yl)methyl)-1-((2R,3S)- 3-(pyrrolidine-1-carbonyl)piperidine-2- carbonyl)-4-((6-(trifluoromethyl)pyridin-3- yl)methyl)pyrrolidine-2-carboxamide | UPLC_pH2_MeCN_QC_ V1, Rt: 3.17 min, m/z 627.3 [M + H]⁺ UPLC_pH10_MeCN_QC_ V1, Rt: 4.16 min, m/z 627.3 [M + H]⁺ ¹H-NMR (400 MHz, DMSO-D₆) δ 8.64-8.69 (1.6H, m), 8.20 (0.4H, t, J = 6.0 Hz), 7.93-7.98 (1H, m), 7.72-7.86 (3H, m), 7.42 (1H, dd, J = 16.0, 9.2 Hz), 5.27-5.29 (0.5H, m), 4.31-4.49 (2.4H, m), 4.28 (3H, d, J = 3.2 Hz), 4.03- 4.07 (0.3H, m), 3.46-3.54 (1H, m), 3.37 (1.2H, dd, J = 11.9, 6.9 Hz), 3.12-3.27 (2.7H, m), 2.73-3.06 (5.6H, m), 2.58-2.67 (1H, m), 2.39-2.46 (0.5H, m), 2.20-2.29 (0.4H, m), 1.95- 1.99 (1.6H, m), 1.50-1.87 (6.9H, m), 1.25-1.40 (1H, m), 1.10-1.15 (0.6H, m). | 25.8 mg, 56%, white solid |
| | S1: (1-methyl-1H-benzo[d][1,2,3]triazol-5- | | |
| | yl)methanamine (Used as the HCl salt) | | |
| | S2: (2S,4R)-1-(tert-butoxycarbonyl)-4-((6- | | |
| | (trifluoromethyl)pyridin-3-yl)methyl)pyrrolidine- | | |
| | 2-carboxylic acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DMF and DIPEA. Purified by | | |
| | column chromatography. Amine used as the | | |
| | HCl salt. | | |
| | Step 2: GM2A. Purification a (SCX-2). | | |
| | Step 3: GM1 with DMF and DIPEA. Reaction | | |
| | mixture directly purified by prep HPLC | | |
| | Step 4: GM3 final purification SCX-2 | | |
| M05874 | 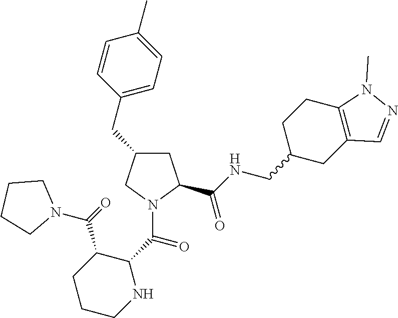 [SMILES: Cc1ccc(C[C@@H]2C[C@@H](C(=O)NCC3CCc4c(cnn4C)C3)N(C(=O)[C@@H]3NCCC[C@@H]3C(=O)N3CCCC3)C2)cc1] | UPLC_pH2_MeCN_QC V1, Rt: 3.32 min, m/z 575.3 [M + H]⁺ UPLC_pH10_MeCN_QC_ V1, Rt: 4.48 min, m/z 575.4 [M + H]⁺ | 11.4 mg, 39%, off-white solid |
| | (2S,4R)-N-((1-methyl-4,5,6,7-tetrahydro-1H- | | |
| | indazol-5-yl)methyl)-4-(4-methylbenzyl)-1- | | |
| | ((2R,3S)-3-(pyrrolidine-1-carbonyl)piperidine-2- | | |
| | carbonyl)pyrrolidine-2-carboxamide | | |
| | S1: (1-methyl-4,5,6,7-tetrahydro-1H-indazol-5- | | |
| | yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DCM and DIPEA. Purified by | | |
| | column chromatography | | |
| | Step 2: GM2A. Purification a (SCX-2) | | |
| | Step 3: GM1 with DCM and DIPEA. Purified by | | |
| | column chromatography | | |
| | Step 4: GM3. final purification prep HPLC. | | |
| | Obtained as a mixture of diastereoisomers | | |
| M05875 | 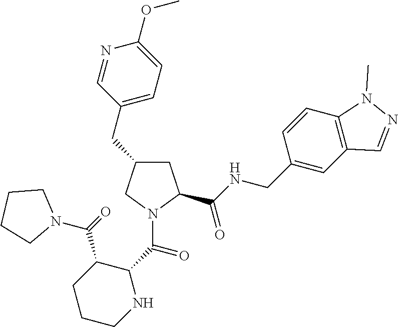 [SMILES: COc1ccc(C[C@@H]2C[C@@H](C(=O)NCc3ccc4c(cnn4C)c3)N(C(=O)[C@@H]3NCCC[C@@H]3C(=O)N3CCCC3)C2)cn1] (2S,4R)-4-((6-methoxypyridin-3-yl)methyl)-N- ((1-methyl-1H-indazol-5-yl)methyl)-1-((2R,3S)- 3-(pyrrolidine-1-carbonyl)piperidine-2- carbonyl)pyrrolidine-2-carboxamide S1: (1-methyl-1H-indazol-5-yl)methanamine | UPLC_pH2_MeCN_QC V1, Rt: 3.02 min, m/z 588.3 [M + H]⁺ UPLC_pH10_MeCN_QC V1, Rt: 3.99 min, m/z 588.4 [M + H]⁺ ¹H-NMR (400 MHz, DMSO-D6) δ 8.57 (t, J = 6.0 Hz, 0.6H), 8.05-8.09 (m, 0.4H), 7.93-8.00 (m, 2H), 7.50-7.63 (m, 3H), 7.21-7.35 (m, 1H), 6.69- 6.75 (m, 1H), 5.20-5.23 (m, 0.4H), 4.19-4.43 (m, 2H), 4.00-4.03 (m, 3H), 3.80-3.83 (m, 3H), 3.43- 3.53 (m, 1H), 3.33-3.41 (m, 2H), 3.11-3.24 (m, 2H), 2.85-3.06 (m, 2H), 2.73-2.74 (m, 0.3H), 2.54- 2.67 (m, 2H), 2.18-2.33 (m, 1H), 1.66-1.98 (m, 7H), 1.52-1.57 (m, 0.5H), 1.26-1.35 (m, 1H), 1.07- 1.14 (m, 0.5H) | 11.0 mg, 64%, white solid |
| | S2: (2S,4R)-1-(tert-butoxycarbonyl)-4-((6- | | |
| | methoxypyridin-3-yl)methyl)pyrrolidine-2- | | |
| | carboxylic acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DCM and DIPEA. Purified by | | |
| | column chromatography | | |
| | Step 2: GM2A. Purification a (SCX-2) | | |
| | Step 3: GM1 with DCM and DIPEA. Reaction | | |
| | mixture directly purified by prep HPLC | | |
| | Step 4: GM3. Final purification SCX-2 | | |
| M05877 | 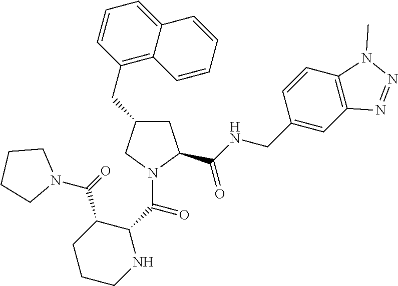 [SMILES: Cn1nnc2cc(CNC(=O)[C@@H]3C[C@@H](Cc4cccc5ccccc45)CN3C(=O)[C@@H]3NCCC[C@@H]3C(=O)N3CCCC3)ccc21] (2S,4R)-N-((1-methyl-1H- benzo[d][1,2,3]triazol-5-yl)methyl)-4- (naphthalen-1-ylmethyl)-1-((2R,3S)-3- | UPLC_pH2_MeCN_QC_ V1, Rt: 3.53 min, m/z 608.3 [M + H]+ UPLC_pH10_MeCN_QC V1, Rt: 4.67 min, m/z 608.5 [M + H]+ ¹H-NMR (400 MHz, DMSO-D6) δ 8.67 (t, J = 6.2 Hz, 0.5H), 8.18 (t, J = 6.0 Hz, 0.3H), 8.10 (m, 1H), 7.94-7.92 (m, 1H), 7.85-7.79 (m, 2H), 7.70- 7.31 (m, 6H), 5.30 (d, J = 7.8 Hz, 0.5H), 4.46-4.33 (m, 2H), 4.30-4.27 (m, 3H), 4.04-3.99 (m, 0.4H), 3.50-3.41 (m, 1H), 3.27- 2.91 (m, 6H), 2.80-2.65 (m, 1H), 2.33-1.54 (m, 11H), 1.37-1.23 (m, 1H), 1.11 (m, 1H) | 28.0 mg, 41%, white solid |
| | (pyrrolidine-1-carbonyl)piperidine-2- | | |
| | carbonyl)pyrrolidine-2-carboxamide | | |
| | S1: (1-methyl-1H-benzo[d][1,2,3]triazol-5- | | |
| | yl)methanamine | | |
| | S2: (2S,4R)-1-(tert-butoxycarbonyl)-4- | | |
| | (naphthalen-1-ylmethyl)pyrrolidine-2-carboxylic | | |
| | acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DMF and DIPEA. Crude | | |
| | used without further purification | | |
| | Step 2: GM2A. Purification a (SCX-2) | | |
| | Step 3: GM1 with DCM and DIPEA. Purified by | | |
| | prep HPLC | | |
| | Step 4: GM3. Final purification prep HPLC | | |
| M05878 | 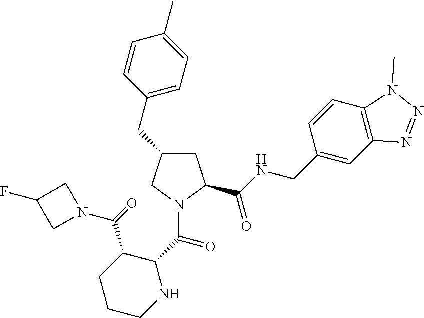 [SMILES: Cc1ccc(C[C@@H]2C[C@@H](C(=O)NCc3ccc4c(c3)nnn4C)N(C(=O)[C@@H]3NCCC[C@@H]3C(=O)N3CC(F)C3)C2)cc1] | UPLC_pH2_MeCN_QC V1, Rt: 3.38 min, m/z 575.3 [M + H]⁺ UPLC_pH10_MeCN_QC V1, Rt: 4.47 min, m/z 575.3 [M + H]⁺ ¹H-NMR (400 MHz, DMSO-D6) δ 8.64 (t, J = 6.0 Hz, 0.4H), 8.16-8.10 (m, 0.3H), 7.97-7.94 (m, 1H), 7.59-7.52 (m, 2H), 7.30-7.21 (m, 1H), 7.11- 7.01 (m, 4H), 5.42 (s, 0.4H), 5.27 (s, 0.4H), 5.12 (t, J = 5.0 Hz, 0.5H), 4.43- 4.06 (m, 4H), 4.06-3.99 (m, 3H), 3.85-3.45 (m, 2H), 3.15 (t, J = 9.4 Hz, 0.4H), 3.01-2.88 (m, 2H), 2.73-2.54 (m, 3H), 2.44- | 4.0 mg, 13%, white solid |
| | (2S,4R)-1-((2R,3S)-3-(3-fluoroazetidine-1- | 2.32 (m, 1H), 2.26-2.08 | |
| | carbonyl)piperidine-2-carbonyl)-N-((1-methyl- | (m, 4H), 1.93-1.60 (m, | |
| | 1H-indazol-5-yl)methyl)-4-(4- | 4H), 1.27-0.99 (m, 2H) | |
| | methylbenzyl)pyrrolidine-2-carboxamide | | |
| | S1: (1-methyl-1H-indazol-5-yl)methanamine | | |
| | S2: (2S,4R)-1-(tert-butoxycarbonyl)-4-(4- | | |
| | methylbenzyl)pyrrolidine-2-carboxylic acid | | |
| | S4: (2R,3S)-3-(3-fluoroazetidine-1- | | |
| | carbonyl)piperidine-2-carboxylic acid | | |
| | Step 1: GM1 with DCM and DIPEA. Purified by | | |
| | column chromatography. | | |
| | Step 2: GM2A. Purified by SCX-2 | | |
| | Step 3: GM1 with DCM and DIPEA. Crude | | |
| | used without further purification | | |
| | Step 4: GM3. final purification prep HPLC. | | |
| M05881 | 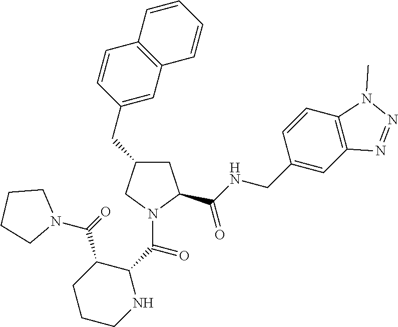 [SMILES: Cn1nnc2cc(CNC(=O)[C@@H]3C[C@@H](Cc4ccc5ccccc5c4)CN3C(=O)[C@@H]3NCCC[C@@H]3C(=O)N3CCCC3)ccc21] | UPLC_pH2_MeCN_QC_ V1, Rt: 3.58 min, m/z 608.3 [M + H]⁺ UPLC_pH10_MeCN_QC_ V1, Rt: 4.74 min, m/z 608.4 [M + H]⁺ ¹H-NMR (400 MHz, DMSO-D₆) δ 8.69 (t, J = 6.2 Hz, 0.5H), 8.22 (t, J = 6.0 Hz, 0.4H), 7.89-7.82 (m, 4H), 7.76-7.68 (m, 2H), 7.51-7.37 (m, 4H), 6.62 (s, 0.1H), 5.30 (s, 0.5H), 4.48-4.23 (m, 6H), 4.06-4.02 (m, 0.3H), 3.50 (m, 1H), 3.28-3.12 (m, 2H), 3.08-2.65 (m, 5H), 2.33-2.20 (m, 1H), 2.01- 1.23 (m, 8H), 1.14-1.10 (m, 1H) | 33.3 mg, 55%, white solid |
| | (2S,4R)-N-((1-methyl-1H- | | |
| | benzo[d][1,2,3]triazol-5-yl)methyl)-4- | | |
| | (naphthalen-2-ylmethyl)-1-((2R,3S)-3- | | |
| | (pyrrolidine-1-carbonyl)piperidine-2- | | |
| | carbonyl)pyrrolidine-2-carboxamide | | |
| | S1: (1-methyl-1H-benzo[d][1,2,3]triazol-5- | | |
| | yl)methanamine | | |
| | S2: (2S,4R)-1-(tert-butoxycarbonyl)-4- | | |
| | (naphthalen-2-ylmethyl)pyrrolidine-2-carboxylic | | |
| | acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1, with DMF and DIPEA. Crude | | |
| | used without further purification | | |
| | Step 2: GM2A. Purification a followed by d | | |
| | (SCX-2 and DCM trituration) | | |
| | Step 3: GM1 with DCM and DIPEA. Purified by | | |
| | prep HPLC | | |
| | Step 4: GM3. Final purification prep HPLC | | |
| M05882 | 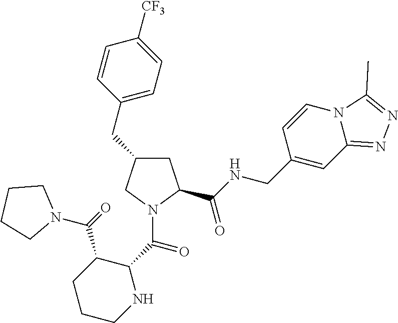 [SMILES: Cc1nnc2cc(CNC(=O)[C@@H]3C[C@@H](Cc4ccc(C(F)(F)F)cc4)CN3C(=O)[C@@H]3NCCC[C@@H]3C(=O)N3CCCC3)ccn12] (2S,4R)-N-((3-methyl-[1,2,4]triazolo[4,3- a]pyridin-7-yl)methyl)-1-((2R,3S)-3-(pyrrolidine- 1-carbonyl)piperidine-2-carbonyl)-4-(4- (trifluoromethyl)benzyl)pyrrolidine-2-carboxamide S1: (3-methyl-[1,2,4]triazolo[4,3-a]pyridin-7-yl)methanamine S2: (2S,4R)-1-(tert-butoxycarbonyl)-4-(4- | UPLC_pH2_MeCN_QC_ V1, Rt: 3.25 min, m/z 626.3 [M + H]⁺ UPLC_pH10_MeCN_QC_ V1, Rt: 4.38 min, m/z 626.3 [M + H]⁺ ¹H-NMR (400 MHz, DMSO-D₆) δ 8.69 (t, J = 6.2 Hz, 0.5H), 8.29-8.31 (m, 0.5H), 8.21-8.24 (m, 1H), 7.64-7.67 (m, 2H), 7.40-7.47 (m, 3H), 6.79 (ddd, J= 12.5, 7.2, 1.5 Hz, 1H), 5.31-5.33 (m, 0.5H), 4.19-4.36 (m, 2H), 4.00-4.05 (m, 0.5H), 3.44- 3.53 (m, 1H), 3.35-3.39 (m, 1H), 3.13-3.24 (m, 2H), 2.91-3.06 (m, 2H), 2.72-2.80 (m, 2H), 2.57- 2.64 (m, 4H), 2.31-2.42 (m, 1H), 1.94-1.98 (m, 1H), 1.66-1.85 (m, 6H), 1.47-1.57 (m, 0.5H), 1.13- 1.28 (m, 1H). Missing protons: 5.5 | 33.3 mg, 55%, white solid |
| | (trifluoromethyl)benzyl)pyrrolidine-2-carboxylic acid | | |
| | S4: (2R,3S)-1-((benzyloxy)carbonyl)-3- | | |
| | (pyrrolidine-1-carbonyl)piperidine-2-carboxylic | | |
| | acid | | |
| | Step 1: GM1 with DMF and DIPEA | | |
| | Step 2: GM2A. Purification a (SCX-2). | | |
| | Step 3: GM1 with DMF and DIPEA. Purified by | | |
| | preparative HPLC | | |
| | Step 4: GM3. Final purification prep HPLC | | |
| M05798 | 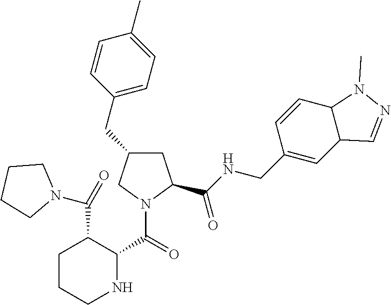 [SMILES: Cc1ccc(C[C@@H]2C[C@@H](C(=O)NCC3=CC4C=NN(C)C4C=C3)N(C(=O)[C@@H]3NCCC[C@@H]3C(=O)N3CCCC3)C2)cc1] (2S,4R)-N-[(1-methylindazol-5-yl)methyl]- 4-(4-methylphenoxy)-1-[(2R,3S)-3- (pyrrolidine-1-carbonyl)piperidine-2- carbonyl]pyrrolidine-2-carboxamide S1: 5-(Aminomethyl)-1-methyl-1H-indazole S2: (2S,4R)-1-tert-butoxycarbonyl-4-(4- methylphenoxy)pyrrolidine-2-carboxylic acid | AnalpH2_MeCN_QC_v 2 Rt: 4.71 min, m/z 573.4 [M + H]+ AnalpH9_MeCN_QC_V 2 Rt: 6.39 min, m/z 573.4 [M + H]+ 1H-NMR (400 MHz, DMSO-D6) δ 8.71-8.55 (m, 0.5H), 8.28 (t, J = 6.0 Hz, 0.5H), 8.00- 7.92 (m, 1H), 7.68-7.51 (m, 2H), 7.41-7.27 (m, 1H), 7.11-7.05 (m, 2H), 6.82-6.70 (m, 2H), 5.20 (t, J = 8.2 Hz, 0.5H), 5.00-4.78 (m, 1H), 4.48-4.23 (m, 2.5H), 4.06-3.99 (m, 3H), 3.73 (dd, J = 31.6, 12.4 Hz, 1H), 3.57-3.40 (m, 2H), 3.29-3.12 (m, 3H), 3.02 (d, J = 12.8 Hz, 1H), 2.86-2.54 (m, 3H), 2.33-2.05 (m, 5H), 1.90-0.98 (m, 7H). Missing protons: 1 | 8.85 mg, 82%, Off- White Solid |
| | S4: (2R,3S)-1-benzyloxycarbonyl-3- | | |
| | (pyrrolidine-1-carbonyl)piperidine-2- | | |
| | carboxylic acid | | |
| | Step 1: GM1 with DMF and DIPEA. Amine | | |
| | used as HCl salt. | | |
| | Step 2: GM2A. No purification | | |
| | Step 3: GM1 with DMF and DIPEA. | | |
| | Purified by prep HPLC | | |
| | Step 4: GM3 | | |
| M05965 | 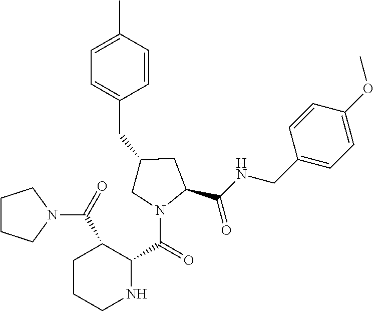 [SMILES: COc1ccc(CNC(=O)[C@@H]2C[C@@H](Cc3ccc(C)cc3)CN2C(=O)[C@@H]2NCCC[C@@H]2C(=O)N2CCCC2)cc1] (2S,4R)-N-[(4-methoxyphenyl)methyl]-4- (p-tolylmethyl)-1-[(2R,3S)-3-(pyrrolidine-1- carbonyl)piperidine-2-carbonyl]pyrrolidine-2-carboxamide S1: 4-Methoxybenzylamine | UPLC_pH2_MeCN_QC_ V1 Rt: 3.63 min, m/z 547.3 [M + H]+ UPLC_pH9_MeCN_QC V1 Rt: 5.0 min, m/z 547.3 [M + H]+ 1H-NMR (400 MHz, DMSO-D6) δ 8.48 (t, J = 6.0 Hz, 0/5H), 7.99 (t, J = 6.0 Hz, 0.5H), 7.15- 7.02 (m, 6H), 6.87-6.81 (m, 2H), 5.18 (d, J = 4.1 Hz, 0.5H), 4.31-3.98 (m, 3H), 3.76-3.69 (m, 2.5H), 3.54-3.36 (m, 2H), 3.27-3.03 (m, 3H), 2.99-2.75 (m, 2H), 2.67-2.52 (m, 3H), 2.35-2.23 (m, 4H), 1.95-1.66 (m, 8H), 1.61-1.44 (m, 1H), 1.28- 1.15 (m, 1H). Missing protons: 2 | 68 mg, 79%, white solid |
| | S2: Boc-(R)-y-(4-methylbenzyl)-L-proline | | |
| | S4: (2R,3S)-1-benzyloxycarbonyl-3- | | |
| | (pyrrolidine-1-carbonyl)piperidine-2- | | |
| | carboxylic acid | | |
| | Step 1: GM1 with DMF and DIPEA. Amine | | |
| | used as HCl salt. | | |
| | Step 2: GM2A. purification a | | |
| | Step 3: GM1 with DMF and DIPEA. | | |
| | Purified by prep HPLC | | |
| | Step 4: GM3. | | |
| M05934 | 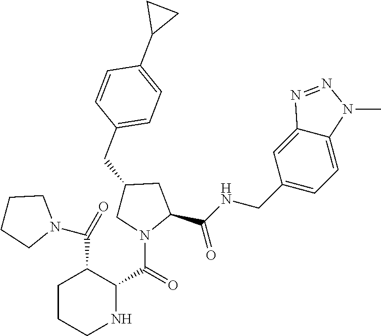 [SMILES: Cn1nnc2cc(CNC(=O)[C@@H]3C[C@@H](Cc4ccc(C5CC5)cc4)CN3C(=O)[C@@H]3NCCC[C@@H]3C(=O)N3CCCC3)ccc21] | UPLC_pH2_MeCN_QC V1 Rt: 3.5 min, m/z 598.4 [M + H]+ UPLC_pH9_MeCN_QC_ V1 Rt: 4.73 min, m/z 598.4 [M + H]+ 1H-NMR (400 MHz, DMSO-D6) δ 8.67 (t, J = 6.2 Hz, 0.5H), 8.19 (t, J = 6.0 Hz, 0.5H), 7.83-7.73 (m, 2H), 7.42 (qd, J = 8.7, 1.0 Hz, 1H), 7.05 (t, J = 8.2 Hz, 2H), 6.98 (q, J = 4.0 Hz, 2H), 5.24 (d, J = 5.0 Hz, 0.5H), 4.49-4.31 (m, 2H), 4.28 (d, J = 1.8 Hz, 3H), 4.00 (dd, J = 9.6, 6.4 Hz, 0.5H), 3.53-3.35 (m, 3H), 3.26-3.05 (m, 3H), 3.00-2.77 (m, 2H), 2.67-2.54 (m, 3H), 2.36- | 34 mg, 48%, White Solid |
| | (2S,4R)-4-(4-cyclopropylbenzyl)-N-((1-methyl- | 2.22 (m, 1H), 1.98-1.70 | |
| | 1H-benzo[d][1,2,3]triazol-5-yl)methyl)-1- | (m, 9H), 1.56 (q, J = 12.5 | |
| | ((2R,3S)-3-(pyrrolidine-1-carbonyl)piperidine-2- | Hz, 0.5H), 1.33 (q, J = | |
| | carbonyl)pyrrolidine-2-carboxamide | 13.4 Hz, 1H), 1.15-1.09 | |
| | S1: (1-methyl-1H-benzo[d][1,2,3]triazol-5- | (m, 0.5H), 0.95-0.88 (m, | |
| | yl)methanamine | 2H), 0.64-0.60 (m, 2H). | |
| | S2: (2S,4R)-1-tert-butoxycarbonyl-4-[(4- | Missing protons:1 | |
| | cyclopropylphenyl)methyl]pyrrolidine-2- | | |
| | carboxylic acid | | |
| | S4: (2R,3S)-1-benzyloxycarbonyl-3- | | |
| | (pyrrolidine-1-carbonyl)piperidine-2-carboxylic | | |
| | acid | | |
| | Step 1: GM1 with DMF and DIPEA. Amine | | |
| | used as HCl salt. | | |
| | Step 2: GM2A. No purification | | |
| | Step 3: GM1 with DMF and DIPEA. Purified by | | |
| | prep HPLC | | |
| | Step 4: GM3. M05935 generated as a by- | | |
| | product of this step. M05934 and M05935 | | |
| | separated by prep HPLC. | | |
| M05935 | 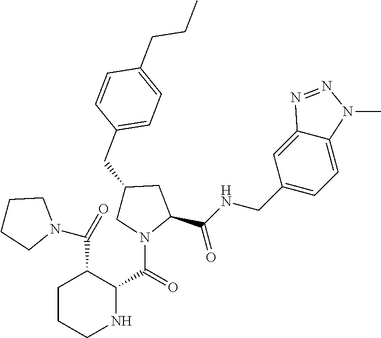 [SMILES: CCCc1ccc(C[C@@H]2C[C@@H](C(=O)NCc3ccc4c(c3)nnn4C)N(C(=O)[C@@H]3NCCC[C@@H]3C(=O)N3CCCC3)C2)cc1] | UPLC_pH2_MeCN_QC_ V1 Rt: 3.72 min, m/z 600.4 [M + H]+ UPLC_pH9_MeCN_QC_ V1 Rt: 5.06 min, m/z 600.5 [M + H]+ | 20 mg, 26.4 %. White solid |

### Synthesis of Alkylated Piperidines (Reductive Alkylation of M05675):

### Synthesis of (2S,4R)—N-[(1-methylindazol-5-yl)methyl]-1-[(2R,3S)-1-methyl-3-(pyrrolidine-I-carbonyl)piperidine-2-carbonyl]-4-(p-tolylmethyl)pyrrolidine-2-carboxamide (M05757)

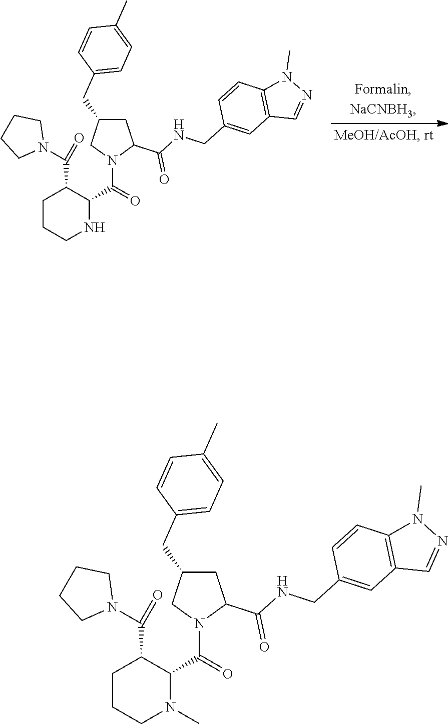 [SMILES: Cc1ccc(C[C@@H]2CC(C(=O)NCc3ccc4c(cnn4C)c3)N(C(=O)[C@@H]3NCCC[C@@H]3C(=O)N3CCCC3)C2)cc1 ; Cc1ccc(C[C@@H]2CC(C(=O)NCc3ccc4c(cnn4C)c3)N(C(=O)[C@H]3[C@@H](C(=O)N4CCCC4)CCCN3C)C2)cc1]

(2S,4R)—N-[(1-methylindazol-5-yl)methyl]-4-(p-tolylmethyl)-1-[(2R,3S)-3-(pyrrolidine-1-carbonyl)piperidine-2-carbonyl]pyrrolidine-2-carboxamide (40 mg, 0.07 mmol), formaldehyde 37% solution (formalin) (0.057 g, 0.70 mmol) and acetic acid (0.10 mL) were dissolved in methanol (3.0 mL) and stirred at rt for 30 minutes. Sodium cyanoborohydride (8.81 mg, 0.140 mmol) was added and the reaction stirred at rt overnight. The solvent was removed, and the residue was dissolved in EtOAc and washed with brine. The organic was dried (MgSO₄), filtered and solvent removed. The residue was purified by preparative HPLC. Solvent removed and the residue lyophilised from 1:1 MeCN/H₂O to give (2S,4R)—N-[(1-methylindazol-5-yl)methyl]-1-[(2R,3S)-1-methyl-3-(pyrrolidine-1-carbonyl)piperidine-2-carbonyl]-4-(p-tolylmethyl)pyrrolidine-2-carboxamide (28 mg, 0.048 mmol, 68%) as a white solid.

AnalpH2_MeOH_QC_v1, Rt: 6.07 min, m/z 585.6 [M+H]⁺

AnalpH9_MeOH_QC_v1, Rt: 8.15 min, m/z 585.6 [M+H]⁺

¹H-NMR (400 MHz, DMSO-D₆) δ 8.26 (t, J=6.2 Hz, 1H), 7.97-7.89 (m, 1H), 7.63 (s, 1H), 7.53 (t, J=8.5 Hz, 1H), 7.28-7.21 (m, 1H), 7.10-7.05 (m, 4H), 4.40-4.29 (m, 3H), 4.05-4.01 (m, 3H), 3.77-3.68 (m, 2H), 3.57-3.47 (m, 2H), 3.30-3.18 (m, 2H), 2.86-2.82 (m, 2H), 2.67-2.55 (m, 3H), 2.33-2.26 (m, 4H), 2.22-2.11 (m, 4H), 1.94-1.77 (m, 5H), 1.68-1.38 (m, 3H).

The following compound was made by analogous methods:

| | | | Mass, % |
|---|---|---|---|
| Example | | | yield, |
| No. | Structure & Conditions | Analytical Data | state |
| M05772 | 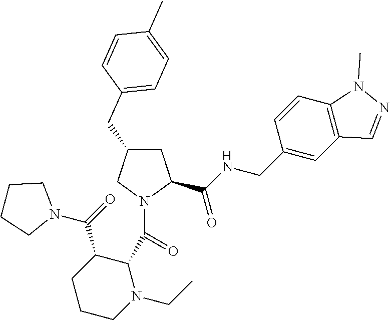 (2S,4R)-1-((2R,3S)-1-ethyl-3-(pyrrolidine-1- carbonyl)piperidine-2-carbonyl)-N-((1-methyl- 1H-indazol-5-yl)methyl)-4-(4- methylbenzyl)pyrrolidine-2-carboxamide Using acetaldehyde, and purified by preparative HPLC | AnalpH2_MeCN_QC_V 2, Rt: 4.87 min, m/z 599.4 [M + H]⁺ AnalpH9_MeCN_QC_v 2, Rt: 6.66 min, m/z 599.4 [M + H]⁺ ¹H-NMR (400 MHz, DMSO-D₆) δ 8.22 (t, J = 6.2 Hz, 1H), 7.97-7.89 (m, 1H), 7.62 (s, 1H), 7.55-7.45 (m, 1H), 7.26 (dd, J = 8.7, 1.4 Hz, 1H), 7.10-7.06 (m, 4H), 4.36-4.29 (m, 3H), 4.02 (s, 3H), 3.76-3.71 (m, 2H), 3.54-3.47 (m, 2H), 3.30-3.15 (m, 3H), 2.86-2.78 (m, 2H), 2.67-2.56 (m, 2H), 2.47-2.42 (m, 1H), 2.33-2.08 (m, 6H), 1.94-1.77 (m, 5H), 1.68-1.58 (m, 2H), 1.53-1.38 (m, 1H), 0.86 (t, J = 7.1 Hz, 3H) | 20.0 mg, 61 %, white solid |

### Synthesis of (2R, 3S)—N-(1-(2-fluoroethyl) piperidin-4-yl)-N-methyl-2-((2S, 4R)-2-(((1-methyl-I H-indazol-5-yl)methyl)carbamoyl)-4-(4-methylbenzyl)pyrrolidine-1-carbonyl)piperidine-3-carboxamide (M06083)

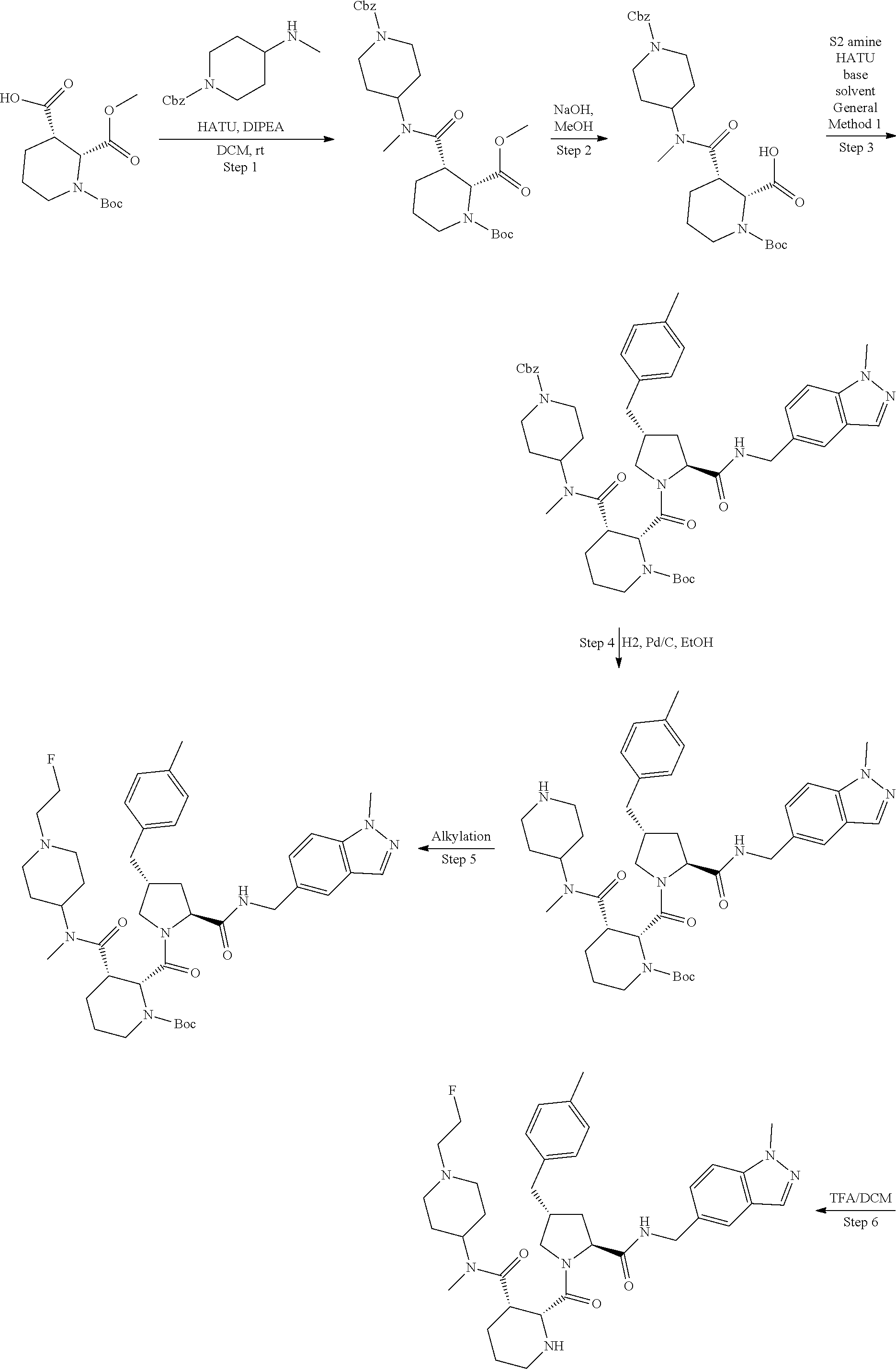

### Step 1: Synthesis of O1-tert-butyl O2-methyl (2R,3S)-3-[(1-benzyloxycarbonyl-4-piperidyl)-methyl-carbamoyl]piperidine-1,2-dicarboxylate

1-Cbz-4-Methylaminopieridine (259 mg, 1.04 mmol) and (2R,3S)-1-(tert-butoxycarbonyl)-2-(methoxycarbonyl)piperidine-3-carboxylic acid (300 mg, 1.04 mmol) was dissolved in dichloromethane (5.0 mL) and N,N-diisopropylethylamine (0.55 mL, 3.13 mmol) followed by HATU (397 mg, 1.04 mmol) were added. The reaction was stirred at rt overnight.

Water (20 mL) was added and the layers separated. The aqueous was extracted with DCM (20 mL) and the combined organics passed though a phase separator and solvent removed. The residue was purified by column chromatography (SiO₂, Biotage 25 g SFAR) 0-100% EtOAc/ihexane. The solvent was removed to give O1-tert-butyl O2-methyl (2R,3S)-3-[(1-benzyloxycarbonyl-4-piperidyl)-methyl-carbamoyl]piperidine-1,2-dicarboxylate (510 mg, 0.99 mmol, 94%) as a colourless gum.

AnalpH9_MeCN_4 min, Rt: 2.73 min, m/z 518.4 [M+H]⁺

### Step 2: Synthesis of (2R,3S)-3-[(1-benzyloxycarbonyl-4-piperidyl)-methyl-carbamoyl]-1-tert-butoxycarbonyl-piperidine-2-carboxylic acid

O1-tert-butyl O2-methyl (2R,3S)-3-[(1-benzyloxycarbonyl-4-piperidyl)-methyl-carbamoyl]piperidine-1,2-dicarboxylate (510 mg, 0.99 mmol) was dissolved in methanol (5.0 mL) and lithium hydroxide (1 M) (4.9 mL, 4.9 mmol) added. The reaction was stirred at rt overnight. Additional lithium hydroxide (1 M) (4.9 mL, 4.93 mmol) was added and the reaction was stirred at rt for 4 h. The solvent was removed and the residue partitioned between EtOAc and water. The aqueous was acidified to pH4 and extracted with 2×20 mL EtOAc. The combined organics were dried (MgSO₄), filtered and solvent removed. The crude was purified by reverse phase chromatography (Teledyne 240 g C18 column). The solvent was removed to give (2R,3S)-3-[(1-benzyloxycarbonyl-4-piperidyl)-methyl-carbamoyl]-1-tert-butoxycarbonyl-piperidine-2-carboxylic acid (197 mg, 0.391 mmol, 40%,) as a white solid.

AnalpH9_MeCN_4 min, Rt: 1.93 min, m/z 504.3 [M+H]⁺

### Step 3: Synthesis of tert-butyl (2R,3S)-3-[(1-benzyloxycarbonyl-4-piperidyl)-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate

(2R,3S)-3-[(1-benzyloxycarbonyl-4-piperidyl)-methyl-carbamoyl]-1-tert-butoxycarbonyl-piperidine-2-carboxylic acid (195 mg, 0.39 mmol) and (2S,4R)—N-[(1-methylindazol-5-yl)methyl]-4-(p-tolylmethyl)pyrrolidine-2-carboxamide (140 mg, 0.39 mmol) were dissolved in N,N-dimethylformamide (3.0 mL). The reaction was cooled to 0° C. after which N,N-diisopropylethylamine (0.2 mL, 1.16 mmol) was added dropwise. After 5 minutes HATU (176 mg, 0.46 mmol) was added. The reaction was stirred at rt for 3 h. The solvent was removed, and the residue partitioned between EtOAc (20 mL) and brine (20 mL). The aqueous was extracted with EtOAc (20 mL) and the combined organics dried (MgSO₄), filtered and solvent removed. The residue was purified by column chromatography (SiO₂, Biotage 25 g SFAR) 0-100% EtOAc followed by 0-10% MeOH/DCM. Fractions containing the desired product were also passed through an SCX-2 cartridge (2 g), washing with MeOH then solvent removed to give tert-butyl (2R,3S)-3-[(1-benzyloxycarbonyl-4-piperidyl)-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate (253 mg, 0.298 mmol, 77%) as a colourless oil.

AnalpH9_MeCN_4 min, Rt: 3.05 min, m/z 848.6 [M+H]⁺

### Step 4: Synthesis of tert-butyl (2R,3S)-2-[(2S,4R)-2-[(1-methylindazol-5-yl)methylcarbamoyl]-4-(p-tolylmethyl)pyrrolidine-1-carbonyl]-3-[methyl(4-piperidyl)carbamoyl]piperidine-1-carboxylate

tert-butyl (2R,3S)-3-[(1-benzyloxycarbonyl-4-piperidyl)-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate (253 mg, 0.3 mmol) was dissolved in methanol (5.0 mL) and palladium 5% wt on Carbon (32 mg, 0.298 mmol) added under N₂. The atmosphere was evacuated and replaced with H₂ (×3) then the reaction was stirred at rt under H₂ overnight. The reaction was place directly onto a SCX-2 cartridge (2 g), washing with MeOH and eluting with 1 M NH₃/MeOH. The solvent was removed to give tert-butyl (2R,3S)-2-[(2S,4R)-2-[(1-methylindazol-5-yl)methylcarbamoyl]-4-(p-tolylmethyl)pyrrolidine-1-carbonyl]-3-[methyl(4-piperidyl)carbamoyl]piperidine-1-carboxylate (160 mg, 0.224 mmol, 75%) as a colourless gum.

AnalpH9_MeCN_4 min. Rt: 2.34 min. m/z 714.6 [M+H]⁺

### Step 5: Synthesis of tert-butyl (2R,3S)-3-[[1-(2-fluoroethyl)-4-piperidyl]-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate

tert-butyl (2R,3S)-2-[(2S,4R)-2-[(1-methylindazol-5-yl)methylcarbamoyl]-4-(p-tolylmethyl)pyrrolidine-1-carbonyl]-3-[methyl(4-piperidyl)carbamoyl]piperidine-1-carboxylate (75 mg, 0.11 mmol) was dissolved in N,N-dimethylformamide (1.0 mL) and N,N-diisopropylethylamine (0.02 mL, 0.12 mmol) and 1-fluoro-2-iodoethane (0.01 mL, 0.12 mmol) was added. The reaction was stirred at 45° C. overnight. Additional 1-fluoro-2-iodoethane (0.01 mL, 0.12 mmol) and N,N-diisopropylethylamine (0.02 mL, 0.12 mmol) were added and the reaction stirred at 60° C. for 2 hours. Brine (10 mL) and water (10 mL) were added and the product extracted with 2×20 mL EtOAc. The combined organics were dried (MgSO₄), filtered and solvent removed. The residue was purified by Preparative HPLC. The solvent was removed to give tert-butyl (2R,3S)-3-[[1-(2-fluoroethyl)-4-piperidyl]-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate (25 mg, 0.033 mmol, 31%) as a white solid.

AnalpH9_MeCN_4 min, Rt: 2.69 min, m/z 760.7 [M+H]⁺

### Step 6: Synthesis of (2R,3S)—N-[1-(2-fluoroethyl)-4-piperidyl]-N-methyl-2-[(2S,4R)-2-[(1-methylindazol-5-yl)methylcarbamoyl]-4-(p-tolylmethyl)pyrrolidine-1-carbonyl]piperidine-3-carboxamide

tert-butyl (2R,3S)-3-[[1-(2-fluoroethyl)-4-piperidyl]-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate (25 mg, 0.03 mmol) was dissolved in dichloromethane (1.0 mL) and trifluoroacetic acid (0.30 mL) added. The reaction was stirred at rt for 3 h. The solvent was removed and the residue purified by SCX-2 (1 g), washing with MeOH and eluting with 1 M NH₃/MeOH. Solvent removed and the residue was lyophilised to give (2R,3S)—N-[1-(2-fluoroethyl)-4-piperidyl]-N-methyl-2-[(2S,4R)-2-[(1-methylindazol-5-yl)methylcarbamoyl]-4-(p-tolylmethyl)pyrrolidine-1-carbonyl]piperidine-3-carboxamide (21 mg, 0.0318 mmol, 97%) as a white solid.

UPLC_pH2_MeCN_QC_V1, Rt: 2.61, m/z 660.5 [M+H]⁺

UPLC_pH9_MeCN_QC_V1, Rt: 4.75, m/z 660.6 [M+H]⁺

1H-NMR (400 MHz, DMSO-D₆) δ 8.56 (d, J=5.0 Hz, 0.5H), 8.05 (d, J=18.3 Hz, 0.5H), 7.95 (d, J=3.2 Hz, 1H), 7.57 (d, J=7.8 Hz, 1H), 7.52 (d, J=5.0 Hz, 1H), 7.29 (dd, J=8.7, 2.7 Hz, 0.5H), 7.22 (d, J=8.7 Hz, 0.5H), 7.10-7.04 (m, 4H), 5.29-5.23 (m, 0.5H), 4.56 (t, J=5.0 Hz, 1H), 4.45-4.17 (m, 4.5H), 3.99 (d, J=18.3 Hz, 4H), 3.51-3.43 (m, 1H), 3.18-3.13 (m, 1H), 3.03-2.83 (m, 4H), 2.79 (s, 1H), 2.73 (s, 1H), 2.68-2.54 (m, 6H), 2.26 (d, J=2.3 Hz, 3H), 2.19-1.23 (m, 12H), 1.15-1.08 (m, 1H)

The following compound was made by analogous methods:

| | | | |
|---|---|---|---|
| M06084 | 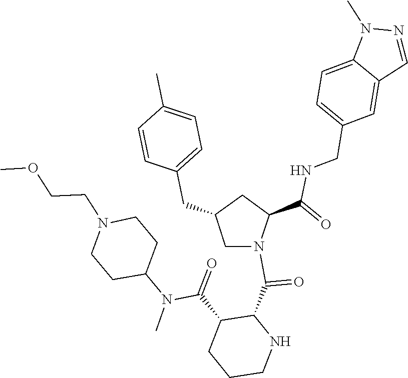 [SMILES: COCCN1CCC(N(C)C(=O)[C@H]2CCCN[C@H]2C(=O)N2C[C@H](Cc3ccc(C)cc3)C[C@H]2C(=O)NCc2ccc3c(cnn3C)c2)CC1] (2R,3S)-N-(1-(2-methoxyethyl)piperidin-4- yl)-N-methyl-2-((2S,4R)-2-(((1-methyl-1H- indazol-5-yl)methyl)carbamoyl)-4-(4- methylbenzyl)pyrrolidine-1- | UPLC_pH2_MeCN_QC_ V1, Rt: 2.61, m/z 672.6 [M + H]+ UPLC_pH9_MeCN_QC_ V1, Rt: 4.72, m/z 672.6 [M + H]+ 1H-NMR (400 MHz, DMSO-D6) δ 8.57-8.54 (m, 0.5H), 8.08-8.03 (m, 0.5H), 7.92 (dd, J = 26.1, 3.7 Hz, 1H), 7.57 (d, J = 8.2 Hz, 1H), 7.52 (d, J = 7.8 Hz, 1H), 7.30-7.27 (m, 0.5H), 7.23 (d, J = 9.2 Hz, 0.5H), 7.10-7.04 (m, 4H), 5.29-5.24 (m, 0.5H), 4.44-4.16 (m, 3H), 4.02-3.97 (m, 3.5H), 3.49-3.36 (m, 3H), 3.22-3.19 (m, 3H), 3.05-2.53 (m, 12H), 2.46-2.40 (m, 1H), 2.36-2.26 (m, 4H), 2.16 (t, J = 11.9 Hz, 1H), 2.07-1.24 (m, 11H), 1.15-1.08 (m, 1H) | 30 mg, 93%, White Solid |
| | carbonyl)piperidine-3-carboxamide | | |
| | Step 5: with 2-bromoethyl methyl ether | | |
| | Step 6: GM2A Final purification a | | |

### Synthesis of (2R,3S)—N-(2-(dimethylamino)ethyl)-N-methyl-2-((2S,4R)-2-(((1-methyl-1H-indazol-5-yl)methyl)carbamoyl)-4-(4-methylbenzyl)pyrrolidine-1-carbonyl)piperidine-3-carboxamide (M05902)

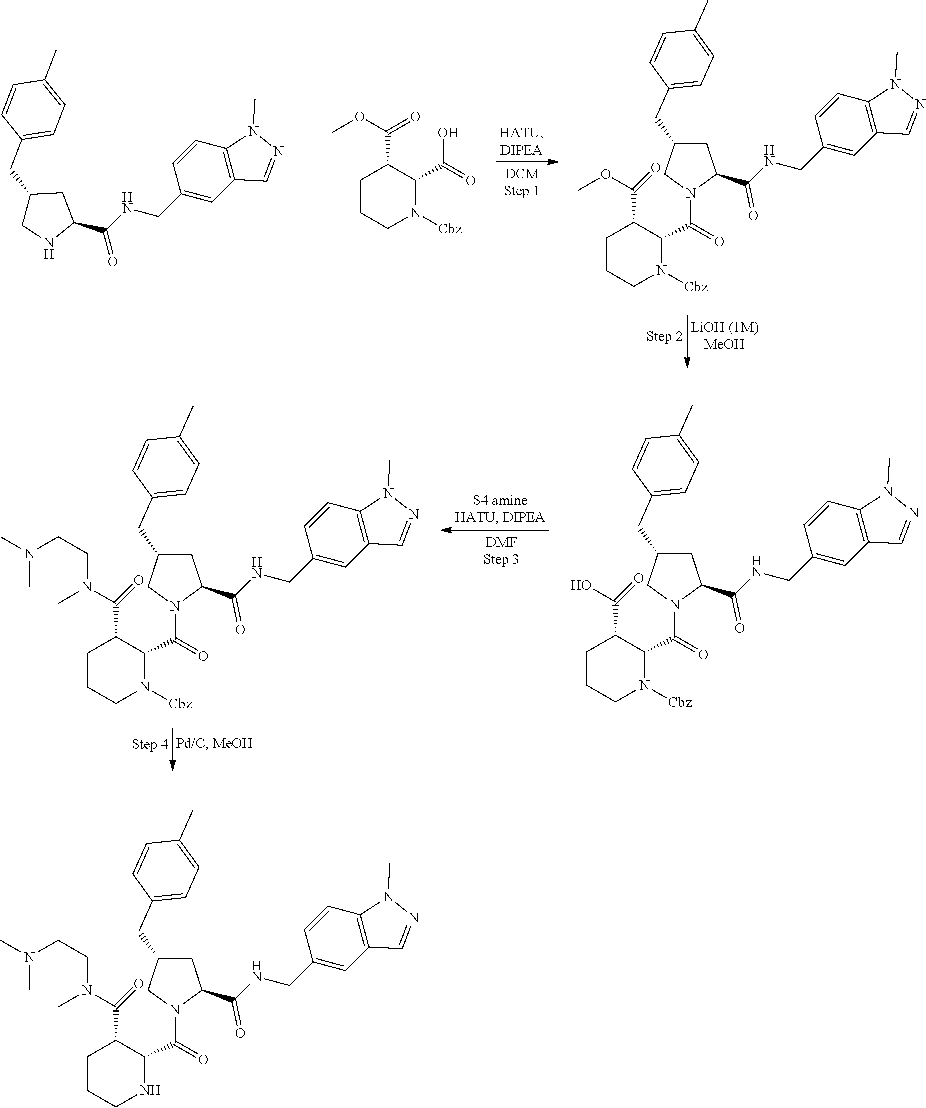

Step 1: (2R,3S)-1-((Benzyloxy)carbonyl)-3-(methoxycarbonyl)piperidine-2-carboxylic acid (123 mg, 0.384 mmol) was dissolved in anhydrous DCM (3 mL) and placed under nitrogen. (2S,4R)—N-[(1-Methylindazol-5-yl)methyl]-4-(p-tolylmethyl)pyrrolidine-2-carboxamide (143 mg, 0.394 mmol) was added to the reaction mixture, after which the reaction mixture was cooled to 0° C. DIPEA (0.2 mL) was added dropwise and the reaction mixture stirred for 5 minutes. HATU (177 mg, 0.466 mmol) was added, the reaction mixture was stirred at 0° C. for 3 hours. The reaction was quenched with saturated aq. sodium hydrogen carbonate solution (10 mL) and extracted with DCM (3×20 mL). The combined organic layers were passed through a hydrophobic frit, and concentrated under reduced pressure to afford 01-benzyl O3-methyl (2R,3S)-2-[(2S,4R)-2-[(1-methylindazol-5-yl)methylcarbamoyl]-4-(p-tolylmethyl)pyrrolidine-1-carbonyl]piperidine-1,3-dicarboxylate as a light yellow oil (461 mg crude, assumed quantitative yield: 249 mg, 0.374 mmol). The crude product was used directly in the subsequent reaction without further purification.

AnalpH9_MeCN_4 min. Rt: 2.84 min. m/z 666.4 [M+H]P

Step 2: O1-Benzyl O3-methyl (2R,3S)-2-[(2S,4R)-2-[(1-methylindazol-5-yl)methylcarbamoyl]-4-(p-tolylmethyl)pyrrolidine-1-carbonyl]piperidine-1,3-dicarboxylate (249 mg, 0.374 mmol) was dissolved in MeOH (6.3 mL). LiOH (1 M, 1.9 mL) was added dropwise to the solution. The reaction mixture was stirred at room temperature for 22.5 hours. The reaction mixture was acidified to pH 6 with 1 M HCl aq. and partially concentrated in vacuo to remove MeOH. The aqueous solution was basified to pH 9 using saturated aq. sodium hydrogen carbonate solution and then extracted with EtOAc (3×20 mL). The combined organic extracts were passed through a hydrophobic frit and concentrated under reduced pressure to afford (2R,3S)-1-benzyloxycarbonyl-2-[(2S,4R)-2-[(1-methylindazol-5-yl)methylcarbamoyl]-4-(p-tolylmethyl)pyrrolidine-1-carbonyl]piperidine-3-carboxylic acid as an off white solid containing the desired product as a mixture of diastereomers (232 mg, Batch A)

AnalpH9_MeCN_4 min, Rt: 2.03 min, m/z 652.4 [M+H]⁺ (28.2%) and Rt: 2.07 min, m/z 652.4 [M+H]⁺ (45.5%)

The aqueous layer was further acidified to pH 4 and extracted with DCM (3×20 mL). The combined DCM layers were passed through a hydrophobic frit, and concentrated under reduced pressure to afford (2R,3S)-1-benzyloxycarbonyl-2-[(2S,4R)-2-[(1-methylindazol-5-yl)methylcarbamoyl]-4-(p-tolylmethyl)pyrrolidine-1-carbonyl]piperidine-3-carboxylic acid as a white foam containing the product as a mixture of diastereomers (106 mg, Batch B).

AnalpH9_MeCN_4 min, Rt: 2.04 min, m/z 652.4 [M+H]⁺ (61.6%) and Rt: 2.08 min, m/z 652.4 [M+H]⁺ (27.6%)

Step 3: (2R,3S)-1-benzyloxycarbonyl-2-[(2S,4R)-2-[(1-methylindazol-5-yl)methylcarbamoyl]-4-(p-tolylmethyl)pyrrolidine-1-carbonyl]piperidine-3-carboxylic acid (Batch B, 106 mg, 0.163 mmol) was dissolved in anhydrous DMF (1.3 mL). N,N,N′-trimethylethylenediamine (0.11 mL) was added dropwise after which DIPEA (0.17 mL) was added dropwise. After 5 minutes of stirring, HATU (157 mg, 0.413 mmol) was added, and the reaction mixture was stirred at room temperature for 1 hour. The reaction mixture was concentrated under reduced pressure to afford a yellow oil that was partitioned between DCM (20 mL) and saturated sodium hydrogen carbonate solution (10 mL). The aqueous phase was extracted an additional 2 times with DCM (3×20 mL). The combined DCM extracts were passed through a hydrophobic frit, and concentrated under reduced pressure.

AnalpH2_MeCN_4 min, Rt=2.03 min, m/z 736.4 [M+H]⁺

Purification via prep HPLC gave the two separated diastereomers: unassigned diastereomer A as a colourless solid (37.4 mg), and unassigned diastereomer B as a colourless solid (25.8 mg) Step 4: Benzyl (2R,3S)-3-[2-(dimethylamino)ethyl-methyl-carbamoyl]-2-[(2S,4R)-2-[(1-methylindazol-5-yl)methylcarbamoyl]-4-(p-tolylmethyl)pyrrolidine-1-carbonyl]piperidine-1-carboxylate (unassigned diastereomer B, 25.8 mg, 0.0351 mmol) was dissolved in MeOH (1.2 mL). The reaction vessel was purged and backfilled with nitrogen (×3) after which Pd/C 10% wt. (5.4 mg) was added. The reaction mixture was purged and filled with nitrogen a further 3 times. after which it was purged and filled with hydrogen (×3). The reaction mixture was stirred at room temperature under hydrogen for 2.75 hours. The reaction mixture was filtered through a Celite cartridge (2.5 g), and the product eluted with methanol, and the product-containing fractions combined and concentrated under reduced pressure. The crude product was purified via prep HPLC, and the product-containing fractions were combined and concentrated under reduced pressure, redissolved in 1:1 MeCN:H₂O and lyophilised to afford (2R,3S)—N-(2-(dimethylamino)ethyl)-N-methyl-2-((2S,4R)-2-(((1-methyl-1H-indazol-5-yl)methyl)carbamoyl)-4-(4-methylbenzyl)pyrrolidine-1-carbonyl)piperidine-3-carboxamide as a white solid (12.1 mg, 0.0201 mmol, 57%).

UPLC_PH9_MECN_QC_V1, Rt: 4.69 min, m/z 602.4 [M+H]⁺

UPLC_PH2_MECN_QC_V1, Rt: 2.65 min, m/z 602.4 [M+H]⁺

1H-NMR (400 MHz, DMSO-d₆) δ 8.58 (s, 0.5H), 8.32 (s, 3H), 8.14 (s, 0.5H), 7.96 (s, 1H), 7.54 (d, J=16.9 Hz, 2H), 7.26 (dd, J=20.1, 8.2 Hz, 1H), 7.07 (s, 4H), 5.26 (s, 0.5H), 4.41-4.31 (m, 2H), 4.01 (s, 3H), 3.62-2.89 (m, 7H), 2.78-2.75 (m, 1H), 2.67-2.62 (m, 1H), 2.40-2.07 (m, 11H), 1.90-1.11 (m, 7H).

### Synthesis of (2R,3S)-2-((2S,4R)-4-(4-cyclopropylbenzyl)-2-(((1-methyl-1H-indazol-5-yl)methyl)carbamoyl)pyrrolidine-1-carbonyl)-N-(2-(dimethylamino)ethyl)-N-methylpiperidine-3-carboxamide (M06060)

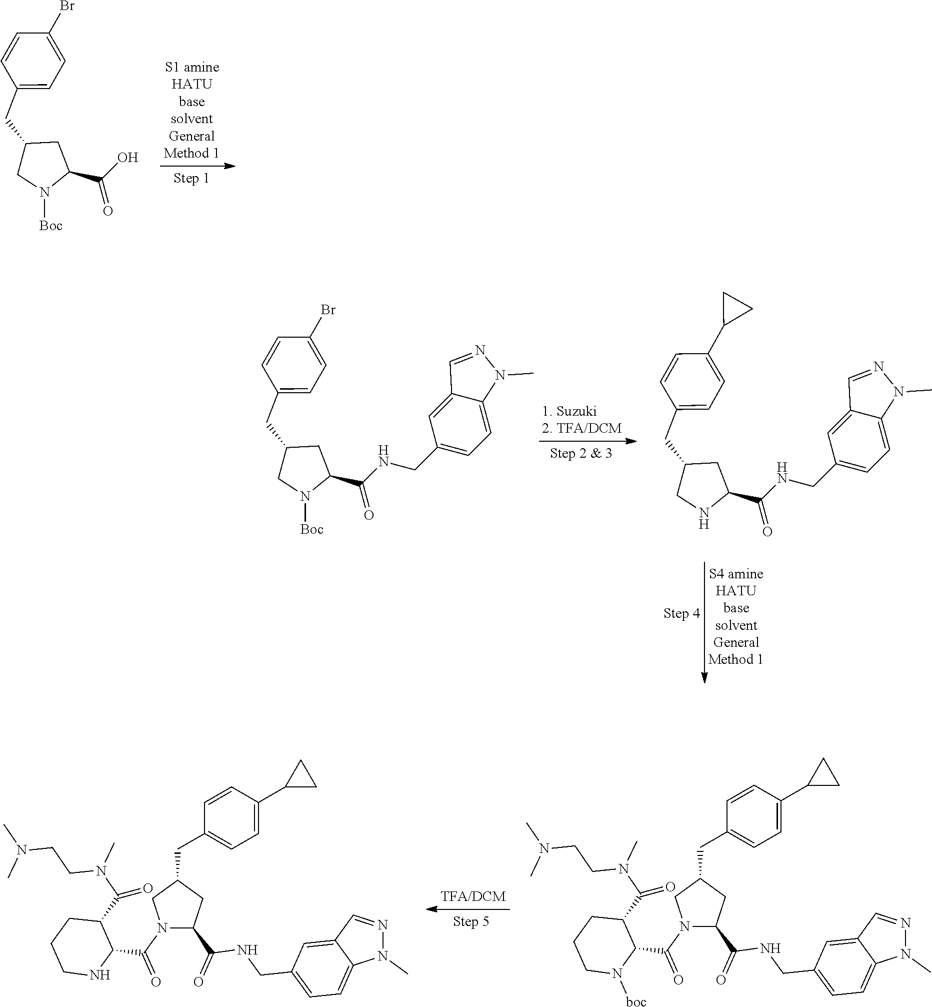

### Step 1: Synthesis of tert-butyl (2S,4R)-4-[(4-bromophenyl)methyl]-2-[(1-methylindazol-5-yl)methylcarbamoyl]pyrrolidine-1-carboxylate

A solution of Boc-(R)-γ-(4-bromo-benzyl)-L-proline (500.mg, 1.3 mmol) and HATU (495 mg, 1.3 mmol) was pre-stirred in N,N-dimethylformamide (2.0 mL) for 5 minutes, then 5-(aminomethyl)-1-methyl-1H-indazole (210 mg, 1.30 mmol) and N,N-diisopropylethylamine (0.68 mL, 3.90 mmol) were added and the mixture stirred at rt for 2 h. The solution was diluted with EtOAc (15 mL), water (15 mL) and brine (15 mL) and the phases separated. The aqueous layer was extracted with EtOAc (3×15 mL), and the combined organics were washed with brine (30 mL), dried through a phase separator and solvents removed in vacuo. The crude was purified by Flash column chromatography (10 g Sfar column, 0-100 EtOAc in iHex over 5 CVs, followed by 0-20% MeOH in DCM) to yield tert-butyl (2S,4R)-4-[(4-bromophenyl)methyl]-2-[(1-methylindazol-5-yl)methylcarbamoyl]pyrrolidine-1-carboxylate (680 mg, 1.29 mmol, 99%) as a yellow oil.

AnalpH9_MeCN_4 min, Rt: 2.69 min, m/z 527.3/529.3 [M+H]⁺

### Step 2: Synthesis of tert-butyl (2S,4R)-4-[(4-cyclopropylphenyl)methyl]-2-[(1-methylindazol-5-yl)methylcarbamoyl]pyrrolidine-1-carboxylate

To a microwave vial was added tert-butyl (2S,4R)-4-[(4-bromophenyl)methyl]-2-[(1-methylindazol-5-yl)methylcarbamoyl]pyrrolidine-1-carboxylate (213 mg, 0.36 mmol), Di(1-adamantyl)-n-butylphosphine (171 mg, 0.45 mmol), Palladium (II) acetate (21 mg, 0.09 mmol), Potassium Phosphate Tribasic (313 mg, 1.45 mmol) and cyclopropylboronic acid (62 mg, 0.73 mmol) then toluene (3.0 mL) and water (0.50 mL). The mixture was degassed with nitrogen for 5 minutes then capped. The reaction mixture was heated at 120° C. in a microwave for 1 hour.

The reaction mixture was passed through a Celite plug, washing through with EtOAc. The filtrate was concentrated under vacuum to give an orange oil. The crude material was purified by flash column chromatography (Gradient: 0 to 100% EtOAc in isohexane). The desired product tert-butyl (2S,4R)-4-[(4-cyclopropylphenyl)methyl]-2-[(1-methylindazol-5-yl)methylcarbamoyl]pyrrolidine-1-carboxylate (141 mg, 0.289 mmol, 79%) was isolated as an orange solid.

UPLC_pH9_MeCN_2 min, Rt: 1.95 min, m/z 389.2 [M+H−boc]⁺

### Step 3: Synthesis of (2S,4R)-4-[(4-cyclopropylphenyl)methyl]-N-[(1-methylindazol-5-yl)methyl]pyrrolidine-2-carboxamide

To a stirred solution of tert-butyl (2S,4R)-4-[(4-cyclopropylphenyl)methyl]-2-[(1-methylindazol-5-yl)methylcarbamoyl]pyrrolidine-1-carboxylate (139 mg, 0.28 mmol) in dichloromethane (3.0 mL) was added TFA (1.mL, 13.07 mmol). The mixture was stirred at rt for 3 hours.

The reaction mixture was concentrated under vacuum then dissolved in MeOH and purified on SCX-2. The cartridge washed with MeOH then the product eluted with 1.4M NH₃ in MeOH. The solution was evaporated under vacuum to afford the desired (2S,4R)-4-[(4-cyclopropylphenyl)methyl]-N-[(1-methylindazol-5-yl)methyl]pyrrolidine-2-carboxamide (88 mg, 0.23 mmol, 80%) as a yellow gum.

UPLC_pH9_MeCN_2 min, Rt: 1.79 min, m/z 389.2 [M+H−boc]⁺

### Step 4: Synthesis of tert-butyl 2-[(2S,4R)-4-[(4-cyclopropylphenyl)methyl]-2-[(1-methylindazol-5-yl)methylcarbamoyl]pyrrolidine-1-carbonyl]-3-[2-(dimethylamino)ethyl-methyl-carbamoyl]piperidine-1-carboxylate

To a stirred solution of (2S,4R)-4-[(4-cyclopropylphenyl)methyl]-N-[(1-methylindazol-5-yl)methyl]pyrrolidine-2-carboxamide (88 mg, 0.23 mmol), (2R,3S)-1-tert-butoxycarbonyl-3-[2-(dimethylamino)ethyl-methyl-carbamoyl]piperidine-2-carboxylic acid (81 mg, 0.23 mmol) in N,N-dimethylformamide (3.0 mL) was added N,N-diisopropylethylamine (0.059 mL, 0.34 mmol) and HATU (103 mg, 0.272 mmol). The mixture was stirred at rt for 30 minutes. The reaction mixture was purified directly by prep HPLC to yield two diastereomers. Fractions containing the desired diastereomer were evaporated under vacuum to afford tert-butyl 2-[(2S,4R)-4-[(4-cyclopropylphenyl)methyl]-2-[(1-methylindazol-5-yl)methylcarbamoyl]pyrrolidine-1-carbonyl]-3-[2-(dimethylamino)ethyl-methyl-carbamoyl]piperidine-1-carboxylate (36 mg, 0.050 mmol, 22%) as a white solid.

AnalpH9_MeCN_4 min, Rt: 2.78 min, m/z 728.7 [M+H]⁺

### Step 5: Synthesis of (2R,3S)-2-((2S,4R)-4-(4-cyclopropylbenzyl)-2-(((1-methyl-1H-indazol-5-yl)methyl)carbamoyl)pyrrolidine-1-carbonyl)-N-(2-(dimethylamino)ethyl)-N-methylpiperidine-3-carboxamide (M06060)

To a stirred solution of tert-butyl (2R,3S)-2-[(2S,4R)-4-[(4-cyclopropylphenyl)methyl]-2-[(1-methylindazol-5-yl)methylcarbamoyl]pyrrolidine-1-carbonyl]-3-[2-(dimethylamino)ethyl-methyl-carbamoyl]piperidine-1-carboxylate (35 mg, 0.05 mmol) in dichloromethane (3.0 mL) was added TFA (1.0 mL, 13 mmol). The reaction mixture was stirred at rt for 8 hours. The reaction mixture was concentrated under vacuum then dissolved in MeOH and purified on SCX-2. Cartridge washed with MeOH then the product eluted with 1.4M NH3 in MeOH. The solution was evaporated under vacuum and the crude purified by preparative HPLC. Fractions were combined and evaporated under vacuum then lyophilised to afford the desired product (2R,3S)-2-[(2S,4R)-4-[(4-cyclopropylphenyl)methyl]-2-[(1-methylindazol-5-yl)methylcarbamoyl]pyrrolidine-1-carbonyl]-N-[2-(dimethylamino)ethyl]-N-methyl-piperidine-3-carboxamide (11 mg, 0.0175 mmol, 36%) as a white solid.

UPLC_pH9_MECN_QC_V1, Rt: 5.74, m/z 628.4 [M+H]⁺

UPLC_pH2_MECN_QC_V1, Rt: 2.7, m/z 628.4 [M+H]⁺

1H-NMR (400 MHz, DMSO-D₆) δ 8.55 (t, J=6.0 Hz, 0.5H), 8.05-8.03 (m, 0.3H), 7.96 (d, J=2.7 Hz, 0.9H), 7.57-7.51 (m, 2H), 7.26 (dd, J=20.6, 9.2 Hz, 1H), 7.06-7.03 (m, 2H), 6.99-6.95 (m, 2H), 5.26-5.26 (m, 0.6H), 4.43-4.25 (m, 2.3H), 4.02 (s, 3.5H), 3.50-3.40 (m, 1.7H), 3.17-3.04 (m, 1.7H), 2.98-2.87 (m, 4.4H), 2.76 (d, J=7.8 Hz, 1.3H), 2.67-2.54 (m, 3.7H), 2.39-2.23 (m, 2.5H), 2.17-2.11 (m, 6.5H), 1.97-1.61 (m, 4.5H), 1.48-1.27 (m, 1.4H), 1.08 (d, J=13.3 Hz, 0.6H), 0.93-0.88 (m, 2H), 0.64-0.60 (m, 2H)

The following compound was made by analogous methods:

| | | | |
|---|---|---|---|
| M06070 | 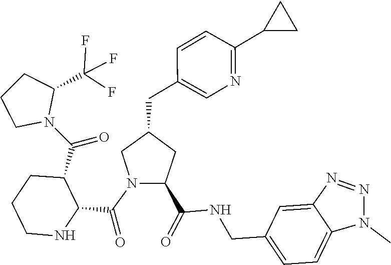 [SMILES: Cn1nnc2cc(CNC(=O)[C@@H]3C[C@@H](Cc4ccc(C5CC5)nc4)CN3C(=O)[C@@H]3NCCC[C@@H]3C(=O)N3CCC[C@@H]3C(F)(F)F)ccc21] | UPLC_pH9_MeCN_QC_ V1 Rt: 4.63 min, m/z 667.5 [M+H]+ UPLC_pH2_MeCN_QC_ V1 Rt: 2.57 min, m/z 667.5 [M + H]+ 1H-NMR (400 MHz, DMSO-D6) δ 8.71 (t, J = 6.0 Hz, 0.5H), 8.23 (dd, J = 13.7, 1.8 Hz, 1H), 8.16 (t, J = 6.0 Hz, 0.5H), 7.84-7.72 (m, 2H), 7.50-7.37 (m, 2H), 7.20 (dd, J = 7.8, 6.0 Hz, 1H), 5.32-5.29 (m, 0.5H), 4.87-4.74 (m, 1H), 4.52-4.32 (m, 2H), | 99 mg, 88%, White Solid |
| | (2S,4R)-4-[(6-cyclopropyl-3- | 4.29 (d, J = 4.6 Hz, | |
| | pyridyl)methyl]-N-[(1-methylbenzotriazol-5- | 3H), 4.16-4.10 (m, | |
| | yl)methyl]-1-[(2R,3S)-3-[(2R)-2- | 0.5H), 3.65-3.59 (m, | |
| | (trifluoromethyl)pyrrolidine-1- | 1H), 3.51-3.40 (m, 2H), | |
| | carbonyl]piperidine-2-carbonyl]pyrrolidine- | 3.19 (t, J = 9.2 Hz, | |
| | 2-carboxamide | 0.5H), 3.02-2.93 (m, | |
| | S1: (1-methyl-1H-benzo[d][1,2,3]triazol-5- | 2H), 2.82 (s, 0.5H), | |
| | yl)methanamine | 2.67-2.54 (m, 4H), | |
| | S2: (2S,4R)-4-[(6-bromo-3- | 2.37-2.16 (m, 1H), | |
| | pyridyl)methyl]-1-tert-butoxycarbonyl- | 2.08-1.79 (m, 7H), | |
| | pyrrolidine-2-carboxylic acid | 1.66-1.32 (m, 2H), 1.12 | |
| | S4: (2R,3S)-1-tert-butoxycarbonyl-3-[(2R)- | (d, J = 11.9 Hz, 1H), | |
| | 2-(trifluoromethyl)pyrrolidine-1- | 0.93-0.84 (m, 4H). | |
| | carbonyl]piperidine-2-carboxylic acid | Missing protons: 2 | |
| | Step 1: GM1 with DMF and DIPEA. Amine | | |
| | used as HCI salt. | | |
| | Step 2: Reverse Phase purification | | |
| | Step 3: GM1 with DMF and DIPEA. | | |
| | Purified by prep HPLC | | |
| | Step 4: GM2A. Final purificatione | | |

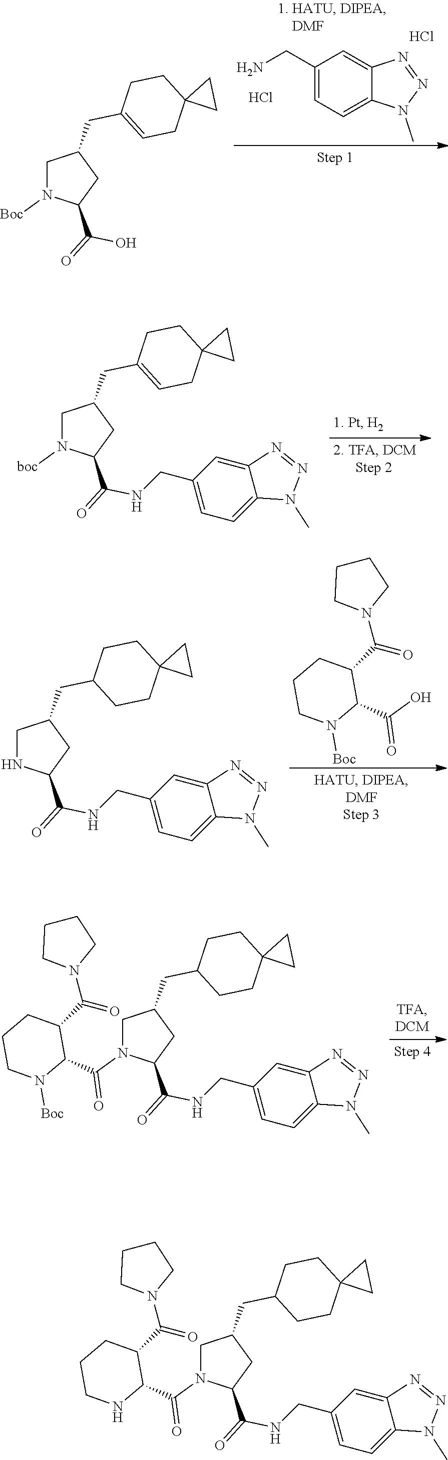

### Step 1: Synthesis of (2S,4R)—N-[(1-methylbenzotriazol-5-yl)methyl]-4-(spiro[2.5]oct-6-en-6-ylmethyl)pyrrolidine-2-carboxamide

(1-methylbenzotriazol-5-yl)methanamine; dihydrochloride (315.41 mg, 1.34 mmol) and (2S,4R)-1-tert-butoxycarbonyl-4-(spiro[2.5]oct-6-en-6-ylmethyl)pyrrolidine-2-carboxylic acid (450.mg, 1.34 mmol) were combined and dissolved in a mixture of N,N-dimethylformamide (5.0 mL) and N,N-Diisopropylethylamine (0.93 mL, 5.37 mmol). To the this solution HATU (765.13 mg, 2.01 mmol) was added and the solution was stirred overnight at room temperature. The crude was purified on a C18 Reverse Phase Column (0 to 80% acetonitrile in water: ammonium bicarbonate-0.1%). The solvent was removed to give the Boc-protected intermediate as a pale-yellow oil.

UPLC_pH9_MeCN_2 min, Rt: 2.00 min, m/z 480.3 [M+H]⁺

### Step 2: Synthesis of tert-butyl (2S,4R)-2-[(1-methylbenzotriazol-5-yl)methylcarbamoyl]-4-(spiro[2.5]octan-6-ylmethyl)pyrrolidine-1-carboxylate

Tert-butyl (2S,4R)-2-[(1-methylbenzotriazol-5-yl)methylcarbamoyl]-4-(spiro[2.5]oct-6-en-6-ylmethyl)pyrrolidine-1-carboxylate (125 mg, 0.26 mmol) was transferred in a 10 mL vial equipped with a stirring bar and ethyl acetate (5.0 mL). Platinum (IV) oxide, Adam's catalyst (30 mg, 0.13 mmol) was added and an hydrogen balloon was bubbled inside the reaction. The reaction was stirred at room temperature for 4 hours and then additional PtO₂ (20 mg, 0.09 mmol) was added and the reaction was stirred for an additional hour. The crude was filtered through a Celite plug (1 cm) and washed with additional EtOAc (5 mL). The solvent was removed in vacuo to give tert-butyl (2S,4R)-2-[(1-methylbenzotriazol-5-yl)methylcarbamoyl]-4-(spiro[2.5]octan-6-ylmethyl)pyrrolidine-1-carboxylate (35 mg, 0.0727 mmol, 27.88%, eDMX266-215-1) as a colourless oil.

AnalpH9_MeCN_2 min, Rt: =2.05 min, m/z 504.2 [M+Na]+

### Step 3: Synthesis of (2S,4R)—N-[(1-methylbenzotriazol-5-yl)methyl]-4-(spiro[2.5]octan-6-ylmethyl)pyrrolidine-2-carboxamide

tert-butyl (2S,4R)-2-[(1-methylbenzotriazol-5-yl)methylcarbamoyl]-4-(spiro[2.5]octan-6-ylmethyl)pyrrolidine-1-carboxylate (35 mg, 0.0727 mmol) was treated with dichloromethane (2 mL) and trifluoroacetic acid (1 mL). The mixture was stirred for two hours and then the solvent was removed. The crude was purified by preparative HPLC to give (2S,4R)—N-[(1-methylbenzotriazol-5-yl)methyl]-4-(spiro[2.5]octan-6-ylmethyl)pyrrolidine-2-carboxamide (23 mg, 0.0603 mmol, 82.96%) as a pale yellow oil.

AnalpH9_MeCN_2 min, Rt: =1.86 min, m/z 382.3 [M+H]+

### Step 4: Synthesis of [(2S,4R)-2-[(1-methylbenzotriazol-5-yl)methylcarbamoyl]-4-(spiro[2.5]octan-6-ylmethyl)pyrrolidine-1-carbonyl]-3-(pyrrolidine-1-carbonyl)piperidine-1-carboxylate

(2S,4R)—N-[(1-methylbenzotriazol-5-yl)methyl]-4-(spiro[2.5]octan-6-ylmethyl)pyrrolidine-2-carboxamide (23 mg, 0.06 mmol) and (2R,3S)-1-tert-butoxycarbonyl-3-(pyrrolidine-1-carbonyl)piperidine-2-carboxylic acid (19.7 mg, 0.06 mmol) were combined and dissolved in a mixture of N,N-dimethylformamide (1.0 mL) and N,N-diisopropylethylamine (0.026 mL, 0.151 mmol) in a 10 ml microwave CEM vial. To the this solution HATU (27.51 mg, 0.07 mmol) was added and the solution was stirred overnight at room temperature. The crude product was purified by preparative HPLC to give tert-butyl (2R,3S)-2-[(2S,4R)-2-[(1-methylbenzotriazol-5-yl)methylcarbamoyl]-4-(spiro[2.5]octan-6-ylmethyl)pyrrolidine-1-carbonyl]-3-(pyrrolidine-1-carbonyl)piperidine-1-carboxylate (21 mg, 0.0304 mmol, 50.5%) as a white solid.

AnalpH9_MeCN_2 min, Rt: =2.06 min, m/z 690.4 [M+H]+

### Step 5: Synthesis of (2S,4R)—N-[(1-methylbenzotriazol-5-yl)methyl]-1-[(2R,3S)-3-(pyrrolidine-1-carbonyl)piperidine-2-carbonyl]-4-(spiro[2.5]octan-6-ylmethyl)pyrrolidine-2-carboxamide

tert-butyl (2R,3S)-2-[(2S,4R)-2-[(1-methylbenzotriazol-5-yl)methylcarbamoyl]-4-(spiro[2.5]octan-6-ylmethyl)pyrrolidine-1-carbonyl]-3-(pyrrolidine-1-carbonyl)piperidine-1-carboxylate (21 mg, 0.03 mmol) was dissolved in dichloromethane (1.0 mL) and trifluoroacetic acid (0.30 mL). The solution was stirred for 1 hour and then the solvent was removed under reduced pressure. The crude was separated by preparative HPLC to give (2S,4R)—N-[(1-methylbenzotriazol-5-yl)methyl]-1-[(2R,3S)-3-(pyrrolidine-1-carbonyl)piperidine-2-carbonyl]-4-(spiro[2.5]octan-6-ylmethyl)pyrrolidine-2-carboxamide (2.5 mg, 0.0042 mmol, 13.7%) as a white solid.

UPLC_pH9_MeCN_QC_V1, Rt: 6.95 min, m/z 590.5 [M+H]+

UPLC_pH2_MeCN_QC_V1. Rt: 4.96 min. m/z 590.5 [M+H]+

1H-NMR (400 MHz, ACETONITRILE-D3) δ 7.82 (s, 1H), 7.59 (d, J=12.8 Hz, 1H), 7.40 (d, J=22.9 Hz, 2H), 5.29 (s, 0.5H), 5.13 (d, J=11.9 Hz, 0.5H), 4.48-4.39 (m, 3H), 4.19 (d, J=15.1 Hz, 4H), 3.63 (d, J=22.4 Hz, 1H), 3.34 (d, J=15.6 Hz, 2H), 3.11 (dd, J=39.2, 18.5 Hz, 4H), 2.92 (d, J=12.4 Hz, 2H), 2.66-2.53 (m, 1H), 2.34 (d, J=14.2 Hz, 1H), 1.85-1.41 (m, 11H), 1.28-1.11 (m, 5H), 1.06-0.82 (m, 3H), 0.23-0.12 (m, 3H). Missing protons: 1

### General Test Methods

The activities of the compounds of the invention have been determined in vitro using the following assays protocols for the screening of activity of FXIIa and other proteases. Each of these assays were performed in a purified system employing the use of chromogenic assays in microplate plate wells. Chromogenic peptide substrates mimicking natural protein substrates are attached via an amide bond to a chromogenic group. Paranitroaniline (pNA) is released from the peptide following catalyses by the proteolytic enzyme; the absorbance increases and can be monitored at 405 nm.

All compounds were dissolved in 100% (v/v) DMSO to a stock concentration of 10 mM, the highest concentration of compound used in each assay is 500 μM. The final concentrations of DMSO were 5% (v/v) in 50 mM Tris 137 mM NaCl pH 7.4. Where no test compound was added a final concentration of 5% DMSO was employed.

### Determination of Factor XIIa Inhibition

Factor XIIa activity was measured using a chromogenic substrate S-2302 (Chromogenix). Various concentrations of compound were incubated with 10 nM of FXIIa and incubated at 37° C. for 10 minutes in 50 mM Tris, 137 mM NaCl, pH 7.4, prior to the addition of the chromogenic substrate S-2302 at a concentration equal the to the experimentally derived KM. Kinetic readings at 405 nm were monitored every 12 secs for a total duration of 3 hours at 37° C. Gradients of initial rates were determined and employed to calculate IC₅₀ values.

The Ki data obtained in the above manner is shown in Table 1 below. The activity of the compounds of the invention has been categorised based on the Ki values, the categories being “†”, “*”, “**” and “***”. The category “†” refers to compounds with a Kᵢ value of 5 μM or more (e.g. a Kᵢ value of 5 μM or more and 10 μM or less). The category “*” refers to compounds with a Ki value of greater than 0.8 μM and less than 5 μM). The category “**” refers to compounds with a Ki value of 0.2 μM to 0.8 μM. The category “***” refers to compounds with a Ki value of less than 0.2 μM.

### Determination of Selectivity

To determine selectivity of test compounds, these test compounds were assayed for inhibitory activity against other serine proteases including FXa and thrombin. Essentially compounds at increasing concentrations were incubated with each enzyme: FXa (5 nM) and thrombin (5 nM), for 10 mins at 37° C. followed by the appropriate chromogenic substrate, S2765 (Chromogenix) and GPR (Bachem) respectively in 50 mM Tris, 137 mM NaCl, pH 7.4. Substrates were used at a concentration equal to the appropriate experimentally derived K_{M}. Kinetic readings at 405 nm were monitored every 12 secs for a total duration of 3 hours at 37° C. Gradients of initial rates were determined and employed to calculate IC₅₀ values. Values of IC₅₀ were converted to Ki values based on the formula:

Kᵢ═IC₅₀/(1+[Substrate]/Km)

Where [Substrate] denotes the concentration of substrate used in the assay and Km is the determined value of each enzyme with its own substrate. Compounds demonstrate competitive inhibition.

The fold selectivity for thrombin and FXa are also shown in Table 1 below. The fold selectivity demonstrates a preferential inhibition of FXIIa over FXa and thrombin. The fold selectivity for FXIIa over thrombin for the compounds of the invention has been categorised based on the fold selectivity values, the categories being “+”, “++” and “+++”. The category “+” refers to fold selectivity values less than 10 (e.g. a fold selectivity value of less than 10 and greater than 1). The category “++” refers to a fold selectivity value of 10 to 100. The category “+++” refers to fold selectivity values greater than 100.

The fold selectivity for FXIIa over FXa for the compounds of the invention has been categorised based on the fold selectivity values, the categories being “o”, “oo” and “ooo”. The category “o” refers to fold selectivity values less than 10 (e.g. a fold selectivity value of less than 10 and greater than 1). The category “oo” refers to a fold selectivity value of 10 to 100. The category “ooo” refers to fold selectivity values greater than 100.

TABLE 1

| | | Thrombin | |
|---|---|---|---|
| Compound | FXIIa alpha | (human)/FXIIa | FXa (human)/FXIIa |
| code | (Human) Ki | alpha (human) | alpha (human) |
| M04490 | *** | +++ | ∘∘∘ |
| M00466 | *** | +++ | ∘∘∘ |
| M00467 | *** | + | ∘∘ |
| M05675 | *** | +++ | ∘∘∘ |
| M05712 | ** | +++ | ∘∘∘ |
| M05720 | *** | +++ | ∘∘∘ |
| M05733 | *** | ++ | ∘∘∘ |
| M05747 | *** | +++ | ∘∘∘ |
| M05748 | * | ++ | ∘∘∘ |
| M05751 | ** | +++ | ∘∘∘ |
| M05752 | *** | ++ | ∘∘∘ |
| M05753 | ** | ++ | ∘∘∘ |
| M05754 | ** | +++ | ∘∘∘ |
| M05757 | *** | +++ | ∘∘∘ |
| M05764 | *** | +++ | ∘∘∘ |
| M05766 | *** | +++ | ∘∘∘ |
| M05770 | ** | +++ | ∘∘∘ |
| M05772 | ** | +++ | ∘∘∘ |
| M05773 | ** | ++ | ∘∘∘ |
| M05775 | * | ++ | ∘∘∘ |
| M05777 | ** | +++ | ∘∘∘ |
| M05778 | ** | +++ | ∘∘∘ |
| M05785 | *** | +++ | ∘∘∘ |
| M05787 | ** | +++ | ∘∘∘ |
| M05788 | *** | +++ | ∘∘∘ |
| M05799 | *** | ++ | ∘∘∘ |
| M05804 | ** | +++ | ∘∘∘ |
| M05806 | *** | +++ | ∘∘∘ |
| M05809 | ** | +++ | ∘∘∘ |
| M05810 | * | ++ | ∘∘ |
| M05811 | * | ++ | ∘∘∘ |
| M05820 | *** | +++ | ∘∘∘ |
| M05836 | * | +++ | ∘∘∘ |
| M05837 | *** | +++ | ∘∘∘ |
| M05838 | ** | +++ | ∘∘∘ |
| M05839 | ** | +++ | ∘∘∘ |
| M05840 | *** | +++ | ∘∘∘ |
| M05843 | ** | +++ | ∘∘∘ |
| M05844 | * | ++ | ∘∘ |
| M05851 | * | +++ | ∘∘∘ |
| M05853 | *** | +++ | ∘∘∘ |
| M05854 | ** | +++ | ∘∘∘ |
| M05855 | ** | +++ | ∘∘∘ |
| M05863 | *** | +++ | ∘∘∘ |
| M05874 | ** | +++ | ∘∘∘ |
| M05875 | ** | +++ | ∘∘∘ |
| M05877 | *** | +++ | ∘∘∘ |
| M05878 | *** | +++ | ∘∘∘ |
| M05881 | *** | +++ | ∘∘∘ |
| M05882 | * | +++ | ∘∘∘ |
| M05899 | *** | + | ∘∘ |
| M05913 | ** | +++ | ∘∘∘ |
| M05902 | *** | +++ | ∘∘∘ |
| M05992 | ** | +++ | ∘∘∘ |
| M06003 | ** | +++ | ∘∘∘ |
| M06006 | ** | +++ | ∘∘∘ |
| M06013 | *** | +++ | ∘∘∘ |
| M06010 | ** | +++ | ∘∘∘ |
| M06030 | ** | ++ | ∘∘∘ |
| M06033 | *** | +++ | ∘∘∘ |
| M06034 | *** | +++ | ∘∘∘ |
| M06039 | *** | +++ | ∘∘∘ |
| M06047 | *** | +++ | ∘∘∘ |
| M06048 | *** | +++ | ∘∘∘ |
| M06060 | *** | +++ | ∘∘∘ |
| M06061 | *** | +++ | ∘∘∘ |
| M06063 | *** | +++ | ∘∘∘ |
| M06079 | ** | +++ | ∘∘∘ |
| M06083 | *** | +++ | ∘∘∘ |
| M06084 | *** | +++ | ∘∘∘ |
| M06085 | *** | +++ | ∘∘∘ |
| M06099 | *** | +++ | ∘∘∘ |
| M06109 | *** | +++ | ∘∘∘ |
| M06112 | *** | +++ | ∘∘∘ |
| M06128 | *** | +++ | ∘∘∘ |
| M06129 | *** | +++ | ∘∘∘ |
| M06131 | *** | +++ | ∘∘∘ |
| M06070 | ** | nd | ∘∘∘ |
| M06068 | * | nd | ∘∘∘ |
| M06049 | *** | +++ | ∘∘∘ |
| M06021 | ** | +++ | ∘∘∘ |
| M06009 | ** | +++ | ∘∘∘ |
| M05985 | * | nd | nd |
| M05997 | ** | +++ | ∘∘∘ |
| M05983 | * | nd | ∘∘∘ |
| M05965 | † | ++ | ∘∘ |
| M06089 | ** | +++ | ∘∘∘ |
| M05798 | * | +++ | ∘∘∘ |
| M05991 | *** | +++ | ∘∘∘ |
| M05934 | *** | +++ | ∘∘∘ |
| M05935 | ** | +++ | ∘∘∘ |
| M06077 | * | nd | nd |

nd refers to entries where test data has not been obtained.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

What is claimed is:

1. A compound according to formula (I) and pharmaceutically acceptable salts thereof: 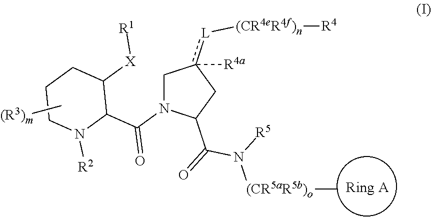
wherein
—X— is —C(O);
R¹ is selected from: —NR^{1a}R^{1b} and a substituted or unsubstituted 3 to 8 membered monocyclic or bicyclic (fused, bridged or spiro) heterocyclic group comprising a nitrogen atom and 0, 1, or 2 additional heteroatoms selected from O, N or S, wherein the heterocyclic ring system is connected to —X— via a nitrogen atom; wherein R^{1a} and R^{1b} are each independently selected from: substituted or unsubstituted C₁₋₆ alkyl, substituted or unsubstituted C₃₋₇ cycloalkyl, a substituted or unsubstituted 3 to 7 membered heterocyclic group having 1, 2 or 3 heteroatoms selected from O, N or S, substituted or unsubstituted —C₁₋₆ alkyl-C₃₋₇ cycloalkyl, a substituted or unsubstituted —C₁₋₆ alkyl-3 to 7 membered heterocyclic group having 1, 2 or 3 heteroatoms selected from O, N or S, and a substituted or unsubstituted —C₁₋₆ alkyl-5-6 membered heteroaromatic group having 1, 2 or 3 heteroatoms selected from O, N or S;
wherein, when substituted, the substituent of R¹ is selected from: halo, ═O, —CN, —OH, C₁₋₆ alkyl, C₃₋₆ cycloalkyl, C₁₋₆ haloalkyl, —O—Ci-s alkyl, —O—C₃₋₆ cycloalkyl, —O—C₁₋₆ haloalkyl, —C₁₋₆ alkyl-O—C₁₋₆ alkyl, —C₁₋₆ alkyl-O—C₃₋₆ cycloalkyl, —C₁₋₆ alkyl-O—C₁₋₆ haloalkyl, —NR^{1c}R^{1d}, —NR^{1c}(SO₂)R^{1d}, —NR^{1c}(C(O))R^{1d}, —C(O)NR^{1c}R^{1d}, —SO₂NR^{1c}R^{1d}, a 5 to 10 membered heteroaryl group having 1, 2 or 3 heteroatoms selected from O, N or S, and a 6 to 10 membered aryl group; wherein R^{1c} and R^{1d} are independently at each occurrence selected from: H, C₁₋₆ alkyl, C₁₋₆ haloalkyl, C₃₋₆ cycloalkyl, C₃₋₆ cyclohaloalkyl, 5 to 10 membered heteroaryl group having 1, 2 or 3 heteroatoms selected from O, N or S, and a 6 to 10 membered aryl group;
wherein, when substituted, the substituent of R^{1a} and R^{1b} is selected from: halo, —CN, —OH, C₁₋₆ alkyl, C₃₋₇ cycloalkyl, C₁₋₆ haloalkyl, C₃₋₇ cyclohaloalkyl, —O—C₁₋₆ alkyl, —O—C₃₋₇ cycloalkyl, —O—C₁₋₆ haloalkyl, —O—C₃₋₇ cyclohaloalkyl, —C₁₋₆ alkyl-O—C₁₋₆ alkyl, —C₁₋₆ alkyl-O—C₃₋₇ cycloalkyl, —C₁₋₆ alkyl-O—C₁₋₆ haloalkyl, —C₁₋₆ alkyl-O—C₃₋₇ cyclohaloalkyl, —C₁₋₆ haloalkyl-O—C₁₋₆ alkyl, —C₁₋₆ haloalkyl-O—C₃₋₇ cycloalkyl, —C₁₋₆ haloalkyl-O—C₁₋₆ haloalkyl, —C₁₋₆ haloalkyl-O—C₃₋₇ cyclohaloalkyl, —NR^{1e}R^{1f}, —NH(═NH)NR^{1e}R^{1f}, —NR^{1e}(SO₂)R^{1f}—NR^{1e}(C(O))R^{1f}, —C(O)NR^{1e}R^{1f}, and —SO₂NR^{1e}R^{1f}; R^{1e} and R^{1f} are independently at each occurrence selected from: H, C₁₋₆ alkyl, C₁₋₆ haloalkyl, C₃₋₆ cycloalkyl, C₃₋₆ cyclohaloalkyl, 5 to 10 membered heteroaryl group having 1, 2 or 3 heteroatoms selected from O, N or S, and a 6 to 10 membered aryl group;
R² is selected from: H, C₁₋₆ alkyl, C₁₋₆ haloalkyl, C₃₋₆ cycloalkyl, and 3 to 6 membered heterocyloalkyl;
R³ is selected from: halo, —CN, —OH, C₁₋₆ alkyl, C₁₋₆ haloalkyl, —O—C₁₋₆ alkyl, —O—C₁₋₆ haloalkyl, —NR^{3a}R^{3b}, —NR^{3a}(C(O))R^{3b}, and —C(O)NR^{3a}R^{3b}; wherein R^{3a} and R^{3b} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
m is selected from 0, 1, 2, or 3;
wherein the residue 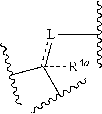
is selected from: 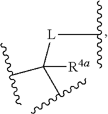
wherein is selected from: bond, —O—, —NR^{4b}—, and —NR^{4c}C(O)—; and 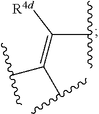
R^{4a} is selected from: H, —OH, halo, C₁₋₄ alkyl or C₁₋₄ haloalkyl;
R^{4b} is H, C₁₋₆ alkyl or —C(O)C₁₋₆ alkyl;
R^{4c} is H or C₁₋₆ alkyl;
R^{4d} is H or C₁₋₆ alkyl;
R^{4e} and R^{4f} are independently at each occurrence selected from: H, —CN, halo, C₁₋₄ alkyl, C₁₋₄ haloalkyl, —OR^{4g}, —NR^{4g}R^{4h}, C₃₋₈ cycloalkyl, 3 to 6 membered heterocyclic ring, 6 to 10 membered aryl, 5 to 10 membered heteroaryl, wherein the C₃₋₈ cycloalkyl, 3 to 6 membered heterocyclic ring, 6 to 10 membered aryl or 5 to 10 membered heteroaryl group is unsubstituted or substituted with 1, 2 or 3 R⁴ⁱ groups; wherein R^{4g} and R^{4h} are independently at each occurrence selected from: H and C₁₋₄ alkyl; and wherein R⁴ⁱ is independently at each occurrence selected from: halo, C₁₋₄ alkyl, C₁₋₄ haloalkyl, C₃₋₆ cycloalkyl, C₃₋₆ cyclohaloalkyl, —OR^{4j}, —NR^{4k}R^{4l}, NR^{4k}(C(O))R^{4l}, —C(O)NR^{4k}R^{4l}, —CN, —C(O)R^{4g}, ═O, —SO₂R^{4g}, benzyl, phenyl, unsubstituted 5 or 6 membered heteroaryl, or methyl substituted 5 or 6 membered heteroaryl; R^{4j} is selected from: H, C₁₋₄ alkyl, C₁₋₄ haloalkyl, phenyl or benzyl; R^{4k} and R^{4l} are independently at each occurrence selected from: H, C₁₋₆ alkyl, C₃₋₆ cycloalkyl, 5 to 10 membered heteroaryl group having 1, 2 or 3 heteroatoms selected from O, N or S, and a 6 to 10 membered aryl group;
n is selected from 0, 1, 2, 3, or 4;
R⁴ is selected from: H, halo, —CN, C₁₋₄ alkyl, C₁₋₄ haloalkyl, —OR^{4g}, —NR^{4g}R^{4h}, a monocyclic or bicyclic 6 to 10 membered aryl, C₃₋₈ cycloalkyl, C₄₋₈ cycloalkenyl, 3 to 6 membered heterocyclic ring comprising 1, 2, or 3 heteroatoms selected from O, N or S, a monocyclic or bicyclic 5 to 10 membered heteroaryl comprising 1, 2, or 3 heteroatoms selected from O, N or S, a bicyclic (fused, bridged, or spiro) 6 to 10 membered cycloalkyl ring system, a bicyclic (fused, bridged, or spiro) 6 to 10 membered cycloalkenyl ring system and a bicyclic (fused, bridged, or spiro) 6 to 10 membered heterocyclic ring system comprising 1, 2, or 3 heteroatoms selected from O, N or S, wherein the C₃₋₈ cycloalkyl, C₄₋₈ cycloalkenyl, 3 to 6 membered heterocyclic ring, 6 to 10 membered aryl, 5 to 10 membered heteroaryl group, bicyclic (fused, bridged, or spiro) 6 to 10 membered cycloalkyl ring system, bicyclic (fused, bridged, or spiro) 6 to 10 membered cycloalkenyl ring system, or bicyclic (fused, bridged, or spiro) 6 to 10 membered heterocyclic ring system is unsubstituted or substituted with 1, 2 or 3 R⁴ⁱ;
R⁵ is H or C₁₋₆ alkyl;
o is selected from 1, 2 or 3;
R^{5a} and R^{5b} are independently at each occurrence selected from: H, substituted or unsubstituted C₁₋₆ alkyl, substituted or unsubstituted C₃₋₆ cycloalkyl, and substituted or unsubstituted C₁₋₆ haloalkyl, wherein each substituent is independently selected from halo, —OH, and —CN;
Ring A is selected from a substituted or unsubstituted 5 to 10 membered monocyclic or bicyclic heteroaryl group having 1, 2 or 3 heteroatoms selected from O, N or S, a substituted or unsubstituted 6 to 10 membered monocyclic or bicyclic aryl group, and a substituted or unsubstituted monocyclic or bicyclic (fused, bridged, or spiro) 6 to 10 membered heterocyclic ring system comprising 1, 2, or 3 heteroatoms selected from O, N or S, wherein, when substituted, the heteroaryl group, aryl group, or heterocyclic ring system are substituted with 1, 2, or 3 substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d};
R^{5c} and R^{5d} are independently at each occurrence selected from: H, substituted or unsubstituted C₁₋₆ alkyl, substituted or unsubstituted C₃₋₆ cycloalkyl, and substituted or unsubstituted C₁₋₆ haloalkyl, wherein each substituent is independently selected from halo, —OH, and —CN

2. (canceled)

3. (canceled)

4. The compound of claim 1, wherein R¹ is a substituted or unsubstituted 4 to 7 membered monocyclic or bicyclic (fused, bridged, or spiro) heterocyclic ring system comprising a nitrogen atom and 0 or 1 additional heteroatoms selected from O, N or S, wherein the heterocyclic ring system is connected to —X— via a nitrogen atom.

5. The compound of claim 1, wherein R¹ is selected from a substituted or unsubstituted:

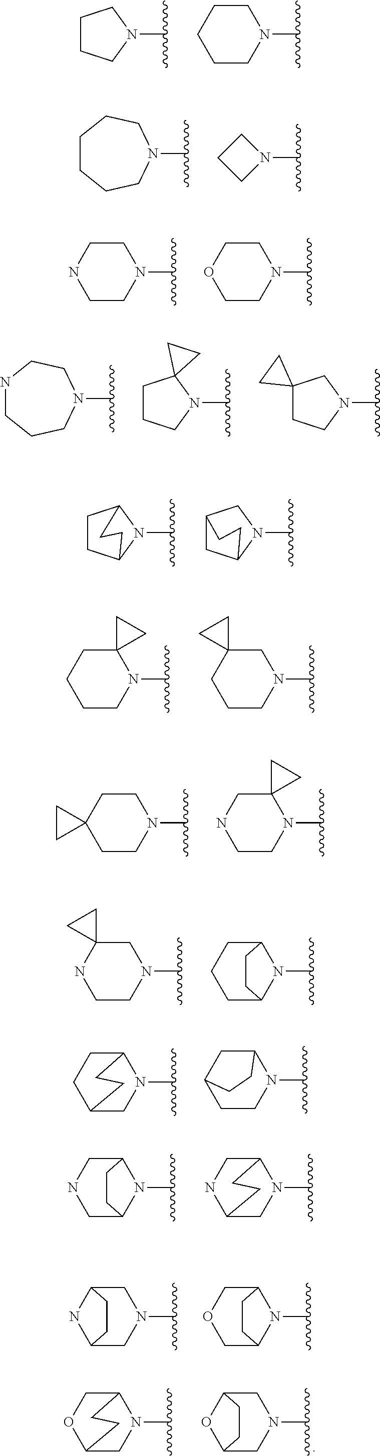

6. The compound of claim 4, wherein, when substituted, the substituent of R¹ is selected from halo, C₁₋₃ alkyl, C₁₋₃ haloalkyl, and —O—C₁₋₃ alkyl.

7. The compound of claim 1, wherein R¹ is —NR^{1a}R^{1b}.

8. The compound of claim 7, wherein Ria and R^{1b} are independently at each occurrence selected from: substituted or unsubstituted C₁₋₃ alkyl, substituted or unsubstituted C₃₋₇ cycloalkyl, a substituted or unsubstituted 3 to 7 membered heterocyclic group having 1, 2 or 3 heteroatoms selected from O, N or S, substituted or unsubstituted —C₁₋₃ alkyl-C₃₋₇ cycloalkyl, a substituted or unsubstituted —C₁₋₃ alkyl-3 to 7 membered heterocyclic group having 1, 2 or 3 heteroatoms selected from O, N or S; or wherein R^{1a} and R^{1b} are independently at each occurrence selected from: substituted or unsubstituted C₁₋₃ alkyl, substituted or unsubstituted C₃ cycloalkyl, a substituted or unsubstituted 3 to 6 membered heterocyclic group having 1, 2 or 3 heteroatoms selected from O, N or S, a substituted or unsubstituted —C₁₋₃ alkyl-3 to 6 membered heterocyclic group having 1, 2 or 3 heteroatoms selected from O, N or S.

9. (canceled)

10. The compound of claim 7, wherein, when R^{1a} and R^{1b} are substituted, each substituent is independently selected from: halo, —CN, —OH, C₁₋₃ alkyl, C₃₋₇ cycloalkyl, C₁₋₃ haloalkyl, —O—C₁₋₃ alkyl, —O—C₃₋₇ cycloalkyl, —O—C₁₋₃ haloalkyl, —C₁₋₃ alkyl-O—C₁₋₃ alkyl, —C₁₋₃ alkyl-O—C₃₋₇ cycloalkyl, —C₁₋₃ alkyl-O—C₁₋₃ haloalkyl, —NR^{1e}R^{1f}, —NR^{1e}(SO₂)R^{1f}, —NR^{1e}(C(O))R^{1f}, —C(O)NR^{1e}R^{1f}, and —SO₂NR^{1e}R^{1f}.

11. The compound of claim 1, wherein R² is selected from H, and C₁₋₃ alkyl.

12. The compound of claim 1, wherein m is 0.

13. The compound of claim 1, wherein the residue 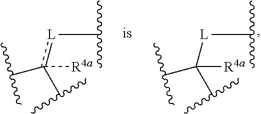
is wherein L is a bond.

14. The compound of claim 1, wherein the residue 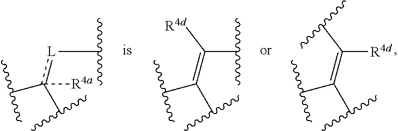
wherein R^{4d} is H.

15. (canceled)

16. The compound of claim 1, wherein n is 1, or wherein n is 0.

17. (canceled)

18. The compound of claim 1, wherein R⁴ is selected from: a monocyclic or bicyclic 6 to 10 membered aryl, a monocyclic or bicyclic 5 to 10 membered heteroaryl, a bicyclic (fused, bridged, or spiro) 6 to 10 membered cycloalkyl ring system, a bicyclic (fused, bridged, or spiro) 6 to 10 membered cycloalkenyl ring system, and a monocyclic or bicyclic (fused, bridged, or spiro) 6 to 10 membered heterocyclic ring system comprising 1, 2, or 3 heteroatoms selected from O, N or S, wherein the monocyclic or bicyclic 6 to 10 membered aryl, the monocyclic or bicyclic 5 to 10 membered heteroaryl, the bicyclic (fused, bridged, or spiro) 6 to 10 membered cycloalkyl ring system, the bicyclic (fused, bridged, or spiro) 6 to 10 membered cycloalkenyl ring system or the monocyclic or bicyclic (fused, bridged, or spiro) 6 to 10 membered heterocyclic ring system is unsubstituted or substituted with 1, 2 or 3 R⁴ⁱ.

19. The compound of claim 1, wherein R⁴ⁱ is independently at each occurrence selected from: halo, C₁ alkyl, C₁ haloalkyl, C₃ cycloalkyl, C₃ cyclohaloalkyl, and —OR^{4j}.

20. The compound of claim 1, wherein R⁴ is H.

21. (canceled)

22. The compound of claim 1, wherein o is 1.

23. The compound of claim 1, wherein R^{4e} and R^{4f} are H; and/or wherein R⁵ is H; and/or wherein R^{5a} and R^{5b} are H.

24. The compound of claim 1, wherein Ring A is selected from a substituted or unsubstituted 9 to 10 membered bicyclic heteroaryl group having 1, 2 or 3 heteroatoms selected from O, N or S, and a substituted or unsubstituted bicyclic (fused, bridged, or spiro) 9 to 10 membered heterocyclic ring system comprising 1, 2, or 3 heteroatoms selected from O, N or S, wherein, when substituted, the bicyclic heteroaryl group or the bicyclic heterocyclic ring system are substituted with 1, 2, or 3 independently substituents selected from: halo, —CN, C₁₋₆ alkyl, C₁₋₆ haloalkyl, C₃₋₆ cycloalkyl, deuterated C₁₋₆ alkyl, —OR^{5c}, —NR^{5c}R^{5d} or C₁₋₄ alkyl substituted by —NR^{5c}R^{5d}; optionally 1, 2, or 3 substituents independently selected from: halo, C₁₋₃ alkyl, C₁₋₃ haloalkyl, deuterated C₁₋₃ alkyl, or —OR^{5c}; optionally wherein Ring A is selected from:

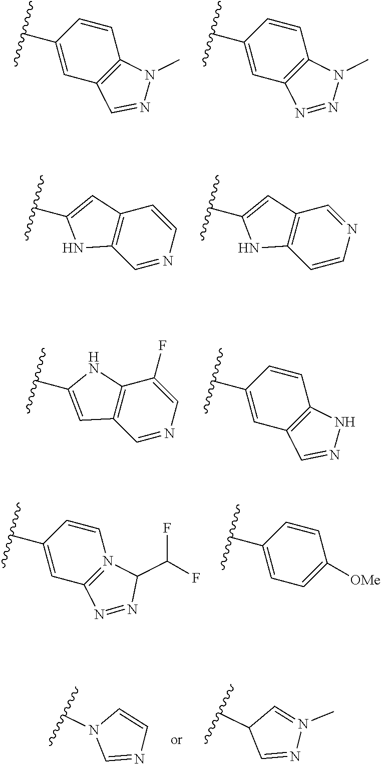

25. (canceled)

26. (canceled)

27. A pharmaceutical formulation comprising a compound of claim 1 and a pharmaceutically acceptable excipient.

28. (canceled)

29. A method of preventing or treating a condition selected from:
thrombosis; deep venous thrombosis; thrombosis related to pregnancy; congenital pro-thrombotic disorders; thrombosis resulting from autoimmune conditions; transitory ischaemic attacks; myocardial infarction; peripheral arterial occlusion disorders; pulmonary embolisms; deep venousmicrovascular disease; stroke, including patients with atrial fibrillation with or without chronic kidney disease; disseminated intravascular coagulation (DIC); other conditions where inhibition of FXIIa could be beneficial such as arthritis, neurological inflammatory disorders, Alzheimer's disease, vascular dementia, macular degeneration, diabetic retinopathy, diabetic macular oedema, cerebral oedema in stroke, other causes of oedema, hereditary angioedema or acquired angioedema;
or a method of reducing the risk of a venous and/or arterial thrombosis in patients with an indication selected from:
viral or bacterial infections, reperfusion injury (also know as ischaemia-reperfusion injury), renal insufficiency, liver diseases, myocardial infarction, angina pectoris (including unstable angina), atherosclerosis, stroke, cancer, silent brain ischaemia, and neurotraumatic disorder;
or a method of reducing the risk of a venous and/or arterial thrombosis occurring during a medical procedure selected from:
complex left-sided ablation (pulmonary vein isolation; VT ablation), transcatheter aortic valve replacement (TAVR) (also known as transcatheter aortic valve implantation (TAVI)), spinal or epidural anaesthesia, lumbar diagnostic puncture, thoracic surgery, abdominal surgery, major orthopaedic surgery, liver biopsy, transurethral prostate resection, kidney biopsy, endoscopy with biopsy, prostate or bladder biopsy, electrophysiological study or radiofrequency catheter ablation for supraventricular tachycardia (including left-sided ablation via single trans-septal puncture), angiography, pacemaker or implantable cardioverter defibrillator (ICD) implantation (unless complex anatomical setting, e.g. congenital heart disease), mechanical valve implantation, prosthetic valve implantation, left ventricular assist device (LVAD), reocclusions and restenoses after angioplasty or aortocoronary bypass, extra corporeal membrane oxygenation (ECMO), extra corporeal circulation such as coronary artert bypass grafting (CABG), and a medical procedure comprising contact with artificial surfaces including renal dialysis;
or a method of reducing the risk of a venous and/or arterial thrombosis occurring in a patient who has undergone a medical procedure selected from:
transcatheter aortic valve replacement (TAVR) (also known as transcatheter aortic valve implantation (TAVI)), major orthopaedic surgery, pacemaker or implantable cardioverter defibrillator (ICD) implantation (unless complex anatomical setting, e.g. congenital heart disease), mechanical valve implantation, prosthetic valve implantation, left ventricular assist device (LVAD), reocclusions and restenoses after angioplasty or aortocoronary bypass, extra corporeal membrane oxygenation (ECMO), and extra corporeal circulation such as coronary artert bypass grafting (CABG)
wherein the method comprises the administration of a therapeutically effective amount of a compound of claim 1 and pharmaceutically acceptable salts thereof or the administration of a therapeutically effective amount of a co-therapy that includes a compound of claim 1 and pharmaceutically acceptable salts thereof.
